(19) United States
(12) Patent Application Publication
(10) Pub. No.: US 2024/0309084 A1
(43) Pub. Date: Sep. 19, 2024

(54) DEGRADATION OF EXTRACELLULAR TARGETS

(71) Applicant: Novartis AG, Basel (CH)

(72) Inventors: Martin ALLAN, Worcester, MA (US); Jeffrey BAGDANOFF, Quincy, MA (US); David Weninger BARNES, Bridgewater, MA (US); John BLANKENSHIP, Acton, MA (US); James BRADNER, Weston, MA (US); Kevin CLAIRMONT, Acton, MA (US); Brian GRANDA, Salisbury, MA (US); Guido JUNGE, Basel (CH); Thomas SMITH, Arlington, MA (US); Elisabetta TRAGGIAI, Basel (CH); Max WARNCKE, Freiburg, Baden-Württemberg (DE)

(21) Appl. No.: 18/272,702

(22) PCT Filed: Jan. 18, 2022
(86) PCT No.: PCT/IB2022/050410

§ 371 Date: Jul. 17, 2023

Related U.S. Application Data
(60) Provisional application No. 63/139,177, filed on Jan. 19, 2021.
(60) Provisional application No. 63/231,829, filed on Aug. 11, 2021.

(51) Int. Cl. C07K 16/28; A61K 47/54; A61K 47/68; C07K 16/40

(52) U.S. Cl. CPC C07K 16/28; A61K 47/549; A61K 47/6849; C07K 16/40; C07K 2317/31; C07K 2317/62; C07K 2317/70

(57) ABSTRACT

The present disclosure features bifunctional compounds for the degradation of extracellular targets, as well as compositions and related methods thereof.

### CROSS-REFERENCE TO RELATED APPLICATIONS

This is an international PCT application which claims priority to and the benefit of U.S. Provisional Application No. 63/139,177 filed on Jan. 19, 2021 and U.S. Provisional Application No. 63/231,829 filed on Aug. 11, 2021, the entire contents of each of which are incorporated herein by reference.

### SEQUENCE LISTING

The instant application contains a Sequence Listing, which has been submitted electronically in ASCII format and is hereby incorporated by reference in its entirety. Said ASCII copy, created on Jan. 13, 2022, is named PAT058899-WO-PCT_SL.txt and is 36,088 bytes in size.

### BACKGROUND

Conventional protein-directed therapeutics may be used to treat diseases by modulating protein function or recruiting immune effectors to a target site. However, many potential therapeutic targets have molecular functions that are either incompletely understood or not readily inhibited, and thus are not druggable by conventional therapeutic approaches. For example, targeted protein degradation (TPD) is an approach for treating disease involving such undruggable disease-causing proteins by selectively degrading a target protein rather than inhibiting its function, such as through traditional chemical inhibition. Examples of targeted protein degradation systems include proteolysis targeting chimeras (PROTACs) (K. M. Sakamoto et al., Proc. Natl. Acad. Sci. (2001) 98: 8554-8559); dTAGs (B. Nabet et al., Nat. Chem. Biol. (2018) 14:431); chaperone-mediated autophagy targeting (X. Fan et al., Nat. Neurosci., (2014) 17:471-480); and SNIPERs (M. Naito et al., Drug Discov. Today Technol., (2019)). However, there is a need for improved strategies for inducing/modulating the degradation of extracellular targets

### SUMMARY

The present disclosure features bifunctional compounds useful, inter alia, for the degradation of extracellular targets, as well as compositions and related methods thereof. These bifunctional compounds may be used to modulate the level of an extracellular target (e.g., an extracellular target molecule) in a subject; for example, the bifunctional compound may bind to an extracellular target associated with a disease in a subject and mediate a reduction of the level of said extracellular target by tagging it for degradation in a degradation pathway (e.g., receptor mediated endocytosis or lysosomal degradation). Such bifunctional compounds may present an attractive therapeutic alternative to traditional methods of chemical inhibition or biological neutralization by eliminating the extracellular target in its entirety; such elimination is desirable with a target whose presence causes disease (e.g., low density lipoprotein), proteins with multiple functions or active sites (e.g., antibodies), or proteins with large active sites difficult to inhibit with small molecules (e.g., PCSK9). Exemplary extracellular targets described herein include proteins (soluble proteins and membrane-associated proteins, such as antibodies, receptors, growth factors, cytokines, chemokines, enzymes, hormones, neurotransmitters, or a combination thereof), lipoproteins (e.g., low density lipoprotein, very low density lipoprotein, chylomicrons), nucleic acids (e.g., oligonucleotides, DNA, RNA), toxins, liposomes, virus particles, and cells (e.g., prokaryotic cells, eukaryotic cells).

The bifunctional compounds of the present disclosure comprise a first moiety capable of binding to an extracellular target and a second moiety capable of binding to a membrane-bound receptor associated with a degradation pathway. In one aspect, a bifunctional compound described herein has the structure of Formula (I): A_{G}-L-R_{G} (I) or a pharmaceutically acceptable salt thereof, wherein A_{G} is a protein moiety that binds to an extracellular target (e.g., an antibody or a fragment thereof, a receptor or a fragment thereof, an antigen protein or a fragment thereof); L is absent or a linker; and R_{G} is a moiety that binds to a membrane-bound receptor associated with a degradation pathway.

In some embodiments, A_{G} binds to a soluble target (e.g., a plasma protein) or a membrane-associated target (e.g., a membrane-associated protein). In some embodiments, A_{G} is an antibody or a fragment thereof, a receptor or a fragment thereof, or an antigen protein or a fragment thereof. In some embodiments, A_{G} is an antibody or fragment thereof (e.g., a Fab, a Fab′, a F(ab′)₂, a F(ab)₂, variable fragment (Fv), a domain antibody (dAb), a single domain antibody, or a single chain variable fragment (scFv)). In some embodiments, the antibody or fragment thereof comprises an antigen-binding domain that binds to an extracellular target. In some embodiments, the antibody is a full-length antibody. In some embodiments, the antibody is an antibody fragment. In some embodiments, the antibody fragment is a Fab, a Fab′, a F(ab′)₂, a F(ab)₂, variable fragment (Fv), a domain antibody (dAb), a single domain antibody, or a single chain variable fragment (scFv). In some embodiments, the antibody is a monospecific antibody or a fragment thereof. In some embodiments, the antibody is a multispecific antibody or a fragment thereof. In some embodiments, the antibody is a bispecific antibody or fragment thereof.

In one aspect, the bispecific antibody or fragment thereof binds to asialoglycoprotein receptor (ASGPR) and proprotein convertase subtilisin/kexin type 9 (PCSK9). In some embodiments, the bispecific antibody is a full-length antibody. In some embodiments, the bispecific antibody fragment thereof comprises a Fab, a Fab′, a F(ab′)₂, a F(ab)₂, variable fragment (Fv), a domain antibody (dAb), a single domain antibody, or a single chain variable fragment (scFv). In some embodiments, the bispecific antibody or fragment thereof comprises a linker. In some embodiments, the linker is a (G4S)ₙ linker, wherein n is an integer from 1 to 20. In some embodiments, n is an integer from 1 to 4 (SEQ ID NO: 42). In some embodiments, n is 4 (SEQ ID NO: 46).

In some embodiments, the bispecific antibody or fragment thereof comprises one or more of the following:
(i) a heavy chain complementarity determining region 1 (HCDR1) amino acid sequence as set forth in SEQ ID NO: 9, a heavy chain complementarity determining region 2 (HCDR2) amino acid sequence as set forth in SEQ ID NO: 10, and a heavy chain complementarity determining region 3 (HCDR3) amino acid sequence as set forth in SEQ ID NO: 11;
(ii) a HCDR1 amino acid sequence as set forth in SEQ ID NO: 21, a HCDR2 amino acid sequence as set forth in SEQ ID NO: 22, and a HCDR3 amino acid sequence as set forth in SEQ ID NO: 11;
(iii) a HCDR1 amino acid sequence as set forth in SEQ ID NO: 31, a HCDR2 amino acid sequence as set forth in SEQ ID NO: 32, and a HCDR3 amino acid sequence as set forth in SEQ ID NO: 33;
(iv) a HCDR1 amino acid sequence as set forth in SEQ ID NO: 15, a HCDR2 amino acid sequence as set forth in SEQ ID NO: 16, and a HCDR3 amino acid sequence as set forth in SEQ ID NO: 17;
(v) a HCDR1 amino acid sequence as set forth in SEQ ID NO: 26, a HCDR2 amino acid sequence as set forth in SEQ ID NO: 27, and a HCDR3 amino acid sequence as set forth in SEQ ID NO: 17
(vi) a HCDR1 amino acid sequence as set forth in SEQ ID NO: 36, a HCDR2 amino acid sequence as set forth in SEQ ID NO: 37, and a HCDR3 amino acid sequence as set forth in SEQ ID NO: 38;
(vii) a light chain complementarity determining region 1 (LCDR1) amino acid sequence as set forth in SEQ ID NO: 12, a light chain complementarity determining region 2 (LCDR2) amino acid sequence as set forth in SEQ ID NO: 13, and a light chain complementarity determining region 3 (LCDR3) amino acid sequence as set forth in SEQ ID NO: 14;
(viii) a LCDR1 amino acid sequence as set forth in SEQ ID NO: 23, a LCDR2 amino acid sequence as set forth in SEQ ID NO: 24, and a LCDR3 amino acid sequence as set forth in SEQ ID NO: 25;
(ix) a LCDR1 amino acid sequence as set forth in SEQ ID NO: 34, a LCDR2 amino acid sequence as set forth in SEQ ID NO: 35, and a LCDR3 amino acid sequence as set forth in SEQ ID NO: 14;
(x) a LCDR1 amino acid sequence as set forth in SEQ ID NO: 18, a LCDR2 amino acid sequence as set forth in SEQ ID NO: 19, and a LCDR3 amino acid sequence as set forth in SEQ ID NO: 20;
(xi) a LCDR1 amino acid sequence as set forth in SEQ ID NO: 28, a LCDR2 amino acid sequence as set forth in SEQ ID NO: 29, and a LCDR3 amino acid sequence as set forth in SEQ ID NO: 30; and/or
(xii) a LCDR1 amino acid sequence as set forth in SEQ ID NO: 39, a LCDR2 amino acid sequence as set forth in SEQ ID NO: 29, and a LCDR3 amino acid sequence as set forth in SEQ ID NO: 20.

In some embodiments, the bispecific antibody or fragment thereof comprises one or more of the following:
(i) a heavy chain variable region (VH) amino acid sequence having at least about 85%, at least about 90%, at least about 95%, at least about 96%, at least about 97%, at least about 98%, or at least about 99% sequence identity to SEQ ID NO: 1;
(ii) a VH amino acid sequence having at least about 85%, at least about 90%, at least about 95%, at least about 96%, at least about 97%, at least about 98%, or at least about 99% sequence identity to SEQ ID NO: 3;
(iii) a light chain variable region (VL) amino acid sequence having at least about 85%, at least about 90%, at least about 95%, at least about 96%, at least about 97%, at least about 98%, or at least about 99% sequence identity to SEQ ID NO: 2; and/or
(iv) a VL amino acid sequence having at least about 85%, at least about 90%, at least about 95%, at least about 96%, at least about 97%, at least about 98%, or at least about 99% sequence identity to SEQ ID NO: 4.

In some embodiments, the bispecific antibody or fragment thereof comprises one or more of the following:
(i) a VH amino acid sequence as set forth in SEQ ID NO: 1;
(ii) a VH amino acid sequence as set forth in SEQ ID NO: 3;
(iii) a VL amino acid sequence as set forth in SEQ ID NO: 2; and/or
(iv) a VL amino acid sequence as set forth in SEQ ID NO: 4.

In some embodiments, the bispecific antibody or fragment thereof comprises an amino acid sequence having at least about 85%, at least about 90%, at least about 95%, at least about 96%, at least about 97%, at least about 98%, or at least about 99% sequence identity to SEQ ID NO: 5, 6, 7, and/or 8. In some embodiments, the bispecific antibody or fragment thereof comprises an amino acid sequence as set forth in SEQ ID NO: 5, 6, 7, and/or 8.

In some embodiments, A_{G} is a receptor (e.g., a full-length receptor, a receptor fragment, or a functional variant thereof). In some embodiments, the receptor is a full-length receptor or a functional variant thereof.

In some embodiments, A_{G} is an antigen protein or a fragment thereof. In some embodiments, A_{G} is an antigen protein or a fragment thereof that recognizes a pathogenic autoantibody. In some embodiments, the antigen protein or a fragment thereof is a disintegrin and metalloproteinase with a thrombospondin type 1 motif, member 13 (ADAMTS13), steroidogenic cytochrome P450 enzyme 21-hydroxylase, N-methyl-d-aspartate-(NMDA)-receptor, erythrocytes, anti-smooth muscle antibodies (ASMAs), actin, platelet, signal recognition particle (SRP), 3-hydroxy-3-methyl-glutaryl-coenzyme A reductase (HMGCR), myosin, sperm, amylase alpha2, type XVII collagen (col17), kallikrein 13, type VII collagen (col7), myeloperoxidase (MPO), type IV collagen, proteinase 3 (PR3), thyrotropin receptor (TSHR), thyroglobulin, thyroid peroxidase (TPO), thyroglobulin, thyroid peroxidase (TPO), platelets, myeloperoxidase (MPO), muscle nicotinic acetylcholine receptors, muscle-specific kinase (MuSK), low-density lipoprotein receptor protein 4 (LRP4), myosin, beta1 adrenergic receptor, adenine-nucleotide translocase, aquaporin-4, myelin oligodendrocyte glycoprotein (MOG), heat shock protein 90 (HSP90), heat shock protein A5 (HSPA5), desmoglein-3, parietal cells, mitochondria, phospholipase A2 receptor (PLA2R), thrombospondin type 1 domain-containing 7A (THSD7A), cyclic citrullinated proteins, RNA binding proteins (Ros), La, double-stranded DNA (dsDNA), angiotensin II type 1 receptor (ATIR), endothelin-1 type A receptor (ETAR), insulin, glutamic acid decarboxylase or protein tyrosine phosphatase.

In some embodiments, R_{G} comprises a small molecule, a carbohydrate or carbohydrate derivative (e.g., a monosaccharide, disaccharide, trisaccharide, tetrasaccharide, pentasaccharide, hexasaccharide, oligosaccharide, a modified saccharide (e.g., a saccharide phosphate, e.g., a monosaccharide phosphate (e.g., mannose-6-phosphate), hexasaccharide diphosphate, triantennary GalNAc)), an antibody molecule or fragment thereof, a peptide, or a pharmaceutically acceptable salt thereof. In some embodiments, R_{G} binds to a receptor associated with a degradation pathway (e.g., receptor mediated endocytosis or lysosomal degradation). In some embodiments, the degradation pathway comprises receptor mediated endocytosis or endosomal degradation. In some embodiments, the degradation pathway comprises lysosomal degradation.

In some embodiments, the degradation pathway is mediated by a mannose-6-phosphate receptor (M6PR), insulin-like growth factor 2 receptor (IGF2R), or an asialoglycoprotein receptor (ASGPR). In some embodiments, R_{G} is a binding moiety for a mannose-6-phosphate receptor (M6PR), insulin-like growth factor 2 receptor (IGF2R), or an asialoglycoprotein receptor (ASGPR). In some embodiments, when R_{G} is a binding moiety for a mannose-6-phosphate receptor (M6PR) or insulin-like growth factor 2 receptor (IGF2R), the binding moiety for said M6PR or IGF2R is a small molecule, a carbohydrate or carbohydrate derivative, or an antibody or fragment thereof. In some embodiments, when R_{G} is a binding moiety for an asialoglycoprotein receptor (ASGPR), the binding moiety for said ASGPR is a small molecule, a carbohydrate or carbohydrate derivative, or an antibody or fragment thereof. In some embodiments, the ASGPR binding moiety is ASGPR1 or ASGPR2. In some embodiments, R_{G} comprises an ASGPR ligand. In some embodiments, when R_{G} is a binding moiety for a mannose 6-phosphate receptor (M6PR) or insulin-like growth factor 2 receptor (IGF2R), R_{G} comprises a hexasaccharide moiety (e.g., a glucose, galactose, mannose, N-acetylglucose, N-acetylgalactose, N-acetylmannose, mannose-6-phosphate, or mannose-6-phosphonate moiety). In some embodiments, R_{G} comprises a plurality of hexasaccharide moieties (e.g., a plurality of glucose, galactose, mannose, N-acetylglucose, N-acetylgalactose, N-acetylmannose, mannose-6-phosphate, or mannose-6-phosphonate moieties). In some embodiments, R_{G} comprises one or more N-acetylgalactose (GalNAc) moieties, e.g., at least 1, 2, 3, 4, 5, or 6 GalNAc moieties. In some embodiments, R_{G} comprises 3 GalNAc moieties.

In some embodiments, R_{G} comprises a compound of Formula (I):

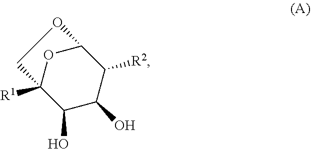

or a pharmaceutically acceptable salt thereof, wherein:
R¹ is —CN, —CH₂—CN, —C≡CH, —CH₂—N₃, —CH₂—NH₂, —CH₂—N(R⁴)—S(O)₂—R⁵, —CH₂—CO₂H, —CO₂H, —CH₂—OH, —CH₂—SH, —CH═CH—R⁵, —CH₂—R⁵, —CH₂—S—R⁵, —CH₂—N(R⁴)—R⁵, —CH₂—N(R⁴)—C(O)—R⁵, —CH₂—N(R⁴)—C(O)—O—R⁵, —CH₂—N(R⁴)—C(O)—N(R⁴)—R⁵, —CH₂—O—R⁵, —CH₂—O—C(O)—R⁵, —CH₂—O—C(O)—N(R⁴)—R⁵, —CH₂—O—C(O)—O—R⁵, CH₂—S(O)—R⁵, —CH₂—S(O)₂—R⁵, —CH₂—S(O)₂—N(R⁴)—R⁵, —C(O)—NH₂, —C(O)—O—R⁵, —C(O)—N(R⁴)—R⁵, or aryl or heteroaryl, wherein the aryl or heteroaryl is optionally substituted with R⁵,
or R¹ is —Z—X—Y, wherein X is a linker or a drug delivery system, Y is absent or is a ligand selected from the group consisting of a small molecule, an amino acid sequence, a nucleic acid sequence, an antibody, an oligomer, a polymer, genetically derived material, a liposome, a nanoparticle, dye, and a fluorescent probe, or a combination thereof, and Z is absent or is —C≡C—, —CH═CH—, —CH₂—, —CH₂—O—, —C(O)—N(R⁴)—, —CH₂—S—, —CH₂—S(O)—, —CH₂—S(O)₂—, —CH₂—S(O)₂—N(R⁴)—, —C(O)—O—, —CH₂—N(R⁴)—, —CH₂—N(R⁴)—C(O)—, —CH₂—N(R⁴)—S(O)₂—, —CH₂—N(R⁴)—C(O)—O—, —CH₂—N(R⁴)—C(O)—N(R⁴)—, —CH₂—O—C(O)—, —CH₂—O—C(O)—N(R⁴)—, —CH₂—O—C(O)—O—, or aryl or heteroaryl, wherein the aryl or heteroaryl is optionally substituted with R⁵;
R² is —OH, —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³; and wherein when R¹ is —CH₂—OH, R² is —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³;
each R³ is independently —H, —(C₁-C₅)alkyl, halo-substituted (C₁-C₅)alkyl, or (C₃-C₆)cycloalkyl, wherein a —CH₂— group of the alkyl or cycloalkyl may be replaced with a heteroatom group selected from —O—, —S—, and —N(R⁴)— and —CH₃ of the alkyl may be replaced with a heteroatom group selected from —N(R⁴)₂, —OR⁴, and —S(R⁴) wherein the heteroatom groups are separated by at least 2 carbon atoms;
each R⁴ is independently —H, —(C₁-C₂₀)alkyl, or (C₃-C₆)cycloalkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴), wherein the heteroatom groups are separated by at least 2 carbon atoms; and wherein the alkyl and cycloalkyl may be substituted with one to six halo atoms; and
each R⁵ is independently —H, (C₃-C₂₀)cycloalkyl or (C₁-C₂₀)alkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴), wherein the heteroatom groups are separated by at least 2 carbon atoms; and wherein the alkyl and cycloalkyl may be substituted with one to six halo atoms.

In some embodiments, R_{G} is selected from:

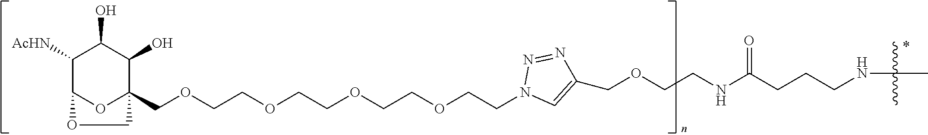

wherein n is 1, 2 or 3;

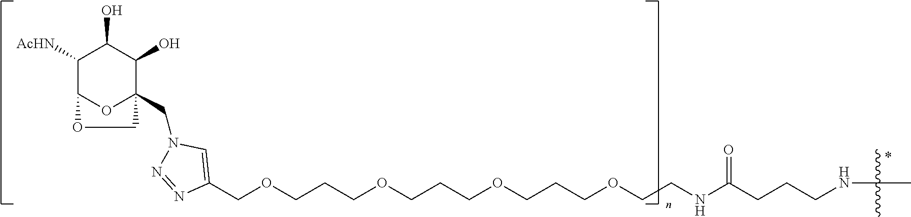

wherein n is 2 or 3; and wherein R_{G} is attached to L at the terminal amine. In some embodiments, R_{G} is selected from any of the following that bind to ASGPR:

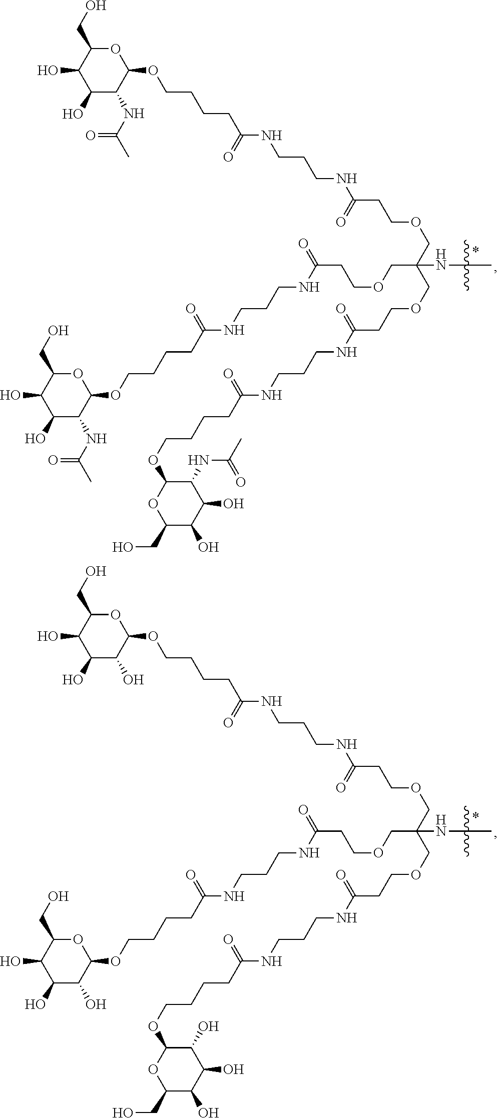 [SMILES: *NC(COCCC(=O)NCCCNC(=O)CCCCO[C@@H]1O[C@H](CO)[C@H](O)[C@H](O)[C@H]1NC(C)=O)(COCCC(=O)NCCCNC(=O)CCCCO[C@@H]1O[C@H](CO)[C@H](O)[C@H](O)[C@H]1NC(C)=O)COCCC(=O)NCCCNC(=O)CCCCO[C@@H]1O[C@H](CO)[C@H](O)[C@H](O)[C@H]1NC(C)=O ; *NC(COCCC(=O)NCCCNC(=O)CCCCO[C@@H]1O[C@H](CO)[C@H](O)[C@H](O)[C@H]1O)(COCCC(=O)NCCCNC(=O)CCCCO[C@@H]1O[C@H](CO)[C@H](O)[C@H](O)[C@H]1O)COCCC(=O)NCCCNC(=O)CCCCO[C@@H]1O[C@H](CO)[C@H](O)[C@H](O)[C@H]1O]

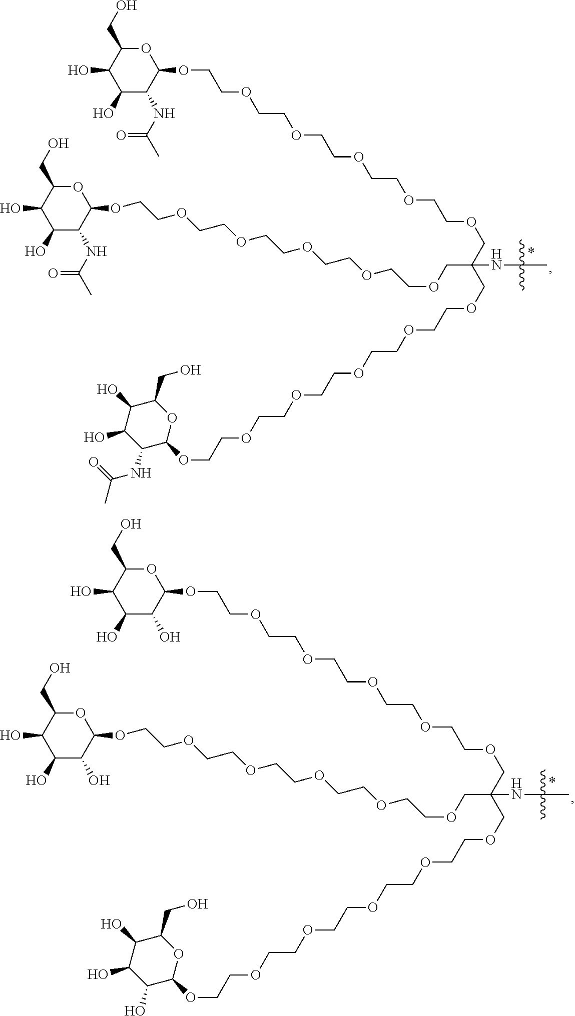

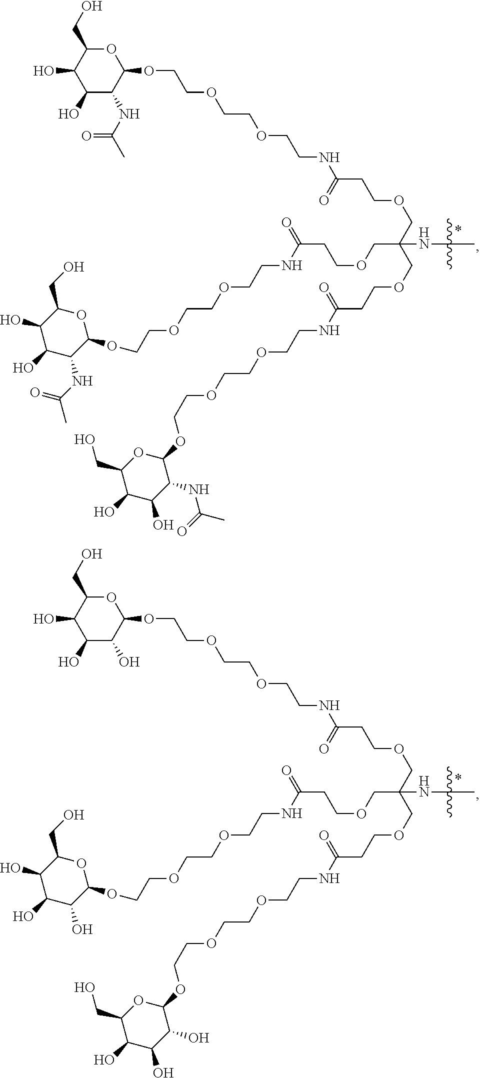 [SMILES: *NC(COCCC(=O)NCCOCCOCCO[C@@H]1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O ; *NC(COCCC(=O)NCCOCCOCCO[C@@H]1O[C@H](CO)[C@H](O)[C@H](O)[C@H]1O)(COCCC(=O)NCCOCCOCCO[C@@H]1O[C@H](CO)[C@H](O)[C@H](O)[C@H]1O)COCCC(=O)NCCOCCOCCO[C@@H]1O[C@H](CO)[C@H](O)[C@H](O)[C@H]1O]

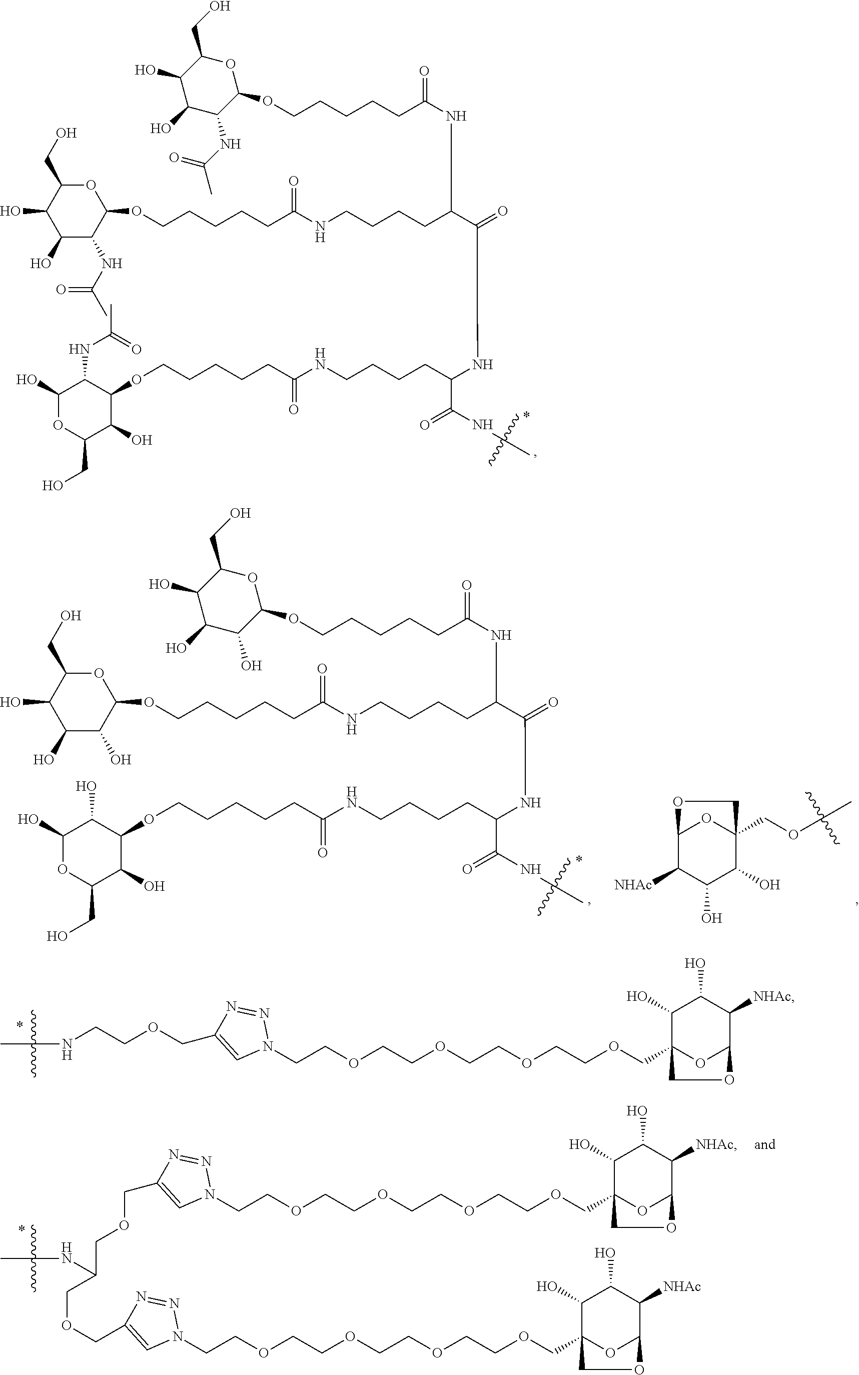

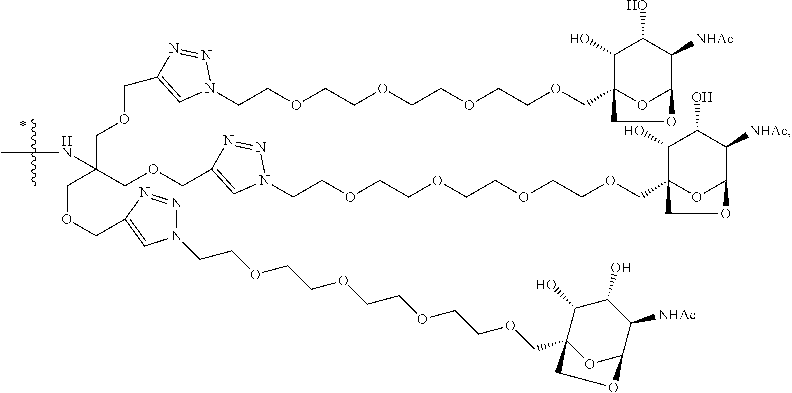 [SMILES: *NC(COCc1cn(CCOCCOCCOCCOC[C@@]23CO[C@@H](O2)[C@H](NC(C)=O)[C@@H](O)[C@H]3O)nn1)(COCc1cn(CCOCCOCCOCCOC[C@@]23CO[C@@H](O2)[C@H](NC(C)=O)[C@@H](O)[C@H]3O)nn1)COCc1cn(CCOCCOCCOCCOC[C@@]23CO[C@@H](O2)[C@H](NC(C)=O)[C@@H](O)[C@H]3O)nn1]

wherein the * of R_{G} indicates the point of attachment to L.

In some embodiments, R_{G} is selected from any of the following that bind to M6PR or IGF2R:

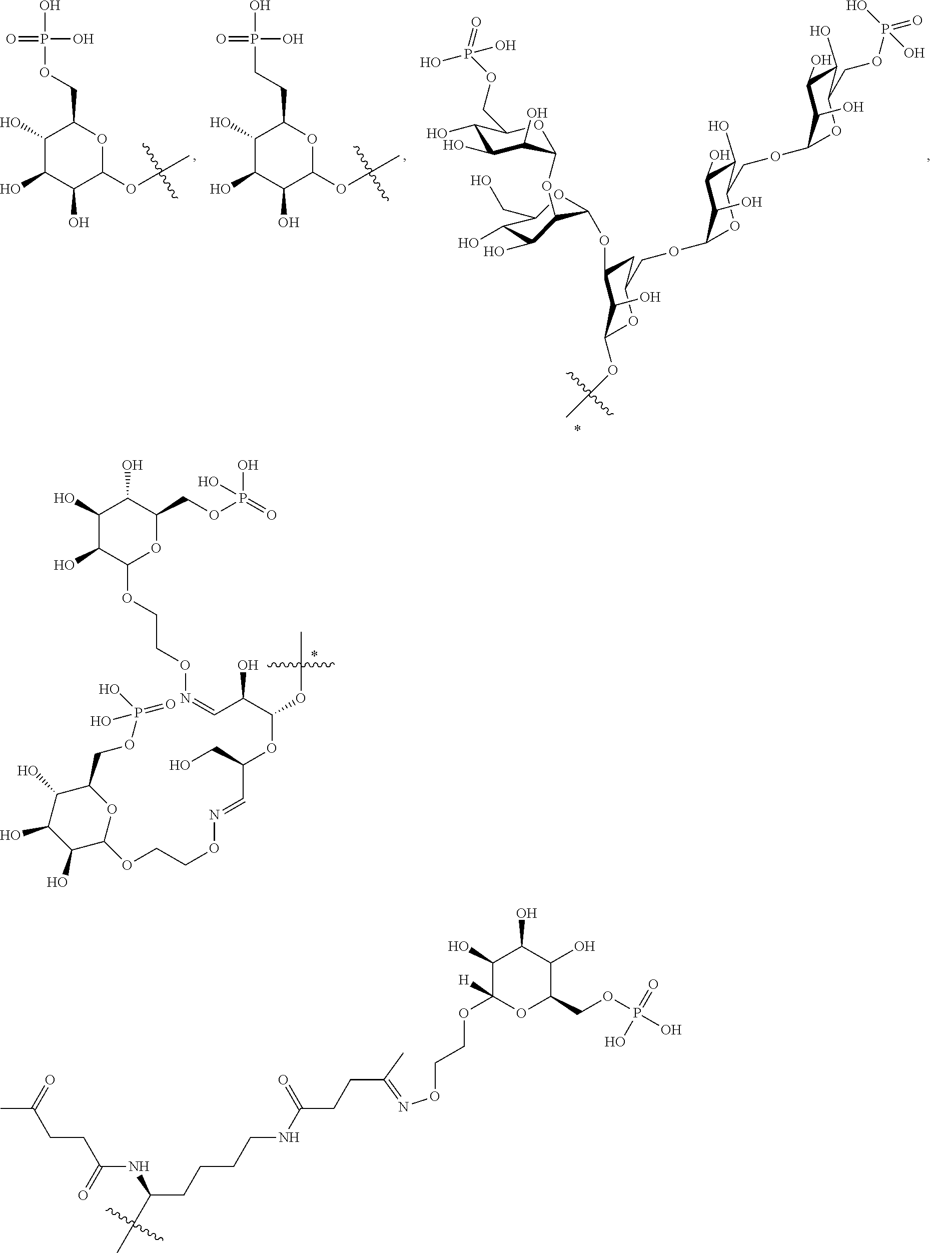

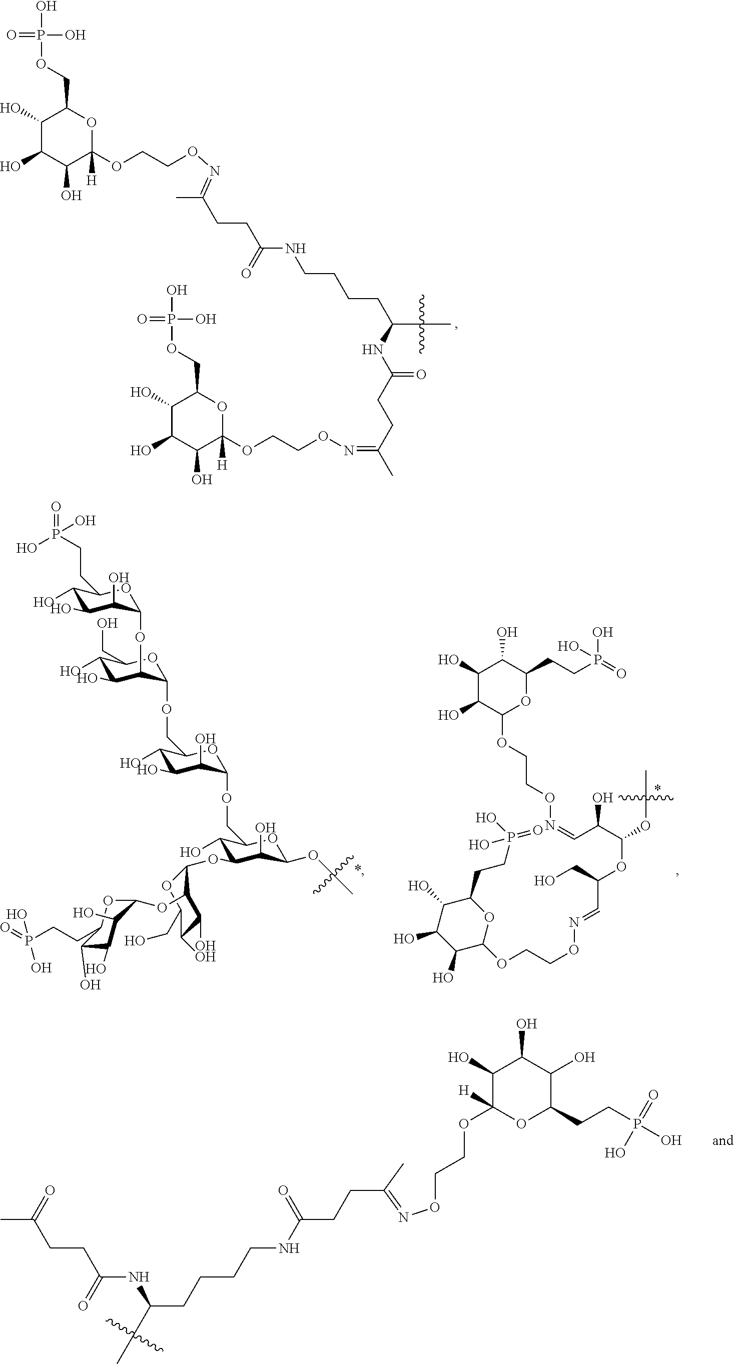

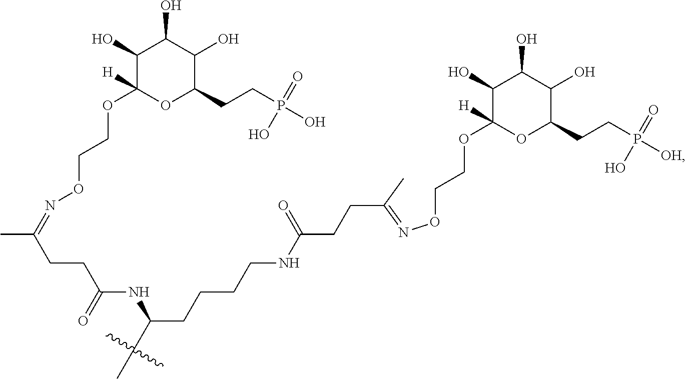 [SMILES: *[C@H](CCCCNC(=O)CC/C(C)=N/OCCO[C@H]1O[C@H](CCP(=O)(O)O)C(O)[C@H](O)[C@@H]1O)NC(=O)CC/C(C)=N\OCCO[C@H]1O[C@H](CCP(=O)(O)O)C(O)[C@H](O)[C@@H]1O]

wherein the * of R_{G} indicates the point of attachment to L.

In some embodiments, R_{G} comprises an M6PR or IGF2R binding moiety and the extracellular target is TNFR1, ILIR, progranulin, Tau, MUC5B, TREM2, or EGFR. In some embodiments, L comprises a heteroalkylene moiety. In some embodiments, the heteroalkylene moiety comprises at least 6, 8, 10, 12, 16, 20, 24, 36, 48, 60, 100, 250, 300, or more carbon atoms. In some embodiments, the heteroalkylene moiety is between 4 and 48 carbon atoms, or 4 and 36 carbon atoms, or 4 and 24 carbon atoms, or 4 and 16 carbon atoms, or 6 and 12 carbon atoms, or 8 and 12 carbon atoms. In some embodiments, the heteroalkylene moiety is polyethylene glycol. In some embodiments, L is a chemical moiety formed by reaction between two reactive groups described in Table 2.

In some embodiments, L does not comprise a structure of Formula (A):

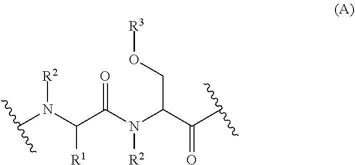

wherein:
R¹ is hydrogen or other amino acid side chain;
each R² is independently hydrogen or C₁-C₆ alkyl;
R³ is a carbohydrate or carbohydrate derivative;
R^{A} is hydrogen, C₁-C₆ alkyl, C₁-C₆ haloalkyl, C₃-C₁₂ cycloalkyl, C₃-C₁₂ heterocyclyl, C₆-C₁₂ aryl, C₆-C₁₂ heteroaryl; and
each “ [symbol] ” is independently a connection to either A_{G}, R_{G}, or another portion of L. In some embodiments, R¹ is C₁-C₆ alkyl, C₁-C₆ heteroalkyl, C₁-C₆ haloalkyl, C₂-C₆ alkenylene, C₂-C₆ alkynylene, C₃-C₁₂ cycloalkyl, C₃-C₁₂ heterocyclyl, C₆-C₁₂ aryl, C₆-C₁₂ heteroaryl, C₁-C₆ alkylene-aryl, C₁-C₆ alkylene-heteroaryl, C₁-C₆ alkylene-cycloalkyl, C₁-C₆ alkylene-heterocyclyl, halogen, or —OR^{A}.

In some embodiments, L does not comprise a structure of Formula (A-I):

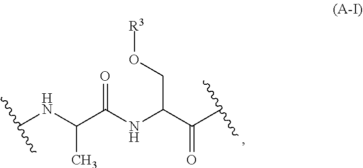

wherein R³ is a carbohydrate or carbohydrate derivative. In some embodiments, —CH₃ is in the (L) or (D) configuration. In some embodiments, R³ is a monosaccharide, disaccharide, oligosaccharide, or polysaccharide, each of which is optionally substituted. In some embodiments, L and/or R_{G} do not comprise a nitrogen-containing heteroaryl or nitrogen-containing heterocyclyl. In some embodiments, the nitrogen-containing heteroaryl is a triazolyl. In some embodiments, L and/or R_{G} do not comprise a structure of Formula (B):

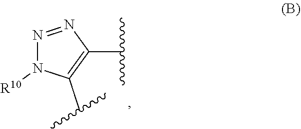

wherein R¹⁰ is a carbohydrate or carbohydrate derivative moiety. In some embodiments, R¹⁰ is a monosaccharide, disaccharide, oligosaccharide, or polysaccharide, each of which is optionally substituted.

In some embodiments, the extracellular target comprises a protein (soluble proteins and membrane-associated proteins, such as antibodies or fragments thereof, receptors, growth factors, cytokines, chemokines, enzymes, or hormones), lipoprotein, liposome, nucleic acid (e.g., oligonucleotides, DNA, RNA), toxin, virus particle, or cell (e.g., prokaryotic cells, eukaryotic cells).

In an embodiment, the extracellular target is a soluble protein (e.g., a plasma protein) or a membrane-associated protein. In some embodiments, the extracellular target is a soluble protein (e.g., an antibody, a soluble receptor, a secreted protein, a growth factor, a cytokine, a hormone, a neurotransmitter, or an enzyme). In some embodiments, A_{G} binds to said soluble protein or a component thereof (e.g., a protein modification (e.g., sugar)). In some embodiments, the soluble protein is proprotein convertase subtilisin/kexin type 9 (PCSK9), complement factor H-related protein 3 (CFHR3), MICA (MHC class I chain-related gene A), or apolipoprotein-B (Apo-B).

In some embodiments, the extracellular target is a membrane-associated protein (e.g., a type I, type II or multipass membrane protein or a glycophosphatidylinositol (GPI) anchored membrane associated protein, e.g., a receptor, a protein channel, or an ion channel). In some embodiments, A_{G} binds to said membrane-associated protein or a component thereof (e.g., a protein modification (e.g., sugar)). In some embodiments, the membrane-associated protein is covalently or non-covalently associated with a cell membrane. In some embodiments, the membrane-associated protein is a transmembrane protein or membrane-anchored protein. In some embodiments, the membrane-associated protein is a type I, type II or multipass membrane protein or a glycophosphatidylinositol (GPI) anchored membrane associated protein. In some embodiments, the GPI anchored membrane associated protein is a receptor, a protein channel, or an ion channel.

In some embodiments, the extracellular target is a lipoprotein; and wherein said A_{G} binds to said lipoprotein or a component thereof. In some embodiments, the component thereof is a lipid or an apolipoprotein. In some embodiments, the lipoprotein is a lipoprotein receptor (LPR) or lipoprotein(a) (Lp(a)).

In some embodiments, the extracellular target is a pathogenic target, e.g., a protein associated with a deleterious or unwanted effect in a sample (e.g., a cell) or a subject. In some embodiments, the pathogenic target is present at an expression level or an activity level that results in a deleterious or unwanted effect in a sample (e.g., a cell) or a subject. In some embodiments, the pathogenic target is an extracellular secreted protein. In some embodiments, the extracellular secreted protein is a soluble protein. In some embodiments, the pathogenic target is a pathogenic autoantibody or a fragment thereof. In some embodiments, the pathogenic target is a cell surface receptor. In some embodiments, the cell surface receptor is selected from TNF receptor 1 (TNFR1), interleukin-1 receptor (ILIR), PD-L1, epidermal growth factor receptor (EGFR), or transferrin. In some embodiments, the pathogenic target is a neurological target. In some embodiments, the neurological target is a Tau protein or aggregate or an immuno-oncology target. In some embodiments, the immuno-oncology target is progranulin. In some embodiments, the pathogenic autoantibody causes a disease, disorder, condition or clinical situation, wherein said disease, disorder, condition or clinical situation is selected from Acquired thrombotic thrombopenic purpura, Addison disease, Anti-NMDA encephalitis, Autoimmune hemolytic anemia (AIHA), Autoimmune hepatitis, Autoimmune idiopathic thrombocytopenia, Autoimmune myopathies, Autoimmune orchitis, Autoimmune pancreatitis, Bulbous pemphigoid, Dry eye disease, epidermolysis bullosa acquisita, eosinophilic granulomatosis with polyangiitis (EGPA), Goodpasture's disease, Granulomatosis with polyangitis, Graves Disease, Hashimoto's thyroiditis, Idiopathic interstitial pneumonias, Idiopathic thrombocytopenic purpura (ITP), microscopic polyangiitis (MPA), Myasthenia Gravis, Myocarditis, Neuromyelitis optica (NMO), Ovarian insufficiency, Pemphigus, Pernicious anemia, Primary biliary cholangitis (PBC), Primary membranous nephropathy, Rheumatoid arthritis, Sjögren's syndrome, Systemic lupus erythematosus (SLE), Systemic sclerosis, or Type I diabetes.

Exemplary extracellular targets include proprotein convertase subtilisin/kexin type 9 (PCSK9), tumor necrosis factor receptor 1 (TNFR1), interleukin-1 receptor (IL1R), low density lipoproteins, very low density lipoproteins, chylomicrons, apolipoprotein B (ApoB), lipoprotein(a) (Lp(a)), apolipoprotein C₃ (ApoCIII), angiopoietin-like 3 (ANGPTL3), angiopoietin-like 4 (ANGPTL4), angiopoietin-like 8 (ANGPTL8), Factor 11, growth differentiation factor 15 (GDF15), lipoprotein lipase (LPL), interleukin 1-beta (IL10), interleukin 17 (IL17), complement Factor B, complement Factor D, myeloperoxidase (MPO), immunoglobulin A (IgA), immunoglobulin E (IgE), programmed cell death protein 1 (PD-1), programmed death-ligand 1 (PD-L1), interleukin 7 (IL7), interleukin 12A (IL12A), interleukin 23 (IL23), tumor necrosis factor A (TNFA), microtubule associated protein tau (MAPT), complement factor H-related protein 3 (FHR3), tissue inhibitor of metalloproteinases 1 (TIMP1), Apelin, bone morphogenetic protein 6 (BMP6), bone morphogenetic protein 9/growth differentiation factor 2 (BMP9/GDF2), colony stimulating factor 1 receptor (CSF-1), erythropoietin (EPO), interleukin 5 (IL5), milk fat globule-EGF Factor 8 protein (MFGE8), thymic stromal lymphopoietin (TSLP), thrombospondin (TSP), complement component 5 (C5), C—X—C motif chemokine ligand 10 (CXCL10), fibroblast growth factor 23 (FGF23), insulin-like growth factor 1 (IGF1), interleukin 10 (IL10), interleukin 13 (IL13), interleukin 2 (IL2), interleukin 6 (IL6), vascular endothelial growth factor A (VEGF-A), adenosine deaminase 2 (ADA2), soluble urokinase-type plasminogen activator receptor (suPAR), transforming growth factor beta 1 (TGF-β1), progranulin, alpha-synuclein, a toxin, a venom, an HBV soluble antigen, a viral antigen, a prion protein, a scFv, an AAV, and an anti-AAV antibody. In some embodiments, the extracellular target is subtilisin/kexin type 9 (PCSK9).

In another aspect, the present disclosure features combinations comprising any of the bifunctional compounds described herein (e.g., a bifunctional compound of Formula (I) or a pharmaceutically acceptable salt thereof) and at least one additional therapeutic agent. In one embodiment, the combination comprises a bispecific antibody or fragment thereof (e.g., anti-ASGPR-PCSK9 bispecific antibody or fragment thereof) and at least one additional therapeutic agent. In one embodiment, the combination comprises an anti-ASGPR-PCSK9 bispecific antibody or fragment thereof an at least one additional therapeutic agent. In some embodiments, the at least one additional therapeutic agent is one or more of the bifunctional compounds (e.g., a bifunctional compound of Formula (I) or a pharmaceutically acceptable salt thereof) as described herein.

In another aspect, the present disclosure features pharmaceutical compositions comprising any of the bifunctional compounds described herein (e.g., a bifunctional compound of Formula (I) or a pharmaceutically acceptable salt thereof) and a pharmaceutically acceptable excipient. In one embodiment, the pharmaceutical composition comprises a bispecific antibody or fragment thereof (e.g., anti-ASGPR-PCSK9 bispecific antibody or fragment thereof) and a pharmaceutically acceptable excipient. In one embodiment, the pharmaceutical composition comprises an anti-ASGPR-PCSK9 bispecific antibody or fragment thereof).

In another aspect, the present disclosure features methods of targeting an extracellular target, e.g., a soluble protein or a membrane-associated protein, for degradation comprising use of any of the bifunctional compounds (e.g., a bifunctional compound of Formula (I) or a pharmaceutically acceptable salt thereof) or related compositions as described herein (e.g., in a sample (e.g., tissue sample, plasma sample, or cell) or subject). In some embodiments, the extracellular target, e.g., a soluble protein or a membrane-associated protein, is targeted to the liver (e.g., of a subject). In another aspect, the present disclosure features methods for modulating, e.g., decreasing, the level and/or activity of an extracellular target (e.g., PCSK9) comprising use of any of the bifunctional compounds (e.g., a bifunctional compound of Formula (I) or a pharmaceutically acceptable salt thereof) or related compositions as described herein (e.g., in a sample (e.g., tissue sample, plasma sample, or cell) or subject). In some embodiments, the method is an in vitro method, an in vivo method, or an ex vivo method.

In some embodiments, any of the methods as described herein comprise contacting a sample (e.g., a tissue sample, plasma sample, or a cell) with any of the bifunctional compounds (e.g., a bifunctional compound of Formula (I) or a pharmaceutically acceptable salt thereof) or related compositions thereof as described herein. In some embodiments, any of the methods as described herein comprise contacting a sample (e.g., a tissue sample, plasma sample, or a cell) with a bispecific antibody or fragment thereof (e.g., anti-ASGPR-PCSK9 bispecific antibody or fragment thereof) as described herein. In some embodiments, any of the methods as described herein comprise contacting a sample (e.g., a tissue sample, plasma sample, or a cell) with an anti-ASGPR-PCSK9 bispecific antibody or fragment thereof. In some embodiments, the sample (e.g., a tissue sample, plasma sample, or a cell) is from a subject in need thereof. In some embodiments, the sample is a tissue sample, plasma sample, or a cell. In some embodiments, the sample is a plasma sample.

In some embodiments, any of the methods as described herein comprise administering any of the bifunctional compounds or related compositions thereof as described herein to a subject, e.g., in need thereof. In some embodiments, the subject is a mammal (e.g., a human). In some embodiments, the level and/or activity of the extracellular target (e.g., PCSK9) is modulated, e.g., decreased, (e.g., in the sample or subject) in response to any of the bifunctional compounds (e.g., a bifunctional compound of Formula (I) or a pharmaceutically acceptable salt thereof) or related compositions thereof as described herein. In some embodiments, the level and/or activity of an extracellular target (e.g., PCSK9) is decreased in response to any of the bifunctional compounds (e.g., a bifunctional compound of Formula (I) or a pharmaceutically acceptable salt thereof) or related compositions thereof as described herein. In one embodiment, the level and/or activity of PCSK9 is decreased in response to any of the bispecific antibodies or fragments thereof (e.g., anti-ASGPR-PCSK9 bispecific antibody or fragment thereof) as described herein. In some embodiments, the level and/or activity of the extracellular target (e.g., PCSK9) is decreased in a sample (e.g., a tissue sample, plasma sample, or a cell) or subject by about 55%, 60%, 65%, 70%, 75%, 80%, 85%, 90%, 91%, 92%, 93%, 94%, 95%, 96%, 97%, 98%, 99%, or more (e.g., 100%).

In some embodiments, any of the methods as described herein comprise determining the level and/or activity of the extracellular target (e.g., PCSK9) in a sample (e.g., a tissue sample, plasma sample, or a cell) or subject after the contacting and/or administering. In some embodiments, the level and/or activity of the extracellular target (e.g., PCSK9) is determined by measuring the level and/or activity of the extracellular target in plasma. In some embodiments, the method further comprises comparing the level and/or activity of the extracellular target (e.g., PCSK9) in the sample (e.g., a tissue sample, plasma sample, or a cell) or subject with a reference value or to the level and/or activity of the extracellular target (e.g., PCSK9) in a sample prior to the contacting and/or administering. In some embodiments, the method comprises comparing the level and/or activity of PCSK9 in a plasma sample relative to a reference value or to the PCSK9 levels and/or activity in a plasma sample prior to the contacting and/or administering.

In another aspect, the present disclosure features methods of preventing and/or treating a disease, disorder, condition, or clinical situation in a subject comprising administering an effective amount of any of the bifunctional compounds (e.g., a bifunctional compound of Formula (I) or a pharmaceutically acceptable salt thereof) or related compositions thereof as described herein to the subject. In yet another aspect, the present disclosure features methods of preventing and/or treating a disease, disorder, condition, or clinical situation in a subject comprising administering an effective amount of any of the bispecific antibodies or fragments thereof (e.g., anti-ASGPR-PCSK9 bispecific antibody or fragment thereof) or related compositions thereof as described herein to the subject. In another aspect, the present disclosure features uses of any of the bifunctional compounds (e.g., a bifunctional compound of Formula (I) or a pharmaceutically acceptable salt thereof) or related compositions thereof for preventing and/or treating a disease, disorder, condition, or clinical situation in a subject. In another aspect, the present disclosure features uses of any of the bispecific antibodies or fragments thereof (e.g., anti-ASGPR-PCSK9 bispecific antibody or fragment thereof) or related compositions thereof for preventing and/or treating a disease, disorder, condition, or clinical situation in a subject.

In some embodiments, the disease, disorder, condition, or clinical situation is a proliferative disease (e.g., cancer), a neurological disease (e.g., Alzheimer's disease, neurodegeneration), a cardiovascular disease, a respiratory disease (e.g., asthma), a dermatological disease, a hematological disease, an inflammatory disease, an autoimmune disease, a metabolic disorder, an infectious disease, or a renal disease or disorder. Exemplary diseases, disorders, conditions, and clinical situations include a cancer, Alzheimer's disease, atherosclerosis, arteriosclerosis, hyperlipidemia, familial hypercholesterolemia (heterozygous or homozygous), familial chylomicronemia syndrome, inflammation, asthma, allergies, urticaria, IgA nephropathy, membranous nephropathy, transplant rejection, an IgE-mediated disorder, or an adverse response to a therapeutic agent. Additional exemplary diseases, disorders, conditions, and clinical situations include acute inflammatory demyelinating polyradiculoneuropathy (Guillain-Barré syndrome), acute liver failure, anti-glomerular basement membrane disease (Goodpasture syndrome), chronic inflammatory demyelinating polyradiculoneuropathy (CIDP), cutaneous T cell lymphoma (CTCL); mycosis fungoides; Sézary syndrome, familial hypercholesterolemia, focal segmental glomerulosclerosis (FSGS), hereditary hemochromatosis, hyperviscosity in hypergammaglobulinemia, hyperviscosity in hypergammaglobulinemia, myasthenia gravis, N-methyl-D-aspartate receptor antibody encephalitis, paraproteinemic demyelinating neuropathies; chronic acquired demyelinating polyneuropathies, polycythemia vera; erythrocytosis; thrombotic microangiopathy, thrombotic thrombocytopenic purpura (TTP); vasculitis; Wilson disease, fulminant; dilated cardiomyopathy; Graft-versus-host disease (GVHD); Lipoprotein(a) hyperlipoproteinemia; multiple sclerosis; neuromyelitis optica spectrum disorders (NMOSD); pediatric autoimmune neuropsychiatric disorders associated with streptococcal infections (PANDAS); Sydenham's chorea; peripheral vascular diseases; sickle cell disease; voltage-gated potassium channel (VGKC) antibody related diseases. In some embodiments, the disease, disorder, condition, or clinical situation is a PCSK9-mediated disease or disorder. In some embodiments, the PCSK9-mediated disease or disorder is selected from hypercholesterolemia, hyperlipidemia, hypertriglyceridemia, sitosterolemia, atherosclerosis, arteriosclerosis, coronary heart disease, peripheral vascular disease (including aortic diseases and cerebrovascular disease), peripheral arterial disease, vascular inflammation, elevated Lp(a), elevated LDL, elevated TRL, elevated triglycerides, sepsis, and xanthoma. In some embodiments, the disease, disorder, condition, or clinical situation is a disease, disorder, condition or clinical situation that is treated via therapeutic apheresis.

In some embodiments, any of the bifunctional compounds (e.g., a bifunctional compound of Formula (I) or a pharmaceutically acceptable salt thereof) or pharmaceutical compositions thereof as described herein is delivered via oral, parenteral, topical, mucosal, nasal, buccal, or ophthalmological administration. In some embodiments, any of the bifunctional compounds (e.g., a bifunctional compound of Formula (I) or a pharmaceutically acceptable salt thereof) or pharmaceutical compositions thereof as described herein is delivered via parenteral administration (e.g., via intravenous, subcutaneous, intracutaneous, intramuscular, intraarticular, intraarterial, intraperitoneal, intrasynovial, intrasternal, intrathecal, intralesional or intracranial injection). In some embodiments, any of the bifunctional compounds (e.g., a bifunctional compound of Formula (I) or a pharmaceutically acceptable salt thereof) or pharmaceutical compositions thereof as described herein is delivered via subcutaneous injection. In some embodiments, any of the bifunctional compounds (e.g., a bifunctional compound of Formula (I) or a pharmaceutically acceptable salt thereof) or pharmaceutical compositions thereof as described herein is formulated for sustained release (e.g., a depot formulation).

In some embodiments, the disease, disorder, condition, or clinical situation is a proliferative disease (e.g., cancer), a neurological disease (e.g., Alzheimer's disease, neurodegeneration), a cardiovascular disease, a respiratory disease, a dermatological disease, a hematological disease, an inflammatory disease, an autoimmune disease, a metabolic disorder, an infectious disease, or a renal disease or disorder. Exemplary diseases, disorders, conditions, and clinical situations include a cancer, Alzheimer's disease, atherosclerosis, arteriosclerosis, hyperlipidemia, familial hypercholesterolemia, familial chylomicronemia syndrome, inflammation, asthma, allergies, urticaria, IgA nephropathy, membranous nephropathy, transplant rejection, an IgE-mediated disorder, or an adverse response to a therapeutic agent.

In some embodiments, any of the bifunctional compounds (e.g., a bifunctional compound of Formula (I) or a pharmaceutically acceptable salt thereof) or the pharmaceutical compositions thereof as described herein is to be delivered via oral, parenteral, topical, mucosal, nasal, buccal, or ophthalmological administration. In some embodiments, any of the bifunctional compounds (e.g., a bifunctional compound of Formula (I) or a pharmaceutically acceptable salt thereof) or the pharmaceutical compositions thereof as described herein is to be delivered via parenteral administration (e.g., via intravenous, subcutaneous, intracutaneous, intramuscular, intraarticular, intraarterial, intraperitoneal, intrasynovial, intrasternal, intrathecal, intralesional or intracranial injection). In some embodiments, any of the bifunctional compounds (e.g., a bifunctional compound of Formula (I) or a pharmaceutically acceptable salt thereof) or the pharmaceutical compositions thereof as described herein is to be delivered via subcutaneous injection. In some embodiments, any of the bifunctional compounds (e.g., a bifunctional compound of Formula (I) or a pharmaceutically acceptable salt thereof) or the pharmaceutical compositions thereof as described herein is formulated for sustained release (e.g., a depot formulation).

In one aspect, the present disclosure features the use of any of the bifunctional compounds (e.g., a bifunctional compound of Formula (I) or a pharmaceutically acceptable salt thereof) or a related compositions as described herein in the manufacture of a medicament for treating a disease, disorder, condition, or clinical situation modulated by an extracellular target described herein. In another aspect, the present disclosure features the use of any of the bispecific antibodies or fragments thereof (e.g., anti-ASGPR-PCSK9 bispecific antibody or fragment thereof) or related compositions as described herein in the manufacture of a medicament for treating a disease, disorder, condition, or clinical situation modulated by an extracellular target described herein. In one embodiment, an anti-ASGPR-PCSK9 bispecific antibody or fragment thereof or a pharmaceutical composition thereof is used in the manufacture of a medicament for treating a disease, disorder, condition, or clinical situation modulated by PCSK9.

In one aspect, the present disclosure features a nucleic acid molecule encoding any of the bifunctional compounds (e.g., a bifunctional compound of Formula (I) or a pharmaceutically acceptable salt thereof) as described herein. In another aspect, the present disclosure features a nucleic acid molecule encoding any of the bispecific antibodies or fragments thereof (e.g., anti-ASGPR-PCSK9 bispecific antibody or fragment thereof) as described herein. In one embodiment, the nucleic acid molecule encodes an anti-ASGPR-PCSK9 bispecific antibody or fragment thereof.

In one aspect, the present disclosure features an expression vector comprising any of the nucleic acid molecules encoding any of the bifunctional compounds (e.g., a bifunctional compound of Formula (I) or a pharmaceutically acceptable salt thereof) as described herein. In another aspect, the present disclosure features an expression vector comprising any of the nucleic acid molecules encoding any of the bispecific antibodies or fragments thereof (e.g., anti-ASGPR-PCSK9 bispecific antibody or fragment thereof) as described herein. In one embodiment, the expression vector comprises a nucleic acid molecule encoding an anti-ASGPR-PCSK9 bispecific antibody or fragment thereof. In yet another aspect, the present disclosure features a host cell comprising any of the expression vectors or nucleic acid molecules as described herein.

In one aspect, the present disclosure features methods of producing any of the bifunctional compounds (e.g., a bifunctional compound of Formula (I) or a pharmaceutically acceptable salt thereof) as described herein. In another aspect, the present disclosure features methods of producing any of the bispecific antibodies or fragments thereof (e.g., anti-ASGPR-PCSK9 bispecific antibody or fragment thereof) as described herein. In one embodiment, the method of producing includes culturing any of the host cells as described herein under conditions suitable for gene expression and thereafter purifying and collecting any of the bispecific antibodies or fragments thereof (e.g., anti-ASGPR-PCSK9 bispecific antibody or fragment thereof) as described herein from the cell culture.

In one aspect, the present disclosure features a kit comprising any of the bifunctional compounds (e.g., a bifunctional compound of Formula (I) or a pharmaceutically acceptable salt thereof), combinations, and/or pharmaceutical compositions as described herein. In one embodiment, the kit further included means for administering (e.g., oral, parenteral, topical, mucosal, nasal, buccal, or ophthalmological administration) to a subject in need thereof any of the bifunctional compounds (e.g., a bifunctional compound of Formula (I) or a pharmaceutically acceptable salt thereof), the combinations, and/or the pharmaceutical compositions as described herein. In one embodiment, the kit includes written instructions for administering to a subject in need thereof any of the bifunctional compounds, the combinations, and/or the pharmaceutical compositions as described herein.

In another aspect, the kit includes any of the bispecific antibodies or fragments thereof (e.g., anti-ASGPR-PCSK9 bispecific antibody or fragment thereof), nucleic acids, expression vectors, and/or host cells as provided herein. In one embodiment, the kit further included means for use or administering (e.g., oral, parenteral, topical, mucosal, nasal, buccal, or ophthalmological administration) to a subject in need thereof any of the bispecific antibodies or fragments thereof (e.g., anti-ASGPR-PCSK9 bispecific antibody or fragment thereof), nucleic acids, expression vectors, and/or host cells as described herein. In one embodiment, the kit includes written instructions for using or administering to a subject in need thereof any of the bispecific antibodies or fragments thereof (e.g., anti-ASGPR-PCSK9 bispecific antibody or fragment thereof), nucleic acids, expression vectors, and/or host cells as described herein.

The details of one or more embodiments of the disclosure are set forth herein. Other features, objects, and advantages of the disclosure will be apparent from the Detailed Description, the Examples, and the Claims.

### BRIEF DESCRIPTION OF THE DRAWINGS

FIG. 1 is a schematic depicting an exemplary bifunctional molecule, e.g., an anti-ASGPR-PCSK9 bispecific antibody.

FIGS. 2A-2D are graphs illustrating the binding specificity of an exemplary bifunctional compound, an anti-ASGPR-PCSK9 bispecific antibody, to ASGPR and PCSK9. FIG. 2A shows binding of the anti-ASGPR-PCSK9 bispecific antibody to immobilized AGSPR1. FIG. 2B shows binding of the anti-ASGPR-PCSK9 bispecific antibody to full length PCSK9. FIG. 2C shows binding of the anti-ASGPR-PCSK9 bispecific antibody bound to PCSK9 to ASGPR1. FIG. 2D shows binding of the anti-ASGPR-PCSK9 bispecific antibody bound to ASGPR1 to full length PCSK9.

FIGS. 3A-3B are graphs from a mouse model depicting the clearance of PCSK9 modulated by administration of an exemplary bifunctional compound, an anti-ASGPR-PCSK9 bispecific antibody. FIG. 3A shows that the overall amount of PCSK9 levels in serum decreased with increasing amount of the anti-ASGPR-PCSK9 bispecific antibody administered. FIG. 3B shows the rate of clearance of PCSK9 when the anti-ASGPR-PCSK9 bispecific antibody was administered at different doses (0, 0.3, 1, and 3 mg/kg) over the span of 4 hours. Statistics by Two Way ANOVA with Dunnett's multiple comparisons test.

FIG. 4 is a schematic depicting an exemplary bifunctional compound, e.g., a GalNAc-labeled PCSK9 antibody conjugate.

FIGS. 5A-5C are graphs illustrating the binding specificity of an exemplary bifunctional compound, GalNAc trimer-conjugated to an anti-PCSK9 antibody, to ASGPR and PCSK9. FIG. 5A shows direct binding of the GalNAc trimer-anti-PCSK9 antibody conjugate to ASGPR1; FIG. 5B shows direct binding of the GalNAc trimer-anti-PCSK9 antibody conjugate to PCSK9; and FIG. 5C shows formation of a ternary complex, in which the GalNAc trimer-anti-PCSK9 antibody conjugate bound to PCSK9 binds to ASGPR1.

FIGS. 6A-6B are graphs depicting the clearance of PCSK9 in a mouse model modulated by administration of an exemplary bifunctional compound, a GalNAc trimer-anti-PCSK9 antibody conjugate. FIG. 6A shows the rate of clearance of PCSK9 when the GalNAc trimer-anti-PCSK9 antibody conjugate was administered at different doses (0, 0.3, 1, and 3 mg/kg) over the span of 6 hours. Statistics by Two Way ANOVA with Dunnett's multiple comparisons test. FIG. 6B shows that the overall amount of PCSK9 cleared increased with increasing amount of the GalNAc trimer-anti-PCSK9 antibody administered.

FIG. 7 is a Western blot analysis of an exemplary bifunctional compound, a GalNAc-labeled anti-ApoB antibody, as visualized by apoB100 IgG.

FIG. 8 is a schematic depicting an exemplary bifunctional compound, a GalNAc trimer-anti-PCSK9 Fab fragment conjugate.

FIG. 9 is an SDS-PAGE depicting the purification of an exemplary bifunctional compound, a GalNAc trimer-anti-PCSK9 Fab fragment conjugate, and related reaction products.

FIGS. 10A-10B are graphs showing that an exemplary bifunctional compound, a GalNAc trimer-anti-PCSK9 Fab fragment conjugate, demonstrated high binding affinity to both PCSK9 and ASGPR. FIG. 10A shows the binding affinity of the GalNAc trimer-anti-PCSK9 Fab fragment conjugate to avidin-bound PCSK9. FIG. 10B shows the binding affinity of the GalNAc-labeled anti-PCSK9 Fab fragment to avidin-bound deglycosylated ASGPR.

FIGS. 11A-11B are graphs depicting the clearance of PCSK9 in a mouse model modulated by administration of an exemplary bifunctional compound, a GalNAc trimer-anti-PCSK9 Fab fragment conjugate. FIG. 11A shows decreased levels of PCSK9 after administration of the GalNAc trimer-anti-PCSK9 Fab fragment conjugate. FIG. 11B shows the rate of clearance of PCSK9 when the GalNAc trimer-anti-PCSK9 Fab fragment conjugate was administered at different doses (0, 0.3, 1, and 3 mg/kg) over the span of 4 hours.

FIGS. 12A-12C are graphs showing that three exemplary bifunctional compounds, GalNAc trimer-anti-MICA Fab fragment conjugates, demonstrated high binding affinity to ASGPR. FIG. 12A MC-68-LS07 is a Fab conjugate of an isotype control antibody (anti-lysozyme); FIG. 12B IC-68-LW07 and FIG. 12C WD-78-AD05 are Fab conjugates from two different anti-MICA antibodies whose affinities for MICA are 69 pM and 2 pM, respectively.

FIGS. 13A-13B are graphs showing that two exemplary bifunctional compounds, GalNAc trimer-anti-MICA Fab fragment conjugates, resulted in increased clearance of MICA from the serum in a mouse model (FIG. 13A) compared with controls (FIG. 13B).

FIG. 14 is an SDS-PAGE depicting the purification of an exemplary bifunctional compound, a GalNAc trimer-anti-LDLR protein conjugate, and related reaction products.

FIGS. 15A-15B are graphs showing that an exemplary bifunctional compound, a GalNAc trimer-anti-LDLR protein, demonstrated high binding affinity to both PCSK9 (FIG. 15A) and ASGPR (FIG. 15B).

FIGS. 16A-16B are graphs showing that an exemplary bifunctional compound, a GalNAc trimer-PCSK9 protein in two batches, FIG. 16A for batch #PCSK9-GalNAc 1 and FIG. 16B for batch #PCSK9-GalNAc 2, demonstrated high binding affinity to PCSK9.

FIGS. 17A-17B are graphs showing that an exemplary bifunctional compound, a GalNAc trimer-PCSK9 protein conjugate, is more rapidly cleared from the serum in a mouse model, as compared with unconjugated PCSK9 (FIG. 17A) and results in rapid clearance of an anti-PCSK9 antibody from circulation compared with PCSK9 protein only (FIG. 17B). FIG. 17A: Statistics by Two Way ANOVA with Sidak's multiple comparisons test. FIG. 17B: Statistics by Two Way ANOVA with Dunnett's multiple comparisons test.

FIGS. 18A and 18B are graphs showing that TriGalNAc-PCSK9 conjugate 4 serves as a heterobifunctional ligand to accelerate PCSK9 Ab clearance in vivo. FIG. 18A: Synthesis of triGalNAc-hPCSK9 4. FIG. 18B: Dose response of triGalNAc-PCSK9 4 at 0, ( [symbol] ), 2 ( [symbol] ), 6 ( [symbol] ), and 20 ( [symbol] ) mg/kg for clearing a PCSK9 Ab from plasma in LDLR (−/−) mice. Statistics by Iwo Way ANOVA with Dunnett's multiple comparisons test.

FIG. 19 is a structural schematic for ASGPR-PCSK9 bispecific antibody 5 and K_{D} as determined from surface plasmon resonance with separately affixed ASGPR and PCSK9 proteins.

FIGS. 20A and 20B are graphs showing synthesis of triGalNAc-PCSK9 Ab conjugate 10 with independently affixed ASGPR and PCSK9. FIG. 20C is a graph showing an alternative format of triGalNAc-PCSK9 Ab conjugate, wherein n is equal to or smaller than 4. In some embodiments, n is equal to 4. In some embodiments, n is smaller than 4.

FIGS. 21A-21E depict the characterization of triGalNAc-PCSK9 Ab conjugate 10. FIG. 21A is a graph showing the LC-MS m/z spectrum for triGalNAc-PCSK9 Ab conjugate 10. FIG. 21B is a RP-HPLC chromatogram (λ=280 nm) for triGalNAc-PCSK9 Ab conjugate 10. FIG. 21C is a graph showing the LC-MS spectrum for triGalNAc-PCSK9 Ab conjugate 10 (top spectrum) and anti-PCKS9 mAb (bottom spectrum). FIG. 21D is a size exclusion chromatogram (λ=280 nm) for triGalNAc-PCSK9 Ab conjugate 10 (top trace). Dulbecco's PBS, pH 7.0 blank was used as a negative control (bottom trace). FIG. 21E is a non-reducing SDS-PAGE depicting the analysis of samples: (1) Novex Sharp MW Markers; (2) anti-PCSK9 mAb; (3) anti-PCSK9 mAb reduced with TCEP (6.0 eq. per mAb) for 1.5 hours at 40° C.; and (4) triGalNAc-PCSK9 Ab conjugate 10.

### DETAILED DESCRIPTION

Described herein are bifunctional compounds for directing the degradation of extracellular targets, as well as compositions and related methods thereof. Said bifunctional compounds may provide a means for reducing the levels of an otherwise undruggable disease-inducing targets in a sample or subject by directing its degradation, for example, through cellular degradation pathways. As no a priori knowledge of the function, structure, or activity of the disease-inducing target is required, the bifunctional compounds and related methods described herein may provide a powerful alternative to traditional therapies such as chemical inhibition, which often relies on compound design based on the target structure. For example, as compared with small molecule inhibitors or neutralizing antibodies, bifunctional compounds described herein eliminate the entire target molecule. The approach described herein (e.g., bifunctional compounds and their use described herein) is critical and provides advantages particularly for certain molecules, for example (1) for molecules whose presence results in disease, (2) for targets with multiple functional sites, or with large areas of interaction with a partner that are difficult to inhibit. In addition, by degrading the target, target activity is restored based on the synthesis rate of the target, rather than pharmacokinetics of the drug, which provides an advantage for long lived molecules. Finally, as degradation is irreversible, stoichiometric clearance of target by drug (or even superstoichiometric clearance if drug recycles) is possible, making low, infrequent dosing possible.

Further, as compared to traditional small molecule inhibitors or neutralizing antibodies, the bifunctional compounds described herein must bind both target and a degradation receptor, optionally, with a linker attaching these two functional moieties. These discrete modules must be properly engineered to allow for desired functions and properties (e.g., target degradation, recycling, oral availability). The approach described in the prior art, however, fails to show any in vivo activity, particularly any in vivo activity appealing for therapeutic use, partially due to non-obvious combinations of the discrete modules represented by the bifunctional compounds described herein, in order to achieve, e.g., (1) simultaneous high affinity binding to both target and degradation receptor; (2) proper dosing (e.g., the dose, the route of administration and frequency) for target clearance; and (3) a rapid and lasting target clearance effect for therapeutic uses. Indeed, the approach described in the prior art requires high daily doses while only resulting in a very delayed and marginal effect on target concentration, which is not a desired property for therapeutic use (e.g., WO2019/199621 and WO2019/199634). In contrast, the bifunctional compounds described herein have demonstrated compelling ASGPR affinity. Compared to the approaches described in the prior art, the bifunctional compounds described herein are carefully designed and properly engineered and have demonstrated excellent binding affinity/specificity to both the target and the receptor in various degradation pathways, as well as in vivo degradation activity. For example, the linker used within the bifunctional compounds improves affinity markedly, which in turn provides excellent target clearance in vivo.

### Definitions

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modem Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-4 alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂-10 alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quarternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₅), cyclooctenyl (C₅), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like.

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substituent having two oxygen atoms bound to a nitrogen atom, e.g., —NO₂.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The bifunctional compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 910% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The bifunctional compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the bifunctional compounds that are prepared with relatively nontoxic acids or bases, depending on the particular substituents found on the compounds described herein. When bifunctional compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When the bifunctional compounds described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66: 1-19 (1977)). Certain specific bifunctional compounds of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

The term “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —(CH₂CH₂O)ₜ*—, where t is 4-40, and where the “-” indicates the end directed toward the self-immolative spacer and the “*-” indicates the point of attachment to a terminal end group R″ where R″ is H, CH₃ or CH₂CH₂C(═O)OH.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive bifunctional compound, or a pharmaceutical composition thereof.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “antibody,” as used herein, refers to a protein, polypeptide, or fragment thereof, having an amino acid sequence derived from an immunoglobulin molecule that specifically binds to an antigen. The amino acid sequence of an antibody or a fragment thereof usually comprises at least one immunoglobulin variable domain sequence. Typically, antibodies are produced in a subject (e.g., human subject) in response to the presence of an antigen and are capable of specifically binding, non-covalently and reversibly, to the antigen (i.e., via an antigen-binding portion), as well as antigen-binding fragments and engineered variants thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as “VH”) and a heavy chain constant region. The heavy chain constant region is comprised of three domains, CH1, CH2 and CH3. Each light chain is comprised of a light chain variable region (abbreviated herein as “VL”) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2, or CH3) confer important biological properties such as secretion, transplacental mobility, Fc receptor binding, complement binding, and the like. By convention, the numbering of the constant region domains increases as they become more distal from the antigen-binding site or amino-terminus of the antibody. The N-terminus is a variable region and at the C-terminus is a constant region; the CH₃ and CL domains actually comprise the carboxy-terminal domains of the heavy and light chain, respectively.

The assignment of amino acids to each variable region domain is in accordance with the definitions of Kabat, Sequences of Proteins of Immunological Interest (National Institutes of Health, Bethesda, M D, 1987 and 1991) or Chothia, Al-Lazikani et al., J. Mol. Biol. 273, 927-948 (1997). Numbering of the heavy chain constant region is via the EU index as set forth in Kabat (Kabat, Sequences of Proteins of Immunological Interest, National Institutes of Health, Bethesda, M D, 1987 and 1991).

The term “antibody” as used herein includes, but is not limited to, for example, whole antibodies, human antibodies, camelid antibodies, intact monoclonal antibodies (e.g., antibodies produced using hybridoma technology), anti-idiotypic (anti-Id) antibodies (including, e.g., anti-Id antibodies to antibodies of the present disclosure), and antigen-binding antibody fragments, such as a F(ab′)₂, a Fv fragment, a diabody, a single-chain antibody, an scFv fragment, or an scFv-Fc. Genetically engineered intact antibodies and fragments thereof, such as chimeric antibodies, humanized antibodies, minibodies, linear antibodies, multivalent or multi-specific (e.g., bispecific) hybrid antibodies, and the like, are also included. The antibodies can be of any isotype/class (e.g., IgG, IgE, IgM, IgD, IgA and IgY), or subclass (e.g., IgG₁, IgG₂, IgG₃, IgG₄, IgA₁ and IgA₂). Thus, the term “antibody” is used expansively to include any protein that comprises an antigen-binding site of an antibody and is capable of specifically binding to its antigen.

In an embodiment, an antibody is a multispecific antibody, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody is a bispecific antibody. A bispecific antibody has specificity for no more than two antigens. A bispecific antibody is characterized by a first immunoglobulin variable domain sequence which has binding specificity for a first epitope and a second immunoglobulin variable domain sequence that has binding specificity for a second epitope. In an embodiment, the bispecific antibody binds to asialoglycoprotein receptor (ASGPR) and proprotein convertase subtilisin/kexin type 9 (PCSK9). In an embodiment, the multispecific antibody is a trispecific antibody, e.g., an antibody having specificity for three antigens.

The phrase “antibody fragment” as used herein refers to one or more portions of an antibody. In some embodiments, these portions are part of the constant domain(s) of an antibody, e.g., fragment crystallizable (Fc), constant (C) domains, etc. In other embodiments, these portion(s) are antigen-binding fragments that retain the ability of binding an antigen non-covalently, reversibly and specifically, sometimes referred to herein as the antigen binding domain. In some embodiments, an antibody fragment is a Fab, a Fab′, a F(ab′)₂, a F(ab)₂, variable fragment (Fv), a domain antibody (dAb), a single domain antibody, or a single chain variable fragment (scFv).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “antigen” refers to a molecule that provokes an immune response and also the molecule or molecules recognized by the components of that immune response (e.g., antibodies and immunologically-competent cells). This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample or might be macromolecule besides a polypeptide. Such a biological sample can include, but is not limited to a tissue sample, a tumor sample, a cell or a fluid with other biological components

Unless specified otherwise, the term “bifunctional compound”, “bifunctional compounds”, “bifunctional compound of the present disclosure” or “bifunctional compounds of the present disclosure” refer to a bifunctional compound or bifunctional compounds of Formula (I), as well as pharmaceutically acceptable salts thereof, as well as all stereoisomers (including diastereoisomers and enantiomers), rotamers, tautomers and isotopically labeled compounds (including deuterium substitutions).

As used herein, the term “biological neutralization” refers to a molecule that binds to a target in such a way as to diminish the target's ability to (e.g., prevent the target's ability to) carry out the target's normal biological function. For example, a neutralizing anti-PCSK9 antibody may bind to PCSK9 to prevent a function of PCSK9, such as binding to LDLR to cause its degradation.

The term “combination” as used herein refers to either a fixed combination in one dosage unit form, or a combined administration where a compound of the present invention and a combination partner (e.g., another drug, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration.

The terms “complementarity determining region” and “CDR,” are used herein interchangeably to refer to the sequences of amino acids of an antibody or fragment thereof, such as within antibody variable regions (VL and/or VH), which confer antigen specificity and binding affinity. In general, there are three CDRs in each light chain variable region (LCDR1, LCDR2 and LCDR3, numbered sequentially from the N-terminus) and three CDRs in each heavy chain variable region (HCDR1, HCDR2 and HCDR3, numbered sequentially from the N-terminus).

The positions of the CDRs and framework regions can be determined using various well-known definitions in the art, e.g., Kabat, Chothia, IMGT, AbM, and combined definitions (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J. Mol. Biol., 273:927-748 (1997); see also the world wide web at bioinf.org.uk/abs/info.html). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

For Example, Table 1 lists CDR definitions according to Chothia, Kabat (Gawinowicz et al. 1991), combined, and IMGT. In a combined Kabat and Chothia numbering scheme, in some embodiments, the CDRs correspond to the amino acid residues that are part of a Kabat CDR, a Chothia CDR, or both. Under IMGT, the CDR regions of an antibody can be determined using the program IMGT/DomainGap Align. IMGT tools are available at World Wide Web imgt.org.

TABLE 1
CDR definitions.

| | Chothia | Kabat | Combined | IMGT* |
|---|---|---|---|---|
| HCDR1 | H26-H32 | H31-H35 | H26-H35 | H26-H33 |
| HCDR2 | H52-H56 | H50-H65 | H50-H65 | H51-H57 |
| HCDR3 | H95-H102 | H95-H102 | H95-H102 | H93-H102 |
| LCDR1 | L26-L32 | L24-L34 | L24-L34 | L27-L32 |
| LCDR2 | L50-L52 | L50-L56 | L50-L56 | L50-52 |
| LCDR3 | L91-L96 | L89-L97 | L89-L97 | L89-L97 |

*Numbering is approximate according to Kabat

As used herein, the terms “condition,” “disease,” “disorder” and “clinical situation” are used interchangeably.

An “effective amount” of a bifunctional compound described herein refers to an amount sufficient to elicit a desired biological response, i.e., treating a condition. As will be appreciated by those of ordinary skill in this art, the effective amount of a bifunctional compound may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor. In an embodiment, the effective amount of a bifunctional compound described herein is equimolar to the concentration of a target or in slight excess in concentration to a target. In an embodiment, the effective amount of a bifunctional compound described herein is the concentration required to achieve a binary complex of the bifunctional compound and a target. In an embodiment, an effective amount of a bifunctional compound described herein does not comprise a concentration in high excess over a target concentration (e.g., 100×, 250×, 500×, or more of a bifunctional compound described herein over a target).

A “therapeutically effective amount” of a bifunctional compound is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a bifunctional compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The term “extracellular,” as used herein, refers to the space outside the plasma membrane of a cell or cells.

The term “extracellular target,” as used herein, refers to an entity, such as a molecule or a plurality of molecules, that is present in the space outside the plasma membrane of a cell or cells. An extracellular target may be a protein or a non-protein entity. Exemplary extracellular targets include proteins (soluble proteins and membrane-associated proteins, such as antibodies, receptors, growth factors, cytokines, chemokines, enzymes, hormones, neurotransmitters, or a combination thereof), lipoproteins, nucleic acids (e.g., oligonucleotides, DNA, RNA), toxins, liposomes, virus particles, and cells (e.g., prokaryotic cells, eukaryotic cells).

The terms “reduction of the extracellular level” or “depression of the extracellular levels”, as used herein, refer to decreasing or lowering the concentration and/or the activity of a target located in the space outside the plasma membrane of a cell or cells compared to its concentration and/or activity in the absence of the bifunctional compounds described herein. In an embodiment, the decrease of concentration and/or activity of a target located in the space outside the plasma membrane of a cell or cells is about 10%, 15%, 20%, 25%, 30%, 35%, 40%, 45%, 50%, 55%, 60%, 65%, 70%, 75%, 80%, 85%, 90%, 95%, 99%, or more compared to the concentration and/or activity of a target in the absence of the bifunctional compounds described herein. In an embodiment, the decrease of concentration and/or activity of a target located outside the plasma membrane of a cell or cells is greater than about 30% (e.g., greater than about 40%, 50%, 60%, 70%, 80%, 90%, 95%, or more) compared to the concentration and/or activity of a target in the absence of the bifunctional compounds described herein.

The term “pathogenic autoantibody” refers to an antibody directed against one or more of a subject's own antigens. A pathogenic autoantibody may cause injury and/or inflammation in a subject. For example, pathogenic autoantibodies play a role in several diseases such as lupus erythematosus, rheumatoid arthritis, primary biliary cirrhosis, membranous nephropathy, IgA nephropathy, IgE-mediated allergic response, type 1 diabetes, and myasthenia gravis.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a bifunctional compound described herein (e.g., a bifunctional compound of Formula (I)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

By “reduces,” “decreases,” or “lowers” is meant a negative alteration of at least about 5%, 10%, 15%, 20%, 25%, 30%, 40%, 50%, 60%, 70%, 75%, 80%, 85%, 90%, 95%, 96%, 97%, 98%, 99%, or 100%. In some embodiments, in the context of a particular disease (e.g., a PCSK9-mediated disease or disorder), “reduces,” “decreases,” or “lowers” means a reduction in the level, expression or activity of an extracellular target (e.g., PCSC9) to a level considered in the literature as below the range of normal for a subject without such disorder, or to a level that reduces or ameliorates symptoms of the disease (e.g., a PCSK9-mediated disease or disorder). The decrease can be, for example, at least about 10%, at least about 20%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more e.g., 100%.

By “reference” is meant a standard or control condition. The “reference level known in the art” refers to an experimentally determined control or threshold level identified or known in the prior art, or to the level identified in a sample from a control patient or in samples from a control patient population. In one embodiment, a reference is an untreated subject, or a subject administered with a placebo or normal saline, medium, buffer, and/or a control. In one embodiment, a reference is a healthy subject.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications (e.g., gaps or overhangs).

In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

The term “single-chain Fv” or “scFv” as used herein typically refers to antibody fragments comprising the VH and VL domains of an antibody, wherein these domains are present in a single polypeptide chain. Preferably, the Fv polypeptide further comprises an internal polypeptide linker between the VH and VL domains, which enables the scFv to form the desired structure for antigen binding. A scFv may also have an engineered internal disulfide bridge that enhances stability. For a review of scFvs, see Plückthun in The Pharmacology of Monoclonal Antibodies, vol. 113, Rosenburg and Moore eds., (1994) Springer-Verlag, New York, pp. 269-315.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

A “subject in need thereof” is a subject or patient that would benefit from administration of a bifunctional compound of the present disclosure used, e.g., for treatment or a disease, disorder, or condition. In some embodiments, a “subject in need thereof” is a subject or patient a diagnosed with, at risk or having, predetermined to have, or suspected of having a disease or disorder (e.g., a PCSK9-mediated disease or disorder). In a nonlimiting example, a “subject in need thereof” is a subject or patient who has, is at risk of developing, or who is susceptible to a PCSK9-mediated disease or disorder.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a bifunctional compound described herein (e.g., a bifunctional compound of Formula (I)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

As used herein, a “vector” refers to a nucleic acid (polynucleotide) molecule capable of transporting another polynucleotide to which it has been linked. In some embodiments, a vector is a “plasmid,” which refers to a circular double stranded DNA loop into which additional DNA segments may be ligated. In other embodiments, a vector is a viral vector (e.g., AAV), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). Expression vectors may include additional nucleic acid sequences to promote and/or facilitate the expression of the introduced sequence, such as start, stop, enhancer, promoter, and secretion sequences. Nonlimited examples of vectors include plasmids, transposons, phages, viruses, liposomes, and episome. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The terms “PCSK9,” “hPCSK9” or “proprotein convertase subtilisin/kexin type 9” interchangeably refer to a naturally occurring human proprotein convertase belonging to the proteinase K subfamily of the secretory subtilase family. PCSK9 is synthesized as a soluble zymogen that undergoes autocatalytic intramolecular processing in the endoplasmic reticulum. PCSK9 plays a role in cholesterol homeostasis and may have a role in the differentiation of cortical neurons. Mutations in the PCSK9 gene are a cause of autosomal dominant familial hypercholesterolemia (Burnett and Hooper, Clin. Biochem. Rev. (2008) 29(1):11-26). “PCSK9” refers to the proprotein convertase subtilsin kexin 9 gene or protein. PCSK9 is also known as FH3, PC9, FHCL3, LDLCQ1, HCHOLA3, NARC-1, or NARC1. The term PCSK9 includes human PCSK9, the amino acid and nucleotide sequence of which may be found in, by way of example only, Gene ID: 255738; mouse PCSK9, the amino acid and nucleotide sequence of which may be found in, by way of example only, Gene ID: 100102; rat PCSK9, the amino acid and nucleotide sequence of which may be found in, by way of example only, Gene ID: 298296.

The terms “PCSK9-mediated disease or disorder” or “disease or disorder associated with PCSK9”, as used herein, refers to a disease or disorder associated with the activity of PCSK9, which include hypercholesterolemia, hyperlipidemia, hypertriglyceridemia, sitosterolemia, atherosclerosis, arteriosclerosis, coronary heart disease, peripheral vascular disease (including aortic diseases and cerebrovascular disease), peripheral arterial disease, vascular inflammation, elevated Lp(a), elevated LDL, elevated TRL, elevated triglycerides, sepsis, and xanthoma.

The terms “hypercholesterolemia” and “dyslipidemia” include, e.g., familial and non-familial hypercholesterolemia. Familial hypercholesterolemia (FH) is an autosomal dominant disorder characterized by elevation of serum cholesterol bound to low density lipoprotein (LDL). Familial hypercholesterolemia includes both heterozygous FH and homozygous FH. Hypercholesterolemia (or dyslipidemia) is the presence of high levels of cholesterol in the blood. It is a form of hyperlipidemia (elevated levels of lipids in the blood) and hyperlipoproteinemia (elevated levels of lipoproteins in the blood). Hyperlipidemia is an elevation of lipids in the bloodstream. These lipids include cholesterol, cholesterol esters, phospholipids and triglycerides. Hyperlipidemia includes for example, type I, IIa, IIb, III, IV and V. Hypertriglyceridemia denotes high blood levels of triglycerides. Elevated levels of triglycerides are associated with atherosclerosis, even in the absence of hypercholesterolemia, and predispose to cardiovascular disease.

The terms “sitosterolemia” or “phytosterolemia” refer to a rare autosomal recessively inherited lipid metabolic disorder characterized by hyperabsorption of sitosterol from the gastrointestinal tract and decreased biliary excretion of dietary sterols (i.e., leading to hypercholesterolemia, tendon and tuberous xanthomas, premature development of atherosclerosis) and altered cholesterol synthesis.

The term “artherosclerosis” includes hardening of arteries associated with deposition of fatty substances, cholesterol, cellular waste products, calcium and fibrin in the inner lining of an artery. The buildup that results is called plaque.

The terms “atherosclerosis” or “arteriosclerotic vascular disease (ASVD)” refer to a specific form of arteriosclerosis involving thickening, hardening and loss of elasticity of the walls of arteries as a result of invasion and accumulation of white blood cells, containing both living, active white blood cells (producing inflammation) and remnants of dead cells, including cholesterol and triglycerides. Atherosclerosis is a syndrome affecting arterial blood vessels due to a chronic inflammatory response of white blood cells in the walls of arteries.

The term “coronary heart disease,” also known as atherosclerotic artery disease, atherosclerotic cardiovascular disease, coronary heart disease, or ischemic heart disease, is the most common type of heart disease and cause of heart attacks. The disease is caused by plaque building up along the inner walls of the arteries of the heart, which narrows the lumen of arteries and reduces blood flow to the heart.

The term “xanthoma” refers to a cutaneous manifestation of lipidosis in which lipids accumulate in large foam cells within the skin. Xanthomas are associated with hyperlipidemias.

The term “elevated Lp(a) concentration”, as used herein, refers to a serum Lp(a) concentration above 30 mg/dl (75 nmol/L). “Elevated serum Lp(a)” means a serum Lp(a) level greater than about 14 mg/dL. In certain embodiments, a patient is considered to exhibit elevated serum Lp(a) if the level of serum Lp(a) measured in the patient is greater than about 15 mg/dL, about 20 mg/dL, about 25 mg/dL, about 30 mg/dL, about 35 mg/dL, about 40 mg/dL, about 45 mg/dL, about 50 mg/dL, about 60 mg/dL, about 70 mg/dL, about 80 mg/dL, about 90 mg/dL, about 100 mg/dL, about 20 mg/dL, about 140 mg/dL, about 150 mg dL, about 180 mg/dL, or about 200 mg/dL The serum Lp(a) level can be measured in a patient post-prandial. In some embodiments, the Lp(a) level is measured after a period of time of fasting (e.g., after fasting for 8 hrs, 8 hrs, 10 hrs, 12 hrs or more). Exemplary methods for measuring serum Lp(a) in a patient include, but are not limited to, rate immunonephelometry, ELISA, nephelometry, immunoturbidimetry, and dissociation-enhanced lanthanide fluorescent immunoassay, although any clinically acceptable diagnostic method can be used in the context of the disclosure.

By “elevated triglyceride levels” or “ETL” is meant any degree of triglyceride levels that is determined to be undesirable or is targeted for modulation.

The term “sepsis” refers to a systemic reaction characterized by arterial hypotension, metabolic acidosis, decreased systemic vascular resistance, tachypnea, and organ dysfunction. Sepsis can result from septicemia (i.e., organisms, their metabolic end-products or toxins in the blood stream), including bacteremia (i.e., bacteria in the blood), as well as toxemia (i.e., toxins in the blood), including endotoxemia (i.e., endotoxin in the blood). The term “sepsis” also encompasses fungemia (i.e., fungi in the blood), viremia (i.e., viruses or virus particles in the blood), and parasitemia (i.e., helminthic or protozoan parasites in the blood). Thus, septicemia and septic shock (acute circulatory failure resulting from septicemia often associated with multiple organ failure and a high mortality rate) may be caused by a number of organisms.

The term “CFHR3” or “complement factor H-related protein 3 gene” interchangeably refer to the gene that encodes the human protein complement factor H-related protein 3 (FHR3).

The term “FHR3” or “complement factor H-related protein 3” interchangeably refer to a naturally occurring human complement factor H-related protein 3 which is a secreted protein, belonging to the complement factor H-related protein family.

The terms “CFHR3-mediated disease or disorder” or disease or disorder associated with CFHR3”, as used herein, refer to a disease or disorder associated with the aberrant activity of CFHR3, which include nephropathy, age-related macular degeneration, atypical hemolytic uremic syndrome and hepatocellular carcinoma (HCC).

The terms “FHR3-mediated disease or disorder” or disease or disorder associated with FHR3”, as used herein, refer to a disease or disorder associated with the activity of FHR3, which include nephropathy, age-related macular degeneration, atypical hemolytic uremic syndrome and hepatocellular carcinoma (HCC).

The term “nephropathy”, as used herein, refers disease or damage of the kidney(s).

The term “age-related macular degeneration”, as used herein, refers an eye disease which affects the macula of the eye causing blindness over time.

The term “atypical hemolytic uremic syndrome”, as used herein, refers a disease which affects kidney function due to abnormal blood clotting in the kidney. Atypical hemolytic-uremic syndrome is characterized by three major features related to abnormal clotting: hemolytic anemia, thrombocytopenia, and kidney failure.

The term “hemolytic anemia”, as used herein, refers to the premature break down of red blood cells.

The term “thrombocytopenia”, as used herein, refers to the reduced level of circulating platelets used in assisting clotting.

The term “hepatocellular carcinoma (HCC)”, as used herein, refers cancer of the liver.

The terms “asialoglycoprotein receptor” or “ASGPR” interchangeably refer to the protein or the gene encoding the ASGPR protein and includes any of the ASGPR naturally occurring forms, homologs, mutants, functional fragments, or variants that maintain the activity of ASGPR (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ASGPR may also be referred to herein as CLEC4H1, C-Type Lectin Domain Family 4 Member H1, Hepatic Lectin H1, or HL-1. In some embodiments, the ASGPR protein is encoded by the nucleic acid identified by the NCBI sequence reference NM_00167, NM_080914, homolog or functional fragment thereof. In some embodiments, the ASGPR protein refers to a polypeptide, a homolog, a variant, a mutant, or a functional fragment as identified by UniProt ID P07306 or P07307. In some embodiments, the ASGPR protein comprises a major (48 kDa) and minor (40 kDa) subunit. ASGPR protein or the polypeptide encoded by the ASGPR gene is involved in the serum glycoprotein homeostasis, e.g., the binding, internalization, and clearance of glycoproteins comprising a terminal galactose (Gal) or N-acetylgalactosamine (GalNAc) residue.

The terms “mannose-6-phosphate receptor,” or “M6PR” interchangeably refer to the protein or the gene that encodes the M6PR protein and includes any of the M6PR naturally occurring forms, homologs, mutants, functional fragments, or variants that maintain the activity of M6PR (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. M6PR may also be referred to herein as CD-MPR, Cation-Dependent Mannose-6-Phosphate Receptor, CD Man-6-P Receptor, CD-M6PR, MPR 46, MPR46, M6PR, MPRD, or SMPR. In some embodiments, the M6PR protein is encoded by the nucleic acid identified by the NCBI sequence reference NM 002355, NM_000876, homolog or functional fragment thereof. In some embodiments, the M6PR protein refers to a polypeptide, a homolog, a variant, a mutant, or a functional fragment as identified by UniProt ID P20645 or P11717. As used herein, M6PR comprises the cation-independent mannose-6-phosphate receptors (CI-M6PR) and cation-dependent mannose-6-phosphate receptors (CD-M6PR), which may require divalent cations to recognize their targets. M6PR protein or the polypeptide encoded by the M6PR gene is involved in transport of mannose-6-phosphate, e.g., binding to glycoproteins bearing a terminal mannose-6-phosphate residue.

The terms “insulin-like growth factor 2 receptor” or “IGF2R” interchangeably refer to the protein or the gene that encodes IGF2R, and includes any of the IGF2R naturally occurring forms, homologs, mutants, functional fragments, or variants that maintain the activity of IGF2R (e.g., within at least 50%, 80%, 90%, 95%, 96%, 97%, 98%, 99% or 100% activity compared to the native protein). In some embodiments, mutants, variants or homologs have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IGF2R may also be referred to herein as MPRI, CI Man-6-P Receptor, CI-M6PR, M6P/IGF2R, IGF-II Receptor, M6P/IGF2 Receptor, CI Man-6-P Receptor, MPR 300, CI-MPR, CD222, CIMPR, M6P-R, MPR1, or Cation-Independent Mannose-6 Phosphate Receptor. In some embodiments, the IGF2R protein is encoded by the nucleic acid identified by the NCBI sequence reference NM_000876, homolog or functional fragment thereof. In some embodiments, the IGF2R protein refers to a polypeptide, a homolog, a variant, a mutant, or a functional fragment as identified by UniProt ID P11717. IGF2R protein or the protein encoded by the IGF2R gene is a protein receptor involved in trans-Golgi network transport.

The terms “tumor necrosis factor A” and “TNFA” interchangeably refer to the protein or the gene that encodes the TNFA protein and includes any of the TNFA naturally occurring forms, homologs, mutants, functional fragments, or variants that maintain the activity of TNFA (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TNFA may also be referred to herein as Tumor Necrosis Factor, TNF-Alpha, TNFSF2, Tumor Necrosis Factor Ligand Superfamily Member 2, Cachectin, TNF-A, DIF, Tumor Necrosis Factor Ligand 1F, TNLG1F, or APC1 Protein. In some embodiments, the TNFA protein is encoded by the nucleic acid identified by the NCBI sequence reference NM_000594, homolog or functional fragment thereof. In some embodiments, the TNFA protein refers to a polypeptide, a homolog, a variant, a mutant, or a functional fragment as identified by UniProt ID P01375. Tumor necrosis factor A protein or the polypeptide encoded by the TNFA gene is a cytokine involved in, for example, the initiation of systemic inflammation.

The terms “tumor necrosis factor receptor 1” and “TNFR1” interchangeably refer to the protein or the gene that encodes the TNFR1 protein and includes any of the TNFR1 naturally occurring forms, homologs, mutants, functional fragments, or variants that maintain the activity of TNFR1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TNFR1 may also be referred to herein as TNFRSF1A, CD120a, FPF, MS5, TBP1, TNF-R, TNF-R-I, TNF-R55, TNFAR, TNFR1, TNFR1-d2, TNFR55, TNFR60, p55, p55-R, p60, tumor necrosis factor receptor superfamily member 1A, TNF receptor superfamily member 1A. In some embodiments, the TNFR1 protein is encoded by the nucleic acid identified by the NCBI sequence reference NM_001065, NM_001346091, NM_001346092, homolog or functional fragment thereof. In some embodiments, the TNFR1 protein refers to a polypeptide, a homolog, a variant, a mutant, or a functional fragment as identified by UniProt ID P19438. TNFR1 protein or the polypeptide encoded by the TNFR1 gene is a membrane receptor that, inter alia, binds to tumor necrosis factor-alpha (TNF-alpha).

The terms “interleukin-1 receptor” or “ILIR” interchangeably refer to the protein or the gene that encodes the IL1R protein and includes any of the IL1R naturally occurring forms, homologs, mutants, functional fragments, or variants that maintain the activity of IL1R (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL1R may also be referred to herein as CD121 Antigen-Like Family Member A, Interleukin-1 Receptor Type 1, Interleukin-1 Receptor Alpha, IL-1R-Alpha, IL-1RT-1, D2S1473, IL-1R-1, IL-1RT1, CD121A, P80, EC 3.2.2.6, D2S1473, Interleukin 1 Receptor Type 2, Interleukin-1 Receptor Beta, IL-1R-Beta, IL-1RT-2, CDw121b, IL-1R-2, CD121b, IL1RB, IL1R2c or IL1R². In some embodiments, the ILIR protein is encoded by the nucleic acid identified by the NCBI sequence reference NM 000877, NM_173343, a homolog or a functional fragment thereof. In some embodiments, the ILIR protein refers to a polypeptide, a homolog, a variant, a mutant, or a functional fragment as identified by UniProt ID P14778 or P27930. As used herein, ILIR comprises the interleukin 1 receptor, type I and interleukin 1 receptor, type II. IL1R protein or the polypeptide encoded by the IL1R gene is a cytokine receptor that binds to, inter alia, interleukin-1.

The terms “interleukin-1 alpha” or “IL1-alpha” interchangeably refer to the protein or the gene that encodes the IL1-alpha protein and includes any of the IL1-alpha naturally occurring forms, homologs, mutants, functional fragments, or variants that maintain the activity of IL1-alpha (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL1-alpha may also be referred to herein as hematopoietin 1, IL1A, IL-1A, IL1, IL1-ALPHA, IL1F1, interleukin 1 alpha, IL-1 alpha, Pro-Interleukin-1-Alpha, or Preinterleukin 1 Alpha. In some embodiments, the IL1-alpha protein is encoded by the nucleic acid identified by the NCBI sequence reference NM 000575, NM_001371554, a homolog or a functional fragment thereof. In some embodiments, the IL1-alpha protein refers to a polypeptide, a homolog, a variant, a mutant, or a functional fragment as identified by UniProt ID P01583. IL1-alpha protein or the polypeptide encoded by the IL1-alpha gene is a cytokine mediator of the inflammatory response.

The terms “interleukin-1 beta” or “IL1-beta” interchangeably refer to the protein or the gene that encodes the IL1-beta protein and includes any of the IL1-beta naturally occurring forms, homologs, mutants, functional fragments, or variants that maintain the activity of IL1-beta (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL1-beta may also be referred to herein Interleukin 1 Beta, IL1F2, Catabolin, Pro-Interleukin-1-Beta, leukocytic pyrogen, leukocytic endogenous mediator, mononuclear cell factor, or lymphocyte activating factor. In some embodiments, the IL1-beta protein is encoded by the nucleic acid identified by the NCBI sequence reference NM_000576, homolog or functional fragment thereof. In some embodiments, the IL1-beta protein refers to a polypeptide, a homolog, a variant, a mutant, or a functional fragment as identified by UniProt ID P01584. IL1-beta protein or the polypeptide encoded by the IL1-beta gene is a cytokine mediator of the inflammatory response.

The terms “interleukin 17” or “IL17” interchangeably refer to the protein or the gene that encodes the IL17 protein and includes any of the IL17 naturally occurring forms, homologs, mutants, functional fragments, or variants that maintain the activity of IL17 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL17 may also be referred to herein as IL17A, CTLA8, IL-17, IL-17A, IL17, CTLA-8, interleukin 17A, Cytotoxic T-Lymphocyte-Associated Antigen 8, or Cytotoxic T-Lymphocyte-Associated Serine Esterase 8. In some embodiments, the IL17 protein is encoded by the nucleic acid identified by the NCBI sequence reference NM 002190, a homolog or a functional fragment thereof. In some embodiments, the IL17 protein refers to a polypeptide, a homolog, a variant, a mutant, or a functional fragment as identified by UniProt ID Q16552. IL17 protein or the polypeptide encoded by the IL17 gene is a cytokine, produced in response to stimulation with IL-23.

The terms “interleukin 12A” or “IL12A” interchangeably refer to the protein or the gene that encodes the IL12A protein and includes any of the IL12A naturally occurring forms, homologs, mutants, functional fragments, or variants that maintain the activity of IL12A (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL12A may also be referred to herein as IL-12 p35, CLMF, IL-12A, NFSK, NKSF1, P35, interleukin 12A, Cytotoxic Lymphocyte Maturation Factor 1, Cytotoxic Lymphocyte Maturation Factor 35 KDa Subunit, or NF Cell Stimulatory Factor Chain 1. In some embodiments, the IL12A protein is encoded by the nucleic acid identified by the NCBI sequence reference NM_000882, NM_001354582, NM_001354583 a homolog or a functional fragment thereof. In some embodiments, the IL12A protein refers to a polypeptide, a homolog, a variant, a mutant, or a functional fragment as identified by UniProt ID P29459. IL12A protein or the polypeptide encoded by the IL12A gene is a subunit of the cytokine IL12 that is required for the T-cell dependent induction of interferon gamma.

The terms “interleukin 12B” or “IL12B” interchangeably refer to the protein or the gene that encodes the IL12B protein and includes any of the IL12A naturally occurring forms, homologs, mutants, functional fragments, or variants that maintain the activity of IL12B (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL12B may also be referred to herein as CLMF, CLMF P40, CLMF2, IL-12B, IMD28, NKSF, NKSF2, IMD29 natural killer cell stimulatory factor 2, cytotoxic lymphocyte maturation factor p40, or interleukin-12 subunit p40. In some embodiments, the IL12B protein is encoded by the nucleic acid identified by the NCBI sequence reference NM_002187, a homolog or a functional fragment thereof. In some embodiments, the IL12B protein refers to a polypeptide, a homolog, a variant, a mutant, or a functional fragment as identified by UniProt ID P29460. IL12B protein or the polypeptide encoded by the IL12B gene is a subunit of the cytokine IL12 that is involved in Th1 cells development.

The terms “interleukin 23” or “IL23” interchangeably refer to a heterodimeric protein comprising an IL23A subunit and a IL12B subunit (that is shared with IL-12) or the genes that encodes the subunits of the IL23 protein and includes any of the IL23 naturally occurring forms, homologs, mutants, functional fragments, or variants that maintain the activity of IL23 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In some embodiments, IL-23 refers to IL-23A. IL23 may also be referred to herein as SGRF, IL23P19, IL-23A, P19, Interleukin-23 Subunit P19, JKA3 Induced Upon T-Cell Activation, or Interleukin 23 P19 Subunit. In some embodiments, the IL23 protein is encoded by the nucleic acid identified by the NCBI sequence NM_016584, a homolog, or a functional fragment thereof. In some embodiments, the IL23 protein refers to a polypeptide, a homolog, a variant, a mutant, or a functional fragment as identified by UniProt ID Q9NPF7. IL23 protein is a cytokine involved in, inter alia, maintenance of Th17 and expansion.

The terms “interleukin 2” or “IL2” interchangeably refer to the protein or the gene that encodes the IL2 protein and includes any of the IL2 naturally occurring forms, homologs, mutants, functional fragments, or variants that maintain the activity of IL2 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL2 may also be referred to herein as TCGF, lymphokine, T Cell Growth Factor, Aldesleukin, or Involved In Regulation Of T-Cell Clonal Expansion. In some embodiments, the IL2 protein is encoded by the nucleic acid identified by the NCBI sequence reference NM_000586, a homolog or a functional fragment thereof. In some embodiments, the IL2 protein refers to a polypeptide, a homolog, a variant, a mutant, or a functional fragment as identified by UniProt ID P60568. IL2 protein or the polypeptide encoded by the IL2 gene is a cytokine signaling molecule.

The terms “interleukin 5” or “IL5” interchangeably refer to the protein or the gene that encodes the IL5 protein and includes any of the IL5 naturally occurring forms, homologs, mutants, functional fragments, or variants that maintain the activity of IL5 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL5 may also be referred to herein as TRF, EDF, Eosinophil Differentiation Factor, Colony-Stimulating Factor, Eosinophil, or B-Cell Differentiation Factor I. In some embodiments, the IL5 protein is encoded by the nucleic acid identified by the NCBI sequence reference NM_000879, a homolog or a functional fragment thereof. In some embodiments, the IL5 protein refers to a polypeptide, a homolog, a variant, a mutant, or a functional fragment as identified by UniProt ID P05113. IL5 protein or the polypeptide encoded by the IL5 gene is a cytokine associated with stimulation of the immune system, e.g., in allergic responses.

The terms “interleukin 6” or “IL6” interchangeably refer to the protein or the gene that encodes the IL6 protein and includes any of the IL6 naturally occurring forms, homologs, mutants, functional fragments, or variants that maintain the activity of IL6 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL6 may also be referred to herein as BSF2, HGF, HSF, IFNB2, IL-6, BSF-2, CDF, IFN-beta-2, B-Cell Stimulatory Factor 2, CTL Differentiation Factor, Hybridoma Growth Factor, B-Cell Differentiation Factor, Interferon, Beta 2, or Interleukin BSF-2. In some embodiments, the IL6 protein is encoded by the nucleic acid identified by the NCBI sequence reference NM_000600, NM_001318095, NM_001371096, a homolog or a functional fragment thereof. In some embodiments, the IL6 protein refers to a polypeptide, a homolog, a variant, a mutant, or a functional fragment as identified by UniProt ID P05231. IL6 protein or the polypeptide encoded by the IL6 gene is a pro-inflammatory cytokine and an anti-inflammatory myokine.

The terms “interleukin 10” or “IL10” interchangeably refer to the protein or the gene that encodes the IL10 protein and includes any of the IL10 naturally occurring forms, homologs, mutants, functional fragments, or variants that maintain the activity of IL10 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL10 may also be referred to herein as CSIF, GVHDS, IL-10, IL10A, TGIF, T-Cell Growth Inhibitory Factor, or Cytokine Synthesis Inhibitory Factor. In some embodiments, the IL10 protein is encoded by the nucleic acid identified by the NCBI sequence reference NM_000572, a homolog or a functional fragment thereof. In some embodiments, the IL10 protein refers to a polypeptide, a homolog, a variant, a mutant, or a functional fragment as identified by UniProt ID P22301. IL10 protein or the polypeptide encoded by the IL10 gene is a cytokine associated in various pathways in the immune system, including enhancement of B cell survival and antibody production.

The terms “interleukin 13” or “IL13” interchangeably refer to the protein or the gene that encodes the IL13 protein and includes any of the IL13 naturally occurring forms, homologs, mutants, functional fragments, or variants that maintain the activity of IL13 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IL13 may also be referred to herein as P600, Bronchial Hyperresponsiveness-1, Allergic Rhinitis, MGC116786, MGC116788, MGC116789, ALRH, BHR1, NC30, or Interleukin-13. In some embodiments, the IL13 protein is encoded by the nucleic acid identified by the NCBI sequence reference NM_002188, a homolog or a functional fragment thereof. In some embodiments, the IL13 protein refers to a polypeptide, a homolog, a variant, a mutant, or a functional fragment as identified by UniProt ID P35225. IL13 protein or the polypeptide encoded by the IL13 gene is a cytokine associated in various pathways in the immune system, including mediation of physiologic changed induced by allergic inflammation.

The terms “immunoglobulin E” or “IgE” interchangeably refer to the or the gene that encodes the IgE protein and includes any of the IgE naturally occurring forms, homologs, mutants, functional fragments, or variants that maintain the activity of IgE (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IgE may also be referred to herein as IGHE or Immunoglobulin Epsilon. In some embodiments, the IgE protein is encoded by the nucleic acid identified by the NCBI sequence reference AH005278.2, a homolog or a functional fragment thereof. In some embodiments, the IgE protein refers to a polypeptide, a homolog, a variant, a mutant, or a functional fragment as identified by UniProt ID P01854. In some embodiments, IgE comprises two heavy chains (ε chain) and two light chains, with the a chain containing 4 Ig-like constant domains (Cε1-Cε4). IgE protein is a type of antibody found in mammals found, e.g., on the surface of mast cells and basophils.

The terms “insulin-like growth factor 1” or “IGF1” interchangeably refer to the protein or the gene that encodes the IGF1 protein and includes any of the IGF1 naturally occurring forms, homologs, mutants, functional fragments, or variants that maintain the activity of IGF1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. IGF1 may also be referred to herein as IGF-I, IGF1A, IGFI, MGF, IBP1, insulin like growth factor 1, IGF, Somatomedin-C, Mechano Growth Factor, or Insulin-Like Growth Factor IB. In some embodiments, the IGF1 protein is encoded by the nucleic acid identified by the NCBI sequence reference NM_000618, NM_001111283, NM_001111284, NM_001111285, a homolog or a functional fragment thereof. In some embodiments, the IGF1 protein refers to a polypeptide, a homolog, a variant, a mutant, or a functional fragment as identified by UniProt ID P05019. IGF1 protein or the polypeptide encoded by the IGF1 gene is an endocrine hormone.

The terms “erythropoietin” or “EPO” interchangeably refer to the protein or the gene that encodes the EPO protein and includes any of the EPO naturally occurring forms, homologs, mutants, functional fragments, or variants that maintain the activity of EPO (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. EPO may also be referred to herein as EP, MVCD2, erythropoietin, haematopoietin, haemopoietin, ECYT5, DBAL, or epoetin. In some embodiments, the EPO protein is encoded by the nucleic acid identified by the NCBI sequence reference NM 000799, a homolog or a functional fragment thereof. In some embodiments, the EPO protein refers to a polypeptide, a homolog, a variant, a mutant, or a functional fragment as identified by UniProt ID P01588. EPO protein or the polypeptide encoded by the EPO gene is a hormone involved in, e.g., red blood cell production.

The terms “programmed death-ligand 1” or “PD-L1” interchangeably refer to the protein or the gene that encodes the PD-L1 protein and includes any of the PD-L1 naturally occurring forms, homologs, mutants, functional fragments, or variants that maintain the activity of PD-L1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. PD-L1 may also be referred to herein as CD274, B7-H, B7H1, PDCD1L1, PDCD1LG1, CD274 molecule, Programmed cell death ligand 1, hPD-L1, cluster of differentiation 274, B7 homolog 1. In some embodiments, the PD-L1 protein is encoded by the nucleic acid identified by the NCBI sequence reference NM_001314029, NM_001267706, NM_014143, a homolog or a functional fragment thereof. In some embodiments, the PD-L1 protein refers to a polypeptide, a homolog, a variant, a mutant, or a functional fragment as identified by UniProt ID Q9NZQ7. PD-L1 protein or the polypeptide encoded by the PD-L1 gene is a transmembrane protein involved in, e.g., suppression of the adaptive immune system as a checkpoint protein.

The terms “programmed cell death protein 1” or “PD-1” interchangeably refer to the protein or the gene that encodes the PD-1 protein and includes any of the PD-1 naturally occurring forms, homologs, mutants, functional fragments, or variants that maintain the activity of PD-1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. PD-1 may also be referred to herein as PDCD1, CD279, PD-1, PD1, SLEB2, hPD-1, hPD-1, hSLE1, HPD-L, Systemic Lupus Erythematosus Susceptibility 2, Programmed Cell Death Protein 1, or Programmed cell death 1. In some embodiments, the PD-1 protein is encoded by the nucleic acid identified by the NCBI sequence reference NM_005018, a homolog or a functional fragment thereof. In some embodiments, the PD-1 protein refers to a polypeptide, a homolog, a variant, a mutant, or a functional fragment as identified by UniProt ID Q15116. PD-1 protein or the polypeptide encoded by the PD-1 gene is a cell surface protein that behaves, e.g., as an immune checkpoint protein.

The terms “microtubule associated protein tau” or “MAPT” interchangeably refer to the protein or the gene that encodes the MAPT protein and includes any of the MAPT naturally occurring forms, isoforms, homologs, mutants, functional fragments, or variants that maintain the activity of MAPT (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. MAPT may also be referred to herein as DDPAC, FTDP-17, MAPTL, MSTD, MTBT1, MTBT2, PPND, PPP1R103, TAU, PHF-Tau, MGC138549, FLJ31424, DDPAC, microtubule associated protein tau, G Protein Beta1/Gamma2 Subunit-Interacting Factor 1, Protein Phosphatase 1 Regulatory Subunit 103, Neurofibrillary Tangle Protein, Paired Helical Filament-Tau, or Tau proteins. In some embodiments, the MAPT protein is encoded by the nucleic acid identified by the NCBI sequence reference NM_001123066, NM_001123067, NM_001203251, NM_001203252, NM_005910, a homolog or a functional fragment thereof. In some embodiments, the MAPT protein refers to a polypeptide, a homolog, a variant, a mutant, or a functional fragment as identified by UniProt ID P10636. MAPT protein or the polypeptide encoded by the MAPT gene is a protein involved in, e.g., modulation of cell stability and recruiting of cell signaling proteins.

The terms “milk fat globule-EGF Factor 8 protein,” “MFGE8,” or “lactadherin” interchangeably refer to the protein or the gene that encodes the MFGE8 protein and includes any of the MFGE8 naturally occurring forms, isoforms, homologs, mutants, functional fragments, or variants that maintain the activity of MFGE8 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. MFGE8 may also be referred to herein as milk fat globule-EGF factor 8 protein, BA46, EDIL1, HMFG, HsT19888, MFG-E8, MFGM, OAcGD3S, SED1, SPAG10, hP47, milk fat globule EGF, Breast Epithelial Antigen BA46, Sperm Associated Antigen 10, Sperm Surface Protein HP47, Medin, or O-Acetyl Disialoganglioside Synthase. In some embodiments, the MFGE8 protein is encoded by the nucleic acid identified by the NCBI sequence reference NM_001114614, NM_001310319, NM_001310320, NM_001310321, NM_005928, a homolog or a functional fragment thereof. In some embodiments, the MFGE8 protein refers to a polypeptide, a homolog, a variant, a mutant, or a functional fragment as identified by UniProt ID Q08431. MFGE8 protein or the polypeptide encoded by the MFGE8 gene is a cell adhesion protein.

The terms “thymic stromal lymphopoietin” or “TSLP” interchangeably refer to the protein or the gene that encodes the TSLP protein and includes any of the TSLP naturally occurring forms, homologs, mutants, functional fragments, or variants that maintain the activity of TSLP (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TSLP may also be referred to herein as thymic stromal lymphopoietin. In some embodiments, the TSLP protein is encoded by the nucleic acid identified by the NCBI sequence reference NM_033035, NM_138551, a homolog or a functional fragment thereof. In some embodiments, the TSLP protein refers to a polypeptide, a homolog, a variant, a mutant, or a functional fragment as identified by UniProt ID Q969D9. TSLP protein or the polypeptide encoded by the TSLP gene is a cytokine involved in the maturation of T cells through activation of antigen-presenting cells.

The terms “thrombospondin” or “TSP” interchangeably refer to the proteins or the genes that encodes the TSP protein and includes any of the TSP naturally occurring forms, homologs, mutants, functional fragments, or variants that maintain the activity of TSP (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TSP may also be referred to herein as thymic stromal lymphopoietin, THBS1, THBS, THBS-1, TSP, TSP-1, TSP1, thrombospondin 1, thrombospondin 2, thrombospondin 3, thrombospondin 4, thrombospondin 5, THBS-2, THBS-3, THBS-4, THBS-5, THBS2, THBS3, THBS4, THBS5, TSP-2, TSP2, TSP-3, TSP3, TSP-4, TSP4, TSP-5, TSP5, or Glycoprotein G. In some embodiments, the TSP protein is encoded by the nucleic acid identified by the NCBI sequence reference NM 003246, XM_011521971.2, NM_001252607.2, NM_001252608.2, NM 007112.5, NM_003248.6, NM_001306212.2, NM 001306213.2, NM_001306214.2, a homolog or a functional fragment thereof. In some embodiments, the TSP protein refers to a polypeptide, a homolog, a variant, a mutant, or a functional fragment as identified by UniProt ID P07996, P35442, P49746 or P35443. TSP proteins or the polypeptides encoded by the TSP gene are a family of proteins involved in formation and maintenance of the extracellular matrix.

The terms “fibroblast growth factor 23” or “FGF23” interchangeably refer to the protein or the gene that encodes the FGF23 protein and includes any of the FGF23 naturally occurring forms, homologs, mutants, functional fragments, or variants that maintain the activity of FGF23 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. FGF23 may also be referred to herein as ADHR, FGFN, HPDR2, HYPF, PHPTC, fibroblast growth factor 23, Tumor-Derived Hypophosphatemia Inducing Factor, or HFTC2. In some embodiments, the FGF23 protein is encoded by the nucleic acid identified by the NCBI sequence reference NM_020638, a homolog or a functional fragment thereof. In some embodiments, the FGF23 protein refers to a polypeptide, a homolog, a variant, a mutant, or a functional fragment as identified by UniProt ID Q9GZV9. FGF23 protein or the polypeptide encoded by the FGF23 gene is a protein that participates in phosphate and vitamin D metabolism and regulation.

The terms “tissue inhibitor of metalloproteinases 1” or “TIMP1” interchangeably refer to the protein or the gene that encodes the TIMP1 protein and includes any of the TIMP1 naturally occurring forms, homologs, mutants, functional fragments, or variants that maintain the activity of TIMP1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TIMP1 may also be referred to herein as CLGI, EPA, EPO, HCl, TIMP, TIMP-1, Tissue Inhibitor Of Metalloproteinases 1, Fibroblast Collagenase Inhibitor, Collagenase Inhibitor, Epididymis Secretory Sperm Binding Protein, Erythroid Potentiating Activity, Erythroid-Potentiating Activity, or TIMP metallopeptidase inhibitor 1. In some embodiments, the TIMP1 protein is encoded by the nucleic acid identified by the NCBI sequence reference NM_003254, a homolog or a functional fragment thereof. In some embodiments, the TIMP1 protein refers to a polypeptide, a homolog, a variant, a mutant, or a functional fragment as identified by UniProt ID P01033 or Q6FGX5. TIMP1 protein or the polypeptide encoded by the TIMP1 gene is a protein involved in, e.g., inhibition of matrix metalloproteinases.

The terms “adenosine deaminase 2” or “ADA2” interchangeably refer to the protein or the gene that encodes the ADA2 protein and includes any of the ADA2 naturally occurring forms, homologs, mutants, functional fragments, or variants that maintain the activity of ADA2 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ADA2 may also be referred to herein as ADGF, Cat Eye Syndrome Chromosome Region, Candidate 1, Cat Eye Syndrome Critical Region Protein 1, CECRI, IDGFL, Adenosine Deaminase CECRI, EC 3.5.4.4, SNEDS, VAIHS, or PAN. In some embodiments, the ADA2 protein is encoded by the nucleic acid identified by the NCBI sequence reference NM_001282225.2, NM 177405.3, NM 001282226.2, NM_001282227.2, NM_001282228.2, NM_001282229.2, a homolog or a functional fragment thereof. In some embodiments, the ADA2 protein refers to a polypeptide, a homolog, a variant, a mutant, or a functional fragment as identified by UniProt ID Q9NZK5. ADA2 protein or the polypeptide encoded by the ADA2 gene is involved in the metabolism of adenosine and related purine molecules.

The terms “apelin” or “APLN” interchangeably refer to the protein or the gene that encodes the APLN protein and includes any of the APLN naturally occurring forms, homologs, mutants, functional fragments, or variants that maintain the activity of APLN (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. APLN may also be referred to herein as APEL, XNPEP2, APJ Endogenous Ligand, or AGTRL1 Ligand. In some embodiments, the APLN protein is encoded by the nucleic acid identified by the NCBI sequence reference NM_017413, a homolog or a functional fragment thereof. In some embodiments, the APLN protein refers to a polypeptide, a homolog, a variant, a mutant, or a functional fragment as identified by UniProt ID Q9ULZ1. APLN protein or the polypeptide encoded by the APLN gene is a protein involved in numerous pathways, including control of blood pressure.

The terms “apolipoprotein B” or “ApoB” interchangeably refer to the protein or the gene that encodes the ApoB protein and includes any of the ApoB naturally occurring forms, homologs, mutants, functional fragments, or variants that maintain the activity of ApoB (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ApoB may also be referred to herein as FLDB, LDLCQ4, apoB-100, apoB-48, apolipoprotein B, FCHL, Apolipoprotein B-100, Apolipoprotein B48, or FCHL2. In some embodiments, the ApoB protein is encoded by the nucleic acid identified by the NCBI sequence reference NM_000384, a homolog or a functional fragment thereof. In some embodiments, the ApoB protein refers to a polypeptide, a homolog, a variant, a mutant, or a functional fragment as identified by UniProt ID P04114. APLN protein or the polypeptide encoded by the ApoB gene is the primary apolipoprotein of various lipoprotein particles, such as low-density lipoprotein (LDL) particles.

The terms “lipoprotein lipase” or “LPL” interchangeably refer to the protein or the gene that encodes the LPL protein and includes any of the LPL naturally occurring forms, homologs, mutants, functional fragments, or variants that maintain the activity of LPL (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. LPL may also be referred to herein as HDLCQ11, LIPD, EC 3.1.1.34, Phospholipase A1, EC 3.1.1.32, or EC 3.1.1. In some embodiments, the LPL protein is encoded by the nucleic acid identified by the NCBI sequence reference NM 000237, a homolog or a functional fragment thereof. In some embodiments, the LPL protein refers to a polypeptide, a homolog, a variant, a mutant, or a functional fragment as identified by UniProt ID P06858. LPL protein or the polypeptide encoded by the LPL gene is an enzyme that hydrolyzes triglycerides in various lipoproteins.

The terms “lipoprotein(a)” or “Lp(a)” interchangeably refer to the protein or the gene that encodes the Lp(a) protein and includes any of the Lp(a) naturally occurring forms, homologs, mutants, functional fragments, or variants that maintain the activity of Lp(a) (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. Lp(a) may also be referred to herein as AK38, APOA, LP, Apolipoprotein(A), Lipoprotein, Lp(A), Apo(A), EC 3.4.21.7, EC 3.4.21, EC 3.4.21, LPA, or Antiangiogenic AK38 Protein. In some embodiments, the Lp(a) protein is encoded by the nucleic acid identified by the NCBI sequence reference NM_005577, a homolog or a functional fragment thereof. In some embodiments, the Lp(a) protein refers to a polypeptide, a homolog, a variant, a mutant, or a functional fragment as identified by UniProt ID P08519. Lp(a) protein or the polypeptide encoded by the Lp(a) gene is a low-density lipoprotein variant containing the apolipoprotein(a). Lipoprotein(a) has been identified as a risk factor for certain cardiovascular diseases and conditions, such as atherosclerosis, coronary heart disease, and stroke.

The terms “apolipoprotein C3” or “ApoCIII” interchangeably refer to the protein or the gene that encodes the ApoCIII protein and includes any of the ApoCIII naturally occurring forms, homologs, mutants, functional fragments, or variants that maintain the activity of ApoCIII (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ApoCIII may also be referred to herein as APOC3, HALP2, apolipoprotein-C3, or Apolipoprotein C-III. In some embodiments, the ApoCIII protein is encoded by the nucleic acid identified by the NCBI sequence reference NM_000040, a homolog or a functional fragment thereof. In some embodiments, the ApoCIII protein refers to a polypeptide, a homolog, a variant, a mutant, or a functional fragment as identified by UniProt ID P02656. ApoCIII protein or the polypeptide encoded by the ApoCIII gene is a protein secreted by the small intestine and liver and found on triglyceride-rich lipoproteins.

The terms “angiopoietin-like 3” or “ANGPTL3” interchangeably refer to the protein or the gene that encodes the ANGPTL3 protein and includes any of the ANGPTL3 naturally occurring forms, homologs, mutants, functional fragments, or variants that maintain the activity of ANGPTL3 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ANGPTL3 may also be referred to herein as ANG-5, ANGPT5, ANL3, FHBL2, Angiopoietin 5, or Angiopoietin-like Protein 3. In some embodiments, the ANGPTL3 protein is encoded by the nucleic acid identified by the NCBI sequence reference NM_014495, a homolog or a functional fragment thereof. In some embodiments, the ANGPTL3 protein refers to a polypeptide, a homolog, a variant, a mutant, or a functional fragment as identified by UniProt ID Q9Y5C1. ANGPTL3 protein or the polypeptide encoded by the ANGPTL3 gene is a secreted factor that behaves as a dual inhibitor of lipoprotein lipase (LPL) and endothelial lipase (EL).

The terms “angiopoietin-like 4” or “ANGPTL4” interchangeably refer to the protein or the gene that encodes the ANGPTL4 protein and includes any of the ANGPTL4 naturally occurring forms, homologs, mutants, functional fragments, or variants that maintain the activity of ANGPTL4 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ANGPTL4 may also be referred to herein as ANGPTL2, ARP4, FIAF, HARP, HFARP, NL2, PGAR, TGQTL, UNQ171, pp 1158, Hepatic Fibrinogen/Angiopoietin-Related Protein, Angiopoietin-Related Protein 4, Peroxisome Proliferator-Activated Receptor (PPAR) Gamma Induced Angiopoietin-Related Protein, Hepatic Angiopoietin-Related Protein, PPARG Angiopoietin Related Protein, Fasting-Induced Adipose Factor, or Angiopoietin-like Protein 4. In some embodiments, the ANGPTL4 protein is encoded by the nucleic acid identified by the NCBI sequence reference NM 001039667, NM_016109, NM 139314, a homolog or a functional fragment thereof. In some embodiments, the ANGPTL4 protein refers to a polypeptide, a homolog, a variant, a mutant, or a functional fragment as identified by UniProt ID Q9BY76. ANGPTL4 protein or the polypeptide encoded by the ANGPTL4 gene is secreted target of certain peroxisome proliferator-activated receptors.

The terms “angiopoietin-like 8,” “ANGPTL8,” or “lipasin,” interchangeably refer to the protein or the gene that encodes the ANGPTL8 protein and includes any of the ANGPTL8 naturally occurring forms, homologs, mutants, functional fragments, or variants that maintain the activity of ANGPTL8 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. ANGPTL8 may also be referred to herein as PRO 1185, PVPA599, RIFL, TD26, C₁₉orf80, Betatrophin, Refeeding-Induced Fat And Liver Protein, Betatrophin, Hepatocellular Carcinoma-Associated Protein TD26, Betatrophin Variant 1, Betatrophin Variant 2, or Angiopoietin-like Protein 8. In some embodiments, the ANGPTL8 protein is encoded by the nucleic acid identified by the NCBI sequence reference NM_018687, a homolog or a functional fragment thereof. In some embodiments, the ANGPTL8 protein refers to a polypeptide, a homolog, a variant, a mutant, or a functional fragment as identified by UniProt ID Q6UXH0. ANGPTL8 protein or the polypeptide encoded by the ANGPTL8 gene is an inhibitor of lipoprotein lipase (LPL).

The terms “bone morphogenetic protein 6” or “BMP6” interchangeably refer to the protein or the gene that encodes the BMP6 protein and includes any of the BMP6 naturally occurring forms, homologs, mutants, functional fragments, or variants that maintain the activity of BMP6 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. BMP6 may also be referred to herein as VGR, VGR1, VGR-1, VG-1-R, VG-1-Related Protein, or Vegetal Related Growth Factor (TGFB-Related). In some embodiments, the BMP6 protein is encoded by the nucleic acid identified by the NCBI sequence reference NM_001718, a homolog or a functional fragment thereof. In some embodiments, the BMP6 protein refers to a polypeptide, a homolog, a variant, a mutant, or a functional fragment as identified by UniProt ID P22004. BMP6 protein or the polypeptide encoded by the BMP6 gene is involved in the induction of osteogenic markers in mesenchymal stem cells.

The terms “bone morphogenetic protein 9/growth differentiation factor 2” or “BMP9/GDF2” interchangeably refer to the protein or the gene that encodes the BMP9/GDF2 protein and includes any of the BMP9/GDF2 naturally occurring forms, homologs, mutants, functional fragments, or variants that maintain the activity of BMP9/GDF2 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. BMP9/GDF2 may also be referred to herein as BMP-9, BMP9, HHT5, or growth differentiation factor 2. In some embodiments, the BMP9/GDF2 protein is encoded by the nucleic acid identified by the NCBI sequence reference NM_016204, a homolog or a functional fragment thereof. In some embodiments, the BMP9/GDF2 protein refers to a polypeptide, a homolog, a variant, a mutant, or a functional fragment as identified by UniProt ID Q9UK05. BMP9/GDF2 protein or the polypeptide encoded by the BMP9/GDF2 gene plays a role in maintenance of neurons and their response to acetylcholine.

The terms “colony stimulating factor 1 receptor” or “CSF-1” interchangeably refer to the protein or the gene that encodes the CSF-1 protein and includes any of the CSF-1 naturally occurring forms, homologs, mutants, functional fragments, or variants that maintain the activity of CSF-1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. CSF-1 may also be referred to herein as, MCSF, CSF1, MGC31930, colony stimulating factor 1, Macrophage Colony Stimulating Factor 1, or Macrophage colony-stimulating factor. In some embodiments, the CSF-1 protein is encoded by the nucleic acid identified by the NCBI sequence reference NM_172212, NM_000757, NM_172210, NM_172211, a homolog or a functional fragment thereof. In some embodiments, the CSF-1 protein refers to a polypeptide, a homolog, a variant, a mutant, or a functional fragment as identified by UniProt ID P09603. CSF-1 protein or the polypeptide encoded by the CSF-1 gene is a hematopoietic growth factor involved in the proliferation, differentiation, and survival of immune cells.

The terms “growth differentiation factor 15” or “GDF15” interchangeably refer to the protein or the gene that encodes the GDF15 protein and includes any of the GDF15 naturally occurring forms, homologs, mutants, functional fragments, or variants that maintain the activity of GDF15 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. GDF15 may also be referred to herein as, GDF-15, MIC-1, MIC1, NRG-1, NAG-1, PDF, PLAB, PTGFB, growth differentiation factor 15, TGF-PL, Macrophage Inhibitory Cytokine 1, NSAID-Activated Gene 1 Protein, NSAID (Nonsteroidal Anti-Inflammatory Drug)-Activated Protein 1, Placental TGF-Beta, Placental Bone Morphogenetic Protein, or Non-Steroidal Anti-Inflammatory Drug-Activated Gene-1. In some embodiments, the GDF15 protein is encoded by the nucleic acid identified by the NCBI sequence reference NM 004864, a homolog or a functional fragment thereof. In some embodiments, the GDF15 protein refers to a polypeptide, a homolog, a variant, a mutant, or a functional fragment as identified by UniProt ID Q99988. GDF15 protein or the polypeptide encoded by the GDF15 gene plays a role in regulation of inflammatory pathways and apoptosis.

The terms “complement Factor B” or “CFB” interchangeably refer to the protein or the gene that encodes the CFB protein and includes any of the CFB naturally occurring forms, homologs, mutants, functional fragments, or variants that maintain the activity of CFB (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. CFB may also be referred to herein as Cfb, AI195813, AI255840, Bf, C2, Fb, H2-Bf, AHUS4, ARMD14, BFD, CFAB, CFBD, FBIl2, GBG, PBF2, Properdin Factor B, C3/C5 Convertase, EC 3.4.21.47, BF, Glycine-Rich Beta Glycoprotein, C3 Proaccelerator, C3 Proactivator, or complement-factor B. In some embodiments, the CFB protein is encoded by the nucleic acid identified by the NCBI sequence reference NM_001710, a homolog or a functional fragment thereof. In some embodiments, the CFB protein refers to a polypeptide, a homolog, a variant, a mutant, or a functional fragment as identified by UniProt ID P00751. CFB protein or the polypeptide encoded by the CFB gene is a component of the alternative pathway of complement activation.

The terms “complement Factor D” or “CFD” interchangeably refer to the protein or the gene that encodes the CFD protein and includes any of the CFD naturally occurring forms, homologs, mutants, functional fragments, or variants that maintain the activity of CFD (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. CFD may also be referred to herein as Cfd, ADN, PFD, DF, EC 3.4.21.46, EC 3.4.21, C3 proactivator convertase, C3 Convertase Activator, properdin factor D esterase, Properdin Factor D, factor D (complement), complement factor D, CFD, adipsin, or complement-factor D. In some embodiments, the CFD protein is encoded by the nucleic acid identified by the NCBI sequence reference NM_001928, a homolog or a functional fragment thereof. In some embodiments, the CFD protein refers to a polypeptide, a homolog, a variant, a mutant, or a functional fragment as identified by UniProt ID P00746. CFD protein or the polypeptide encoded by the CFD gene is a component of the alternative pathway of complement activation.

The terms “complement component 5” or “C5” interchangeably refer to the protein or the gene that encodes the C5 protein and includes any of the C5 naturally occurring forms, homologs, mutants, functional fragments, or variants that maintain the activity of C5 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. C5 may also be referred to herein as CSD, C5a, C5b, CPAMD4, ECLZB, complement component 5, complement C5, C5a Anaphylatoxin, Prepro-C5, or C3 And PZP-Like Alpha-2-Macroglobulin Domain-Containing Protein 4. In some embodiments, the C5 protein is encoded by the nucleic acid identified by the NCBT sequence reference NM_001735. NM 001317163. NM_001317164, a homolog or a functional fragment thereof. In some embodiments, the C5 protein refers to a polypeptide, a homolog, a variant, a mutant, or a functional fragment as identified by UniProt ID P01031. In some embodiments, C5 protein comprises C5a, and C5b. C5 protein or the polypeptide encoded by the C5 gene is a component of the complement system, and is cleaved into C5a and C5b.

The terms “C—X—C motif chemokine ligand 10” or “CXCL10” interchangeably refer to the protein or the gene that encodes the CXCL10 protein and includes any of the CXCL10 naturally occurring forms, homologs, mutants, functional fragments, or variants that maintain the activity of CXCL10 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. CXCL10 may also be referred to herein as IFI10, INP10, IP-10, SCYB10, crg-2, gIP-10, GIP-10, Mob-1, C7, mob-1, Gamma IP10, Gamma-IP10, small-inducible cytokine B10, Interferon-Inducible Cytokine IP-10, 10 KDa Interferon Gamma-Induced Protein, C—X—C motif chemokine ligand 10, C—X—C motif chemokine 10, Small Inducible Cytokine Subfamily B (Cys-X-Cys), Member 10, or Interferon gamma-induced protein 10. In some embodiments, the CXCL10 protein is encoded by the nucleic acid identified by the NCBI sequence reference NM_001565, a homolog or a functional fragment thereof. In some embodiments, the CXCL10 protein refers to a polypeptide, a homolog, a variant, a mutant, or a functional fragment as identified by UniProt ID P02778. CXCL10 protein or the polypeptide encoded by the CXCL10 gene is a cytokine secreted in response to interferon-gamma and binds to the cell surface chemokine receptor CXCR3.

The terms “myeloperoxidase” or “MPO” interchangeably refer to the protein or the gene that encodes the MPO protein and includes any of the MPO naturally occurring forms, homologs, mutants, functional fragments, or variants that maintain the activity of MPO (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. MPO may also be referred to herein as EC 1.11.1.7, EC 1.11.2.2 or EC 1.11.1. In some embodiments, the MPO protein is encoded by the nucleic acid identified by the NCBI sequence reference NM 000250, a homolog or a functional fragment thereof. In some embodiments, the MPO protein refers to a polypeptide, a homolog, a variant, a mutant, or a functional fragment as identified by UniProt ID P05164. MPO protein or the polypeptide encoded by the MPO gene is a peroxidase in the XPO subfamily of peroxidases. Elevated levels of MPO have been associated with the severity of coronary artery disease.

The terms “progranulin” refers to the protein or the gene that encodes the progranulin protein and includes any of the progranulin naturally occurring forms, homologs, mutants, functional fragments, or variants that maintain the activity of progranulin (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. progranulin may also be referred to herein as GRN, CLN11, GEP, GP88, PCDGF, PEPI, PGRN, PGranulin, granulin precursor, proepithelin, acrogranin, Epithelin Precursor, Granulin-Epithelin, Granulins, or PC Cell-Derived Growth Factor. In some embodiments, the progranulin protein is encoded by the nucleic acid identified by the NCBI sequence reference NM_001012479, NM_002087, a homolog or a functional fragment thereof. In some embodiments, the progranulin protein refers to a polypeptide, a homolog, a variant, a mutant, or a functional fragment as identified by UniProt ID P28799. Progranulin protein or the polypeptide encoded by the progranulin gene is a protein involved in regulation of cell growth, survival, repair and inflammation, e.g., in the central nervous system.

The terms “transforming growth factor beta 1” or “TGF-beta 1” interchangeably refer to the protein or the gene that encodes the TGF-beta 1 protein and includes any of the TGF-beta 1 naturally occurring forms, homologs, mutants, functional fragments, or variants that maintain the activity of TGF-beta 1 (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. TGF-beta 1 may also be referred to herein as CED, DPD1, LAP, IBDIMDE, TGFB, TGFbeta, transforming growth factor beta 1, IBDIMDE, TGFB1, TGFB-1, TGF-beta1, Latency-Associated Peptide, Diaphyseal Dysplasia 1, Progressive, or Prepro-Transforming Growth Factor Beta-1. In some embodiments, the TGF-beta 1 protein is encoded by the nucleic acid identified by the NCBI sequence reference NM_000660, a homolog or a functional fragment thereof. In some embodiments, the TGF-beta 1 protein refers to a polypeptide, a homolog, a variant, a mutant, or a functional fragment as identified by UniProt ID P01137. TGF-beta 1 protein or the polypeptide encoded by the TGF-beta 1 gene is a cytokine involved in many regulatory pathways.

The terms “vascular endothelial growth factor A” or “VEGF-A” interchangeably refer to the protein or the gene that encodes the VEGF-A protein and includes any of the VEGF-A naturally occurring forms, homologs, mutants, functional fragments, or variants that maintain the activity of VEGF-A (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. VEGF-A may also be referred to herein as MVCD1, VEGFA, VEGF, VPF, vascular endothelial growth factor A, Vascular Endothelial Growth Factor A121, Vascular Endothelial Growth Factor A165, or Vascular Permeability Factor. In some embodiments, the VEGF-A protein is encoded by the nucleic acid identified by the NCBI sequence reference NM_003376, NM_001025366, NM_001025367, NM_001025368, NM_001025369, a homolog or a functional fragment thereof. In some embodiments, the VEGF-A protein refers to a polypeptide, a homolog, a variant, a mutant, or a functional fragment as identified by UniProt ID P015692. VEGF-A protein or the polypeptide encoded by the VEGF-A gene is a member of the platelet-derived growth factor/vascular endothelial growth factor family.

The terms “urokinase-type plasminogen activator receptor” or “suPAR” interchangeably refer to the protein or the gene that encodes the suPAR protein and includes any of the suPAR naturally occurring forms, homologs, mutants, functional fragments, or variants that maintain the activity of suPAR (e.g., within at least 50%, 80%, 90%, 95%, 96%, 97%, 98%, 99% or 100% activity compared to the native protein). In some embodiments, homologs, mutants, or variants have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. suPAR may also be referred to herein as PLAUR, CD87, CD87 antigen, M03, U-PAR, UPAR, URKR, plasminogen activator, urokinase receptor, Monocyte Activation Antigen Mo3, U-Plasminogen Activator Receptor Form 2, or Urokinase Plasminogen Activator Surface Receptor. In some embodiments, the suPAR protein is encoded by the nucleic acid identified by the NCBI sequence reference NM_001005376, NM_001005377, NM_001301037, NM_002659, a homolog or a functional fragment thereof. In some embodiments, the suPAR protein refers to a polypeptide, a homolog, a variant, a mutant, or a functional fragment as identified by UniProt ID Q03405. suPAR protein or the polypeptide encoded by the uPAR gene is the soluble form of the urokinase-type plasminogen activator receptor (uPAR) and a biomarker for the activation of the inflammatory and immune systems.

The term “therapeutic apheresis” used herein refers to an extracorporeal treatment that selectively removes abnormal cells or substances in the blood that are associated with or causative of certain disease states.

### Bifunctional Compounds

The present disclosure features bifunctional compounds comprising a first moiety capable of binding to an extracellular target (i.e., A_{G}) and a second moiety capable of binding to a membrane-bound receptor associated with a degradation pathway (i.e., R_{G}). In some embodiments, the bifunctional compound described herein has the structure of Formula (I):
A_{G}-L-R_{G} (I) or a pharmaceutically acceptable salt thereof, wherein:
A_{G} is a protein moiety (e.g., an antibody or a fragment thereof, a receptor or a fragment thereof, or an antigen protein or a fragment thereof that binds to an extracellular target;
L is absent or a linker; and
R_{G} is a moiety that binds to a membrane-bound receptor associated with a degradation pathway.

Each of the component moieties is described in more detail herein.

### Targeting Binding Moiety (A_{G})

The targeting binding moiety (i.e., A_{G}) may be a protein moiety that binds to an extracellular target molecule. In some embodiments, A_{G} is the portion of the bifunctional moiety that confers target specificity to the bifunctional compound. A_{G} may be an antibody molecule or a fragment thereof, a receptor molecule, or an antigen protein or a fragment thereof.

In some embodiments, A_{G} is an antibody or fragment thereof. In some embodiments, the antibody or fragment thereof comprises an antigen-binding domain that binds to an extracellular target. In an embodiment, A_{G} is a full-length antibody. In an embodiment, A_{G} is an antibody fragment. The antibody fragment may comprise a Fab, a Fab′, a F(ab′)₂, a F(ab)₂, variable fragment (Fv), a domain antibody (dAb), a single domain antibody, or a single chain variable fragment (scFv). The antibody may further be a monospecific antibody or a multispecific, e.g., bispecific or trispecific, antibody molecule. In an embodiment, A_{G} comprises an anti-ApoB antibody, an anti-PCSK9 antibody, anti-MICA antibody, an anti-EGFR antibody, or an anti-LDL antibody. In an embodiment, A_{G} comprises anti-ApoB antibody. In an embodiment, A_{G} comprises an anti-PCSK9 antibody. In an embodiment, A_{G} comprises an anti-MICA antibody. In an embodiment, A_{G} comprises an anti-EGFR antibody. In an embodiment, A_{G} comprises an anti-LDL antibody. In an embodiment, A_{G} comprises an antibody fragment comprising anti-ApoB Fab fragment, an anti-PCSK9 Fab fragment, and anti-MICA Fab fragment, an anti-EGFR Fab fragment, or an anti-LDL Fab fragment. In an embodiment, A_{G} comprises anti-ApoB Fab fragment. In an embodiment, A_{G} comprises an anti-PCSK9 Fab fragment. In an embodiment, A_{G} comprises an anti-MICA Fab fragment. In an embodiment, A_{G} comprises an anti-EGFR Fab fragment. In an embodiment, A_{G} comprises an anti-LDL Fab fragment.

In some embodiments, A_{G} comprises an antigen protein or a fragment thereof (e.g., peptide). An antigen protein may comprise a fragment of a cell surface antigen, such as a human cluster of differentiation (CD) protein. For example, an antigen protein may comprise one or more sequence within CD1a, CD1b, CD1c, CD1d, CD1e, CD2, CD3, CD4, CD5, CD6, CD7, CD8, CD9, CD10, CD11, CD13, CD14, CD15, CD16, CD17, CD18, CD19, CD20, CD21, CD2, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49, CD50, CD51, CD52, CD53, CD54, CD55, CD56, CD57, CD58, CD59, CD60, CD61, CD62, CD63, CD64. CD65, CD66, CD67, CD68, CD69, CD70, CD71, CD72, CD73, CD74, CD75, CD77, CD79, CD80, or other CD protein. In some embodiments, the antigen protein or a fragment thereof recognizes a pathogenic autoantibody. Exemplary antigen proteins include a disintegrin and metalloproteinase with a thrombospondin type 1 motif, member 13 (ADAMTS13), steroidogenic cytochrome P450 enzyme 21-hydroxylase, N-methyl-d-aspartate-(NMDA)-receptor, erythrocytes, anti-smooth muscle antibodies (ASSMAs), actin, platelet, signal recognition particle (SRP), 3-hydroxy-3-methyl-glutaryl-coenzyme A reductase (HMGCR), myosin, sperm, amylase alpha2, type XVII collagen (col17), kallikrein 13, type VII collagen (col7), myeloperoxidase (MPO), type IV collagen, proteinase 3 (PR3), thyrotropin receptor (TSHR), thyroglobulin, thyroid peroxidase (TPO), thyroglobulin, thyroid peroxidase (TPO), platelets, myeloperoxidase (MPO), muscle nicotinic acetylcholine receptors, muscle-specific kinase (MuSK), low-density lipoprotein receptor protein 4 (LRP4), myosin, beta1 adrenergic receptor, adenine-nucleotide translocase, aquaporin-4, myelin oligodendrocyte glycoprotein (MOG), heat shock protein 90 (HSP90), heat shock protein A5 (HSPA5), desmoglein-3, parietal cells, mitochondria, phospholipase A2 receptor (PLA2R), thrombospondin type 1 domain-containing 7A (THSD7A), cyclic citrullinated proteins, RNA binding proteins (Ros), La, double-stranded DNA (dsDNA), angiotensin II type 1 receptor (ATIR), endothelin-1 type A receptor (ETAR), insulin, glutamic acid decarboxylase, protein tyrosine phosphatase, and proprotein convertase subtilisin kexin type 9 (PCSK9).

In some embodiments, A_{G} is a receptor or a fragment thereof. A_{G} may be a full-length receptor, a receptor fragment, or a functional variant thereof. In some embodiments, A_{G} is a cell surface receptor or a fragment thereof. Exemplary receptors include an epidermal growth factor receptor, low density lipoprotein receptor (LDLR), an integrin, an immune receptor (e.g., a Toll-like receptor, a T cell receptor), a fibroblast growth factor receptor, an insulin receptor, a receptor tyrosine kinase, an olfactory receptor, an adrenergic receptor, or an ephrin receptor. In an embodiment, A_{G} comprises the LDLR or a fragment thereof.

The A_{G} binds to the extracellular target, which may be a soluble (e.g., plasma) protein, a membrane-associated protein, or a lipoprotein. In some embodiments, the extracellular target molecule is an antibody, a lipoprotein, a plasma membrane protein, a capsid, a virus, a soluble receptor, a secreted protein, a growth factor, a cytokine, a chemokine, a hormone, a neurotransmitter, an exosome, a cell, or a combination thereof. In some embodiments, the extracellular target is a soluble protein, or a component thereof (e.g., a protein modification, e.g., sugar). The soluble protein may be an antibody, a soluble receptor, a secreted protein, a growth factor, a cytokine, a hormone, a neurotransmitter, or an enzyme. The soluble protein may be a monomer or oligomer (e.g., a dimer, trimer, tetramer, or higher order species). In some embodiments, the soluble protein may comprise a posttranslational modification, such as a sugar (e.g., a monosaccharide, oligosaccharide, phosphate group, lipid, or additional amino acid). In some embodiments, the soluble protein may be found in the blood, lymph, or fat tissue. Exemplary soluble proteins include proprotein convertase subtilisin/kexin type 9 (PCSK9), complement factor H-related protein 3 (CFHR3), MICA (MHC class I chain-related gene A), and apolipoprotein-B (Apo-B).

In some embodiments, the extracellular target is a membrane-associated protein, or a component thereof (e.g., a protein modification, e.g., sugar). A membrane-associated protein may be covalently or non-covalently associated with a cell membrane, e.g., is a transmembrane protein or membrane-anchored protein. In some embodiments, the membrane-associated protein comprises a transmembrane domain, e.g., an alpha helix or loop. In some embodiments, the membrane-associated protein comprises a channel or pore. In some embodiments, the membrane-associated protein is chosen from a type I, type II, or multipass membrane protein or a glycophosphatidylinositol (GPI) anchored membrane associated protein, e.g., a receptor, a protein channel, or an ion channel. Exemplary membrane-associated proteins include TNFR1 (tumor necrosis factor receptor 1), IL1R (interleukin 1 receptor), EGFR (epidermal growth factor receptor), CXCR4 (C—X—C chemokine receptor type 4), TSP1 (thrombospondin 1), NKG2D (encoded by KLRK1), uPAR (urokinase plasminogen activator receptor), GFRAL, IL4R, thymic stromal lymphopoietin receptor, IL7R, TGFβ receptor, PD-L1, or sToll receptor.

In some embodiments, the extracellular target is a lipoprotein. A lipoprotein may comprise lipids and proteins, and can include various plasma lipoprotein particles. Exemplary lipoproteins include lipoprotein(a), low-density lipoproteins (LDL), chylomicrons, very-low-density lipoproteins (VLDL), intermediate-density lipoproteins (IDL), and high-density lipoproteins (HDL).

In some embodiments, the extracellular target is a pathogenic target (i.e., a target associated with (e.g., causes) a deleterious or unwanted effect in a cell or a subject). A pathogenic target may be associated with a certain disease, disorder, condition, or clinical situation, or symptom thereof. In some embodiments, the pathogenic target is an extracellular secreted protein, e.g., soluble protein (PCSK9), extracellular lipoprotein (e.g., LDLR), infectious agent (virus, bacteria, or parasite). In some embodiments, the pathogenic target is a pathogenic autoantibody. In some embodiments, the pathogenic target is an anti-drug antibody. In some embodiments, the pathogenic target is a membrane-associated protein (e.g., TSP1, uPAR). In some embodiments, the pathogenic target is a cell surface receptor, e.g., TNF receptor 1 (TNFR1), interleukin-1 receptor (IL1R), PD-L1, epidermal growth factor receptor (EGFR), CXCR4, NKG2D, GFRAL, IL4R, thymic stromal lymphopoietin receptor, IL-7R, TGFβ receptor or sToll receptor. In some embodiments, the pathogenic target is a neurological target, e.g., a Tau protein or aggregate; or an immunooncology target, e.g., progranulin. In some embodiments, the pathogenic target is a toxin or a drug (e.g., a reversal agent, e.g., any agent used to reverse the deleterious effect of a previously administered drug or treatment, such as sedation or a narcotic).

### Receptor Binding Moiety (R_{G})

The receptor binding moiety (R_{G}) is a moiety that binds to a membrane-bound receptor associated with a degradation pathway.

In some embodiments, R_{G} comprises a small molecule, a carbohydrate or carbohydrate derivative, an antibody molecule, a peptide, or a pharmaceutically acceptable salt thereof. In some embodiments, R_{G} comprises a small molecule. In some embodiments, R_{G} comprises a carbohydrate or carbohydrate derivative. In some embodiments, R_{G} comprises an antibody molecule. In some embodiments, R_{G} comprises a peptide. In some embodiments, R_{G} comprises a hexasaccharide moiety (e.g., a glucose, galactose, mannose, N-acetylglucose, N-acetylgalactose, N-acetylmannose, mannose-6-phosphate, or mannose-6-phosphonate moiety). In some embodiments, R_{G} comprises a plurality of hexasaccharide moieties (e.g., a plurality of glucose, galactose, mannose, N-acetylglucose, N-acetylgalactose, N-acetylmannose, mannose-6-phosphate, or mannose-6-phosphonate moieties). In some embodiments, R_{G} comprises one or more N-acetylgalactose (GalNAc) moieties, e.g., at least 1, 2, 3, 4, 5, or 6 GalNAc moieties. In some embodiments, R_{G} comprises 3 GalNAc moieties.

In some embodiments, R_{G} comprises a binding moiety for the asialoglycoprotein receptor (ASGPR). ASGPR is a C-type lectin expressed on the surface of hepatocytes which regulates levels of plasma glycoproteins terminated with galactose (Gal) or N-acetylgalactosamine (GalNAc) sugars. The ASGPR binds the glycoproteins terminated with galactose (Gal) or N-acetylgalactosamine (GalNAc) sugars and is internalized via receptor mediated endocytosis primarily in coated pits on the basolateral membrane of hepatocytes. Upon internalization, the ligand-receptor complex is transported to endo-lysosomal compartments. Calcium sequestration and subsequent acidification of endosomal compartments promotes dissociation of the ligand-receptor complex, and the receptor is recycled back to the plasma membrane while the cargo (ligand) is sorted to lysosomes for degradation. In some embodiments, the binding moiety for the ASGPR comprises a carbohydrate or carbohydrate derivative, a small molecule, an antibody molecule or a fragment thereof, a peptide, or a pharmaceutically acceptable salt thereof (e.g., R_{G} comprises a Gal, GalNAc, small molecule, an antibody molecule or fragment thereof, a peptide, or a pharmaceutically acceptable salt thereof). In some embodiments, the binding moiety for the ASGPR comprises galactose (Gal) or N-acetylgalactosamine (GalNAc) sugar. In some embodiments, the binding moiety for the ASGPR is a GalNAc trimer or a bridged GalNAc trimer and monomer (e.g., R_{G} comprises a GalNAc trimer or a bridged GalNAc trimer and monomer). In some embodiments, R_{G} comprises a GalNAc trimer or bridged GalNAc trimer.

Given its ability to efficiently assist in the delivery of glycoproteins terminated with galactose (Gal) or N-acetylgalactosamine (GalNAc) sugars to lysosomes, the asialoglycoprotein receptor (ASGPR) may be utilized for the degradation of extracellular target molecules (such as growth factors, cytokines, chemokines, hormones, neurotransmitters, capsids, soluble receptors, extracellular secreted proteins, antibodies, lipoproteins, exosomes, viruses, cells), where the extracellular target molecule is bound to the A_{G} moiety of a bifunctional compound described herein and the receptor binding moiety (R_{G}) of the bifunctional compound comprises one or more galactose (Gal) groups or one or more N-acetylgalactosamine (GalNAc) groups. Such receptor binding moieties (R_{G}) bind to the asialoglycoprotein receptor (ASGPR), whereby the extracellular target molecules are delivered to lysosomes and degraded via lysosomal degradation.

In some embodiments, R_{G} comprises a moiety selected from:

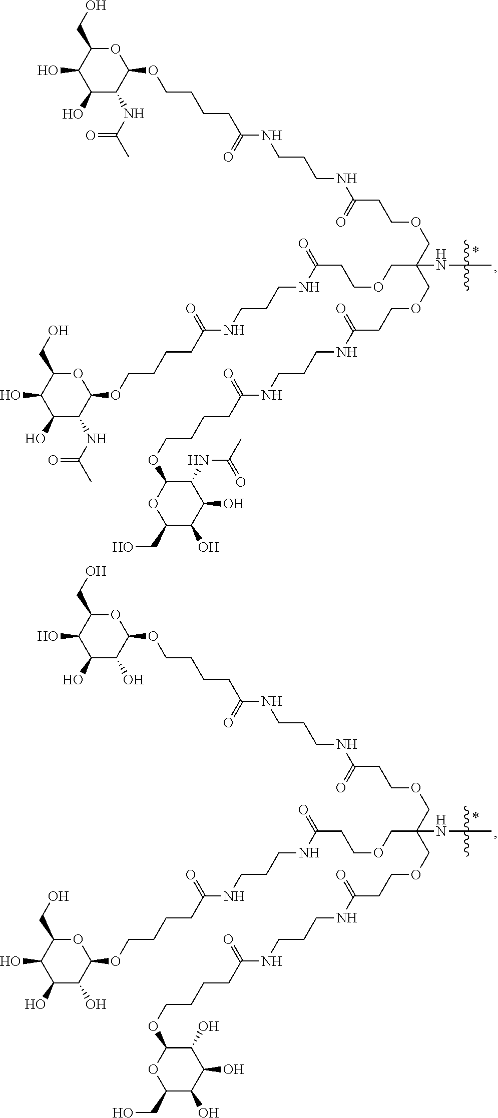 [SMILES: *NC(COCCC(=O)NCCCNC(=O)CCCCO[C@@H]1O[C@H](CO)[C@H](O)[C@H](O)[C@H]1NC(C)=O)(COCCC(=O)NCCCNC(=O)CCCCO[C@@H]1O[C@H](CO)[C@H](O)[C@H](O)[C@H]1NC(C)=O)COCCC(=O)NCCCNC(=O)CCCCO[C@@H]1O[C@H](CO)[C@H](O)[C@H](O)[C@H]1NC(C)=O ; *NC(COCCC(=O)NCCCNC(=O)CCCCO[C@@H]1O[C@H](CO)[C@H](O)[C@H](O)[C@H]1O)(COCCC(=O)NCCCNC(=O)CCCCO[C@@H]1O[C@H](CO)[C@H](O)[C@H](O)[C@H]1O)COCCC(=O)NCCCNC(=O)CCCCO[C@@H]1O[C@H](CO)[C@H](O)[C@H](O)[C@H]1O]

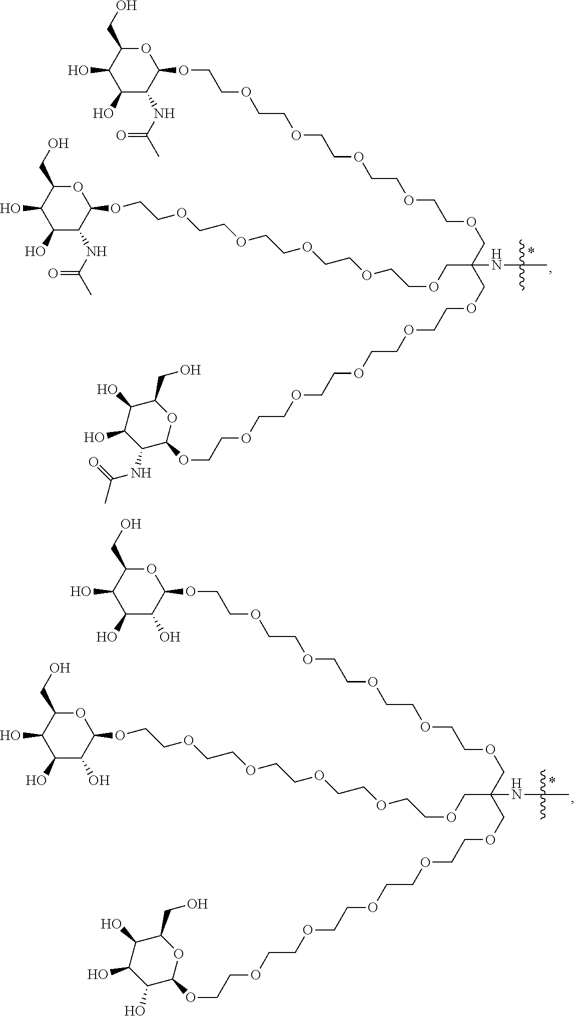

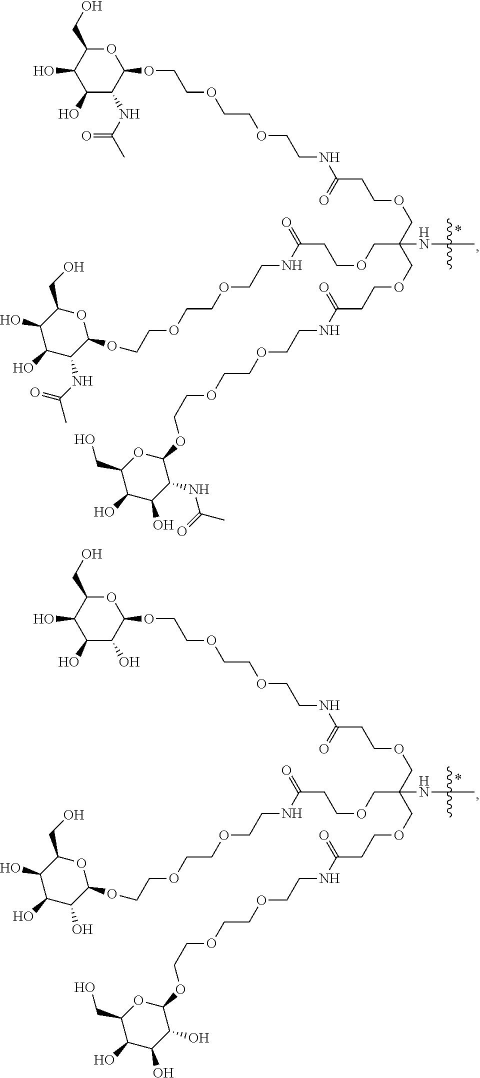 [SMILES: *NC(COCCC(=O)NCCOCCOCCO[C@@H]1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O ; *NC(COCCC(=O)NCCOCCOCCO[C@@H]1O[C@H](CO)[C@H](O)[C@H](O)[C@H]1O)(COCCC(=O)NCCOCCOCCO[C@@H]1O[C@H](CO)[C@H](O)[C@H](O)[C@H]1O)COCCC(=O)NCCOCCOCCO[C@@H]1O[C@H](CO)[C@H](O)[C@H](O)[C@H]1O]

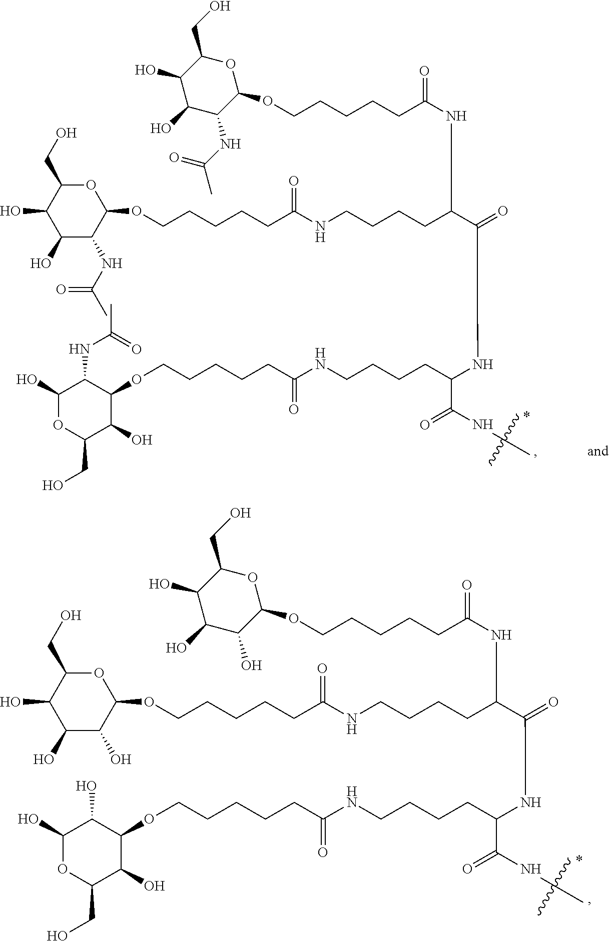 [SMILES: *NC(=O)C(CCCCNC(=O)CCCCCO[C@H]1[C@@H](O)[C@@H](CO)O[C@@H](O)[C@@H]1NC(C)=O)NC(=O)C(CCCCNC(=O)CCCCCO[C@@H]1O[C@H](CO)[C@H](O)[C@H](O)[C@H]1NC(C)=O)NC(=O)CCCCCO[C@@H]1O[C@H](CO)[C@H](O)[C@H](O)[C@H]1NC(C)=O ; *NC(=O)C(CCCCNC(=O)CCCCCO[C@@H]1[C@@H](O)[C@H](O)O[C@H](CO)[C@@H]1O)NC(=O)C(CCCCNC(=O)CCCCCO[C@@H]1O[C@H](CO)[C@H](O)[C@H](O)[C@H]1O)NC(=O)CCCCCO[C@@H]1O[C@H](CO)[C@H](O)[C@H](O)[C@H]1O]

where the * of R_{G} indicates the point of attachment to a linker (L).

In some embodiments, R_{G} comprises a galactose (Gal) group or N-acetylgalactosamine (GalNAc) group, where the Gal group or GalNAc group comprise a bridged ketal moiety. In some embodiments, R_{G} comprises a moiety selected from:

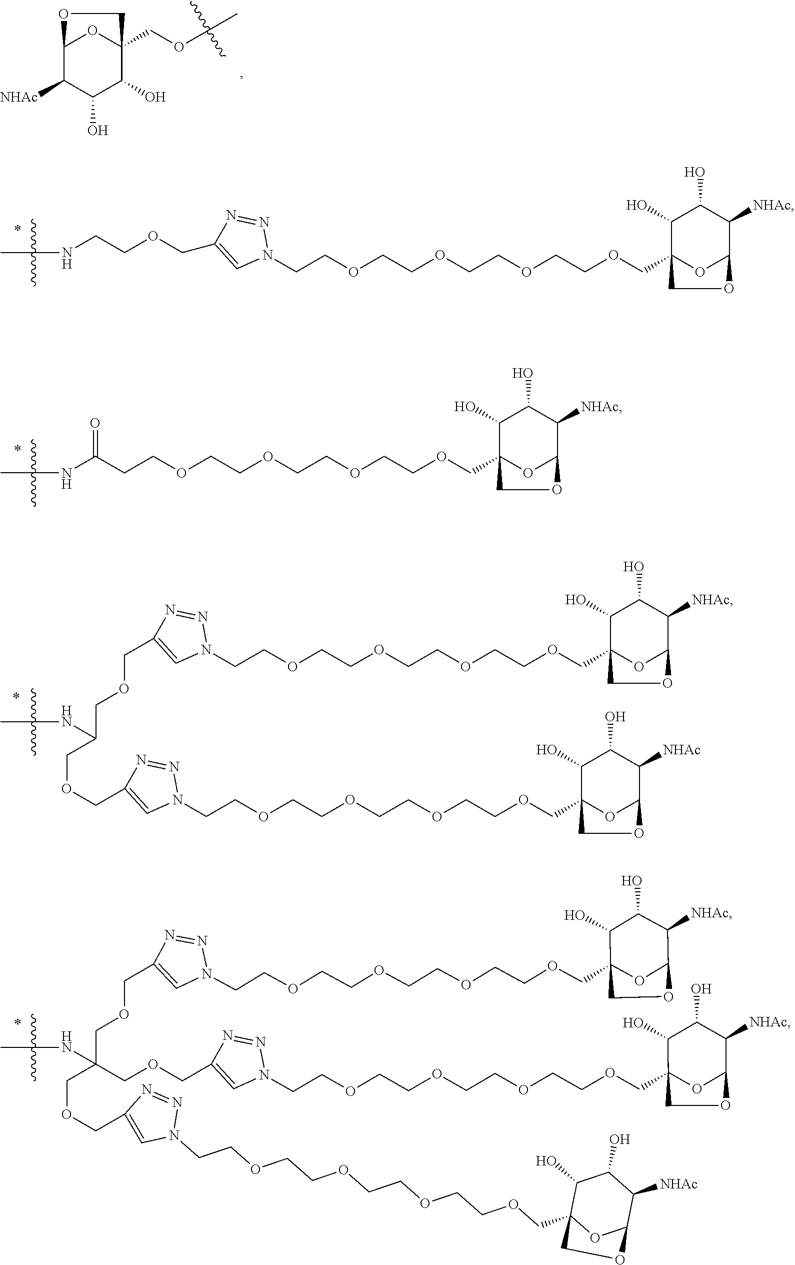

where the * of R_{G} indicates the point of attachment to a linker (L).

In some embodiments, R_{G} comprises a binding moiety for the mannose-6-phosphate receptor (M6PR) or insulin-like growth factor 2 receptor (IGF2R). The mannose-6-phosphate (M6P) group is added to N-linked oligosaccharides of lysosomal hydrolases, as these proteins move through the cis-Golgi network. The M6P groups may then be recognized by two independent transmembrane M6P receptors (MPRs), present in the trans-Golgi network: the cation-independent M6P receptor (CI-MPR, also known as the insulin-like growth factor 2 receptor (IGF2R)) and/or the cation-dependent M6P receptor (CD-MPR, also known as M6PR). In the trans-Golgi network, the M6P receptors may bind the M6P groups on a tagged lysosomal hydrolases at pH 6.5-6.7 and then help package the hydrolases into transport vesicles for their delivery to late endosomes. The cation-independent M6P receptor (CI-MPR, also known as the insulin-like growth factor 2 receptor (IGF2R)) also exists on the cell surface, where it may bind lysosomal enzymes that have escaped the cell, delivering them to the late endosome. Once inside the endosomes, which is typically pH 6, the lysosomal hydrolases dissociate from the MPRs and during endosomal maturation into lysosomes the pH drops to pH 5 where hydrolases begin to digest endocytosed material delivered from early endosomes. Subsequently, the MPRs may recycle from the endosome to the cell surface and then back to the Golgi complex.

In some embodiments, R_{G} comprises a binding moiety for the M6PR, where the binding moiety comprises a small molecule, a carbohydrate or carbohydrate derivative, an antibody molecule or a fragment thereof, a peptide, or a pharmaceutically acceptable salt thereof. In some embodiments, R_{G} comprises a binding moiety for the M6PR, where the binding moiety comprises a saccharide (e.g., a monosaccharide, disaccharide, trisaccharide, tetrasaccharide, pentasaccharide, or hexasaccharide (e.g., a galactose (Gal), N-acetylgalactosamine (GalNAc), bridged GalNAc, mannose-6-phosphate (M6P), mannose-6-phosphonate, a phosphonate dimer or polymer), an antibody molecule or a fragment thereof, a peptide, or a pharmaceutically acceptable salt thereof (e.g., R_{G} comprises a small molecule, a saccharide (e.g., a hexasaccharide), a phosphonate dimer or polymer, an antibody molecule or a fragment thereof, a peptide, or a pharmaceutically acceptable salt thereof).

Given its ability to efficiently assist in the delivery of M6P tagged proteins to lysosomes, the M6PRs and/or IGF2Rs, may be utilized for the degradation of extracellular target molecules (such as growth factors, cytokines, chemokines, hormones, neurotransmitters, capsids, soluble receptors, extracellular secreted proteins, antibodies, lipoproteins, exosomes, viruses, cells), where the extracellular target molecule is bound to the A_{G} moiety of a bifunctional compound described herein and the receptor binding moiety (R_{G}) of the bifunctional compound comprises a high affinity ligands for the M6PR and/or IGF2R. Such receptor binding moiety (R_{G}) groups bind to the M6PR or IGF2R, whereby extracellular target molecules are delivered to lysosomes and degraded via lysosomal degradation.

In some embodiments, R_{G} comprises a moiety selected from:

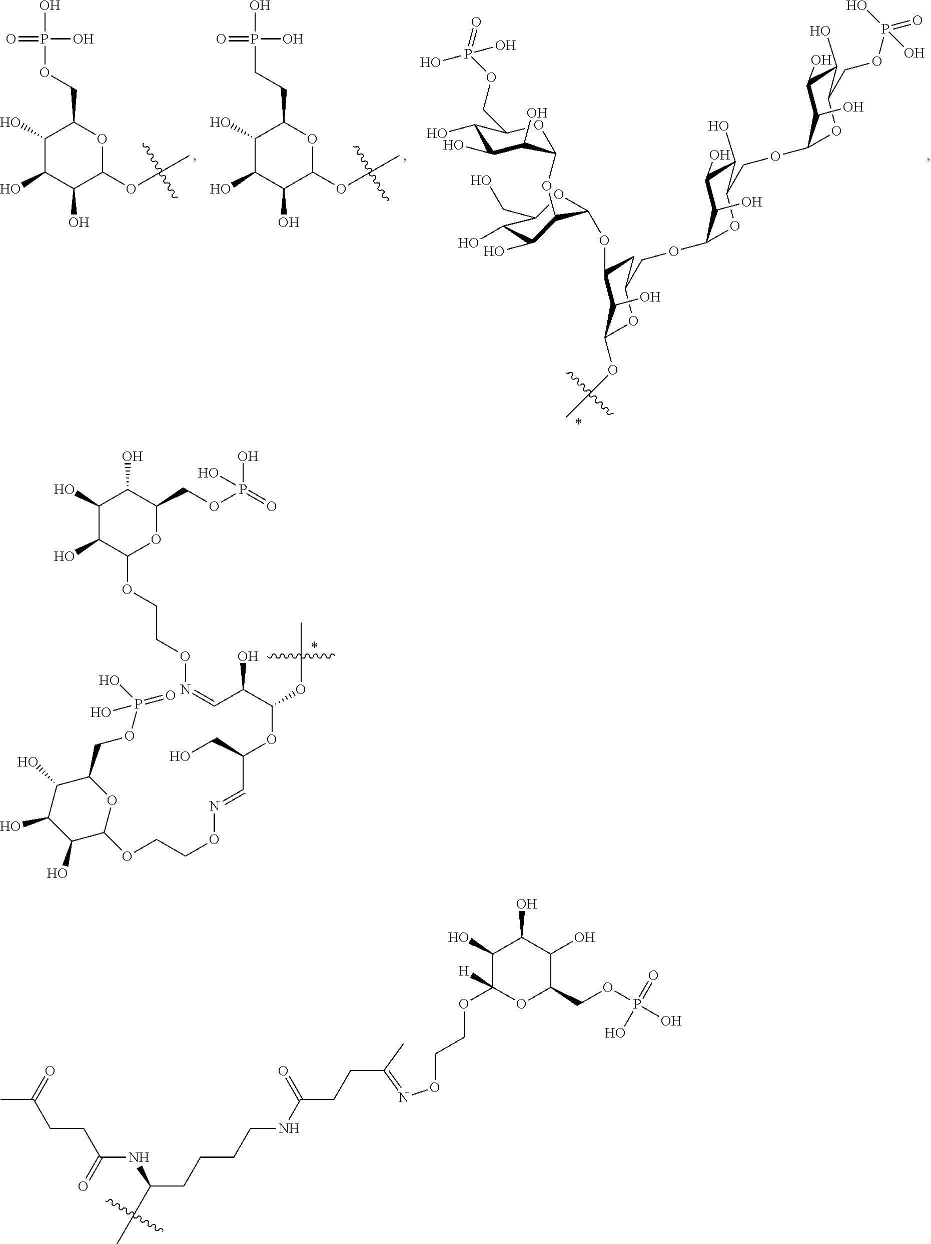

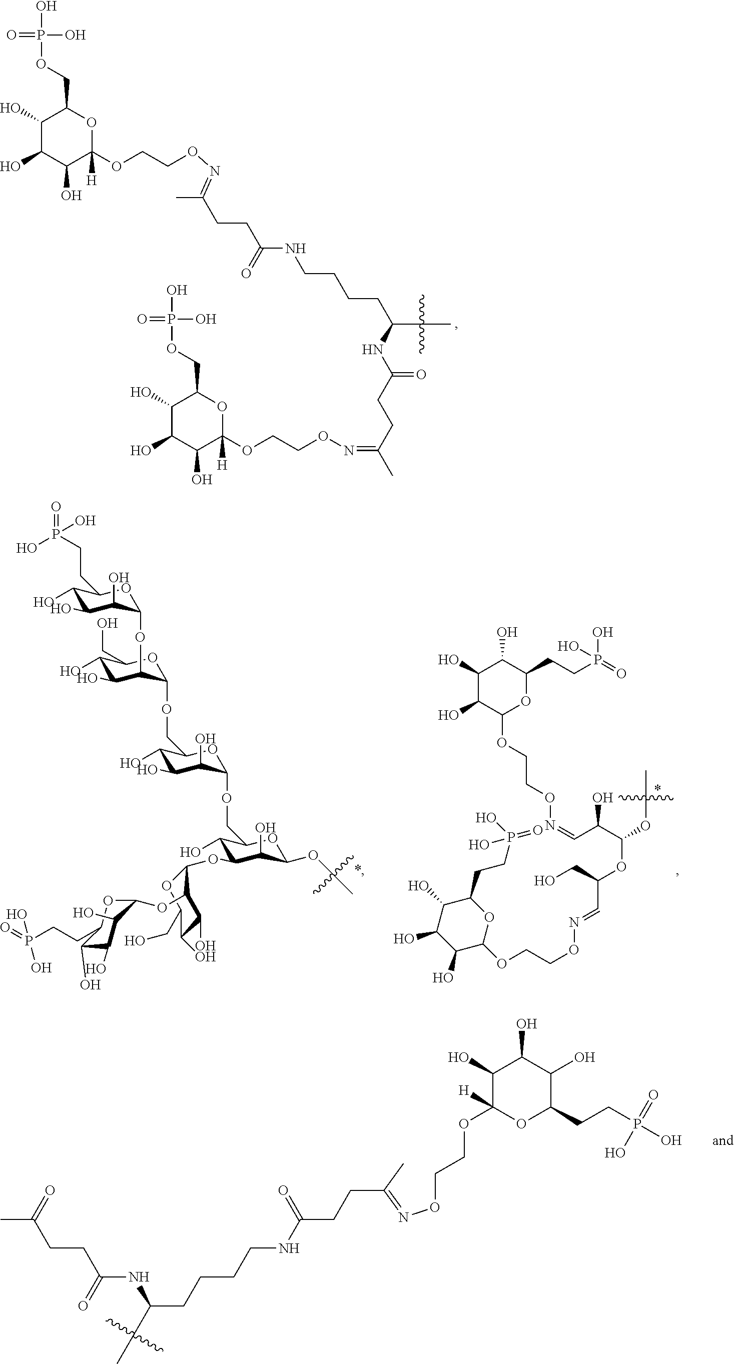

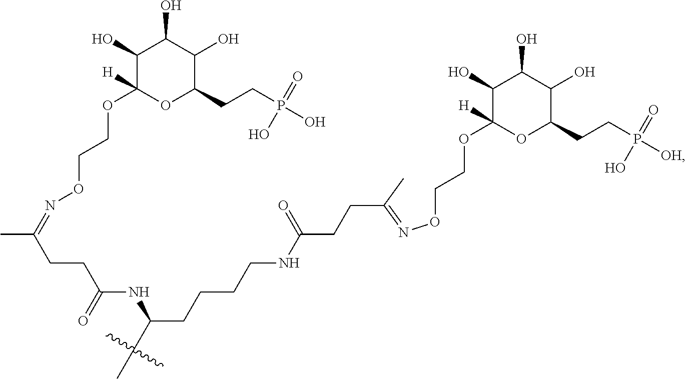 [SMILES: *[C@H](CCCCNC(=O)CC/C(C)=N/OCCO[C@H]1O[C@H](CCP(=O)(O)O)C(O)[C@H](O)[C@@H]1O)NC(=O)CC/C(C)=N\OCCO[C@H]1O[C@H](CCP(=O)(O)O)C(O)[C@H](O)[C@@H]1O]

where the * of R_{G} indicates the point of attachment to a linker (L).

In some embodiments, R_{G} comprises a compound of Formula (A):

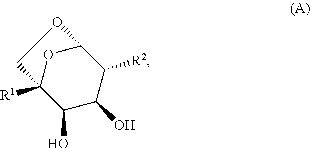

or a pharmaceutically acceptable salt thereof, wherein:
R¹ is —CN, —CH₂—CN, —C≡CH, —CH₂—N₃, —CH₂—NH₂, —CH₂—N(R⁴)—S(O)₂—R⁵, —CH₂—CO₂H, —CO₂H, —CH₂—OH, —CH₂—SH, —CH═CH—R⁵, —CH₂—R⁵, —CH₂—S—R⁵, —CH₂—N(R⁴)—R⁵, —CH₂—N(R⁴)—C(O)—R⁵, —CH₂—N(R⁴)—C(O)—O—R⁵, —CH₂—N(R⁴)—C(O)—N(R⁴)—R⁵, —CH₂—O—R⁵, —CH₂—O—C(O)—R⁵, —CH₂—O—C(O)—N(R⁴)—R⁵, —CH₂—O—C(O)—O—R⁵, CH₂—S(O)—R⁵, —CH₂—S(O)₂—R⁵, —CH₂—S(O)₂—N(R⁴)—R⁵, —C(O)—NH₂, —C(O)—O—R⁵, —C(O)—N(R⁴)—R⁵, or aryl or heteroaryl, wherein the aryl or heteroaryl is optionally substituted with R⁵,
or R¹ is —Z—X—Y wherein X is a linker or a drug delivery system, Y is absent or is a ligand selected from the group consisting of a small molecule, an amino acid sequence, a nucleic acid sequence, an antibody, an oligomer, a polymer, genetically derived material, a liposome, a nanoparticle, dye, fluorescent probe, or a combination thereof, and Z is absent or is —C≡C—, —CH═CH—, —CH₂—, —CH₂—O—, —C(O)—N(R⁴)—, —CH₂—S—, —CH₂—S(O)—, —CH₂—S(O)₂—, —CH₂—S(O)₂—N(R⁴)—, —C(O)—O—, —CH₂—N(R⁴)—, —CH₂—N(R⁴)—C(O)—, —CH₂—N(R⁴)—S(O)₂—, —CH₂—N(R⁴)—C(O)—O—, —CH₂—N(R⁴)—C(O)—N(R⁴)—, —CH₂—O—C(O)—, —CH₂—O—C(O)—N(R⁴)—, —CH₂—O—C(O)—O—, or aryl or heteroaryl, wherein the aryl or heteroaryl is optionally substituted with R⁵;
R² is —OH, —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³; and wherein when R¹ is —CH₂—OH, R² is —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³;
each R³ is independently —H, —(C₁-C₅)alkyl, halo-substituted (C₁-C₅)alkyl, or (C₃-C₆)cycloalkyl, wherein a —CH₂— group of the alkyl or cycloalkyl may be replaced with a heteroatom group selected from —O—, —S—, and —N(R⁴)— and —CH₃ of the alkyl may be replaced with a heteroatom group selected from —N(R⁴)₂, —OR⁴, and —S(R⁴) wherein the heteroatom groups are separated by at least 2 carbon atoms;
each R⁴ is independently —H, —(C₁-C₂₀)alkyl, or (C₃-C₆)cycloalkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴) wherein the heteroatom groups are separated by at least 2 carbon atoms; and wherein the alkyl and cycloalkyl may be substituted with one to six halo atoms; and
each R⁵ is independently —H, (C₃-C₂₀)cycloalkyl or (C₁-C₂₀)alkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴) wherein the heteroatom groups are separated by at least 2 carbon atoms; and wherein the alkyl and cycloalkyl may be substituted with one to six halo atoms.

In some embodiments, R_{G} is selected from:

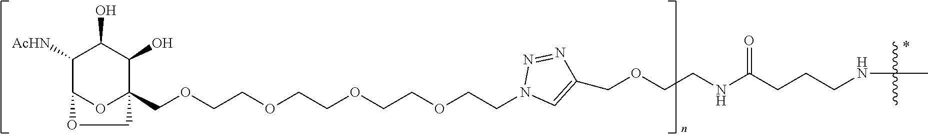

wherein n is 1, 2, or 3; and

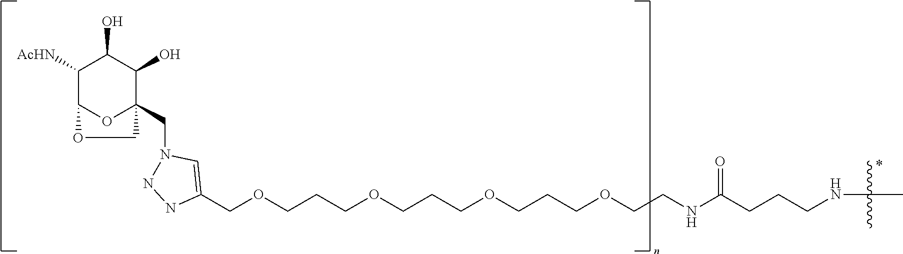

wherein n is 2 or 3; wherein R_{G} is attached to L at the terminal amine.

In some embodiments, R_{G} is any compound or moiety described in U.S. Pat. Nos. 9,617,293 and 10,039,778, each of which is incorporated herein by reference in its entirety.

### Linkers (L)

The linker moiety (L) is an optional moiety within the bifunctional compounds described herein that connects the target binding moiety (A_{G}) with the receptor binding moiety (R_{G}). In some embodiments, the linker moiety is absent. In some embodiments, the linker moiety is present.

In some embodiments, the linker is a (G4S)ₙ linker, wherein n is an integer from 1 to 20 (see e.g., Hust, M., et al. Single chain Fab (scFab) fragment. BMC Biotechnol 7, 14 (2007); Koerber J. T., et al. An improved single-chain Fab platform for efficient display and recombinant expression. J Mol Biol. 427(2):576-586 (2015)). In some embodiments, n is an integer from 1 to 4 (SEQ ID NO: 42). In some embodiments, the linker is a G4S linker (SEQ ID NO: 43). In some embodiments, the linker is a (G4S)₂ linker (SEQ ID NO: 44). In some embodiments, the linker is a (G4S)₃ linker (SEQ ID NO: 45). In some embodiments, the linker is a (G4S)₄ linker (SEQ ID NO: 46).

In an embodiment, the linker moiety has Formula (L-I):

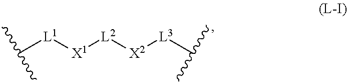

or a pharmaceutically acceptable salt, hydrate, solvate, prodrug, stereoisomer, or tautomer thereof,

wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand in Formula (I);
X¹ and X² are each independently selected from the group consisting of a bond, carbocyclyl, heterocyclyl, and heteroaryl;
L² is selected from the group consisting of a bond, 0, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I);
wherein no more than 2 of L¹, X¹, X², L², and L³ can simultaneously be a bond; and
R′ is hydrogen or C₁₋₆ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X and X² is not a bond. In an embodiment, one of X¹ and X² is a bond, and the other is a carbocyclyl or heterocyclyl.

In an embodiment, one of X¹ and X² is a bond, and the other is a heterocyclyl. In an embodiment, X¹ and X² are each independently selected from piperidinyl and piperazinyl. In an embodiment, X¹ and X² are both piperidinyl. In an embodiment, —X¹-L²-X²— is:

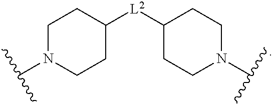

In an embodiment, the Linker is a compound having the following formula:

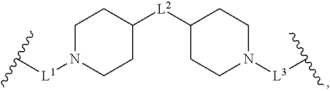

or a pharmaceutically acceptable salt, hydrate, solvate, prodrug, stereoisomer, or tautomer thereof.

In an embodiment, —X¹-L²-X²— forms a spiroheterocyclyl having the structure,

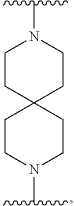 [SMILES: *N1CCC2(CC1)CCN(*)CC2]

substituted with 0-4 occurrences of R^{a}, wherein each R^{a} is independently selected from C₁₋₆ alkyl, C₁₋₆ alkoxyl, and C₁₋₆ hydroxyalkyl.

In an embodiment, —X¹-L²-X²— forms a spiroheterocyclyl having the structure,

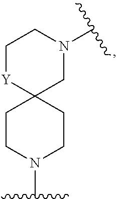

substituted with 0-4 occurrences of R^{b}, wherein Y is selected from CH₂, oxygen, and nitrogen; and each R^{b} is independently selected from C₁₋₆ alkyl, C₁₋₆ alkoxyl, and C₁₋₆ hydroxyalkyl.

In an embodiment, X¹ and X² are each a bond. In an embodiment, L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O). In an embodiment, L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene. In an embodiment, L³ is —C(O)— or C₁₋₆ heteroalkylene; and L¹ is C₁₋₈ alkylene or C₁₋₈ heteroalkylene. In an embodiment, L³ is a bond or —O—; and L¹ is —C(O)— or C₁₋₈ heteroalkylene. In an embodiment, L³ is selected from the group consisting of —O—, —C(O)—, —S(O)₂—, and C₁₋₆ heteroalkylene; and L¹ is C₁₋₈ alkylene or C₁₋₈ heteroalkylene. In an embodiment, L² is —C(O)—, —NR′—, or C₁₋₆ alkylene. In an embodiment, L² is —C(O)—, —O—, or C₁₋₆ alkylene. In an embodiment, L² is C₁₋₆ alkylene. In an embodiment, L² is selected from the group consisting of —C(O)—, C₁₋₆ alkylene, C₁₋₆ heteroalkylene, and *C(O)NR′—C₁₋₆ alkylene. In an embodiment, Y is CH₂, CH(C₁₋₃ alkyl), C(C₁₋₃ alkyl)₂, oxygen, NH, or N(C₁₋₃ alkyl).

In some embodiments, the linker comprises a peptide. In some embodiments, the peptide sequence is comprised of naturally occurring amino acids. In some embodiments, the peptide sequence comprises at least one synthetically derived amino acids, e.g., at least 2, at least 3, at least 4, at least 5, at least 8, at least 10, at least 15, at least 20, or more synthetically derived amino acids. In some embodiments, the peptide has a linear structure. In some embodiments, the peptide has a branched structure. In some embodiments, the peptide has a branched structure with, e.g., at least 2, at least 3, at least 4, at least 5, at least 6, at least 7, or at least 8 branching points. In some embodiments, the peptide has a cyclic structure.

In some embodiments, the linker comprises a peptide, and the peptide sequence comprises at least 2 amino acid residues. In some embodiments, the peptide sequence comprises at least 3, at least 4, at least 5, at least 6, at least 7, at least 8, at least 9, or at least 10 amino acid residues. In some embodiments, the peptide sequence is from about 1 to about 10 amino acid residues. In some embodiments, the peptide sequence is from about 1 to about 15, about 20, about 25, about 30, about 40, about 50, about 60, about 70, about 80, about 90, or about 100 amino acid residues. In some embodiments, the peptide sequence is from about 10 to about 100 amino acid residues. In some embodiments, the peptide sequence is from about 25 to about 100 amino acid residues. In some embodiments, the peptide sequence is from about 50 to about 100 amino acid residues.

In some embodiments, the linker comprises an alkylene, alkenylene, alkynylene, heteroalkylene, haloalkylene, carbonyl, ester, carbonate, amine, amide, carbamate, urea, hydroxyl, aryl ring, heteroaryl ring, cycloalkyl ring, or heterocyclyl ring. In some embodiments, the linker comprises a heteroalkylene moiety. Exemplary heteroalkylene moieties include a polyethylene oxide (PEO), a polypropylene glycol (PPG), a polyglycerol (PG), a poloxamine (POX), a polybutylene oxide (PBO), polylactic acid (PLA), polyglycolic acid (PGA), poly(lactic-co-glycolic acid) (PLGA), polycaprolactone (PCL), polydioxanone (PDO), a polyanhydride, a polyacrylide, a polyvinyl, a polyorthoester, a dextran, a cyclodextran, chitosan, or other carbohydrate based polymer. In some embodiments, the heteroalkylene moiety comprises at least 6, 8, 10, 12, 16, 20, 24, 36, 48, 60, 100, 250, 300, or more carbon atoms. In some embodiments, the heteroalkylene moiety is between 4 and 48 carbon atoms, or 4 and 36 carbon atoms, or 4 and 24 carbon atoms, or 4 and 16 carbon atoms, or 6 and 12 carbon atoms, or 8 and 12 carbon atoms.

In some embodiments, the linker comprises a polyethylene glycol moiety (i.e., “PEG”). Polyethylene glycols include ethylene glycol polymer containing various numbers of linked monomers, e.g., PEG1, PEG2, PEG3, PEG4, PEG6, PEG8, PEG10, PEG12, PEG14, PEG16, PEG18, PEG20, PEG30, PEG40, PEG60, PEG80, PEG100, PEG115, PEG200, PEG300, PEG400, PEG500, PEG600, PEG1000, PEG1500, PEG2000, PEG3350, PEG4000, PEG4600, PEG5000, PEG6000, PEG8000, PEG11000, PEG12000, or PEG2000000 and any mixtures thereof. In some embodiments, the linker comprises a polyethylene glycol moiety with between 4 and 48 carbon atoms, or 4 and 36 carbon atoms, or 4 and 24 carbon atoms, or 4 and 16 carbon atoms, or 6 and 12 carbon atoms, or 8 and 12 carbon atoms.

The linker of the bifunctional compound described herein, or a component thereof, may be readily formed by reaction between two reactive groups. Non-limiting examples of such chemical moieties are given in Table 2. In some embodiments, the linker is prepared via reaction of two or more reactive groups described in Table 2. In some embodiments, the linker comprises a chemical moiety described in Table 2.

TABLE 2
Exemplary reactive groups and components of linker (L)

| Reactive Group 1 | Reactive Group 2 | Chemical Moiety |
|---|---|---|
| (RG1) | (RG2) | (component of L) |
| a thiol | a thiol | —S—S— |
| a thiol | a maleimide | 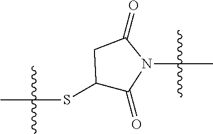 [SMILES: *SC1CC(=O)N(*)C1=O] |
| a thiol | a haloacetamide | 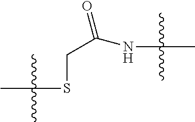 [SMILES: *NC(=O)CS*] |
| an azide | an alkyne | 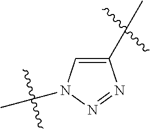 [SMILES: *c1cn(*)nn1] |
| an azide | a triaryl phosphine | 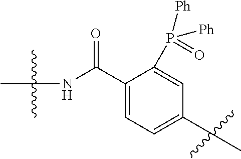 [SMILES: *NC(=O)c1ccc(*)cc1P(=O)(c1ccccc1)c1ccccc1] |
| an azide | a cyclooctyne | 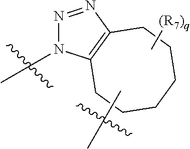 |
| | | or |
| | | 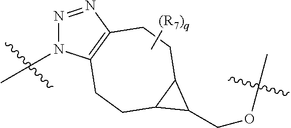 |
| | | or |
| | | 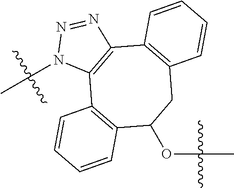 [SMILES: *OC1Cc2ccccc2-c2nnn(*)c2-c2ccccc21] |
| an azide | an oxanobornadiene | 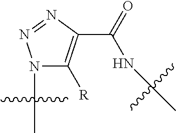 |
| a triaryl phosphine | an azide | 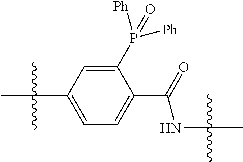 [SMILES: *NC(=O)c1ccc(*)cc1P(=O)(c1ccccc1)c1ccccc1] |
| an oxanobornadiene | an azide | 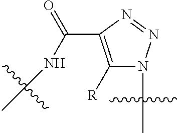 |
| an alkyne | an azide | 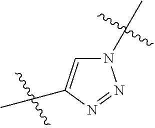 [SMILES: *c1cn(*)nn1] |
| a cyclooctyne | azide | 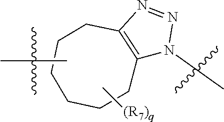 |
| | | or |
| | | 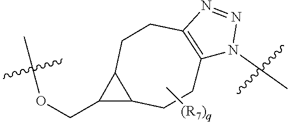 |
| | | or |
| | | 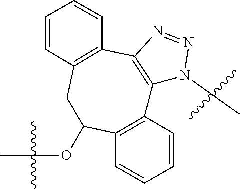 [SMILES: *OC1Cc2ccccc2-c2nnn(*)c2-c2ccccc21] |
| a cyclooctene | a diaryl tetrazine | 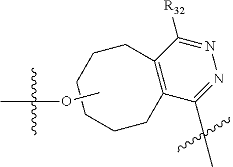 |
| | | or |
| | | 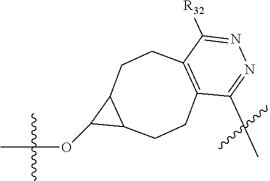 |
| a diaryl tetrazine | a cyclooctene | 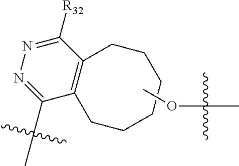 |
| | | or |
| | | 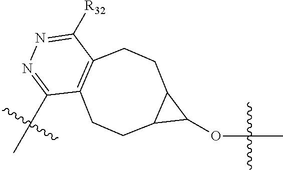 |
| a monoaryl tetrazine | a norbornene | 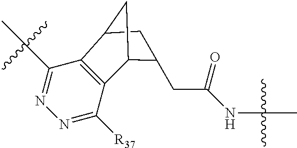 |
| a norbornene | a monoaryl tetrazine | 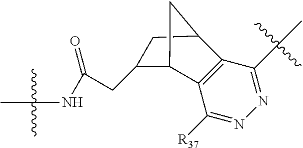 |
| an aldehyde | a hydroxylamine | 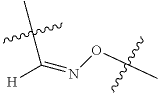 [SMILES: */C=N\O*] |
| an aldehyde | a hydrazine | 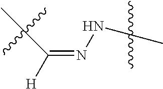 [SMILES: */C=N\N*] |
| an aldehyde | NH₂—NH—C(═O)— | 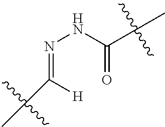 [SMILES: */C=N/NC(*)=O] |
| a ketone | a hydroxylamine | 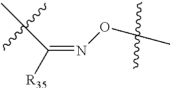 |
| a ketone | a hydrazine | 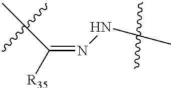 |
| a ketone | NH₂—NH—C(═O)— | 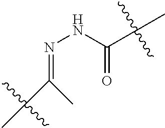 [SMILES: *C(=O)N/N=C(/*)C] |
| a hydroxylamine | an aldehyde | 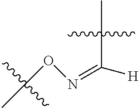 [SMILES: */C=N\O*] |
| a hydroxylamine | a ketone | 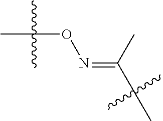 [SMILES: *O/N=C(/*)C] |
| a hydrazine | an aldehyde | 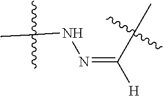 [SMILES: */C=N\N*] |
| a hydrazine | a ketone | 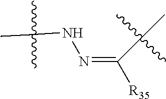 |
| NH₂—NH—C(═O)— | an aldehyde | 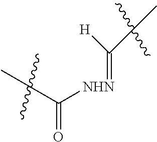 |
| NH₂—NH—C(═O)— | a ketone | 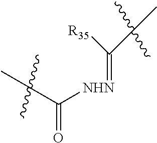 |
| a haloacetamide | a thiol | 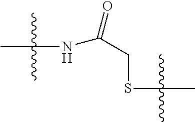 [SMILES: *NC(=O)CS*] |
| a maleimide | a thiol | 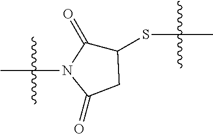 [SMILES: *SC1CC(=O)N(*)C1=O] |
| a vinyl sulfone | a thiol | 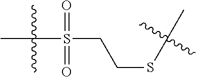 [SMILES: *SCCS(*)(=O)=O] |
| a thiol | a vinyl sulfone | 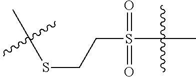 [SMILES: *SCCS(*)(=O)=O] |
| an aziridine | a thiol | 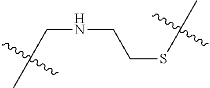 [SMILES: *CNCCS*] |
| | | or |
| | | 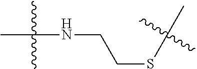 [SMILES: *NCCS*] |
| a thiol | an aziridine | 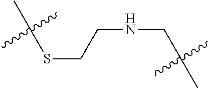 [SMILES: *CNCCS*] |
| | | or |
| | | 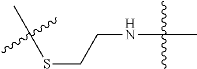 [SMILES: *NCCS*] |
| 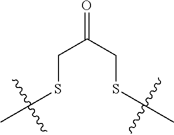 [SMILES: *SCC(=O)CS*] | hydroxylamine | 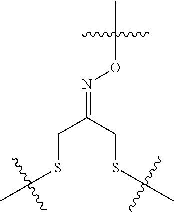 [SMILES: *ON=C(CS*)CS*] |
| 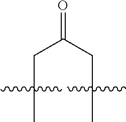 [SMILES: *CC(=O)C*] | hydroxylamine | 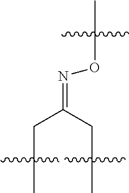 [SMILES: *CC(C*)=NO*] |
| 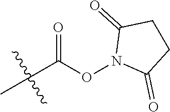 [SMILES: *C(=O)ON1C(=O)CCC1=O] | —NH2, | amide |
| 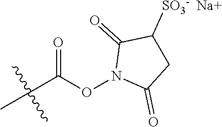 | | |
| 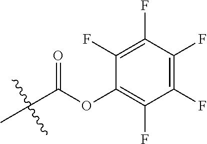 [SMILES: *C(=O)Oc1c(F)c(F)c(F)c(F)c1F] | | |
| 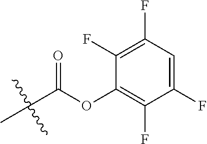 [SMILES: *C(=O)Oc1c(F)c(F)cc(F)c1F] | | |
| 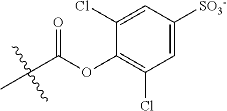 | | |
| —NH2, | 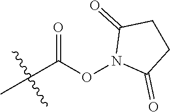 [SMILES: *C(=O)ON1C(=O)CCC1=O] | amide |
| | 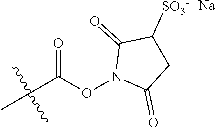 | |
| | 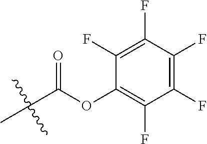 [SMILES: *C(=O)Oc1c(F)c(F)c(F)c(F)c1F] | |
| | 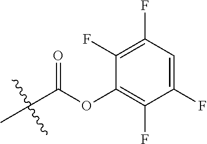 [SMILES: *C(=O)Oc1c(F)c(F)cc(F)c1F] | |
| | 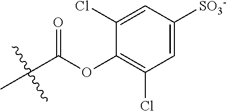 | |
| pyridyldithiol | thiol | disulfide |

wherein: R³² in Table 2 is H, C₁₋₄ alkyl, phenyl, pyrimidine or pyridine; R³⁵ in Table 2 is H, C₁₋₆ alkyl, phenyl or C₁₋₄ alkyl substituted with 1 to 3 —OH groups; each R₇ in Table 2 is independently selected from H, C₁₋₆ alkyl, fluoro, benzyloxy substituted with —C(═O)OH, benzyl substituted with —C(═O)OH, C₁₋₄ alkoxy substituted with —C(═O)OH and C₁₋₄alkyl substituted with —C(═O)OH; R³⁷ in Table 2 is independently selected from H, phenyl and pyridine; and q in Table 2 is 0, 1, 2 or 3.

In some embodiments, the linker comprises a moiety described in Table 3.

TABLE 3
Exemplary components of linker (L)

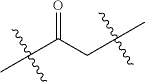 [SMILES: *CC(*)=O]

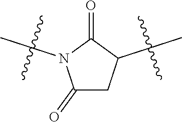 [SMILES: *C1CC(=O)N(*)C1=O]

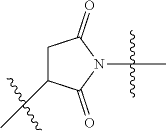 [SMILES: *C1CC(=O)N(*)C1=O]

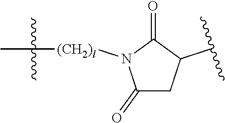

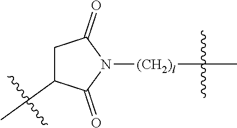

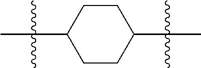 [SMILES: *C1CCC(*)CC1]

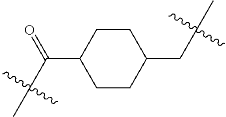 [SMILES: *CC1CCC(C(*)=O)CC1]

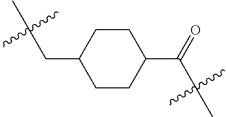 [SMILES: *CC1CCC(C(*)=O)CC1]

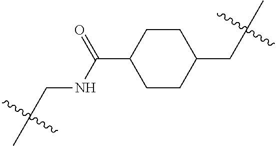 [SMILES: *CNC(=O)C1CCC(C*)CC1]

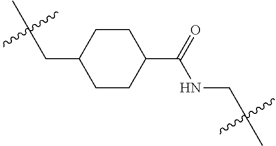 [SMILES: *CNC(=O)C1CCC(C*)CC1]

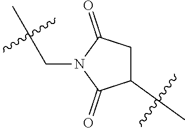 [SMILES: *CN1C(=O)CC(*)C1=O]

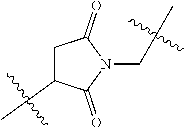 [SMILES: *CN1C(=O)CC(*)C1=O]

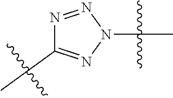 [SMILES: *c1nnn(*)n1]

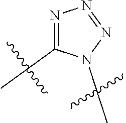 [SMILES: *c1nnnn1*]

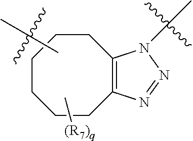

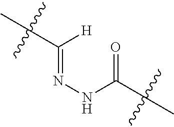 [SMILES: */C=N/NC(*)=O]

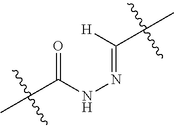 [SMILES: */C=N/NC(*)=O]

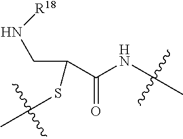

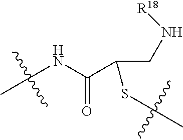

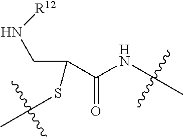

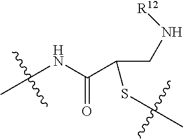

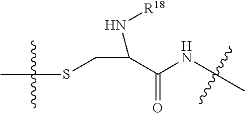

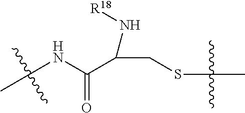

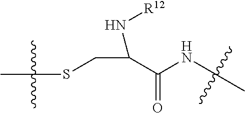

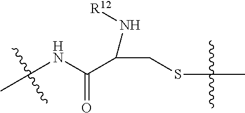

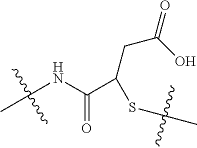 [SMILES: *NC(=O)C(CC(=O)O)S*]

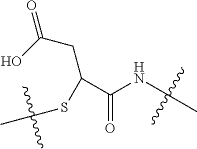 [SMILES: *NC(=O)C(CC(=O)O)S*]

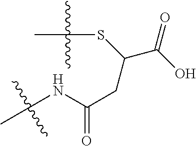 [SMILES: *NC(=O)CC(S*)C(=O)O]

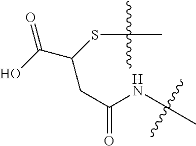 [SMILES: *NC(=O)CC(S*)C(=O)O]

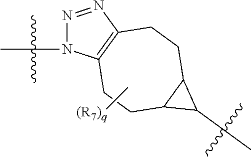

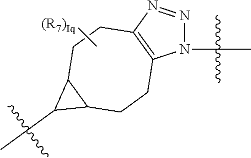

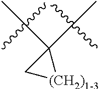 [SMILES: *C1(*)CC1]

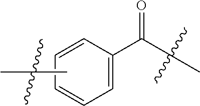

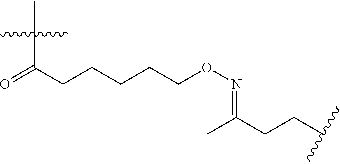 [SMILES: *CC/C(C)=N/OCCCCCC(*)=O]

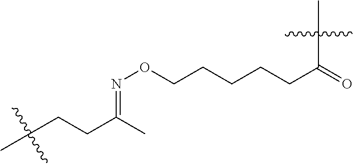 [SMILES: *CC/C(C)=N/OCCCCCC(*)=O]

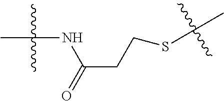 [SMILES: *NC(=O)CCS*]

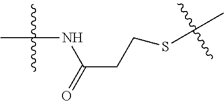 [SMILES: *NC(=O)CCS*]

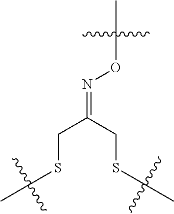 [SMILES: *ON=C(CS*)CS*]

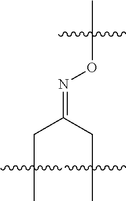 [SMILES: *CC(C*)=NO*]

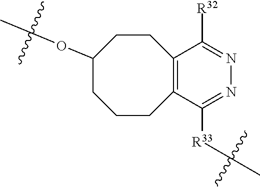

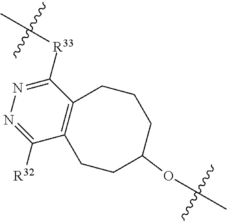

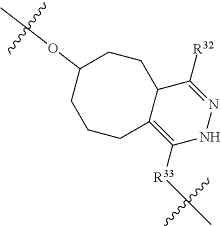

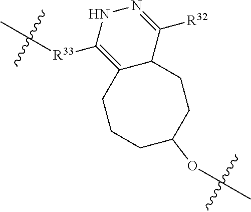

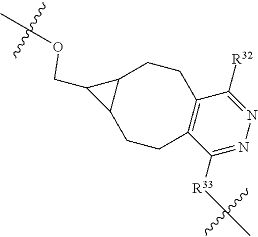

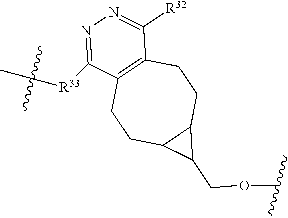

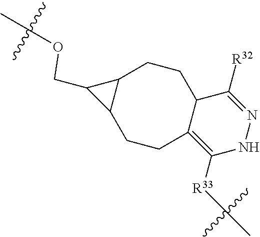

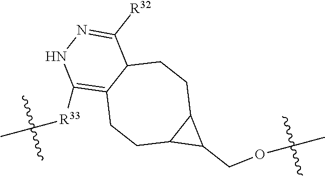

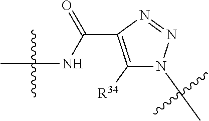

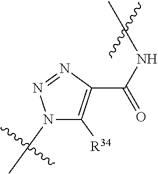

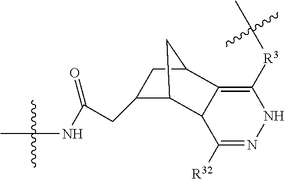

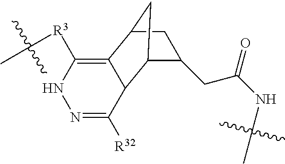

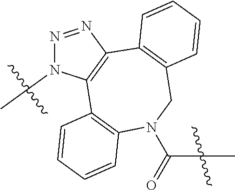 [SMILES: *C(=O)N1Cc2ccccc2-c2nnn(*)c2-c2ccccc21]

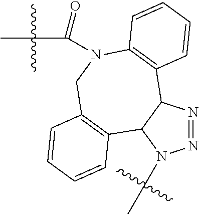 [SMILES: *C(=O)N1Cc2ccccc2C2C(N=NN2*)c2ccccc21]

each R⁷ is independently selected from H, C₁₋₆ alkyl, fluoro, benzyloxy substituted with —C(═O)OH, benzyl substituted with —C(═O)OH, C₁₋₄ alkoxy substituted with —C(═O)OH and C₁₋₄ alkyl substituted with —C(═O)OH;
each R¹² is independently selected from H and C₁₋₆ alkyl;
each R²⁵ is independently selected from H or C₁₋₄ alkyl;
each R¹⁸ is independently selected from a C₁₋₆ alkyl, a C₁₋₆ alkyl which is substituted with azido and a C₁₋₆ alkyl which is substituted with 1 to 5 hydroxyl;
q is 0, 1, 2 or 3; 1 is 1, 2, 3, 4, 5 or 6;
R³² is independently selected from H, C₁₋₄ alkyl, phenyl, pyrimidine and pyridine;
R³³ is independently selected from 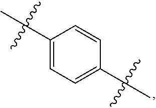 [SMILES: *c1ccc(*)cc1] 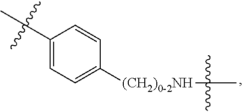 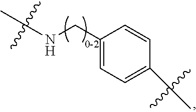 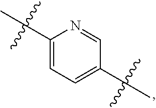 [SMILES: *c1ccc(*)nc1] 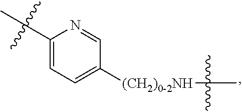 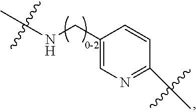 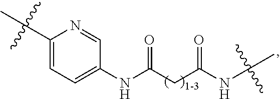
and 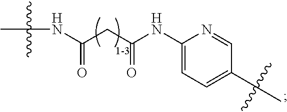
and R³⁴ is independently selected from H, C₁₋₄ alkyl, and C₁₋₆ haloalkyl.

In some embodiments, the linker is selected from:
*—(CH2)nC(═O)NHNHC(═O)(CH2)nON═CH2X1C(═O)—**;
*—(CH2)nX3C(═O)—**;
*—(CH2)nC(═O)—**;
*—(CH2)nC(═O)NHNHC(═O)(CH2)nON═CH2X1C(═O)NH(CH2)nCH(C═(O)NH2)-**; *—(CH2)nX3C(═O)NH(CH2)nCH(C═(O)NH2)-**;
*—(CH2)nC(═O)NH(CH2)nCH(C═(O)NH2)-**;
*—((CH2)nO)t(CH2)mC(═O)—**; *—((CH2)nO)t(CH2)m-**;
*—(CH2)nC(═O)NH((CH2)nO)t(CH2)m-**;
*—(CH2)n-; *—(CH2)nNHC(═O)(CH2)m-**;
*—(CH2)nNHC(═O)(CH2)nC(═O)NH(CH2)m-**;
*—((CH2)nO)t(CH2)nNHC(═O)(CH2)m-**;
*—((CH2)nO)tCH2)mC(═O)NH(CH2)m-**;
*—((CH2)nO)t(CH2)nNHC(═O)(CH2)m-**;
*—(CH2)nO(CH2)m-**;
*—(CH2)nNH(CH2)n-**;
*—(CH2)nNH(CH2)mC(═O)—**;
*—(CH2)nX3(CH2)m-**;
*—((CH2)nO)t(CH2)nX3(CH2)m-**;
*—(CH2)nNHC(═O)(CH2)nX3(CH2)m-**;
*—((CH2)nO)t(CH2)nNHC(═O)(CH2)nX3(CH2)m-**;
*—((CH2)nO)t(CH2)nC(═O)NH(CH2)m-**;
*—(CH2)mNHC(═O)((CH2)nO)t(CH2)m-**;
*—(CH2)nC(═O)NH(CH2)m-**;
*—(CH2)nNHC(═O)((CH2)nO)t(CH2)m-**;
*—(CH2)nNHC(═O)(CH2)nO(CH2)m-**;
*—(CH2)nNH(CH2)m-**;
*—((CH2)nO)tCH2)nC(═O)NH(CH2)m-**;
*—(CH2)nNHC(═O(CH2)nX3(CH2)m-**;
*—C(═O)—;
*—C(═O)(CH2)nC(═O)—**;
*—C(═O)((CH2)nO)t(CH2)mC(═O)—**;
*—C(═O)((CH2)nO)t(CH2)m-**;
*—((CH2)nO)t(CH2)mX3(CH2)nO(CH2)nNHC(═O)((CH2)nO)t(CH2)mC(═O)—**,
*—C(═O)(CH2)nC(═O)NH((CH2)nO)t(CH2)m-**;
*—C(═O)NHNHC(═O)(CH2)nON═CH2X1C(═O)NH(CH2)nCH(C═(O)NH2)-**;
*—X3C(═O)NH(CH2)nCH(C═(O)NH2)-**;
*—C(═O)(CH2)nC(═O)NHNHC(═O)(CH2)nON═CH2X1C(═O)—**;
*—C(═O)(CH2)nX3C(═O)—**;
*—C(═O)(CH2)nNHC(═O)(CH2)nC(═O)NH(CH2)m-**;
*—C(═O)((CH2)nO)t(CH2)nNHC(═O)(CH2)m-**;
*—C(═O)((CH2)nO)tCH2)mC(═O)NH(CH2)m-**;
*—C(═O)(CH2)nO(CH2)m-**;
*—C(═O)(CH2)n-**;
*—C(═O)NH((CH2)nO)t(CH2)m-**;
*—C(═O)(CH2)nNH(CH2)n-**;
*—C(═O)(CH2)nNH(CH2)mC(═O)—**;
*—C(═O)(CH2)nX3(CH2)m-**;
*—C(═O)((CH2)nO)t(CH2)nX3(CH2)m-**;
*—C(═O)(CH2)nNHC(═O)(CH2)m-**;
*—C(═O)(CH2)nNHC(═O)((CH2)nO)t(CH2)m-**;
*—C(═O)(CH2)nNHC(═O)(CH2)nO(CH2)m-**;
*—C(═O)(CH2)nNH(CH2)m-**;
*—C(═O)((CH2)nO)tCH2)nC(═O)NH(CH2)m-**;
*—C(═O)(CH2)nNHC(═O(CH2)nX3(CH2)m-**;
*—C(═O)NH(CH2)nX3(CH2)m-**;
*—C(═O)NH(CH2)nNHC(═O)(CH2)m-**;
*—C(═O)NH(CH2)nNHC(═O)(CH2)nO(CH2)m-**;
*—C(═O)NH(CH2)nNHC(═O)(CH2)nX3(CH2)m-**;
*—C(═O)NH(CH2)nNHC(═O)—**;
*—C(═O)NH((CH2)nO)t(CH2)nX3(CH2)m-**;
*—C(═O)(CH2)nNHC(═O)(CH2)nX3(CH2)m-**;
*—C(═O)((CH2)nO)t(CH2)nNHC(═O)(CH2)nX3(CH2)m-**;
*—C(═O)((CH2)nO)t(CH2)nC(═O)NH(CH2)m-**;
*—C(═O)(CH2)mNHC(═O)((CH2)nO)t(CH2)m-**, or
*—C(═O)(CH2)nC(═O)NH(CH2)m-**;
   wherein
X1 is

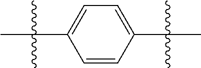 [SMILES: *c1ccc(*)cc1]

### and X3 is

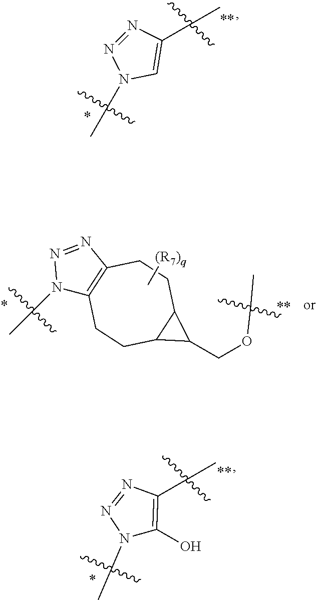

where for X3, the * indicates point of attachment toward R_{G} and the ** indicates point of attachment toward A_{G}, and where for all other species, the * indicates the point of attachment to R_{G}, and the ** of indicates the point of attachment to A_{G}.

In some embodiments, the linker is selected from:
*—(CH2)nC(═O)NHNHC(═O)(CH2)nON═CH2X1C(═O)—**;
*—(CH2)nX3C(═O)—**; *—((CH2)nO)t(CH2)mC(═O)—**;
*—(CH2)nC(═O)—**;
*—(CH2)nC(═O)NHNHC(═O)(CH2)nON═CH2X1C(═O)NH(CH2)nCH(C═(O)NH2)-**; *—(CH2)nX3C(═O)NH(CH2)nCH(C═(O)NH2)-**;
*—C(═O)—;
*—C(═O)(CH2)nC(═O)—**;
*—C(═O)((CH2)nO)t(CH2)mC(═O)—**;
*—((CH2)nO)t(CH2)mX3(CH2)nO(CH2)nNHC(═O)((CH2)nO)t(CH2)mC(═O)—**;
*—C(═O)((CH2)nO)t(CH2)m-**; and
*—C(═O)(CH2)nC(═O)NH((CH2)nO)t(CH2)m-**;
   wherein
X1 is

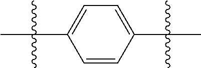 [SMILES: *c1ccc(*)cc1]

### and X3 is

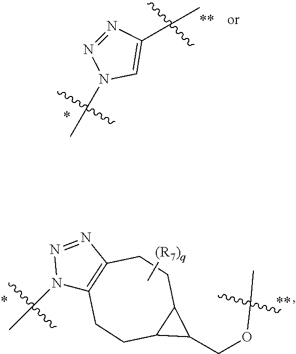

where for X3, the * indicates point of attachment toward R_{G} and the ** indicates point of attachment toward A_{G}, and where for all other species, the * indicates the point of attachment to R_{G}, and the ** of indicates the point of attachment to A_{G}.

In some embodiments, the linker is not a peptide. In some embodiments, the linker is a peptide, but does not comprise an amino acid residue selected from an alanine, glycine, serine, or threonine. In some embodiments, the linker is an unmodified peptide (e.g., a peptide that does not comprise a modification, such as a chemical modification). In some embodiments, the linker is not a glycopeptide.

In some embodiments, the linker does not comprise a structure of Formula (B):

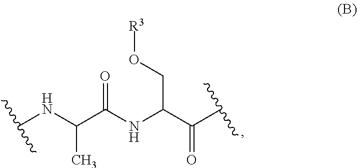

wherein R³ is a carbohydrate or carbohydrate derivative (e.g., a monosaccharide, disaccharide, oligosaccharide, or polysaccharide, each of which is optionally substituted). In some embodiments, —CH₃ is in the (L) or (D) configuration.

In some embodiments, the linker does not comprise an aryl, heteroaryl, cycloalkyl, or heterocyclyl. In some embodiments, the linker does not comprise a heteroaryl or heterocyclyl (e.g., a nitrogen-containing heteroaryl or nitrogen-containing heterocyclyl). In some embodiments, the linker does not comprise a nitrogen-containing heteroaryl (e.g., a triazolyl) or nitrogen-containing heterocyclyl. In some embodiments, the linker does not comprise a structure of Formula (C):

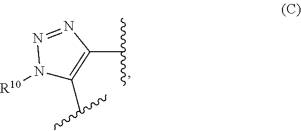

wherein R¹⁰ is a carbohydrate or carbohydrate derivative moiety (e.g., a monosaccharide, disaccharide, oligosaccharide, or polysaccharide, each of which is optionally substituted). In some embodiments, R¹⁰ comprises a glucose, galactose, mannose, glucosamine, galactosamine, or mannosamine moiety.

In some embodiments, the linker does not comprise a compound or moiety disclosed in WO 2019/199621, which is incorporated herein by reference in its entirety.

In some embodiments, the bifunctional compound of Formula (I) is a bispecific antibody. In some embodiment, the bifunctional compound of Formula (I) is a bispecific antibody, where R_{G} comprises an anti-ASGPR antibody or a fragment thereof. In some embodiments, the bifunctional compound of Formula (I) is an anti-ASGPR-PCSK9 bispecific antibody. In an embodiment, A_{G} comprises an antibody or fragment thereof (an anti-PCSK9 antibody or fragment thereof), L is absent, and R_{G} is an antibody or fragment thereof (e.g., an anti-ASGPR antibody or fragment thereof). In an embodiment, the bifunctional compound comprises an antibody described in Example 2 and, e.g., FIGS. 1-3.

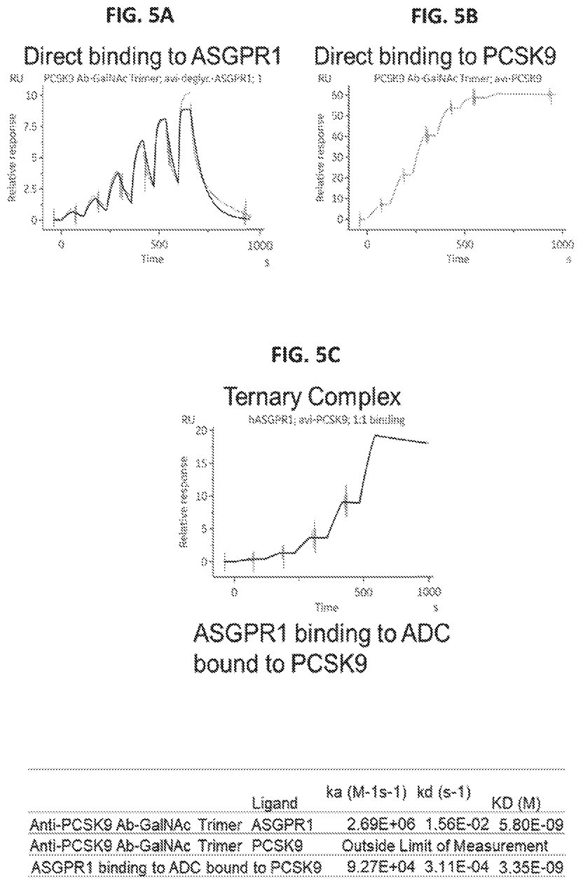

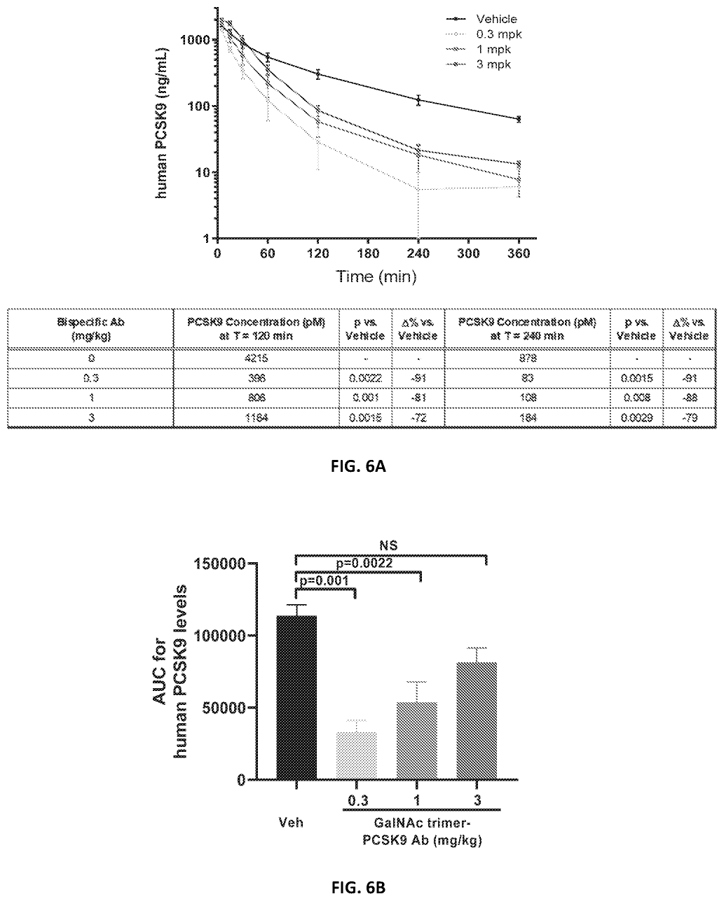

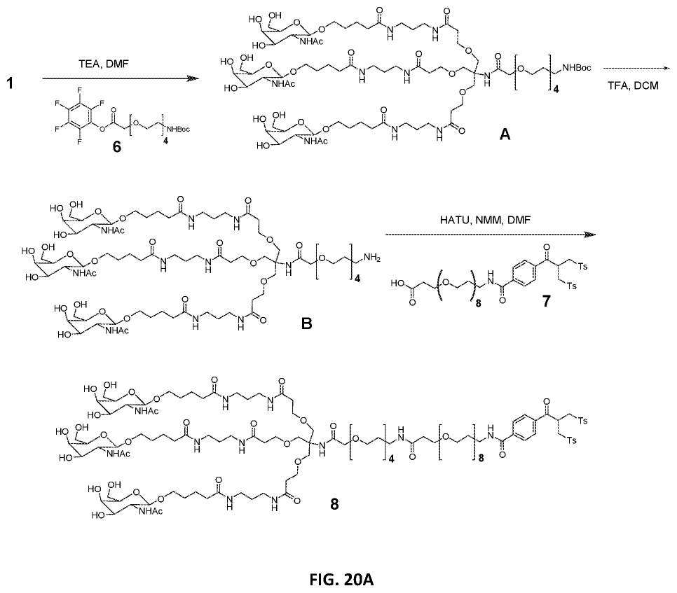

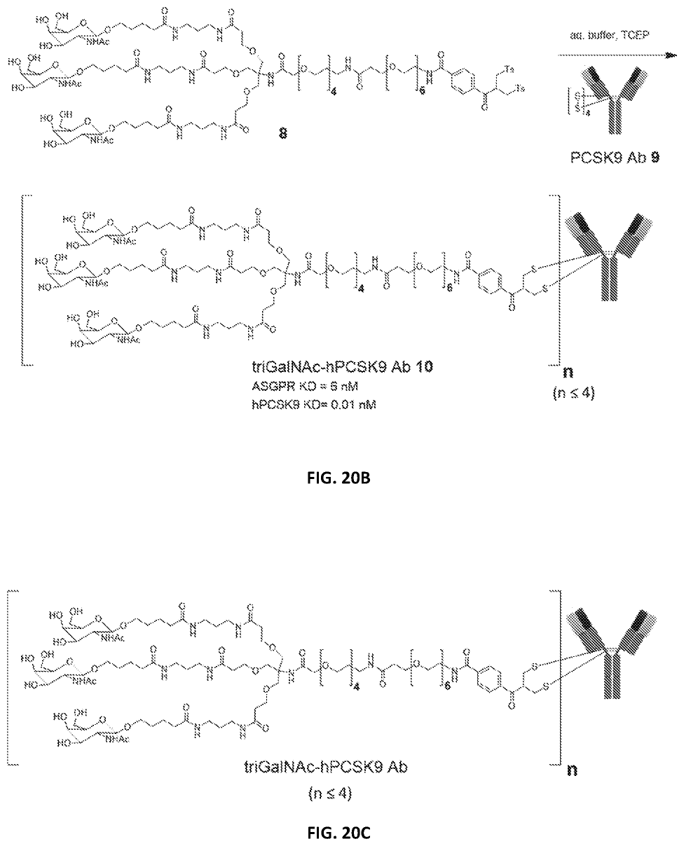

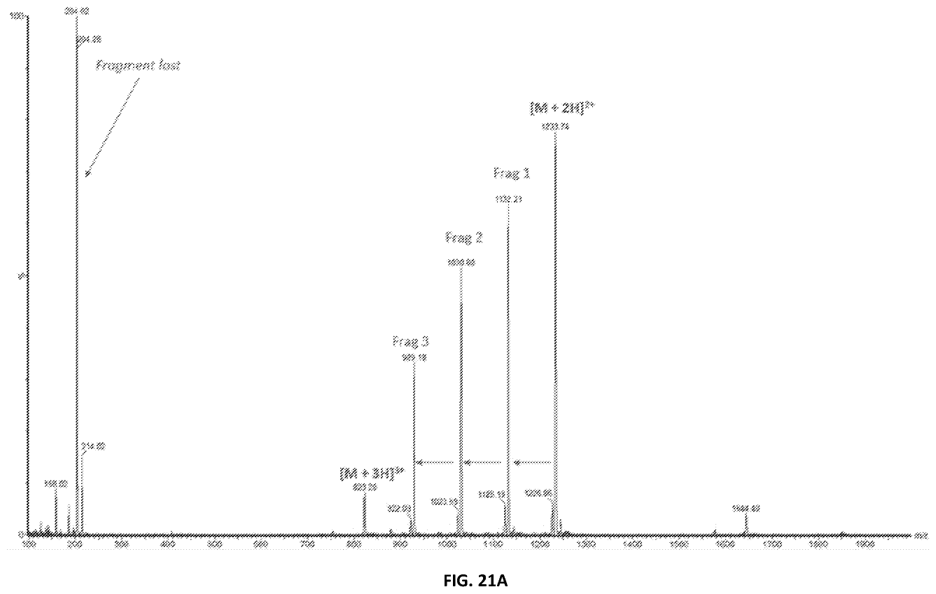

In some embodiments, the bifunctional compound of Formula (I) comprises an anti-PCSK9 antibody, e.g., a modified anti-PCSK9 antibody. In an embodiment, A_{G} comprises an anti-PCSK9 antibody), L comprises a polyethylene glycol moiety (e.g., a PEG3, PEG4, PEG6, or PEG8 moiety), and R_{G} comprises an GalNAc moiety (e.g., a plurality of GalNAc moieties, e.g., 2, 3, or 4 GalNAc moieties). In an embodiment, the bifunctional compound of Formula (I) comprises a GalNAc-labeled anti-PCSK9 antibody, e.g., as depicted as triGalNAc functionalized PCSK9 antibody 10 in FIG. 20B, e.g., as described in Examples 3 and 9-11, and FIGS. 5-6. In an embodiment, the bifunctional compound of Formula (I) comprises a GalNAc-labeled anti-PCSK9 antibody, e.g., as depicted in FIG. 20C.

In some embodiments, the bifunctional compound of Formula (I) is a bifunctional compound described in Table 4.

TABLE 4
Exemplary A_{G}-L-R_{G} bifunctional compounds

| Extracellular target | A_{G} | L | R_{G} |
|---|---|---|---|
| A protein (e.g., soluble protein, a | a binding moiety | absent or as | a binding moiety (e.g., an antibody or a |
| membrane-associated protein), a | (e.g., an antibody | defined in the | fragment thereof, a carbohydrate or |
| pathogenic target, or a non- | or a fragment | disclosure, as | carbohydrate derivative, a small molecule or |
| protein (e.g., lipoprotein, | thereof, a protein | disclosed herein | a protein/peptide) that binds to ASGPR or |
| liposome, nucleic acid (e.g., | or a fragment | | M6PR or IGF2R, as disclosed herein |
| oligonucleotides, DNA, RNA), | thereof, or a | | a binding moiety (e.g., an antibody or a |
| toxin, virus particle, or cell (e.g., | receptor or a | | fragment thereof, a carbohydrate or |
| prokaryotic cells, eukaryotic | fragment thereof) | | carbohydrate derivative, a small molecule or |
| cells)), as disclosed herein | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| a protein (e.g., soluble protein | a binding moiety | absent or as | a binding moiety (e.g., an antibody or a |
| or membrane-associated | (e.g., an antibody | defined in the | fragment thereof, a carbohydrate or |
| protein), as disclosed herein | or a fragment | disclosure, as | carbohydrate derivative, a small molecule or |
| | thereof, a protein | disclosed herein | a protein/peptide) that binds to ASGPR or |
| | or a fragment | | M6PR or IGF2R, as disclosed herein |
| | thereof, or a | | a binding moiety (e.g., an antibody or a |
| | receptor or a | | fragment thereof, a carbohydrate or |
| | fragment thereof) | | carbohydrate derivative, a small molecule or |
| | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| A soluble protein (e.g., | a binding moiety | absent or as | a binding moiety (e.g., an antibody or a |
| antibodies or fragment thereof | (e.g., an antibody | defined in the | fragment thereof, a carbohydrate or |
| (e.g., autoantibodies), soluble | or a fragment | disclosure, as | carbohydrate derivative, a small molecule or |
| receptors, secreted proteins, | thereof, a protein | disclosed herein | a protein/peptide) that binds to ASGPR or |
| growth factors, cytokines, | or a fragment | | M6PR or IGF2R, as disclosed herein |
| chemokines, enzymes, or | thereof, or a | | a binding moiety (e.g., an antibody or a |
| hormones), as disclosed herein | receptor or a | | fragment thereof, a carbohydrate or |
| | fragment thereof) | | carbohydrate derivative, a small molecule or |
| | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| A membrane-associated protein | a binding moiety | absent or as | a binding moiety (e.g., an antibody or a |
| (e.g., a type I, type II or | (e.g., an antibody | defined in the | fragment thereof, a carbohydrate or |
| multipass membrane protein or a | or a fragment | disclosure, as | carbohydrate derivative, a small molecule or |
| glycophosphatidylinositol (GPI) | thereof, a protein | disclosed herein | a protein/peptide) that binds to ASGPR or |
| anchored membrane associated | or a fragment | | M6PR or IGF2R, as disclosed herein |
| protein, a receptor, a protein | thereof, or a | | a binding moiety (e.g., an antibody or a |
| channel, or an ion channel), as | receptor or a | | fragment thereof, a carbohydrate or |
| disclosed herein | fragment thereof) | | carbohydrate derivative, a small molecule or |
| | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| A membrane-associated protein | a binding moiety | absent or as | a binding moiety (e.g., an antibody or a |
| (e.g., TNFR1 (tumor necrosis | (e.g., an antibody | defined in the | fragment thereof, a carbohydrate or |
| factor receptor 1), ILIR | or a fragment | disclosure, as | carbohydrate derivative, a small molecule or |
| (interleukin 1 receptor), EGFR | thereof, a protein | disclosed herein | a protein/peptide) that binds to ASGPR or |
| (epidermal growth factor | or a fragment | | M6PR or IGF2R, as disclosed herein |
| receptor), CXCR4 (C-X-C | thereof, or a | | a binding moiety (e.g., an antibody or a |
| chemokine receptor type 4), | receptor or a | | fragment thereof, a carbohydrate or |
| TSP1 (thrombospondin 1), | fragment thereof) | | carbohydrate derivative, a small molecule or |
| NKG2D (encoded by KLRK1), | that binds to an | | a protein/peptide) that binds to ASGPR, as |
| suPAR (urokinase plasminogen | extracellular | | disclosed herein |
| activator receptor), GFRAL, | target, as | | |
| IL4R-4 receptor, thymic stromal | disclosed herein | | |
| lymphopoietin receptor, IL7R, | an antibody or a | | an antibody or a fragment thereof that binds |
| TGFa receptor, PD-L1, or sToll | fragment thereof | | to ASGPR, as disclosed herein |
| receptor) | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| Proprotein convertase | a binding moiety | absent or as | a binding moiety (e.g., an antibody or a |
| subtilisin/kexin type 9 (PCSK9), | (e.g., an antibody | defined in the | fragment thereof, a carbohydrate or |
| tumor necrosis factor receptor 1 | or a fragment | disclosure, as | carbohydrate derivative, a small molecule or |
| (TNFR1), interleukin-1 receptor | thereof, a protein | disclosed herein | a protein/peptide) that binds to ASGPR or |
| (IL1R), low density lipoproteins, | or a fragment | | M6PR or IGF2R, as disclosed herein |
| such as apolipoprotein B (LDL | thereof, or a | | a binding moiety (e.g., an antibody or a |
| (ApoB), lipoprotein(a) (Lp(a)), | receptor or a | | fragment thereof, a carbohydrate or |
| apolipoprotein C3 (ApoCIII), | fragment thereof) | | carbohydrate derivative, a small molecule or |
| angiopoietin-like 3 (ANGPTL3), | that binds to an | | a protein/peptide) that binds to ASGPR, as |
| angiopoietin-like 4 (ANGPTL4), | extracellular | | disclosed herein |
| angiopoietin-like 8 (ANGPTL8), | target, as | | |
| Factor 11, growth differentiation | disclosed herein | | |
| factor 15 (GDF15), lipoprotein | an antibody or a | | an antibody or a fragment thereof that binds |
| lipase (LPL), interleukin 1-beta | fragment thereof | | to ASGPR, as disclosed herein |
| (IL1β), interleukin 17 (IL17), | that binds to an | | a carbohydrate or carbohydrate derivative |
| Complement Factor B, | extracellular | | that binds to ASGPR, as disclosed herein |
| complement Factor D, | target, as | | a small molecule that binds to ASGPR, as |
| myeloperoxidase (MPO), | disclosed herein | | disclosed herein |
| immunoglobulin E (IgE), | | | a protein/peptide that binds to ASGPR, as |
| epidermal growth factor receptor | | | disclosed herein |
| (EGFR), programmed cell death | a protein or a | | a binding moiety (e.g., an antibody or a |
| protein 1 (PD-1), programmed | fragment thereof | | fragment thereof, a carbohydrate or |
| death-ligand 1 (PD-L1), | that binds to an | | carbohydrate derivative, a small molecule or |
| interleukin 7 (IL7), interleukin | extracellular | | a protein/peptide) that binds to M6PR or |
| 12A (IL12A), interleukin 23 | target, as | | IGF2R, as disclosed herein |
| (IL23), tumor necrosis factor A | disclosed herein | | an antibody or a fragment thereof that binds |
| (TNFA), C-X-C chemokine | | | to M6PR or IGF2R, as disclosed herein |
| receptor 4 (CXCR4), | a receptor or a | | a carbohydrate or carbohydrate derivative |
| microtubule associated protein | fragment thereof | | that binds to M6PR or IGF2R, as disclosed |
| tau (MAPT), complement factor | that binds to an | | herein |
| H-related protein 3 (FHR3), | extracellular | | a small molecule that binds to M6PR or |
| Tissue inhibitor of | target, as | | IGF2R, as disclosed herein |
| metalloproteinases 1 (TIMP1), | disclosed herein | | a protein/peptide that binds to M6PR or |
| Apelin, bone morphogenetic | | | IGF2R, as disclosed herein |
| protein 6 (BMP6), bone | | | |
| morphogenetic protein 9/growth | | | |
| differentiation factor 9 | | | |
| (BMP9/GDF2), colony | | | |
| stimulating factor 1 receptor | | | |
| (CSF-1), erythropoietin (EPO), | | | |
| interleukin 5 (IL5), milk fat | | | |
| globule-EGF Factor 8 protein | | | |
| (MFGE8), thymic stromal | | | |
| lymphopoietin (TSLP), | | | |
| thrombospondin (TSP), | | | |
| complement component 5 (C5), | | | |
| C-X-C motif chemokine ligand | | | |
| 10 (CXCL10), fibroblast growth | | | |
| factor 23 (FGF23), insulin-like | | | |
| growth factor 1 (IGF1), | | | |
| interleukin 10 (IL-10), | | | |
| interleukin 13 (IL-13), | | | |
| interleukin 2 (IL-2), interleukin | | | |
| 6 (IL-6), IL-17, vascular | | | |
| endothelial growth factor A | | | |
| (VEGF-A), natural killer group | | | |
| 2D receptor (NKG2D), | | | |
| adenosine deaminase 2 (ADA2), | | | |
| soluble urokinase-type | | | |
| plasminogen activator receptor | | | |
| (suPAR), transforming growth | | | |
| factor beta 1 (TGF-β1), | | | |
| interleukin 4 (IL-4) receptor, | | | |
| sToll receptor, Tau, proglanulin, | | | |
| alpha-synuclein, a toxin, a | | | |
| venom, an HBV soluble antigen, | | | |
| a viral antigen, a prion protein, a | | | |
| scFv, an AAV, and an anti-AAV | | | |
| antibody | | | |
| A pathogenic target (e.g., an | a binding moiety | absent or as | a binding moiety (e.g., an antibody or a |
| extracellular secreted protein, a | (e.g., an antibody | defined in the | fragment thereof, a carbohydrate or |
| pathogenic autoantibody, an | or a fragment | disclosure, as | carbohydrate derivative, a small molecule or |
| anti-drug antibody, a cell surface | thereof, a protein | disclosed herein | a protein/peptide) that binds to ASGPR or |
| receptor, a neurological target, | or a fragment | | M6PR or IGF2R, as disclosed herein |
| an immunooncology target, a | thereof, or a | | a binding moiety (e.g., an antibody or a |
| toxin or drug), as disclosed | receptor or a | | fragment thereof, a carbohydrate or |
| herein | fragment thereof) | | carbohydrate derivative, a small molecule or |
| | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |
| non-protein (e.g., lipoprotein, | a binding moiety | absent or as | a binding moiety (e.g., an antibody or a |
| liposome, nucleic acid (e.g., | (e.g., an antibody | defined in the | fragment thereof, a carbohydrate or |
| oligonucleotides, DNA, RNA), | or a fragment | disclosure, as | carbohydrate derivative, a small molecule or |
| toxin, virus particle, or cell (e.g., | thereof, a protein | disclosed herein | a protein/peptide) that binds to ASGPR or |
| prokaryotic cells, eukaryotic | or a fragment | | M6PR or IGF2R, as disclosed herein |
| cells)), as disclosed herein | thereof, or a | | a binding moiety (e.g., an antibody or a |
| | receptor or a | | fragment thereof, a carbohydrate or |
| | fragment thereof) | | carbohydrate derivative, a small molecule or |
| | that binds to an | | a protein/peptide) that binds to ASGPR, as |
| | extracellular | | disclosed herein |
| | target, as | | |
| | disclosed herein | | |
| | an antibody or a | | an antibody or a fragment thereof that binds |
| | fragment thereof | | to ASGPR, as disclosed herein |
| | that binds to an | | a carbohydrate or carbohydrate derivative |
| | extracellular | | that binds to ASGPR, as disclosed herein |
| | target, as | | a small molecule that binds to ASGPR, as |
| | disclosed herein | | disclosed herein |
| | | | a protein/peptide that binds to ASGPR, as |
| | | | disclosed herein |
| | a protein or a | | a binding moiety (e.g., an antibody or a |
| | fragment thereof | | fragment thereof, a carbohydrate or |
| | that binds to an | | carbohydrate derivative, a small molecule or |
| | extracellular | | a protein/peptide) that binds to M6PR or |
| | target, as | | IGF2R, as disclosed herein |
| | disclosed herein | | an antibody or a fragment thereof that binds |
| | | | to M6PR or IGF2R, as disclosed herein |
| | a receptor or a | | a carbohydrate or carbohydrate derivative |
| | fragment thereof | | that binds to M6PR or IGF2R, as disclosed |
| | that binds to an | | herein |
| | extracellular | | a small molecule that binds to M6PR or |
| | target, as | | IGF2R, as disclosed herein |
| | disclosed herein | | a protein/peptide that binds to M6PR or |
| | | | IGF2R, as disclosed herein |

### Pharmaceutical Compositions, Kits, and Administration

The present disclosure provides compositions (e.g., pharmaceutical compositions) comprising a bifunctional compound, e.g., a bifunctional compound of Formula (I) or a pharmaceutically acceptable salt thereof, as described herein, and optionally a pharmaceutically acceptable excipient. In certain embodiments, the pharmaceutical composition described herein comprises a bifunctional compound of Formula (I) or a pharmaceutically acceptable salt thereof, and optionally a pharmaceutically acceptable excipient. In certain embodiments, the bifunctional compound of Formula (I) or a pharmaceutically acceptable salt thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the bifunctional compound of Formula (I) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the bifunctional compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally (including subcutaneous, intramuscular, intravenous and intradermal), by inhalation spray, topically, rectally, nasally, buccally, vaginally or via an implanted reservoir. In some embodiments, the bifunctional compounds or compositions thereof are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal, intralesional, and intracranial injection or infusion techniques. Preferably, the compositions are administered orally, subcutaneously, intraperitoneally, or intravenously. In an embodiment, the compositions described herein are administered by subcutaneous injection (e.g., depot subcutaneous injection). Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release.

In some embodiments, the composition is suitable for buccal or sublingual administration, including tablets, lozenges and pastilles. A provided compound can also be in micro-encapsulated form. Alternatively, pharmaceutically acceptable compositions of this disclosure may be administered in the form of suppositories for rectal administration. Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle or hydrogel vehicle. In an embodiment, the compositions described herein are formulated for sustained release, e.g., as a depot formulation (e.g., subcutaneous depot injection). Exemplary depot formulations may include excipients such as a hydrogel, peptide, phospholipid, or polyethylene glycol.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Bifunctional compounds provided herein may be formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a bifunctional compound required to achieve an effective amount will vary from subject to subject, depending, for example, on species, age, and general condition of a subject, severity of the side effects or disorder, identity of the particular bifunctional compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a bifunctional compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the bifunctional compound of Formula (I) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a bifunctional compound or compositions thereof, as described herein, can be administered in combination with one or more additional pharmaceutical agents. The bifunctional compound or compositions thereof can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The bifunctional compound or compositions thereof can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the bifunctional compound or compositions thereof described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the disclosure are kits (e.g., pharmaceutical packs). The inventive kits may be useful for preventing and/or treating a disease, disorder, condition, or clinical situation, e.g., as described herein. The kits provided may comprise an inventive pharmaceutical composition or bifunctional compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a bifunctional compound described herein, or a pharmaceutically acceptable salt, solvate, hydrate, tautomer, or stereoisomer thereof, or a pharmaceutical composition thereof. In certain embodiments, the kit of the disclosure includes a first container comprising a bifunctional compound described herein, or a pharmaceutically acceptable salt thereof, or a pharmaceutical composition thereof. In certain embodiments, the kits are useful in preventing and/or treating a disease, disorder, condition, or a clinical situation described herein in a subject (e.g., a proliferative disease or a non-proliferative disease). In certain embodiments, the kits further include instructions for administering the bifunctional compound, or a pharmaceutically acceptable salt, solvate, hydrate, tautomer, or stereoisomer thereof, or a pharmaceutical composition thereof, to a subject to prevent and/or treat a disease, disorder, condition, or clinical situation described herein.

### Methods of Use

Described herein are bifunctional compounds useful for the degradation of extracellular targets. In some embodiments, the bifunctional compounds, e.g., the bifunctional compounds of Formula (I), may be used to reduce or depress the level of an extracellular target by activating a degradation pathway (e.g., receptor mediated endocytosis, endosomal degradation, or lysosomal degradation). The bifunctional compounds may be useful as research tools and/or as treatments for a disease, disorder, condition or clinical situation, e.g., as described herein.

Conventional therapeutics, for instance protein-directed therapeutics, often provide desirable effects by obstructing protein function, such as enzyme inhibition, or by recruiting immune effectors, as in the case of many monoclonal antibody drugs. Typically, because of the reversible nature of conventional drug/target interaction, the efficacy of such conventional therapeutics require a superstoichiometric drug concentration to maintain inhibition, which can be lost over time as drug concentrations decrease. However, the methods described herein comprising use of a bifunctional compound for targeting protein degradation, may show improved efficacy at sub-stoichiometric, stoichiometric, or superstoichiometric, concentrations, where efficacy is limited by the re-synthesis of target molecule (e.g., protein) rather than on drug concentration. In an embodiment, the methods described herein comprise use of a bifunctional compound show improved efficacy at stoichiometric concentrations.

In one aspect, the present disclosure features a method of targeting an extracellular target molecule, e.g., a soluble protein or a membrane-associated protein in a cell, for degradation, comprising administering the bifunctional compound described herein (e.g., a bifunctional compound of Formula (I)). The method may be an in vitro method, an in vivo method, or an ex vivo method. In some embodiments, the method comprises contacting a sample (e.g., a cell or tissue sample) with a bifunctional compound described herein. In some embodiments, the method comprises administering a bifunctional compound described herein to a subject.

In some embodiments, the extracellular target molecule is associated with a disease, disorder, condition, or clinical situation in a subject. In some embodiments, the method comprises modulating the level and/or activity of the extracellular target molecule, in the subject in response to the bifunctional compound. In some embodiments, the method comprises decreasing the level and/or activity of the extracellular target molecule in a subject in response to administration of the bifunctional compound described herein compared to the level and/or activity of the extracellular target molecule in a subject without administration of the bifunctional compound described herein. In some embodiments, the method comprises decreasing the level and/or activity of the extracellular target molecule in a subject in response to administration of the bifunctional compound, e.g., by about 10%, 15%, 20%, 25%, 30%, 35%, 40%, 45%, 50%, 55%, 60%, 65%, 70%, 75%, 80%, 85%, 90%, 95%, 99%, or more, e.g., relative to a reference or the level and/or activity of the extracellular target molecule in a subject in the absence of administration of the bifunctional compound described herein (e.g., by about greater than 50%, 60%, 70%, 80%, 90%, 95%, 99% or more). In some embodiments, the method comprises decreasing a symptom associated with a disease, disorder, condition, or clinical situation in a subject in response administration of the bifunctional compound described herein compared to a reference or the symptom associated with a disease, disorder, condition, or clinical situation in a subject in the absence of administration of the bifunctional compound described herein. In some embodiments, the method comprises decreasing a symptom associated with a disease, disorder, condition, or clinical situation in a subject in response administration of the bifunctional compound, e.g., by about 10%, 15%, 20%, 25%, 30%, 35%, 40%, 45%, 50%, 55%, 60%, 65%, 70%, 75%, 80%, 85%, 90%, 95%, 99%, or more, e.g., relative to a reference or the symptom associated with a disease, disorder, condition, or clinical situation in a subject in the absence of administration of the bifunctional compound described herein.

In some embodiments, the disease, disorder, condition, or clinical situation is associated with an elevated level and/or activity of an extracellular target molecule (e.g., a soluble protein or membrane-associated protein, such as an antibody, lipoprotein, receptor, growth factor, cytokine, chemokine, enzyme, hormone, neurotransmitter, or a combination thereof). The level and/or activity of the extracellular target molecule may be increased, e.g., by about 10%, 15%, 20%, 25%, 30%, 35%, 40%, 45%, 50%, 55%, 60%, 65%, 70%, 75%, 80%, 85%, 90%, 95%, 99%, or more, e.g., relative to a reference. In some embodiments, the disease, disorder, condition, or clinical situation is associated with an elevated level and/or activity of an extracellular target molecule, and administration of the bifunctional compound described herein results in return of levels of the extracellular target molecule to normal (e.g., healthy levels).

In some embodiments, the disease, disorder, condition, or clinical situation is a proliferative disease (e.g., cancer), a neurological disease (e.g., Alzheimer's disease, neurodegeneration), a cardiovascular disease, a respiratory disease, a dermatological disease, a hematological disease, an inflammatory disease, a metabolic disorder, an infectious disease, or a renal disease or disorder. Exemplary diseases, disorders, conditions, and clinical situations include acute inflammatory demyelinating polyradiculoneuropathy (Guillain-Barré syndrome), acute liver failure, anti-glomerular basement membrane disease (Goodpasture syndrome), chronic inflammatory demyelinating polyradiculoneuropathy (CIDP), cutaneous T cell lymphoma (CTCL), mycosis fungoides, Sézary syndrome, familial hypercholesterolemia, focal segmental glomerulosclerosis (FSGS), hereditary hemochromatosis, hyperviscosity in hypergammaglobulinemia, hyperviscosity in hypergammaglobulinemia, myasthenia gravis, N-methyl-D-aspartate receptor antibody encephalitis, paraproteinemic demyelinating neuropathies; chronic acquired demyelinating polyneuropathies, polycythemia vera; erythrocytosis, thrombotic microangiopathy, thrombotic thrombocytopenic purpura (TTP), vasculitis, Wilson disease, fulminant, dilated cardiomyopathy, Graft-versus-host disease (GVHD), Lipoprotein(a) hyperlipoproteinemia, multiple sclerosis, neuromyelitis optica spectrum disorders (NMOSD), pediatric autoimmune neuropsychiatric disorders associated with streptococcal infections (PANDAS), Sydenham's chorea, peripheral vascular diseases, sickle cell disease, voltage-gated potassium channel (VGKC) antibody related diseases.

In some embodiments, the disease, disorder, condition, or clinical situation comprises cancer, Alzheimer's disease, atherosclerosis, arteriosclerosis, hyperlipidemia, familial hypercholesterolemia, familial chylomicronemia syndrome, inflammation, asthma, urticaria, IgA nephropathy, and membranous nephropathy.

In some embodiments, the disease, disorder, condition, or clinical situation comprises removal of a pathogenic antibody. The pathogenic antibody may be responsible for an autoimmune disease, e.g., membranous nephropathy, IgF nephropathy, myasthenia gravis, and allergies.

In some embodiments, the disease, disorder, condition, or clinical situation comprises removal of an anti-drug antibody, e.g., wherein clearance of an anti-drug antibody allows for effective treatment.

In some embodiments, the disease, disorder, condition, or clinical situation comprises atherosclerosis, and the target is a low-density lipoprotein, very low-density lipoprotein, a chylomicron, PCSK9, ANGPTL3, ANGPTL4, ANGPTL8. In some embodiments, the disease, disorder, condition, or clinical situation comprises a central nervous system disease (e.g., Alzheimer's disease, Parkinson's disease, muscular dystrophy) and the target is tau or TNFR1. In some embodiments, the disease, disorder, condition, or clinical situation comprises a respiratory disease (e.g., cancer) and the target is EGFR or MICA.

In some embodiments, the disease, disorder, condition, or clinical situation is caused by or relates to a pathogenic autoantibody (e.g., presence of a pathogenic autoantibody). Exemplary diseases, disorders, conditions, or clinical situations related to said pathogenic autoantibody include acquired thrombotic thrombopenic purpura, Addison disease, anti-NMDA encephalitis, autoimmune hemolytic anemia (AIHA), autoimmune hepatitis, autoimmune idiopathic thrombocytopenia, autoimmune myopathies, autoimmune orchitis, autoimmune pancreatitis, bulbous pemphigoid, dry eye disease, epidermolysis bullosa acquisita, eosinophilic granulomatosis with polyangiitis (EGPA), Goodpasture's disease, granulomatosis with polyangitis, Graves Disease, Hashimoto's thyroiditis, idiopathic interstitial pneumonias, idiopathic thrombocytopenic purpura (ITP), microscopic polyangiitis (MPA), myasthenia gravis, myocarditis, neuromyelitis optica (NMO), ovarian insufficiency, pemphigus, pernicious anemia, primary biliary cholangitis (PBC), primary membranous nephropathy, rheumatoid arthritis, Sjögren's syndrome, systemic lupus erythematosus (SLE), systemic sclerosis, and Type I diabetes.

In some embodiments, the methods described herein comprise decreasing proprotein convertase subtilisin/kexin type 9 (PCSK9) levels in a subject, e.g., by about 55%, 60%, 65%, 70%, 75%, 80%, 85%, 90%, 91%, 92%, 93%, 94%, 95%, 96%, 97%, 98%, 99%, or more, e.g., relative to a reference or the PCSK9 levels in a subject in the absence of administration of the bifunctional compound described herein. PCSK9 has pronounced effects on plasma low density lipoprotein cholesterol (LDL-C) levels via its modulation of hepatic low density lipoprotein receptors (LDLR), the main route by which cholesterol is removed from the circulation. PCSK9 binds the LDLR and directs it to lysosomal degradation, thereby increasing plasma LDL-C levels and, in turn, coronary heart disease risk. (Maxwell K. N., Proc. Natl. Acad. Sci., 101, 2004, 7100-7105; Park, S. W., J. Biol. Chem. 279, 2004, 50630-50638; Lagace T. A., et al. J. Clin. Invest. 2006, 116(11):2995-3005). Overexpression of mouse or human PCSK9 in mice has been shown to elevate total and LDL-C levels and dramatically reduce hepatic LDLR protein, without an observed effect on the levels of mRNA, SREBP, or SREBP protein nuclear to cytoplasmic ratio. (Maxwell K. N., Proc. Natl. Acad. Sci. 101, 2004, 7100-7105). Moreover, mutations in PCSK9 that cause loss of PCSK9 function in mouse models have also been shown to lower total and LDL-C levels. (Cohen, J. C., et al., N. Engl. J. Med., 354, 2006, 1264-1272). Thus, indicating that modulation of PCSK9 results in a reduction of LDLR protein levels. Exemplary diseases, disorders, conditions, and clinical situations relating to PCSK9 include hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL (e.g., elevated VLDL and/or chylomicrons), elevated triglycerides, sepsis, and xanthoma.

In some embodiments, the methods described herein comprise decreasing complement factor H-related protein 3 (FHR3) levels in a subject, e.g., by about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, e.g., relative to a reference or the FHR3 levels in a subject in the absence of administration of the bifunctional compounds described herein. Complement-mediated immune responses are tightly regulated by a number of endogenously produced proteins to regulate activity and discriminate between healthy self (non-activating) and damaged or non-self, pathogenic activating cells. These complement control proteins range from cell surface bound (i.e., CR1, MCP, DAF) to circulating proteins (i.e., factor H and C4BP) that are recruited to self-surfaces by binding to polysaccharides such as glycosaminoglycans on host self-surfaces to inactivate complement (Mol Immuno 47(13): 2187-2197). Complement regulation is tightly controlled to maintain homeostasis and whose dysregulation and deficiency causing it to target host cells, have been implicated in many diseases.

Factor H (FH), a major negative regulator of alternative complement pathway activation, belongs to a family that also includes five other related family members thought to have arisen from nonallelic homologous recombination and interlocus gene conversion: complement factor H-related protein 1 (FHR1), complement factor H-related protein 2 (FHR2), complement factor H-related protein 3 (FHR3), complement factor H-related protein 4 with isoforms 4A and 4B (FHR4A and FHR4B) and complement factor H-related protein 5 (FHR5). FHR3, unlike factor H, lacks the complement regulatory domains essential for complement inactivation and also competes with factor H, resulting in complement over-activation. Thus the present invention provides bifunctional compounds for use in modulating the concentration of complement factor H-proteins, specifically FHR3, to remove factor H's competitor and thereby restore factor H-mediated regulation to treat disorders caused by excessive complement activation. Exemplary diseases, disorders, conditions, and clinical situations relating to FHR3 include nephropathy, age-related macular degeneration, atypical hemolytic uremic syndrome and hepatocellular carcinoma (HCC).

### EXAMPLES

In order that the disclosure described herein may be more fully understood, the following examples are set forth. The examples described in this application are offered to illustrate the bifunctional compounds, pharmaceutical compositions, and methods provided herein and are not to be construed in any way as limiting their scope.

The bifunctional compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Abbreviations used in the following examples and elsewhere herein are provided in Table 5.

TABLE 5
Abbreviations

| | | | |
|---|---|---|---|
| aq.: aqueous | EDCI: 1-Ethyl-3- (3- | LC and LCMS: liquid | PE: Petroleum |
| | dimethylaminopropyl) | chromatography and | ether |
| | carbodiimide | liquid chromatography- | |
| | | mass spectrometry | |
| BnOH: Benzyl alcohol | eq.: equivalent | min: minute(s) | Ph: phenyl |
| BSA: bovine serum | ESI-MS: electrospray | Me: methyl | RP: reverse |
| albumin | ionization mass | | phase |
| | spectrometry | | |
| DCM: dichloromethane | Et and EtOAc: ethyl | MS: mass | rpm: revolutions |
| | and ethyl acetate | | per minute |
| DIC: N,N′- | Et₃N: triethylamine | m/z: mass to charge | rt: room |
| Diisopropylcarbodiimide | | ratio | temperature |
| DMAP: 4- | HCl: hydrochloric acid | M and mM: molar | sat.: saturated |
| Dimethylaminopyridine | | and millimolar | |
| DMF: N,N- | HOBt: | mg: milligram | TLC: Thin-layer |
| dimethylformamide | Hydroxybenzotriazole | | chromatography |
| DMSO: dimethylsulfoxide | HPLC: high pressure | μL, mL and L: | TMSOTf: |
| | liquid chromatography | microliter(s), | trimethylsilyl |
| | | milliliter(s) and | trifluoromethane- |
| | | liter(s) | sulfonate |
| DIEA or DIPEA: N,N- | h, hr: hour(s) | N: equivalent per liter | tᵣ: retention |
| diisopropylethylamine | | | time |
| EA: Ethyl acetate | IC50: half maximal | NMR: nuclear | wt: weight |
| | inhibitory concentration | magnetic resonance | |

### Example 1: Synthesis of Exemplary ASGPR Receptor Ligands

### Synthesis of Intermediates

### Type A:

### Synthesis of benzyl 5-hydroxypentanoate (int-A1

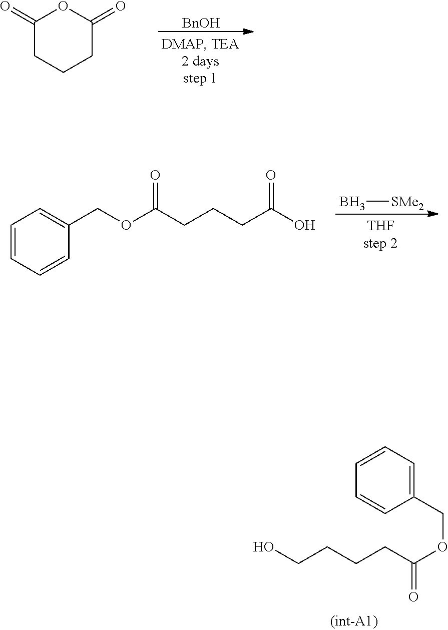

Step 1: To a solution of dihydro-2H-pyran-2,6(3H)-dione (20 g, 175.44 mmol) and BnOH (20.8 g, 192.98 mmol) in DCM (150 mL) were added DMAP (0.32 g, 2.62 mmol) and Et₃N (29 mL, 210.5 mmol) at 0° C. The reaction mixture was allowed to warm to room temperature and then stirred for two days. The reaction mixture was evaporated to dryness and the resulting residue dissolved in DCM (200 mL) and washed with 3M HCl (100 mL×2). The organic layer was dried over anhydrous Na₂SO₄ and concentrated. The residue was purified by silica gel column chromatography (elute: PE:EA=20:1-10:1) to obtain 5-(benzyloxy)-5-oxopentanoic acid as a colorless oil. ¹H NMR (400 MHz, CDCl₃) δ ppm 7.38-7.28 (m, 5H), 5.12 (s, 2H), 2.46-2.40 (m, 4H), 2.00-1.93 (m, 2H).

Step 2: To a solution of 5-(benzyloxy)-5-oxopentanoic acid (30 g, 135.1 mmol) in THF (200 mL) was added BH₃—SMe₂ (20.2 ml, 202.7 mmol) dropwise at 0° C. under N₂ protection. The mixture was allowed to warm to room temperature and stirred for 16 hours. TLC showed the start material was completely consumed. The reaction was carefully quenched with H₂O (8 mL). The resulting mixture was filtered and the filtrate was concentrated. The residue was purified by silica gel column chromatography (elute: PE:EA=20:1-2:1) to afford benzyl 5-hydroxypentanoate (int-A1). ¹H NMR (400 MHz, CDCl₃) δ ppm 7.41-7.33 (m, 5H), 5.14 (s, 2H), 3.66 (t, 2H, J=6 Hz), 2.43 (t, 2H, J=7.2 Hz), 1.80-1.73 (m, 2H), 1.65-1.58 (m, 2H).

### Type B:

### Synthesis of (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate (int-B1

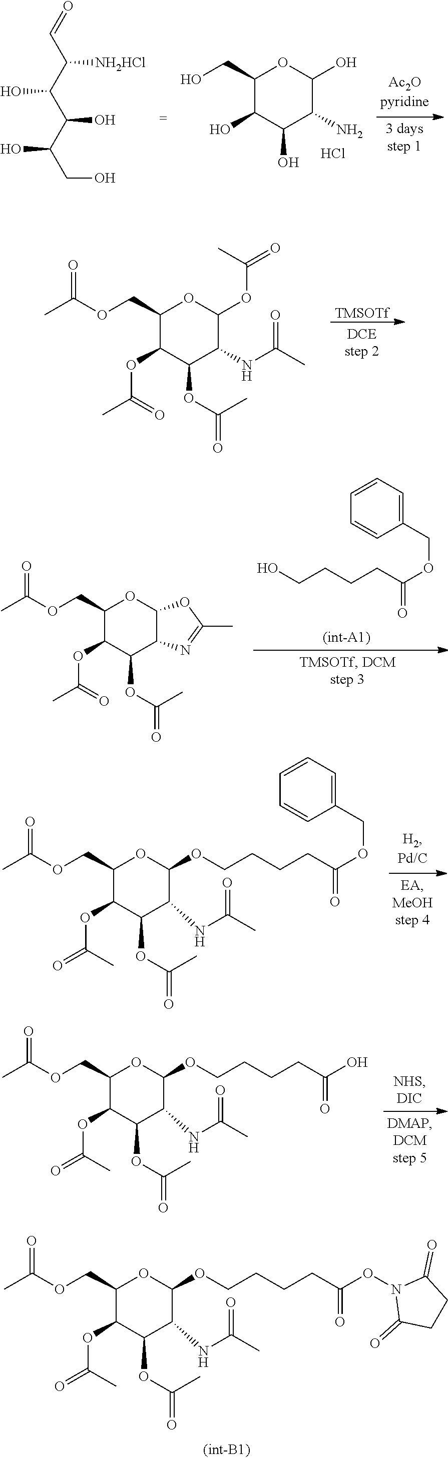

Step 1: To a solution of (2R,3R,4R,5R)-2-amino-3,4,5,6-tetrahydroxyhexanal hydrochloride (100.0 g, 0.132 mol) in pyridine (1 L) was added acetic anhydride (473 g, 4.64 mol) at 0° C. and the reaction mixture stirred for 72 hours at room temperature. The resultant precipitate was collected and washed with H₂O (200 mL×2), dried in vacuum to give (3R,4R,5R,6R)-3-acetamido-6-(acetoxymethyl)tetrahydro-2H-pyran-2,4,5-triyl triacetate. ¹H NMR (400 MHz, CDCl₃) δ ppm 5.68 (d, 1H, J=8.8 Hz), 5.46 (d, 1H, J=9.2 Hz), 5.35 (d, 1H, J=3.2 Hz), 5.07 (dd, 1H, J1=11.2 Hz, J2=3.2 Hz), 4.47-4.39 (m, 1H), 4.18-4.07 (m, 2H), 4.02-3.98 (m, 1H), 2.16 (s, 3H), 2.11 (s, 3H), 2.03 (s, 3H), 2.00 (s, 3H), 1.93 (s, 3H).

Step 2: To a solution of (3R,4R,5R,6R)-3-acetamido-6-(acetoxymethyl)tetrahydro-2H-pyran-2,4,5-triyl triacetate (100 g, 0.257 mol) in 1,2-dichloroethane (500 mL) cooled to 0° C. was added TMSOTf (85.5 g, 0.385 mol), the mixture was stirred for 10 min, then heated to 50° C. and stirred for 3 hours. TLC showed the starting material was completely consumed. After cooling, the resultant mixture was treated with sat. aqueous NaHCO₃ (1000 mL) at 0° C., extracted with DCM (500 mL×2). The combined organic layers were dried over Na₂SO₄ and concentrated. The residue was dried under high vacuum overnight to give (3aR,5R,6R,7R,7aR)-5-(acetoxymethyl)-2-methyl-3a,6,7,7a-tetrahydro-5H-pyran[3,2-d]oxazole-6,7-diyl diacetatel. ¹H NMR (400 MHz, CDCl₃) δ ppm 6.00 (d, 1H, J=2.8 Hz), 5.47-5.46 (m, 1H), 4.93-4.90 (m, 1H), 4.27-4.18 (m, 2H), 4.13-4.09 (m, 1H), 4.02-3.98 (m, 1H), 2.13 (s, 3H), 2.07 (s, 6H), 2.06 (s, 3H).

Step 3: (3aR,5R,6R,7R,7aR)-5-(acetoxymethyl)-2-methyl-3a,6,7,7a-tetrahydro-5H-pyrano[3,2-d]oxazole-6,7-diyl diacetate (65 g, 197.4 mmol) and benzyl 5-hydroxypentanoate (int-A1) (41 g, 197.4 mmol) were dissolved in DCM (600 mL). Molecular sieves (50 g) were added and the reaction was stirred for 30 min, followed by addition of TMSOTf (6.5 g, 29.6 mmol). The reaction mixture was then stirred at room temperature overnight. TLC showed the start material was completely consumed. The reaction mixture was filtered to remove the molecular sieves. The filtrate was treated with saturated aqueous NaHCO₃ (500 mL) and extracted with DCM (500 mL×2). The combined organic layer was dried over anhydrous Na₂SO₄ and concentrated. The residue was purified by silica gel column chromatography (elute: PE:EA=2:1-1:2) to obtain (2R,3R,4R,5R,6R)-5-acetamido-2-(acetoxymethyl)-6-((5-(benzyloxy)-5-oxopentyl)oxy)tetrahydro-2H-pyran-3,4-diyl diacetate. ¹H NMR (400 MHz, CDCl₃) δ ppm 7.37-7.32 (m, 5H), 5.60 (d, 1H, J=8.4 Hz), 5.35 (d, 1H, J=2.4 Hz), 5.25 (dd, 1H, J1=11.6 Hz, J2=3.6 Hz), 5.11 (s, 2H), 4.63 (d, 1H, J1=8.4 Hz), 4.15-4.11 (m, 2H), 3.98-3.86 (m, 3H), 3.55-3.45 (m, 1H), 2.41-2.36 (m, 2H), 2.14 (s, 3H), 2.03 (s, 3H), 2.00 (s, 3H), 1.91 (s, 3H), 1.72-1.55 (m, 4H).

Step 4: (2R,3R,4R,5R,6R)-5-acetamido-2-(acetoxymethyl)-6-((5-(benzyloxy)-5-oxopentyl)oxy)tetrahydro-2H-pyran-3,4-diyl diacetate (90 g, 167.4 mmol) was dissolved in a mixture of EtOAc (250 mL) and MeOH (250 mL) and wet Pd/C (4.5 g, 10%) was then added. The reaction mixture was degassed, hydrogenated under hydrogen gas and then stirred overnight. TLC showed the start material was completely consumed. The reaction mixture was filtered and the filtrate was concentrated to dryness to give 5-(((2R,3R,4R,5R,6R)-3-acetamido-4,5-diacetoxy-6-(acetoxymethyl)tetrahydro-2H-pyran-2-yl)oxy)pentanoic acid. ¹H NMR (400 MHz, CDCl₃) δ ppm 5.95 (d, 1H, J=8.4 Hz), 5.35 (d, 1H, J=2.4 Hz), 5.25 (dd, 1H, J1=11.6 Hz, J2=3.6 Hz), 4.66 (d, 1H, J1=8 Hz), 4.16-4.11 (m, 2H), 4.01-3.90 (m, 3H), 3.56-3.49 (m, 1H), 2.40-2.34 (m, 2H), 2.16 (s, 3H), 2.06 (s, 3H), 2.01 (s, 3H), 1.98 (s, 3H), 1.72-1.55 (m, 4H).

Step 5: To a solution of 5-(((2R,3R,4R,5R,6R)-3-acetamido-4,5-diacetoxy-6-(acetoxymethyl)tetrahydro-2H-pyran-2-yl)oxy)pentanoic acid (69 g, 154.2 mmol) and NHS—OH (19.5 g, 169.62 mmol) in DCM (600 mL) was added DIC (19.4 g, 154.2 mmol) and DMAP (36 mg, 0.29 mmol). The reaction mixture was stirred at room temperature for 3 hours. TLC showed the start material was completely consumed. The resultant mixture was filtered and the filtrate was concentrated. The residue was purified by silica gel column chromatography (elute: PE:EA=2:1-1:4) to obtain (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate (int-B1). ¹H NMR (400 MHz, CDCl₃) δ ppm 5.83 (d, 1H, J=8.4 Hz), 5.33 (d, 1H, J=2.4 Hz), 5.25 (dd, 1H, J1=11.6 Hz, J2=3.6 Hz), 4.67 (d, 1H, J1=8 Hz), 4.13-4.07 (m, 2H), 4.00-3.87 (m, 3H), 2.86-2.82 (m, 4H), 2.73-2.55 (m, 2H), 2.14 (s, 3H), 2.02 (s, 3H), 1.97 (s, 3H), 1.91 (s, 3H), 1.72-1.55 (m, 4H).

### Type C:

### Synthesis of (2R,2′R,3R,3′R,4R,4′R,5R,5′R,6R,6′R)-((16-((3-((3-(5-(((3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-16-(((benzyloxy)carbonyl)amino)-5,11,21,27-tetraoxo-14,18-dioxa-6,10,22,26-tetraazahentriacontane-1,31-diyl)bis(oxy))bis(5-acetamido-2-(acetoxymethyl)tetrahydro-2H-pyran-6,3,4-triyl) tetraacetate (int-C1

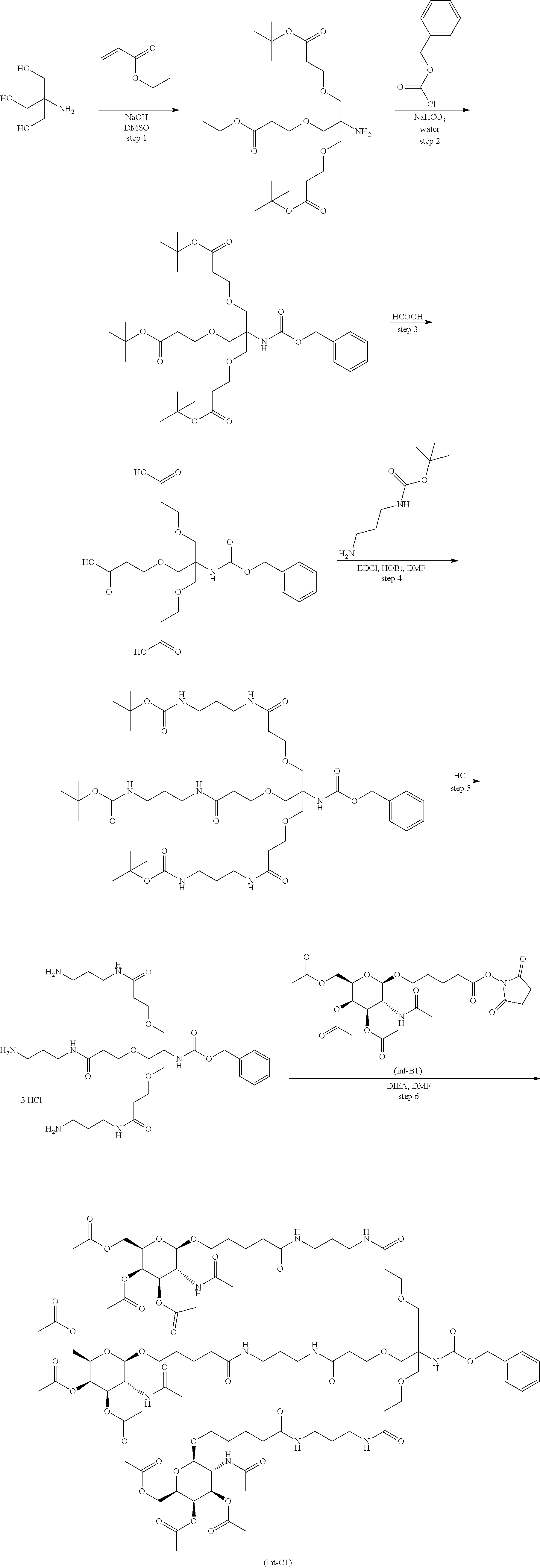

Step 1. Aqueous NaOH (3.2 mL, 5 M) was added to a mixture of 2-amino-2-(hydroxymethyl)propane-1,3-diol (20 g, 165.28 mmol) in DMSO (32 mL) at 20° C., then tert-butyl acrylate (74 g, 578.48 mmol) was added dropwise. The mixture was stirred at 20° C. for 24 hours. The resulting mixture was diluted with EtOAc (1 L), washed with H₂O (800 mL). The organic layer was dried over Na₂SO₄ and concentrated to give crude di-tert-butyl 3,3′-((2-amino-2-((3-(tert-butoxy)-3-oxopropoxy)methyl)propane-1,3-diyl)bis(oxy))dipropionate, which was used in next step directly. tᵣ=1.308 min, [M+H]+ 506.3

Step 2: Aqueous NaHCO₃ (1 L) was added to a solution of di-tert-butyl 3,3′-((2-amino-2-((3-(tert-butoxy)-3-oxopropoxy)methyl)propane-1,3-diyl)bis(oxy))dipropionate (160 g, 316.20 mmol, crude) in EtOAc (1 L), then benzyl carbonochloridate (53.9 g, 316.20 mmol) was added dropwise. The reaction mixture was stirred at room temperature for 3 hours. TLC showed the reaction was complete. The organic layer was separated, washed with H₂O (500 mL), dried over Na₂SO₄ and concentrated. The residue was purified by silica gel column chromatography (elute: PE:EA=20:1 to 5:1) to give di-tert-butyl 3,3′-((2-(((benzyloxy)carbonyl)amino)-2-((3-(tert-butoxy)-3-oxopropoxy)methyl)propane-1,3-diyl)bis(oxy))dipropionate. ¹H NMR (400 MHz, CDCl₃) δ ppm 7.36-7.26 (m, 5H), 5.29 (brs, 1H), 5.02 (s, 2H), 3.65-3.61 (m, 12H), 2.43 (t, 6H, J=6.4 Hz), 1.43 (s, 27H).

Step 3: A solution of di-tert-butyl 3,3′-((2-(((benzyloxy)carbonyl)amino)-2-((3-(tert-butoxy)-3-oxopropoxy)methyl)propane-1,3-diyl)bis(oxy))dipropionate (37 g, 57.83 mmol) in formic acid (100 mL) was stirred for 8 hours at room temperature. The resultant mixture was concentrated to dryness to give 3,3′-((2-(((benzyloxy)carbonyl)amino)-2-((2-carboxyethoxy)methyl)propane-1,3-diyl)bis(oxy))dipropionic acid. ¹H NMR (400 MHz, CDCl₃) δ ppm 7.39-7.30 (m, 5H), 5.35 (brs, 1H), 5.06 (s, 2H), 3.71-3.65 (m, 12H), 2.59 (t, 6H, J=6.8 Hz).

Step 4: HOBT (38.6 g, 286.34 mmol), EDCI (54.9 g, 286.34 mmol) and Et₃N (28.9 g, 286.34 mmol) were added to a mixture of 3,3′-((2-(((benzyloxy)carbonyl)amino)-2-((2-carboxyethoxy)methyl)propane-1,3-diyl)bis(oxy))dipropionic acid (27 g, 57.27 mmol) and tert-butyl (3-aminopropyl)carbamate (49.8 g, 286.34 mmol) in DMF (600 mL), and the reaction mixture stirred at room temperature for 6 hours. TLC showed the reaction was complete. The resulting mixture was diluted with H₂O (1 L) and extracted with DCM (800 mL×3). The combined organic layer was washed with aqueous NH₄C₁ (1 L), dried over Na₂SO₄ and concentrated. The residue was purified by silica gel column chromatography (elute: DCM:MeOH=50:1-20:1) to obtain benzyl di-tert-butyl (10-(13,13-dimethyl-5,11-dioxo-2,12-dioxa-6,10-diazatetradecyl)-5,15-dioxo-8,12-dioxa-4,16-diazanonadecane-1,10,19-triyl)tricarbamate. ¹H NMR (400 MHz, CDCl₃) δ ppm 7.37-7.29 (m, 5H), 6.85 (brs, 3H), 5.55 (brs, 1H), 5.14 (brs, 3H), 5.02 (s, 2H), 3.69-3.64 (m, 12H), 3.31-3.23 (m, 6H), 3.15-3.08 (m, 6H), 2.43-2.36 (m, 6H), 1.65-1.56 (m, 6H), 1.42 (s, 27H).

Step 5: To a solution of benzyl di-tert-butyl (10-(13,13-dimethyl-5,11-dioxo-2,12-dioxa-6,10-diazatetradecyl)-5,15-dioxo-8,12-dioxa-4,16-diazanonadecane-1,10,19-triyl)tricarbamate (35 g, 37.23 mmol) in MeOH (500 mL) was added a solution of HCl in 1,4-dioxane (46 mL, 186.17 mmol, 4M). The reaction mixture was stirred at room temperature for 3 hours. The resultant mixture was concentrated to dryness to give the HCl salt of benzyl (1,19-diamino-10-((3-((3-aminopropyl)amino)-3-oxopropoxy)methyl)-5,15-dioxo-8,12-dioxa-4,16-diazanonadecan-10-yl)carbamate. ¹H NMR (400 MHz, MeOD) δ ppm 7.41-7.32 (m, 5H), 5.06 (s, 2H), 3.70-3.64 (m, 12H), 3.33-3.29 (m, 6H), 2.97-2.93 (m, 6H), 2.48-2.45 (m, 6H), 1.88-1.82 (m, 6H).

Step 6: (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate (int-B1) (24 g, 44.05 mmol) was added to a mixture of benzyl (1,19-diamino-10-((3-((3-aminopropyl)amino)-3-oxopropoxy)methyl)-5,15-dioxo-8,12-dioxa-4,16-diazanonadecan-10-yl)carbamate (10 g, 13.35 mmol) and DIPEA (17.2 g, 133.5 mmol) in DMF (250 mL) and the reaction mixture stirred at room temperature for 12 hours. TLC showed the reaction was complete. The resulting mixture was diluted with H₂O (500 mL) and extracted with DCM (300 mL×3). The combined organic layer was washed with aqueous HCl (300 mL, 4M), dried over Na₂SO₄ and concentrated. The residue was purified by silica gel column chromatography (elute: DCM:MeOH=30:1-15:1) to give (2R,2′R,3R,3′R,4R,4′R,5R,5′R,6R,6′R)-((16-((3-((3-(5-(((3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-16-(((benzyloxy)carbonyl)amino)-5,11,21,27-tetraoxo-14,18-dioxa-6,10,22,26-tetraazahentriacontane-1,31-diyl)bis(oxy))bis(5-acetamido-2-(acetoxymethyl)tetrahydro-2H-pyran-6,3,4-triyl) tetraacetate (int-C1). ¹H NMR (400 MHz, DMSO-d6) δ ppm 7.85-7.80 (m, 6H), 7.72 (brs, 3H), 7.34-7.30 (m, 5H), 6.52 (s, 1H), 5.19 (d, 3H, J=3.2 Hz), 4.95-4.92 (m, 5H), 4.46 (d, 3H, J=8.4 Hz), 4.00 (brs, 9H), 3.86-3.84 (m, 3H), 3.69-3.67 (m, 3H), 3.54-3.41 (m, 15H), 3.01 (brs, 12H), 2.27-2.24 (m, 6H), 2.08.

### Type D:

### Synthesis of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-(aminooxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide) (int-D1

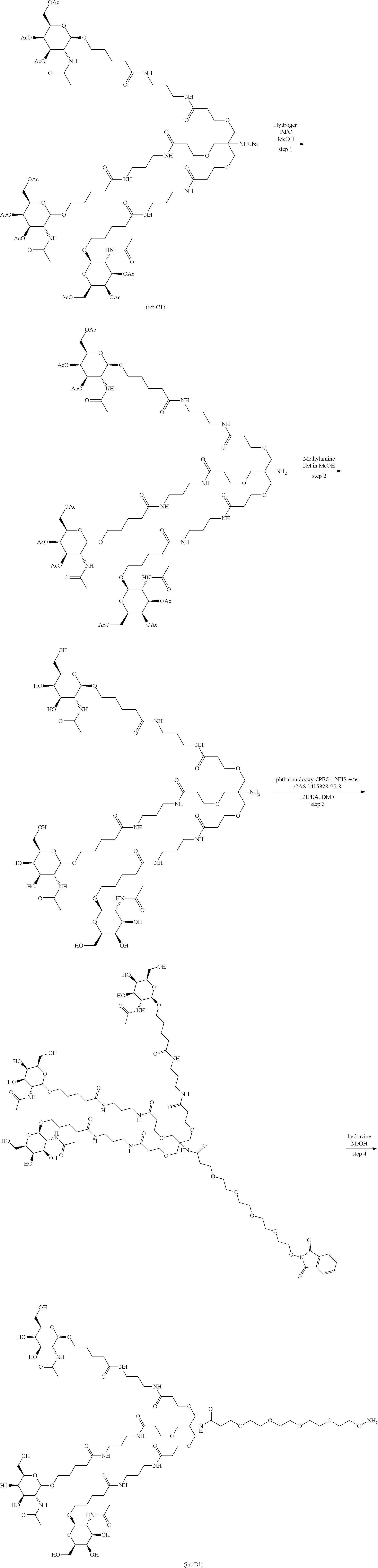

Step 1: Synthesis of (2R,2′R,3R,3′R,4R,4′R,5R,5′R,6R,6′R)-((16-((3-((3-(5-(((3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-16-amino-5,11,21,27-tetraoxo-14,18-dioxa-6,10,22,26-tetraazahentriacontane-1,31-diyl)bis(oxy))bis(5-acetamido-2-(acetoxymethyl)tetrahydro-2H-pyran-6,3,4-triyl) tetraacetate.

To a solution of (2R,2′R,3R,3′R,4R,4′R,5R,5′R,6R,6′R)-((16-((3-((3-(5-(((3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-16-(((benzyloxy)carbonyl)amino)-5,11,21,27-tetraoxo-14,18-dioxa-6,10,22,26-tetraazahentriacontane-1,31-diyl)bis(oxy))bis(5-acetamido-2-(acetoxymethyl)tetrahydro-2H-pyran-6,3,4-triyl) tetraacetate (int-C1) (142.8 mg, 0.074 mmol) in MeOH (5 mL) was added Pd/C (10 wt %, 30 mg) and acetic acid (100 μL). The reaction mixture was hydrogenated under hydrogen gas at 50 PSI for 18 h. At completion, the reaction was filtered and the filter cake was washed with MeOH and the combined organics were concentrated to provide the title product (135 mg). LCMS m/z (M+1)⁺=1794.9.

Step 2: Synthesis of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-amino-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide).

A solution of (2R,2′R,3R,3′R,4R,4′R,5R,5′R,6R,6′R)-((16-((3-((3-(5-(((3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-16-amino-5,11,21,27-tetraoxo-14,18-dioxa-6,10,22,26-tetraazahentriacontane-1,31-diyl)bis(oxy))bis(5-acetamido-2-(acetoxymethyl)tetrahydro-2H-pyran-6,3,4-triyl) tetraacetate (343 mg, 0.191 mmol) in MeOH (1 mL) was treated with a solution of methylamine (2.0 M in MeOH, 2 mL). The reaction was maintained at room temperature for 2 h, then concentrated to provide the title product (310 mg). LCMS m/z [(M+2H)/2]⁺=708.9.

Step 3: Synthesis of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-((1,3-dioxoisoindolin-2-yl)oxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide). To a solution of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-amino-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide) (70.8 mg, 0.05 mmol) in DMF (1 mL) was added 2,5-dioxopyrrolidin-1-yl 1-((1,3-dioxoisoindolin-2-yl)oxy)-3,6,9,12-tetraoxapentadecan-15-oate (38.1 mg, 0.075 mmol) and N,N-diisopropylethylamine. The mixture was stirred at room temperature for 2 h, then purified by reverse phase silica gel chromatography to provide the title product (57 mg) following lyophilization. LCMS m/z [(M+2H)/2]⁺=905.5.

Step 4: Synthesis of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-(aminooxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide (int-D1). To a room temperature solution of N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-((1,3-dioxoisoindolin-2-yl)oxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide) (57 mg, 0.032 mmol) in MeOH (1 mL) was added hydrazine hydrate (63.1 mg, 1.26 mmol). The mixture was stirred at room temperature for 2 h, then purified by reverse phase silica gel chromatography to provide N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-(aminooxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide (int-D1) (18.1 mg). LCMS m/z [M+H]⁺=1679.8.

### Example 2: Preparation and Analysis of an Anti-ASGPR-PCSK9 Bispecific Antibody

A bispecific antibody comprising both an anti-ASGPR and an anti-PCSK9 domain was prepared; see, e.g., FIG. 1. Each domain was designed based on published antibodies targeting ASGPR and PCSK9; see, e.g., Bon et al. (2017) MABS 9(8):1360-1369 and Chaparro-Riggers et al. (2012) J Biol Chem 287(14):11090-11097. Exemplary ASGPR-PCSK9 bispecific antibody sequences are provided below:

PCSK9 VH
(SEQ ID NO: 1)
QVQLVQSGAEVKKPGASVKVSCKASGYTFTSYYMHWVRQAPGQGLEWMG
EISPFGGRTNYNEKFKSRVTMTRDTSTSTVYMELSSLRSEDTAVYYCAR
ERPLYASDLWGQGTTVTVSS
PCSK9 VL
(SEQ ID NO: 2)
DIQMTQSPSSLSASVGDRVTITCRASQGISSALAWYQQKPGKAPKLLIY
SASYRYTGVPSRFSGSGSGTDFTFTISSLQPEDIATYYCQQRYSLWRTF
GQGTKLEIK
ASGPR VH
(SEQ ID NO: 3)
DIQMTQSPSSLSASVGDRVTITCRASQGISSALAWYQQKPGKAPKLLIY
SASYRYTGVPSRFSGSGSGTDFTFTISSLQPEDIATYYCQQRYSLWRTF
GQGTKLEIK
ASGPR VL
(SEQ ID NO: 4)
SSELTQDPAVSVALGQTVRITCQGDSLRSYYASWYQQKPGQAPVLVIYG
KNNRPSGIPDRFSGSSSGNTASLTITGAQAEDEADYYCNSLERIGYLSY
VFGGGTKLTVL
ASGPR scFv (VH-VL with (G4S)₄ linker (SEQ ID
NO: 46))*
(SEQ ID NO: 5)
EVQLLESGGGLVQPGGSLRISCAASGFTFSSYAMSWVRQAPGKGLEWVS
AISGSGGSTYYADSVKGRFTISRDNSKNTLYLQMNSLRAEDTAVYYCAK
DFSSRRWYLEYWGQGTLVTVSSGGGGSGGGGSGGGGSGGGGSSSELTQD
PAVSVALGQTVRITCQGDSLRSYYASWYQQKPGQAPVLVIYGKNNRPSG
IPDRFSGSSSGNTASLTITGAQAEDEADYYCNSLERIGYLSYVFGGGTK
LTVL
ASGPR_4F3_scFv-PCSK9_Fab_DANAPA_hIgG1_KiH_0
(SEQ ID NO: 6)
DIQMTQSPSSLSASVGDRVTITCRASQGISSALAWYQQKPGKAPKLLIY
SASYRYTGVPSRFSGSGSGTDFTFTISSLQPEDIATYYCQQRYSLWRTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
ASGPR_4F3_scFv-PCSK9_Fab_DANAPA_hIgG1_KiH_1*
(SEQ ID NO: 7)
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DFSSRRWYLEYWGQGTLVTVSSGGGGSGGGGSGGGGSGGGGSSSELTQD
PAVSVALGQTVRITCQGDSLRSYYASWYQQKPGQAPVLVIYGKNNRPSG
IPDRFSGSSSGNTASLTITGAQAEDEADYYCNSLERIGYLSYVFGGGTK
LTVLGGGGSDKTHTCPPCPAPELLGGPSVFLFPPKPKDTIMISRTPEVT
CVVVAVSHEDPEVKFNWYVDGVEVHNAKTKPREEQYASTYRVVSVLTVL
HQDWLNGKEYKCKVSNKALAAPIEKTISKAKGQPREPQVYTLPPCREEM
TKNQVSLWCLVKGFYPSDIAVEWESNGQPENNYKTTPPVLDSDGSFFLY
SKLTVDKSRWQQGNVFSCSVMHEALHNRYTQKSLSLSPGK
ASGPR_4F3_scFv-PCSK9_Fab_DANAPA_hIgG1_KiH_2
(SEQ ID NO: 8)
QVQLVQSGAEVKKPGASVKVSCKASGYTFTSYYMHWVRQAPGQGLEWMG
EISPFGGRTNYNEKFKSRVTMTRDTSTSTVYMELSSLRSEDTAVYYCAR
ERPLYASDLWGQGTTVTVSSASTKGPSVFPLAPSSKSTSGGTAALGCLV
KDYFPEPVTVSWNSGALTSGVHTFPAVLQSSGLYSISSVVTVPSSSLGT
QTYICNVNHKPSNTKVDKRVEPKSCDKTHTCPPCPAPELLGGPSVFLFP
PKPKDTLMISRTPEVTCVVVAVSHEDPEVKFNWYVDGVEVHNAKTKPRE
EQYASTYRVVSVLIVLHQDWLNGKEYKCKVSNKALAAPIEKTISKAKGQ
PREPQVCTLPPSREEMTKNQVSLSCAVKGFYPSDIAVEWESNGQPENNY
KTTPPVLDSDGSFFLVSKLTVDKSRWQQGNVFSCSVMHEALHNHYTQKS
LSLSPGK
*(G4S)ₙ linkers are indicated with underlined font (G4S (SEQ ID NO: 43); (G4S)₄ (SEQ ID NO: 46)).

Exemplary ASGPR-PCSK9 bispecific antibody CDR sequences are provided below in Table 6.

TABLE 6
ASGPR-PCSK9 bispecific antibody CDR sequences
Kabat CDR Definition

| Label | CDR1 | CDR2 | CDR3 |
|---|---|---|---|
| ASGPR VH | SYAMS | AISGSGGSTYYADSVKG | DFSSRRWYLEY |
| | (SEQ ID NO: 9) | (SEQ ID NO: 10) | (SEQ ID NO: 11) |
| ASGPR VL | QGDSLRSYYAS | GKNNRPS | NSLERIGYLSYV |
| | (SEQ ID NO: 12) | (SEQ ID NO: 13) | (SEQ ID NO: 14) |
| PCSK9 VH | SYYMH | EISPFGGRTNYNEKFKS | ERPLYASDL |
| | (SEQ ID NO: 15) | (SEQ ID NO: 16) | (SEQ ID NO: 17) |
| PCSK9 VL | RASQGISSALA | SASYRYT | QQRYSLWRT |
| | (SEQ ID NO: 18) | (SEQ ID NO: 19) | (SEQ ID NO: 20) |

Chothia CDR Definition

| Label | CDR1 | CDR2 | CDR3 |
|---|---|---|---|
| ASGPR VH | GFTFSSY | SGSGGS | DFSSRRWYLEY |
| | (SEQ ID NO: 21) | (SEQ ID NO: 22) | (SEQ ID NO: 11) |
| ASGPR VL | DSLRSYY | GKN | LERIGYLSY |
| | (SEQ ID NO: 23) | (SEQ ID NO: 24) | (SEQ ID NO: 25) |
| PCSK9 VH | GYTFTSY | SPFGGR | ERPLYASDL |
| | (SEQ ID NO: 26) | (SEQ ID NO: 27) | (SEQ ID NO: 17) |
| PCSK9 VL | SQGISSA | SAS | RYSLWR |
| | (SEQ ID NO: 28) | (SEQ ID NO: 29) | (SEQ ID NO: 30) |

IMGT CDR Definition

| Label | CDR1 | CDR2 | CDR3 |
|---|---|---|---|
| ASGPR VH | GFTFSSYA | ISGSGGST | AKDFSSRRWYLEY |
| | (SEQ ID NO: 31) | (SEQ ID NO: 32) | (SEQ ID NO: 33) |
| ASGPR VL | SLRSYY | IYGKN | NSLERIGYLSYV |
| | (SEQ ID NO: 34) | (SEQ ID NO: 35) | (SEQ ID NO: 14) |
| PCSK9 VH | GYTFTSYY | ISPFGGRT | ARERPLYASDL |
| | (SEQ ID NO: 36) | (SEQ ID NO: 37) | (SEQ ID NO: 38) |
| PCSK9 VL | QGISSA | SAS | QQRYSLWRT |
| | (SEQ ID NO: 39) | (SEQ ID NO: 29) | (SEQ ID NO: 20) |

Anti-PCSK9 heavy chain was synthesized as fusion of the variable domain to constant silent hIgG1 domains (changes could be made to hIgG1 domains to facilitate heterodimerization). PCSK9 light chain plasmid was also synthesized. For the anti-ASGPR arm, this was produced as single chain fragment variable (scFv) in the VH-VL orientation with 4×G4S (SEQ ID NO: 46) linker between the variable domains fused to constant hIgG1 domains (changes could be made to hIgG1 domains to facilitate heterodimerization). Bispecific antibodies were co-expressed transiently in Expi293F (Thermo-Fisher Scientific) cells. Briefly, transfection was performed using PEI Max as transfection reagent. Cells were grown in shake flasks on an orbital shaker (115 rpm) in a humidified incubator (85%) at 5% CO₂. Anti-PCSK9 light and heavy chain plasmids were combined with ASGPR plasmid at 3:2:2 ratio with PEI at a final ratio of 1 DNA:3 PEI. 1 mg/L culture of plasmid was used for transfection at 0.5 million cells/mL serum media. After 5 days of expression, the antibody was harvested by clarification of the media via centrifugation and filtration. Purification was performed via batch binding and elution using Protein A affinity batch chromatography (MabSelect® SuRe, GE Healthcare Life Sciences, Uppsala, Sweden). Resin was added at a ratio of 1 mL resin for every 100 mL supernatant and allowed to batch bind for up to 4 hours. Disposable columns were loaded with supernatant allowed to drain via gravity and washed with 20 column volumes of PBS. Antibody was eluted with 20 column volumes of 20 mM citrate, 125 mM NaCl, 50 mM sucrose pH 3.2. The eluted IgG protein was adjusted to pH 5.5 with 1 M sodium citrate. Preparative size exclusion chromatography was performed using Hi Load 16/60 Superdex 200 grade column (GE Healthcare Life Sciences, Uppsala, Sweden) in a buffer composed of 20 mM citrate, 125 mM NaCl, 50 mM sucrose pH 5.5 as a final polishing step.

The construct was checked for identity and purity by determining the intact mass using HPLC-MS and assessing overall monomeric heterodimer via analytical size exclusion chromatography. The resulting mass spectra were deconvoluted using MaxENT to determine the intact mass of the bispecific antibody construct. The percent monomeric heterodimer was determined via analytical SEC HPLC. The resulting chromatogram was compared to that run on the same day for a gel filtration standard (Bio-Rad, Hercules, California)

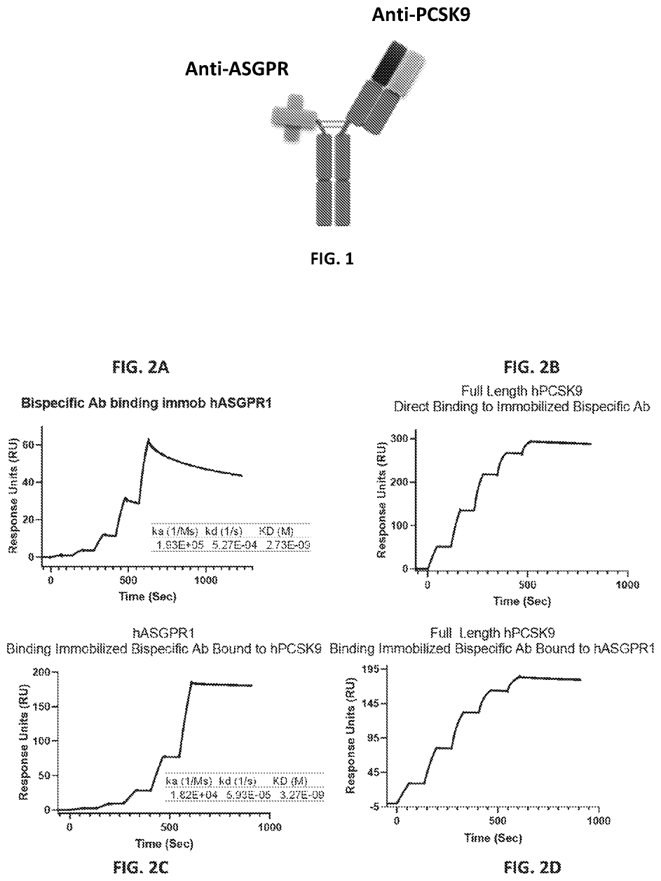

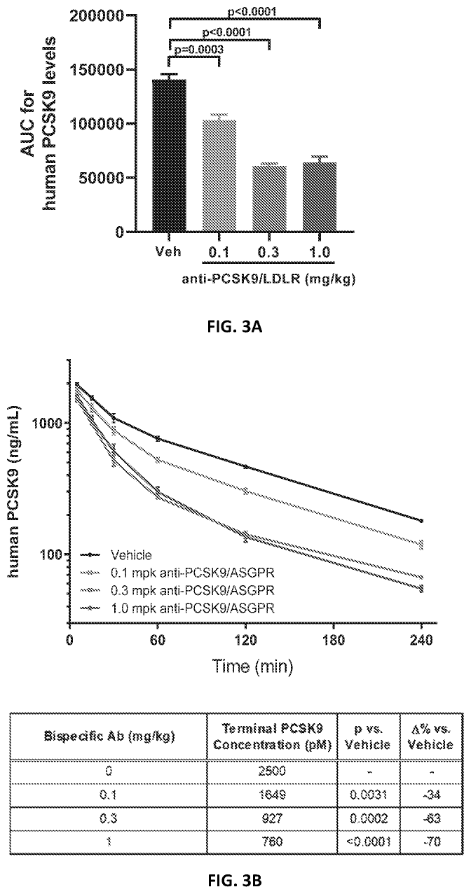

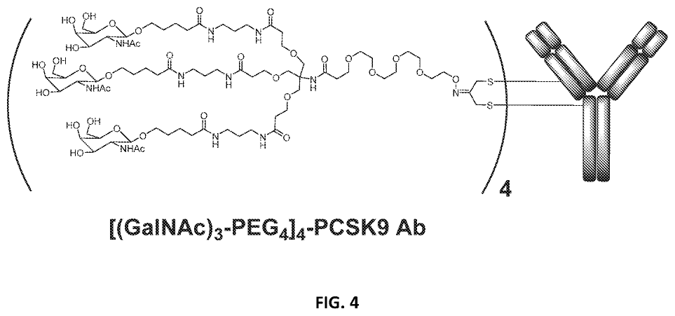

Binding specificity of the anti-ASGPR-PCSK9 bispecific antibody was interrogated by affinity assay and demonstrated high affinity for its targets. As shown in FIGS. 2A-B, immobilized hASBPR1 or full length hPCSK9 incubated with the anti-ASGPR-PCSK9 bispecific antibody demonstrated high affinity. Additionally, immobilized full length hPCSK9 complexed with the anti-ASGPR-PCSK9 bispecific antibody was shown to bind ASGPR with high affinity, as did immobilized hASBPR1 complexed with the anti-ASGPR-PCSK9 bispecific antibody incubated with full length hPCSK9 (FIGS. 2C-D). In addition, pharmacokinetic studies of the anti-ASGPR-PCSK9 bispecific antibody in LDLR knock out mice demonstrated accelerated clearance of PCSK9 in the presence of the antibody at dosages of 0.1, 0.3, and 1 mg/kg (FIGS. 3A-B).

### Example 3: Preparation and Analysis of a GalNAc-Labeled Anti-PCSK9 Antibody

An anti-PCSK9 antibody labeled with a trimeric N-acetylgalactosamine (GalNAc) moiety was designed according to the schematic in FIGS. 20A and 20B, and prepared as described in Example 10 and Example 11. The PCSK9 antibody was based on the anti-PCSK9 antibodies described in Chan et al. (2009) Proc Natl Acad Sci USA 106(24):9820-9825.

Binding specificity of the GalNAc-labeled anti-PCSK9 antibody was evaluated by direct binding studies with each of ASGPR1 and PCSK9 (FIGS. 5A-5B), as well as analysis of the ASGPR1 binding to a complex of the GalNAc-labeled anti-PCSK9 antibody to immobilized PCSK9 (FIG. 5C). As shown, the GalNAc-labeled anti-PCSK9 antibody exhibits high affinity to both ASGPR and PCSK9. In addition, pharmacokinetic studies of the GalNAc-labeled anti-PCSK9 antibody in LDLR knock out mice demonstrated accelerated clearance of PCSK9 from plasma in the presence of the antibody at dosages of 0.3, 1, and 3 mg/kg (FIGS. 6A-6B).

### Example 4: Preparation and Analysis of a GalNAc-Labeled Anti-ApoB Antibody

An anti-ApoB antibody labeled with a trimeric N-acetylgalactosamine (GalNAc) moiety was prepared as follows. The anti-ApoB antibody (6.7 mg/mL) in 20 mM sodium phosphate buffer, pH 7.5/150 mM NaCl/20 mM EDTA was mixed with a 5 mM solution of TCEP and the reduction was allowed to proceed at 40° C. for 1.5 hours with a final antibody concentration of 5.0 mg/mL. After 1.5 hours, the reduction mixture was diluted with 20 mM sodium phosphate buffer, pH 7.5/150 mM NaCl/20 mM EDTA and allowed to cool to 22° C. A 12.0 mg/mL (4.83 mM) solution of trimeric GalNAc described in Example 10 (see compound 8 in FIG. 20A) in DMF was prepared by dissolving 1.26 mg (512 nmol) of trimeric GalNAc reagent into 106 μL DMF. The trimeric GalNAc reagent was added to the reduced anti-ApoB antibody solution resulting in a final concentration of 5% DMF and a final antibody concentration of 4.0 mg/mL. The conjugation reaction was incubated at 22° C. for 22 hours, at which point the reaction mixture was buffer exchanged into PBS by ultrafiltration (Vivaspin20, 10 kDa MWCO, 3×) and gel filtration using an Illustra NAP-10 column. The eluted conjugate was sterile filtered (0.22 μM), and then characterized by size exclusion chromatography, LC-MS, and SDS-PAGE.

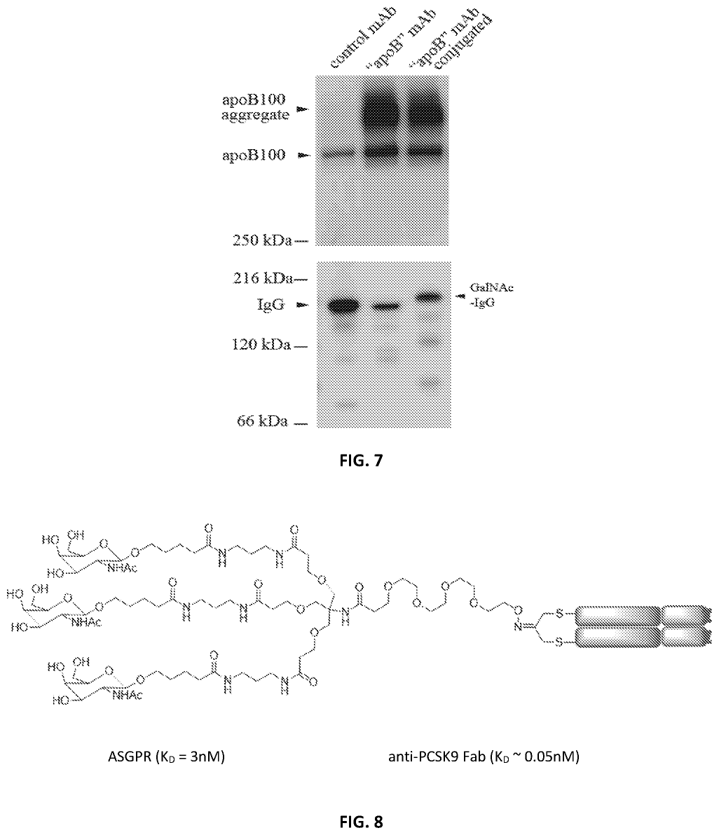

To interrogate binding specificity, a mixture of human serum spiked with purified LDL (1 mL; 75% PBS, 25% serum, 100 μg LDL, protease inhibitor cocktail) was first cleared of Protein A and G-binding elements, then incubated with a control antibody, anti-ApoB antibody, or the GalNAc-labeled anti-ApoB antibody. Each mixture was then precipitated using a commercially available Protein A/G agarose kit and subject to immunoprecipitation and Western blotting with apoB100 IgG (FIG. 7), which indicated that the GalNAc-labeled anti-ApoB antibody exhibits affinity for apoB.

### Example 5: Preparation and Analysis of a GalNAc-Labeled Anti-PCSK9 Fab Fragment

An anti-PCSK9 Fab fragment labeled with a trimeric N-acetylgalactosamine (GalNAc) moiety was designed (see FIG. 8) and prepared as follows. The anti-PCSK9 Fab fragment was based on the anti-PCSK9 antibodies described in Chan et al. (2009) Proc Natd Acad Sci USA 106(24):9820-9825.

The anti-PCSK9 Fab fragment (3.92 mg, 0.084 mol) in 500 μL of Tris (0.1M pH 7.4) was first activated by mixing with 1,3-dichloroacetone in DMSO (6.65 μL, 4.19 μmol) at 4° C. The mixture was agitated at 4° C. for 2 minutes, and then TCEP·HCl in water (11.98 μL, 0.838 μmol) was added dropwise. The resulting mixture was agitated at 20° C. for 20 hours and then passed through Zeba 7K MWCO spin columns pre-equilibrated with succinate buffer (0.2M pH 4.5) three consecutive times to give modified anti-PCSK9 Fab. LCMS ESI: 46892.

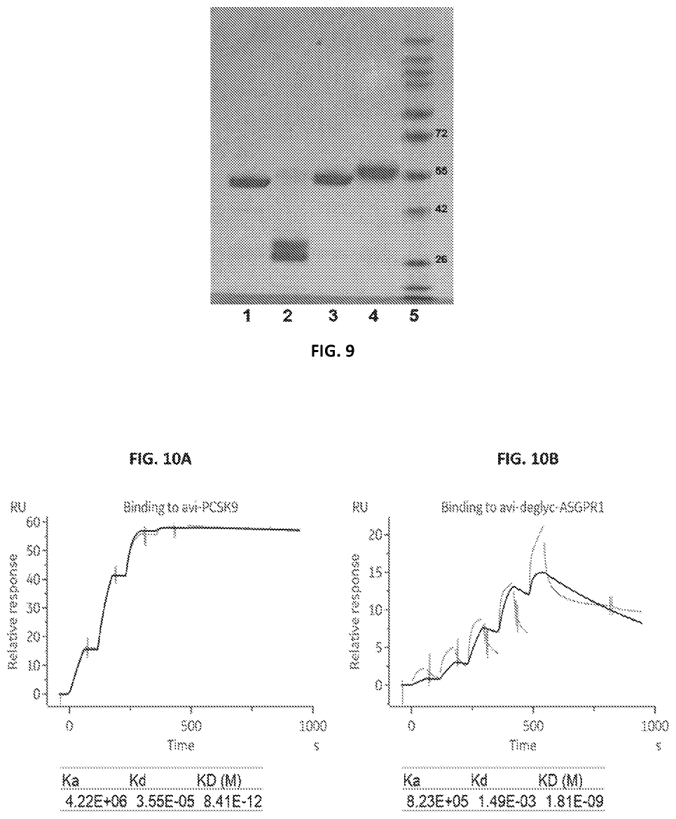

To conjugate the activated GalNAc moiety to the activated PCSK9 Fab fragment, the GalNAc moiety (N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-(aminooxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide (int-D1); 98 μL of a 57 mg/mL solution in DMSO, 5.61 mg, 3.34 mol) was added to a solution of modified anti-PCSK9 Fab (3.92 mg, 0.084 mol) in 0.5 mL of succinate buffer (0.2M, pH 4.5) at room temperature. The mixture was agitated at 20° C. for 2 days, and then concentrated by using Amicon ultra 30K MWCO centrifugal filter with PBS as the washing buffer to give the desired GalNAc-labeled anti-PCSK9 Fab conjugate (330 μL, 9.7 mg/mL, 3.2 mg, 78% over 2 steps). LCMS ESI: 48608. FIG. 9 depicts an SDS PAGE of the final product, wherein lane 1 corresponds to the anti-PCSK9 Fab, lane 2 corresponds to the anti-PCSK9 Fab (reduced), lane 3 corresponds to the GalNAc-labeled anti-PCSK9 Fab, lane 4 corresponds to the GalNAc-labeled anti-PCSK9 Fab (reduced), and lane 5 is a protein ladder reference.

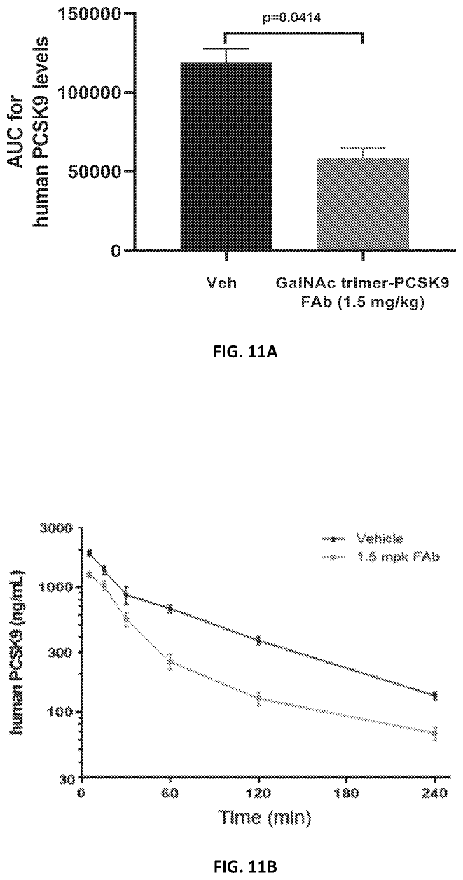

Binding studies with both immobilized PCSK9 and immobilized ASGPR1 demonstrate high affinity of the GalNAc-labeled anti-PCSK9 Fab (FIGS. 10A-B). In addition, in vivo studies in mice indicated accelerated clearance of PCSK9 upon administration of the GalNAc-labeled anti-PCSK9 Fab (FIGS. 11A-B).

### Example 6: Preparation and Analysis of GalNAc-Labeled Anti-MICA Fab Fragments

A set of anti-MICA Fab fragments labeled with a trimeric N-acetylgalactosamine (GalNAc) moiety was designed and prepared as follows.

The anti-MICA Fab fragments (0.72 mg, 0.015 mol) in 160 μL of Tris (0.1M pH 7.4) were first activated by mixing with 1,3-dichloroacetone in DMSO (2.4 μL, 0.756 μmol) at 4° C. The mixture was agitated at 4° C. for 2 minutes, and then TCEP·HCl in water (2.162 L, 0.151 μmol) was added dropwise. The resulting mixture was agitated at 20° C. for 4 hours and then passed through Zeba 7K MWCO spin columns pre-equilibrated with succinate buffer (0.2M pH 4.5) three consecutive times to give modified anti-MICA Fab fragments 1, 2, and 3. LCMS ESI activated Fab fragments 1, 2 and 3: 47239, 46824 and 47498 respectively.

To conjugate the activated GalNAc moiety to the activated MICA Fab fragments, the GalNAc moiety (N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(1-(aminooxy)-3,6,9,12-tetraoxapentadecan-15-amido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide (int-D1); 23.5 μL of a 36 mg/mL solution in DMSO, 0.845 mg, 0.503 μmol) was added to a solution of each of the modified anti-MICA Fab fragments (0.6 mg, 0.013 μmol) in 150 μL of succinate buffer (0.2M pH 4.5) at room temperature. The mixtures were agitated at 20° C. for 16 hours, and then concentrated by using Amicon ultra 30K MWCO centrifugal filter with PBS as the washing buffer to give the GalNAc-labeled anti-MICA Fab fragments. Fab conjugate 1: (125 L, 2.10 mg/mL, 0.26 mg, 36% over 2 steps). LCMS ESI: 48901. Fab conjugate 2: (125 μL, 1.57 mg/mL, 0.20 mg, 27% over 2 steps). LCMS ESI: 48485. Fab conjugate 3: (125 μL, 3.25 mg/mL, 0.41 mg, 56% over 2 steps). LCMS ESI: 49159.

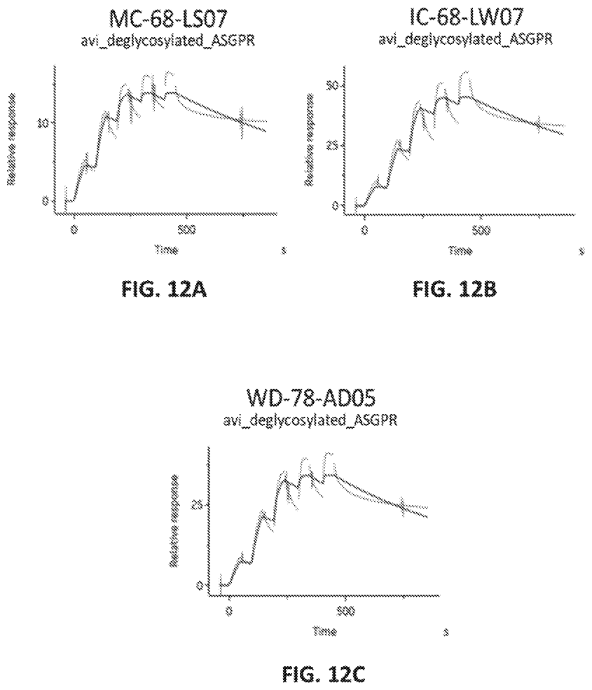

Binding studies with both immobilized ASGPR and immobilized MICA demonstrate high affinity of the GalNAc-labeled anti-MICA Fab fragments (FIGS. 12A-C) and Table 7 below.

TABLE 7
Binding affinity of exemplary GalNAc-
labeled anti-MICA Fab fragments

| | | MICA | ASGPR |
|---|---|---|---|
| Conjugate | MW | affinity (pM) | binding |
| Anti-lysozyme conjugate | 49349 | NA (isotype) | FIG. 12A |
| Fab conjugate 1 | 48901 | 69 | FIG. 12B |
| Fab conjugate 2 | 48485 | 2 | FIG. 12C |

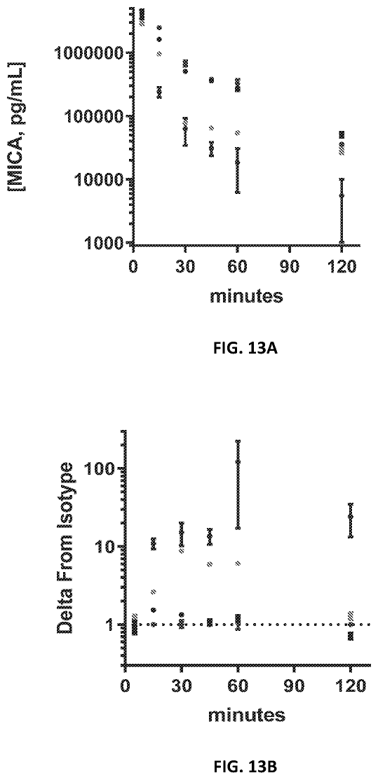

In addition, in vivo studies in mice indicated accelerated clearance of MICA upon administration of the GalNAc-labeled anti-MICA Fab fragments (FIGS. 13A-13B; 2 pM in blue and 69 μM in green), as compared to vehicle (black) or an isotype control (anti-lysozyme in red).

### Example 7: Preparation and Analysis of a GalNAc-Labeled LDLR

The extracellular domain of the LDLR protein labeled with a trimeric N-acetylgalactosamine (GalNAc) moiety was designed and prepared as follows. The extracellular domain of the LDLR protein has one of the following amino acid sequences:

LDLR (LDLR193 (193-720)(N515Q)(N657Q)_SrtA_CHis_pRS5a)
(SEQ ID NO: 40)
DSSPCSAFEFHCLSGECIHSSWRCDGGPDCKDKSDEENCAVATCRPDEFQCSDGNCIHGSRQCDREYD
CKDMSDEVGCVNVTLCEGPNKFKCHSGECITLDKVCNMARDCRDWSDEPIKECGTNECLDNNGGCSHV
CNDLKIGYECLCPDGFQLVAQRRCEDIDECQDPDTCSQLCVNLEGGYKCQCEEGFQLDPHTKACKAVG
SIAYLFFTNRHEVRKMTLDRSEYTSLIPNLRNVVALDTEVASNRIYWSDLSQRMICSTQLDRAHGVSS
YDTVISRDIQAPDGLAVDWIHSNIYWTDSVLGTVSVADTKGVKRKTLFREQGSKPRAIVVDPVHGFMY
WTDWGTPAKIKKGGLNGVDIYSLVTENIQWPNGITLDLLSGRLYWVDSKLHSISSIDVNGGNRKTILE
DEKRLAHPFSLAVFEDKVFWTDIINEAIFSANRLTGSDVNLLAENLLSPEDMVLFHQLTQPRGVNWCE
RTTLSNGGCQYLCLPAPQINPHSPKFTCACPDGMLLARDMRSCLTEAEAAVALPETGGHHHHHH
LDLR reduced (LDLR193 (193-720)(N515Q)(N657Q)_CHis_pRS5a)
(SEQ ID NO: 41)
DSSPCSAFEFHCLSGECIHSSWRCDGGPDCKDKSDEENCAVATCRPDEFQCSDGNCIHGSRQCDREYD
CKDMSDEVGCVNVTLCEGPNKFKCHSGECITLDKVCNMARDCRDWSDEPIKECGTNECLDNNGGCSHV
CNDLKIGYECLCPDGFQLVAQRRCEDIDECQDPDTCSQLCVNLEGGYKCQCEEGFQLDPHTKACKAVG
SIAYLFFTNRHEVRKMTLDRSEYTSLIPNLRNVVALDTEVASNRIYWSDLSQRMICSTQLDRAHGVSS
YDTVISRDIQAPDGLAVDWIHSNIYWTDSVLGTVSVADTKGVKRKTLFREQGSKPRAIVVDPVHGFMY
WTDWGTPAKIKKGGLNGVDIYSLVTENIQWPNGITLDLLSGRLYWVDSKLHSISSIDVNGGNRKTILE
DEKRLAHPFSLAVFEDKVFWTDIINEAIFSANRLTGSDVNLLAENLLSPEDMVLFHQLTQPRGVNWCE
RTTLSNGGCQYLCLPAPQINPHSPKFTCACPDGMLLARDMRSCLTEAEAAVAHHHHHH

The purified LDLR protein was then conjugated to GalNAc as follows. N,N′-(10-((3-((3-(5-(((3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)methyl)-10-(2-(2-(2-aminoacetamido)acetamido)acetamido)-5,15-dioxo-8,12-dioxa-4,16-diazanonadecane-1,19-diyl)bis(5-(((2R,3R,4R,5R,6R)-3-acetamido-4,5-dihydroxy-6-(hydroxymethyl)tetrahydro-2H-pyran-2-yl)oxy)pentanamide; 76 μL of a 40 mg/mL solution in DMSO, 3.03 mg, 1.91 mol) was added to a solution of LDLR (5.75 mg, 0.096 μmol) in 1.2 mL of Tris buffer (0.1M pH 8) at room temperature, followed by addition of Sortase A (0.311 mg, 0.014 μmol) in 76 μL of Tris buffer (0.1M pH 8). The mixture was agitated at 20° C. for 16 hours, and then purified by preparative size exclusion chromatography (HiLoad 16/600 Superdex 200 pg). The desired fractions were combined and concentrated by using Amicon ultra 30K MWCO centrifugal filter to give the GalNAc-labeled LDLR conjugate (300 μL, 7.69 mg/mL, 2.3 mg, 39%). LCMS ESI (after deglycosylation with PNGase F): 61849.

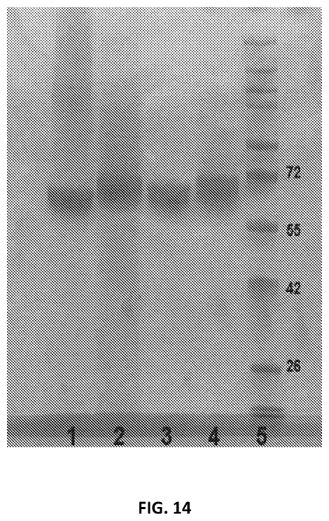

FIG. 14 depicts the SDS-PAGE of the reaction profile, wherein lane 1 corresponds to the LDLR starting material, lane 2 corresponds to the LDLR starting material (reduced), lane 3 corresponds to the GalNAc-labeled LDLR, lane 4 corresponds to the GalNAc-labeled LDLR (reduced), and lane 5 is a protein ladder reference.

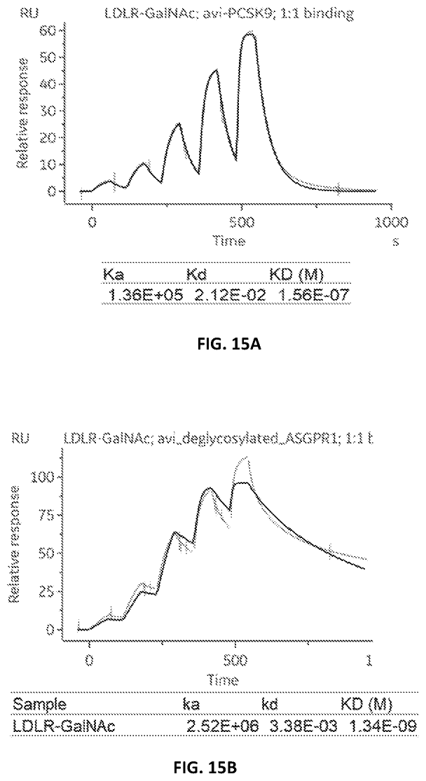

Binding studies with both immobilized PCSK9 and immobilized ASGPR1 demonstrate high affinity of the GalNAc-labeled LDLR (FIGS. 15A-B).

### Example 8: Preparation and Analysis of a GalNAc-Labeled PCSK9

The PCSK9 protein labeled with a trimeric N-acetylgalactosamine (GalNAc) moiety was designed and prepared as follows. The PCSK9 protein was prepared as described below.

Full-length cDNA encoding human PCSK9 corresponding to GenBank Accession #NM_174936 was cloned into the mammalian expression vector pcDNA3.1(−)Neo (Invitrogen). The cDNA included a Kozak (CACC) sequence immediately preceding the start codon and the 3′ end contained sequences encoding a C-terminal Gly-Gly linker followed by an Avi-tag and 6× histidine-tag (SEQ ID NO: 47). HEK293T cells were grown in suspension culture in a humidified atmosphere at 37° C. with 8% CO₂ using FreeStyle 293 Expression Medium (ThermoFisher Scientific). For 1 L transfections, 1×10⁶ cells/mL were grown in 934 mL of FreeStyle 293 medium in 2.5 L baffled shake flasks. Transfections were done using polyethyleneimine (PEI) and FreeStyle 293 medium as follows. Mixture A containing 1 mg of pcDNA3.1(−)Neo-hPCSK9-Avi-His or pcDNA3.1(−)Neo-mPCSK9-Avi-His plasmid in 33 mL medium and mixture B containing 3 mL of PEI (1 mg/mL) in 33 mL medium were each incubated for 5 min at room temperature, and then the mixtures were passed through a 0.2 m syringe filter. Mixture B was then slowly added to mixture A, and after a 15 min incubation at room temperature the transfection mixture (69 mL) was added to the HEK293T cells. The cells were grown at 37° C. with 8% CO₂ and shaking at 100 rpm. At 72 h post-transfection, the cells were harvested by centrifugation at 3000×g for 30 min and the conditioned medium was collected and filtered through a 0.22 um filter unit for purification.

Filtered conditioned medium was loaded at 4 mL/min onto a pre-packed Ni-sepharose affinity column (5 mL HisTrap HP, Cytiva, Cat #17524801) equilibrated in buffer A (20 mM Tris, pH 7.4, 150 mM NaCl, 20 mM imidazole) using an AKTA Express FPLC system (GE Healthcare). The column was washed with 10 column volumes of buffer A and proteins were eluted by applying a gradient of 0-100% buffer B (20 mM Tris, pH 7.4, 150 mM NaCl, 500 mM imidazole) in buffer A. Human PCSK9 was dialyzed into PBS/pH 7.4 and mouse PCSK9 was dialyzed into 20 mM Tris pH 8.0, 0.3 M NaCl and stored at 4° C. Protein concentrations were determined by UV-spectrophotometry at 280 nm using their theoretical extinction coefficients. Purity and homogeneity of proteins was analyzed by standard, reducing SDS-PAGE and by analytical size exclusion chromatography using a Superdex 200 column.

Purified PCSK9 proteins were buffer exchanged into PBS and 10 mM ATP, 10 mM Magnesium Acetate, 50 μM D-Biotin and 2.5 μg biotin ligase per 10 nmol PCSK9 were added from the BirA biotin-protein ligase reaction kit (Bulk BirA from Avidity, LLC) and incubated overnight at 4° C. To remove biotinylation reagents PCSK9 proteins were bound to a Ni-sepharose column and washed with 20 mM Tris pH7.4, 300 mM NaCl, 1 mM CaCl₂), 2 mM β-mercaptoethanol (BME). Proteins were eluted in 20 mM Tris pH7.4, 300 mM NaCl, 1 mM CaCl₂), 2 mM BME, 500 mM imidazole. The eluted proteins were immediately buffer exchanged into storage buffer. Biotinylation was confirmed by liquid chromatography-mass spectrometry.

The purified PCSK9 protein was then conjugated to GalNAc as follows. 2,3,5,6-tetrafluorophenyl 33-(((2R,3R,4R,5R,6R)-3-acetamido-4,5-dihydroxy-6-(hydroxymethyl)-tetrahydro-2H-pyran-2-yl)oxy)-18-((3-((3-(5-(((2R,3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)-methyl)-18-((3-((3-(5-(((3R,4R,5R,6R)-3-acetamido-4,5-dihydroxy-6-(hydroxymethyl)-tetrahydro-2H-pyran-2-yl)oxy)pentanamido)propyl)amino)-3-oxopropoxy)methyl)-16,23,29-trioxo-4,7,10,13,20-pentaoxa-17,24,28-triazatritriacontanoate in DMSO (550 μL, 1.854 μmol) was added to a solution of PCSK9 (27.44 mg, 0.37 mol) in 2000 μL of PBS (0.1M pH 7.4) at 20° C. The mixture was agitated at 20° C. for 16 hours, and then another portion of 2,3,5,6-tetrafluorophenyl 33-(((2R,3R,4R,5R,6R)-3-acetamido-4,5-dihydroxy-6-(hydroxymethyl)-tetrahydro-2H-pyran-2-yl)oxy)-18-((3-((3-(5-(((2R,3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)-methyl)-18-((3-((3-(5-(((3R,4R,5R,6R)-3-acetamido-4,5-dihydroxy-6-(hydroxymethyl)-tetrahydro-2H-pyran-2-yl)oxy)pentanamido)propyl)amino)-3-oxopropoxy)methyl)-16,23,29-trioxo-4,7,10,13,20-pentaoxa-17,24,28-triazatritriacontanoate in DMSO (550 μL, 1.854 μmol) was added dropwise. The resulting mixture was agitated at 20° C. for 20 hours and then concentrated by using Amicon ultra 30K MWCO centrifugal filter with PBS as the washing buffer to give the GalNAc-labeled PCSK9 conjugate (1100 μL, 24 mg/mL, 26 mg, 95%).

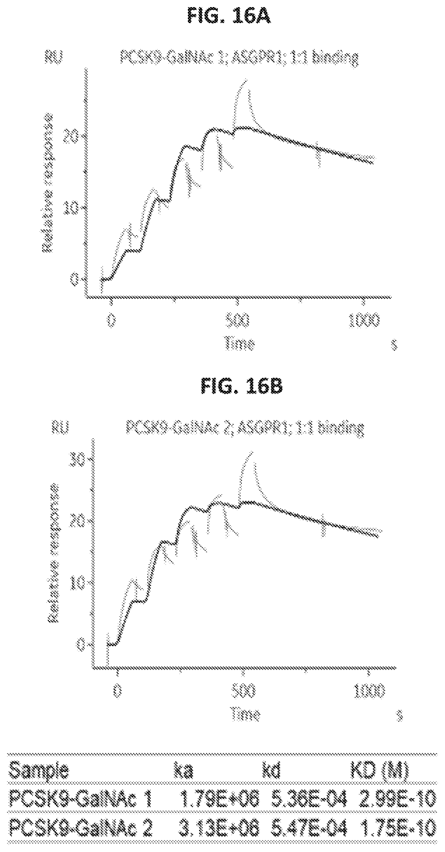

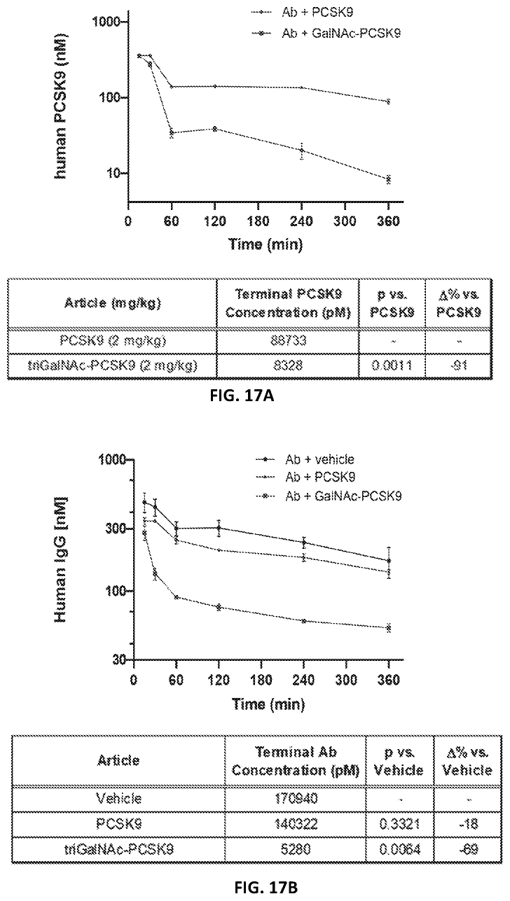

Binding studies with immobilized ASGPR1 demonstrates high affinity of the GalNAc-labeled PCSK9 (FIGS. 16A-B). In addition, pharmacokinetic studies demonstrated that the GalNAc-labeled PCSK9 is cleared more rapidly than native PCSK9 in mice, and results in rapid clearance of an anti-PCSK9 antibody from circulation (FIGS. 17A-B).

### Example 9: Targeted Plasma Protein Degradation (TPPD) Mediated by Bifunctional Molecules Binding to Asialoglycoprotein Receptor (ASGPR) and Proprotein Convertase Subtilisin/Kexin Type 9 (PCSK9)

This example describes a series of protein conjugates and small molecule-based bifunctional compounds that trigger the rapid and extensive removal of the disease-relevant protein PCSK9 from circulation after a single dose in vivo.

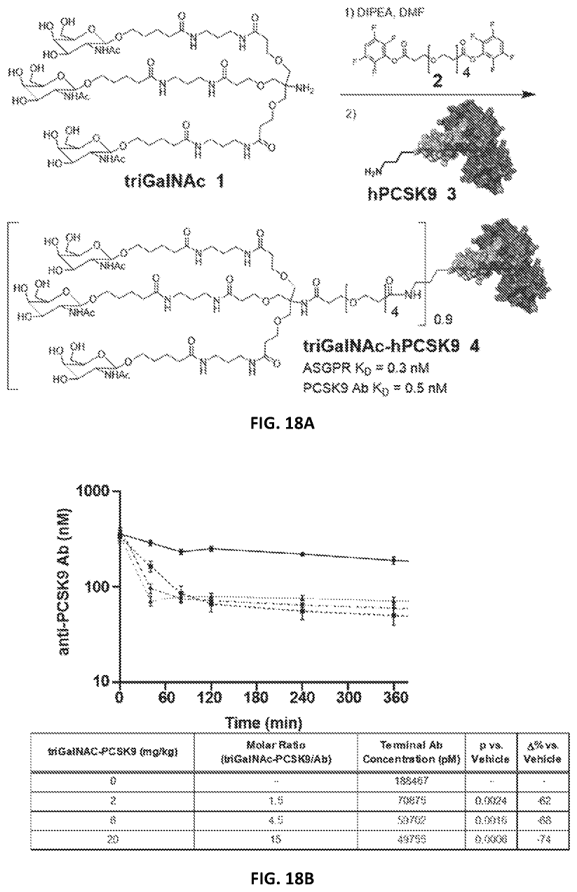

As a preliminary step towards demonstrating that an ASGPR ligand could accelerate the clearance of plasma hPCSK9, a conjugate of hPCSK9 with triGalNAc was generated (FIG. 18A). Specifically, triGalNAc 1 was reacted with the symmetrical activated ester 2 followed by conjugation with recombinant hPCSK9 protein 3 at a single solvent exposed lysine to provide the triGalNAc-PCSK9 conjugate 4 bridged by a short polyethylene glycol (PEG) linker. It was confirmed using surface plasmon resonance (SPR) that the triGalNAc-PCSK9 conjugate 4 bound both the ASGPR and an anti-PCSK9 antibody with high affinity. Following IV administration to LDLR (−/−) mice (mice devoid of the LDLR, the main clearance receptor of PCSK9), triGalNAc-hPCSK9 4 demonstrated enhanced clearance versus the unmodified hPCSK9 protein 3; at the terminal time point, the plasma levels of triGalNAc-hPCSK9 4 was reduced by 91% as compared to unmodified PCSK9 3 (FIG. 17A).

With the demonstration that triGalNAc-hPCSK9 conjugate 4 showed accelerated plasma clearance, it was examined whether conjugate 4 could function as a bifunctional molecule mediating the clearance of a hPCSK9 binding antibody. Co-administration of escalating doses of 4 with a fixed hPCSK9 Ab dose (FIG. 18B) produced rapid depletion of the hPCSK9 Ab as compared to mice receiving only hPCSK9 Ab. Even at the lowest dose studied (2 mg/kg), the bifunctional 4 produced a 62% reduction in circulating PCSK9 Ab versus vehicle control, with clearance occurring within 45 minutes following administration. The clearance was highly efficient, with only a slight molar excess of triGalNAc-hPCSK9 bifunctional 4 required (1.5-fold molar excess @ 2 mg/kg 4). In a second study (FIG. 17B), bifunctional 4 again rapidly cleared hPCSK9 Ab from circulation, while unmodified hPCSK9 3 did not. Though PCSK9 antibody clearance is not a clinically useful approach to treat a known human disease, these results provided a proof-of-concept that an ASGPR-targeted bifunctional agent could clear a plasma protein.

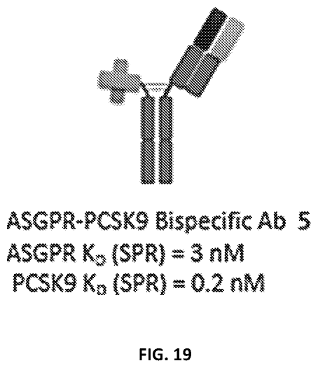

Applying the bifunctional approach to the acceleration of PCSK9 clearance from plasma, bispecific ASGPR-PCSK9 Ab 5 was constructed from published sequences (Bon, C. et al., MAbs 9, 1360-1369 (2017); Chaparro-Riggers, J. et al. J Biol Chem 287, 11090-7 (2012)). As shown in FIG. 19, ASGPR-PCSK9 bispecific antibody binding was confirmed by SPR, demonstrating productive binding to independently immobilized ASGPR (K_{D}=3 nM) and PCSK9 (K_{D}=0.2 nM). Bolus IV co-administration of the ASGPR-PCSK9 bispecific Ab 5 with hPCSK9 protein to LDLR (−/−) mice rapidly and significantly accelerated the clearance of hPCSK9 as compared to vehicle control at all dose levels tested with terminal concentrations reduced 34% (p=0.0031) at 0.1 mg/kg, 63% (p=0.0002) at 0.3 mg/kg, and 70% (p=0.0001) at 1 mg/kg (FIG. 3B). These results motivated a wider investigation of bifunctional molecules consisting of ligands for ASGPR and PCSK9.

Towards reduction of the bispecific Ab to a small molecule based heterobifunctional ligand, the ASGPR antibody component was replaced with the ASGPR ligand triGalNAc (1) (FIG. 18A), which has found clinical utility in liver selective ASO and siRNA delivery (Fitzgerald, K. et al. N Engl J Med 376, 41-51 (2017); Debacker, A. J. et al. Mol Ther 28, 1759-1771 (2020)). Presenting branched N-Acetyl galactose monomers, which individually present a low affinity interaction with ASGPR (K_{D}˜40 uM), as a branched trimeric ligand, 1 leverages an avidity effect, resulting a dramatic increase in the binding affinity to the trimeric ASGPR protein (K_{D}˜3 nM). Elaboration of triGalNAc (1) to a reagent suitable for antibody conjugation was effected by reaction with activated ester 6 (FIG. 20A). Following deprotection, peptide coupling to the Thiobridge (Badescu, G. et al. Bioconjug Chem 25, 1124-36 (2014)) reagent 7 provided compound 8, which was conjugated to the PCSK9 Ab 9 (Chaparro-Riggers, J. et al. J Biol Chem 287, 11090-7 (2012)) following reduction of the four inter-chain disulfides bridging the heavy and light chains of the PCSK9 Ab, to provide triGalNAc functionalized PCSK9 Ab 10 with the drug-to-antibody ratio of 3.8 (FIGS. 20A and 20B; n=3.8 in FIG. 20B). The synthesis of triGalNAc functionalized PCSK9 Ab 10 is described in Example 10 and Example 11. Binding of the triGalNAc-PCSK9 Ab bifunctional 10 was confirmed by SPR, demonstrating productive binding to independently immobilized ASGPR (K_{D}=6 nM) and PCSK9 (K_{D}=10 μM). Intravenous administration of hPCSK9 to LDLR (−/−) mice demonstrated significantly accelerated clearance with co-administration of triGalNAc-PCSK9 Ab conjugate 10 at all dose levels as compared to administration with a vehicle control (FIG. 6A). At the lowest dose studied (0.3 mg/kg) the triGalNAc-PCSK9 Ab 10 conjugate produced rapid in vivo clearance of hPCSK9 protein, resulting in a 91% depletion of hPCSK9 relative to vehicle control by 6 hours. The higher doses were similarly effective by 6 hours despite reduced clearance early in the time course. This profile is likely due to an effect where high initial bifunctional concentrations favor binary complex formation over the ternary complex formation required for bifunctional mediated clearance, a phenomenon known as the “Hook Effect” which has been reported in TPD-based bifunctional studies (Pettersson, M. & Crews, C. M. Drug Discov Today Technol 31, 15-27 (2019)). However, as the bifunctional is cleared from circulation, lower blood concentrations favor ternary complex formation and increased bifunctional mediated clearance.

### Example 10: Synthesis of GalNAc-Thiobridge Reagent

The synthesis of GalNAc-Thiobridge Reagent is shown in FIG. 20A.

To a solution of triGalNAc (compound 1 in FIG. 18A) (261 mg, 0.137 mmol) in DMF was added compound 6 (212 mg, 0.411 mmol) and the reaction was maintained overnight (FIG. 20A). The reaction was concentrated, and purified by preparative reverse phase HPLC to provide intermediate A (52 mg, 0.030 mmol). LCMS, m/z (M+1): 1748.5. Intermediate A (0.52 mg, 0.030 mmol) was dissolved in DCM (1 mL) and treated with TFA (0.5 mL) (FIG. 20A). The reaction was maintained at room temperature for 1 h, then concentrated and lyophilized to provide intermediate B (0.50 mg, 0.030 mmol) LCMS, m/z (M+1): 1648.5 (FIG. 20A).

Bis-sulfone-PEG(6u)-COOH (25.1 mg, 30.0 μmol, 2.0 eq.), prepared following previously described methods (Badescu, G. et al., Bridging disulfides for stable and defined antibody drug conjugates. Bioconjugate Chem. 2014, 25, 1124-36), was added to a round bottom flask equipped with a magnetic stirrer, followed by the addition of N-[(Dimethylamino)-1H-1,2,3-triazolo-[4,5-b]pyridin-1-ylmethylene]-N-methyl-methanaminium hexafluorophosphate N-oxide (HATU, 11.4 mg, 30.0 μmol, 2.0 eq.). Anhydrous N,N-dimethylformamide (DMF, 1.0 mL) was added and the solution was cooled to 4° C. with an ice bath. 4-methylmorpholine (NMM, 4.95 μL, 45.0 μmol, 3 eq.) was added and the reaction mixture was stirred at 4° C. for 30 min. In a separate flask, intermediate B was added (24.7 mg, 15.0 μmol, 1.0 eq.) and dissolved in anhydrous DMF (1.0 mL) (FIG. 20A). The solution of intermediate B in DMF was added to the reaction mixture containing bis-sulfone-PEG(6u)-COOH and HATU and allowed to stir at 4° C. (FIG. 20A). After 3 h, the reaction mixture was quenched by addition of trifluoroacetic acid (TFA, 1.49 μL) and concentrated under vacuum. The crude mixture was dissolved in 3:1 H₂O:MeCN (0.05% TFA, 2.0 mL), filtered with a syringe filter (0.45 m, PTFE), purified by reverse-phase (C-18) chromatography (gradient elution with H₂O/MeCN buffers containing 0.05% TFA from 0% to 100% buffer B) and lyophilized to afford the desired compound 8 (21.0 mg, 8.51 μmol, 57%) as a white solid. Purity by HPLC at 280 nm: 94.5%. ESI-MS, m/z: 1233.7 (80%), [M+2H]2+; 823.2 (10%) [M+3H]3+(FIG. 20A).

### Example 11: Synthesis of GalNAc-PCSK9 Ab

The synthesis of GalNAc-PCSK9 Ab is shown in FIG. 20B.

Anti-PCSK9 mAb at 6.7 mg/mL in 20 mM sodium phosphate buffer, pH 7.5, 150 mM NaCl, 20 mM EDTA (1.49 mL; 10 mg; 68.1 nmol; 1.0 eq.) was diluted with 20 mM sodium phosphate buffer, pH 7.5, 150 mM NaCl, 20 mM EDTA (425.8 μL) (FIG. 20B). A 5 mM solution of TCEP in endotoxin-free water (81.7 μL; 408 nmol; 6.0 eq.) was added to the dilute anti-PCSK9 mAb solution (FIG. 20B). The reduction was allowed to proceed at 40° C. for 1.5 hours with a final antibody concentration of 5.0 mg/mL.

After 1.5 hours at 40° C., the reduction mixture was diluted with 20 mM sodium phosphate buffer, pH 7.5, 150 mM NaCl, 20 mM EDTA (375.0 μL) and allowed to cool down to 22° C. A 12.0 mg/mL (4.83 mM) solution of ThioBridge® 8 (FIG. 20B) in DMF was prepared by dissolving 1.26 mg (512 nmol) of ThioBridge® 8 (MW=2466.8 g·mol-1) into 106 μL of DMF. The ThioBridge® 8 solution in DMF (98.0 μL; 1.18 mg; 476 nmol; 7.0 eq.) and DMF (27.0 μL) was added to the reduced anti-PCSK9 mAb solution resulting in a final concentration of 5% DMF and a final antibody concentration of 4.0 mg/mL. The conjugation reaction was incubated at 22° C. for 22 h.

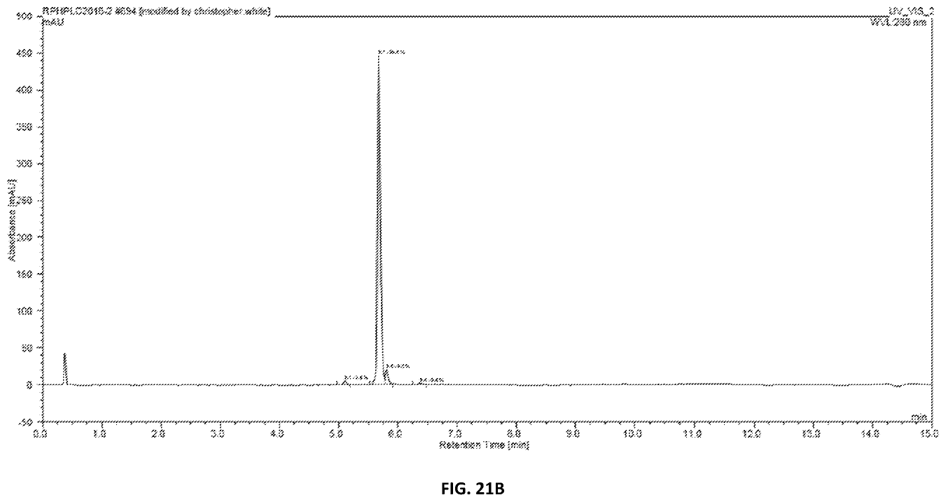

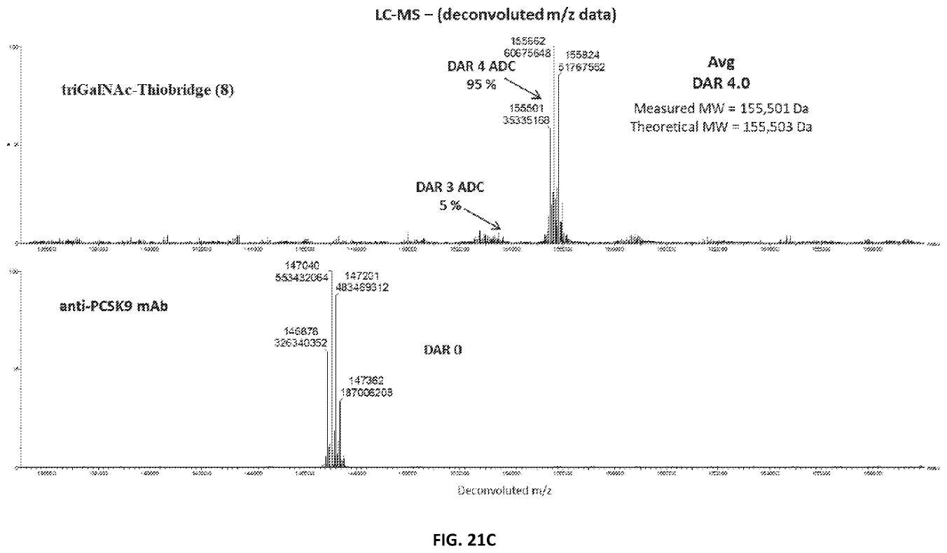

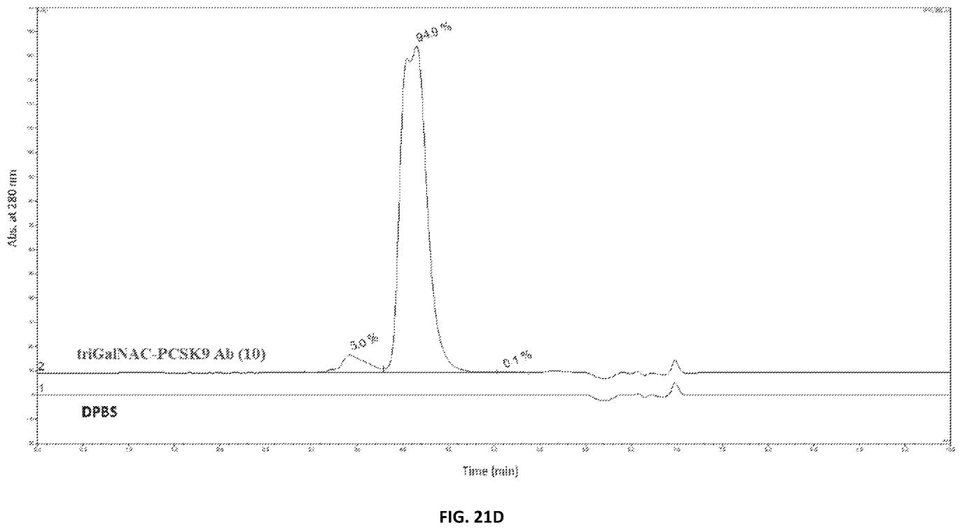

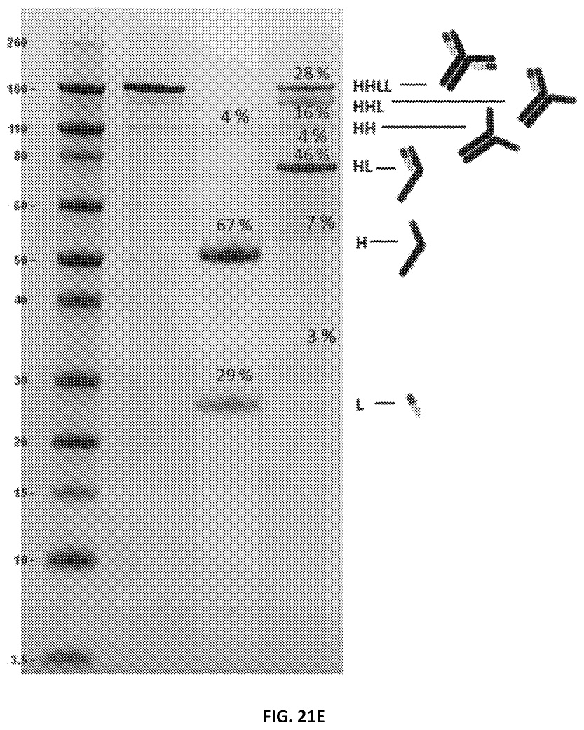

After 22 hours at 22° C., the reaction mixture was buffer exchanged into Dulbecco's PBS, pH 7.0 by means of 3 cycles of ultra-filtration using a Vivaspin20 centrifugal concentrator (10 kDa MWCO) and by gel filtration using an Illustra™ NAP-10 column equilibrated in Dulbecco's PBS, pH 7.0, per manufacturer's instructions. Recovered conjugate sample (9.9 mg; 1.19 mL) was sterile filtered through a 0.22 m pore size, PVDF membrane filter. The GalNAc-PCSK9 Ab was characterized by SEC, LC-MS, SDS-PAGE and quantified by UV and endotoxin levels were determined (See Table 8 and FIGS. 21A-21E).

TABLE 8
Analytical Results for GalNAc-PCSK9 Ab

| Appearance | Clear solution | |
|---|---|---|
| Drug-to-antibody ratio (DAR) | DAR 3 (n = 3 in FIG. 20B): 5% | |
| variants (LC-MS) | DAR 4 (n = 4 in FIG. 20B): 95% | |
| | Average DAR: 4.0 | |
| % Purity (SEC) | 94.9% monomeric | |
| Endotoxin (EU/mg) | 0.22 | |
| Concentration (UV) | 8.3 | mg/mL |
| Amount (by UV Analysis) | 9.9 | mg |
| Average MW | 155,680 | Da |

### Example 12: Methods

This example provides methods used in Examples 1 to 11.

### Surface Plasmon Resonance (SPR) Methods

Surface Plasmon Resonance (SPR) was used to obtain the binding data for ASGPR ligand compounds, either alone or as part of the bifunctional compounds.

ASGPR SPR experiments were performed on either Biacore T200 or Biacore 8K. The assay was optimized for Biacore SA chip in running buffer consisting of 20 mM HEPES, 300 mM NaCl, 2 mM CaCl₂), 0.01% tween-20, 2% DMSO, pH 7.4. Recombinant extracellular domain of human ASGPR1 with N-terminal biotinylated Avitag was diluted to 20 μg/mL in running buffer, and immobilized onto SA chip after it was preconditioned with at least four 60-second injections at 30 μL/min of 1 M NaCl/40 mM NaOH solution. Protein immobilization level varied from 500 RU to 3500 RU dependent on analytes' molecular weight, for targeted Rmax of 30 RUs. Direct binding of analytes to ASGR1 was observed when a dose response titration of analytes in 2% DMSO was flown over the immobilized protein. Data analysis was done on either Biacore T200 Evaluation Software or Biacore Insight Evaluation Software.

PCSK9 SPR Binding experiments were performed on a Proteon XPR36 instrument (Bio-Rad Laboratories). Purified recombinant full length human PCSK9 with C-terminal biotinylated Avitag was used for the assay. Biotinylated human PCSK9 was incubated with fivefold molar excess of compound, and immobilized onto a Neutravidin coated sensor chip (NLC chip, Bio-Rad Laboratories, Inc.). After conditioning the chip by injecting running buffer (20 mM Tris pH 7.5, 300 mM NaCl, 25 mM CaCl₂), 2 mM beta-mercaptoethanol, 2% DMSO and 0.05% Tween-20) at a flow rate of 0.1 ml/min for 240 s, PCSK9 protein was injected at a protein concentration of 0.02 mg/ml in running buffer with fivefold molar excess of bifunctional compound and a flow rate of 0.03 ml/min followed by 4 washes with running buffer with 1 μM of bifunctional compound (0.1 ml/min for 60 seconds each).

To perform sandwich SPR binding assays, His-ASGPR1(62-291) protein stocks were diluted with running buffer to a desired concentration. ASGPR was injected at a flow rate of 0.1 ml/min for 240 s and allowed to dissociate for 1200 s. Sensorgrams were recorded for association and dissociation phases. All sensorgrams were processed by subtracting the binding response recorded from a blank control surface (or interspot surface), followed by subtracting buffer blank injections from the reaction surface. Data were processed with the ProteOn Manager software (version 3.1.0.6, Bio-Rad, Inc.).

### In Vivo Pharmacology Methods Materials

All mice were received at between 7 and 10 weeks of age and acclimated for at least 3 days prior to experimentation. The animals were maintained on a 12 hour light/dark cycle at 70° F. and 50% humidity. Animals were provided water and food ad libitum. All mice were maintained on normal mouse chow.

Male LDLR(−/−) mice were obtained from Jackson Laboratories, Bar Harbor, ME (cat. #002207). On the morning of the study, mice were weighed and sorted into treatment groups so that each group had similar mean body weights. The compounds were then formulated based on the mean body weight of the mice so that the desired doses could be delivered in a fixed volume of 0.1 mL. Vehicle composed of 10% polyethylene glycol 300 (Sigma-Aldrich, Saint Louis, MO), 25% Kolliphor HS 15 (20% stock solution; Sigma-Aldrich, Saint Louis, MO) and 65% PBS (Gibco/ThermoFisher, Waltham, MA), compounds formulated in vehicle and human PCSK9 protein at 33.3 μg/mL in PBS were made immediately before dosing.

Animals were warmed under a heat lamp for 3 minutes then restrained in a rotating tail injector (Braintree Scientific Inc., Braintree, MA) and injected i.v. via the lateral tail vein with a 0.2 mL bolus containing both compound at the specified dose and 3.3 μg of wild type human PCSK9 protein. At 0.083, 0.25, 0.5, 1, 2 and 4 hours post-dose, animals were mechanically restrained and 25 μL of blood collected via tail snip into EDTA collection tubes (Sarstedt AG, Sarstedt, Germany) and stored on ice. Blood samples were centrifuged at 15,000 g, 4° C. for 10 minutes with resulting plasma aliquoted and frozen at −80° C. for later PD measurements.

### Determination of Human PCSK9 Plasma Protein Levels

Plasma concentrations of PCSK9 were assessed using a commercially available human (Human Proprotein Convertase 9/PCSK9 Quantikine ELISA Kit, DPC900; R&D Systems, Minneapolis, MN) ELISA kit. For the measurement of PCSK9 levels, plasma samples were diluted (1:25 or 1:50) and analyzed according to the manufacturer's instructions, with the exception that all incubations were performed on a Titer Plate Shaker (Lab-Line Instruments, Melrose Park, Illinois) at 300 rpm.

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

1. A bifunctional compound having the structure of Formula (I):
A_{G}-L-R_{G} (I)
or a pharmaceutically acceptable salt thereof, wherein:
A_{G} is a protein moiety that binds to an extracellular target, wherein said A_{G} is an antibody or a fragment thereof, a receptor or a fragment thereof, or an antigen protein or a fragment thereof;
L is absent or linker; and
R_{G} is a moiety that binds to a membrane-bound receptor associated with a degradation pathway,
wherein said membrane-bound receptor is a mannose-6-phosphate receptor (M6PR) or insulin-like growth factor 2 receptor (IGF2R), and said R_{G} is a small molecule, a carbohydrate or carbohydrate derivative molecule, or an antibody or fragment thereof that binds to M6PR and/or IGF2R; or
wherein said membrane-bound receptor is an asialoglycoprotein receptor (ASGPR), and said R_{G} is a small molecule, a carbohydrate or carbohydrate derivative molecule, or an antibody or fragment thereof that binds to ASGPR.

2. The bifunctional compound of claim 1, wherein A_{G} is an antibody or fragment thereof.

3. (canceled)

4. The bifunctional compound of claim 2, wherein the antibody is a full-length antibody, a multispecific antibody, a monospecific antibody, a bispecific antibody, or a fragment thereof.

5. The bifunctional compound of claim 2, wherein the antibody fragment comprises a Fab, a Fab′, a F(ab′)₂, a F(ab)₂, variable fragment (Fv), a domain antibody (dAb), a single domain antibody, or a single chain variable fragment (scFv).

6.-8. (canceled)

9. The bifunctional compound of claim 1, wherein said extracellular target is a pathogenic autoantibody or a fragment thereof.

10. The bifunctional compound of claim 9, wherein said A_{G} comprises an antigen protein or a fragment thereof that recognizes said pathogenic autoantibody.

11. The bifunctional compound of claim 10, wherein said antigen protein or a fragment thereof is a disintegrin and metalloproteinase with a thrombospondin type 1 motif, member 13 (ADAMTS13), steroidogenic cytochrome P450 enzyme 21-hydroxylase, N-methyl-d-aspartate-(NMDA)-receptor, erythrocytes, anti-smooth muscle antibodies (ASMAs), actin, platelet, signal recognition particle (SRP), 3-hydroxy-3-methyl-glutaryl-coenzyme A reductase (HMGCR), myosin, sperm, amylase alpha2, type XVII collagen (col17), kallikrein 13, type VII collagen (col7), myeloperoxidase (MPO), type IV collagen, proteinase 3 (PR3), thyrotropin receptor (TSHR), thyroglobulin, thyroid peroxidase (TPO), thyroglobulin, thyroid peroxidase (TPO), platelets, myeloperoxidase (MPO), muscle nicotinic acetylcholine receptors, muscle-specific kinase (MuSK), low-density lipoprotein receptor protein 4 (LRP4), myosin, beta1 adrenergic receptor, adenine-nucleotide translocase, aquaporin-4, myelin oligodendrocyte glycoprotein (MOG), heat shock protein 90 (HSP90), heat shock protein A5 (HSPA5), desmoglein-3, parietal cells, mitochondria, phospholipase A2 receptor (PLA2R), thrombospondin type 1 domain-containing 7A (THSD7A), cyclic citrullinated proteins, RNA binding proteins (Ros), La, double-stranded DNA (dsDNA), angiotensin II type 1 receptor (AT1R), endothelin-1 type A receptor (ETAR), insulin, glutamic acid decarboxylase or protein tyrosine phosphatase.

12. The bifunctional compound of claim 1, wherein A_{G} is a receptor or a fragment thereof.

13. (canceled)

14. The bifunctional compound of claim 1, wherein said extracellular target is a protein, a pathogenic target, or a non-protein.

15. The bifunctional compound of claim 14, wherein said extracellular target is a protein selected from a soluble protein or a membrane-associated protein.

16. The bifunctional compound of claim 14, wherein said extracellular target is a non-protein selected from a lipoprotein, liposome, nucleic acid, toxin, virus particle, or cell.

17.-20. (canceled)

21. The bifunctional compound of claim 15, wherein the soluble protein comprises an antibody, a soluble receptor, a secreted protein, a growth factor, a cytokine, a hormone, or an enzyme.

22. The bifunctional compound of claim 15, wherein the soluble protein is proprotein convertase subtilisin/kexin type 9 (PCSK9), complement factor H-related protein 3 (CFHR3), MICA (MHC class I chain-related gene A), or apolipoprotein-B (Apo-B).

23.-26. (canceled)

27. The bifunctional compound of claim 15, wherein the membrane-associated protein is a type I, type II or multipass membrane protein or a glycophosphatidylinositol (GPI) anchored membrane associated protein.

28.-30. (canceled)

31. The bifunctional compound of claim 16, wherein said lipoprotein is a lipoprotein receptor (LPR) or lipoprotein(a) (Lp(a)).

32. The bifunctional compound of claim 1, wherein the extracellular target is selected from: proprotein convertase subtilisin/kexin type 9 (PCSK9), tumor necrosis factor receptor 1 (TNFR1), interleukin-1 receptor (IL1R), low density lipoproteins, apolipoprotein B (ApoB), lipoprotein(a) (Lp(a)), apolipoprotein C3 (ApoCIII), angiopoietin-like 3 (ANGPTL3), angiopoietin-like 4 (ANGPTL4), angiopoietin-like 8 (ANGPTL8), Factor 11, growth differentiation factor 15 (GDF15), lipoprotein lipase (LPL), interleukin 1-beta (IL10), interleukin 17 (IL17), complement Factor B, complement Factor D, myeloperoxidase (MPO), immunoglobulin E (IgE), programmed cell death protein 1 (PD-1), programmed death-ligand 1 (PD-L1), interleukin 7 (IL7), interleukin 12A (IL12A), interleukin 23 (IL23), tumor necrosis factor A (TNFA), microtubule associated protein tau (MAPT), complement factor H-related protein 3 (FHR3), tissue inhibitor of metalloproteinases 1 (TIMP1), Apelin, bone morphogenetic protein 6 (BMP6), bone morphogenetic protein 9/growth differentiation factor 2 (BMP9/GDF2), colony stimulating factor 1 receptor (CSF-1), erythropoietin (EPO), interleukin 5 (IL5), milk fat globule-EGF Factor 8 protein (MFGE8), thymic stromal lymphopoietin (TSLP), thrombospondin (TSP), complement component 5 (C5), C—X—C motif chemokine ligand 10 (CXCL10), fibroblast growth factor 23 (FGF23), insulin-like growth factor 1 (IGF1), interleukin 10 (IL10), interleukin 13 (IL13), interleukin 2 (IL2), interleukin 6 (IL6), vascular endothelial growth factor A (VEGF-A), adenosine deaminase 2 (ADA2), soluble urokinase-type plasminogen activator receptor (suPAR), transforming growth factor beta 1 (TGF-β1), progranulin, alpha-synuclein, a toxin, a venom, an HBV soluble antigen, a viral antigen, a prion protein, a scFv, an AAV, and an anti-AAV antibody.

33. (canceled)

34. The bifunctional compound of claim 14, wherein the pathogenic target is associated with or causes a deleterious or unwanted effect in a sample, a cell or a subject, and/or wherein the pathogenic target is present at an expression level or an activity level that results in a deleterious or unwanted effect in a sample, a cell or a subject.

35.-37. (canceled)

38. The bifunctional compound of claim 14, wherein the pathogenic target is a pathogenic autoantibody or a fragment thereof, a cell surface receptor, or a neurological target.

39. (canceled)

40. The bifunctional compound of claim 38, wherein the cell surface receptor is selected from TNF receptor 1 (TNFR1), interleukin-1 receptor (IL1R), PD-L1, epidermal growth factor receptor (EGFR), or transferrin.

41. (canceled)

42. The bifunctional compound of claim 38, wherein the neurological target is a Tau protein or aggregate or an immuno-oncology target.

43. The bifunctional compound of claim 42, wherein the immuno-oncology target is progranulin.

44.-46. (canceled)

47. The bifunctional compound of claim 1, wherein R_{G} comprises a binding moiety for a M6PR or IGF2R, a binding moiety for an ASGPR, or an ASGPR ligand.

48. (canceled)

49. The bifunctional compound of claim 47, wherein the ASGPR binding moiety is ASGPR1 or ASGPR2.

50. (canceled)

51. The bifunctional compound of claim 1, wherein R_{G} comprises a compound of Formula (I): 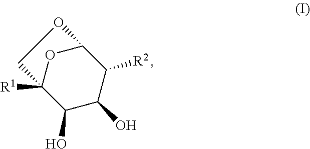
or a pharmaceutically acceptable salt thereof, wherein:
R¹ is —CN, —CH₂—CN, —C≡CH, —CH₂—N₃, —CH₂—NH₂, —CH₂—N(R⁴)—S(O)₂—R⁵, —CH₂—CO₂H, —CO₂H, —CH₂—OH, —CH₂—SH, —CH═CH R⁵, —CH₂—R⁵, —CH₂—S—R⁵, —CH₂—N(R⁴)—R⁵, —CH₂—N(R⁴)—C(O)—R⁵, —CH₂—N(R⁴)—C(O)—O—R⁵, —CH₂—N(R⁴)—C(O)—N(R⁴)—R⁵, —CH₂—O—R⁵, —CH₂—O—C(O)—R⁵, —CH₂—O—C(O)—N(R⁴)—R⁵, —CH₂—O—C(O)—O—R⁵, CH₂—S(O)—R⁵, —CH₂—S(O)₂—R⁵, —CH₂—S(O)₂—N(R⁴)—R⁵, —C(O)—NH₂, —C(O)—O—R⁵, —C(O)—N(R⁴)—R⁵, or aryl or heteroaryl, wherein the aryl or heteroaryl is optionally substituted with R⁵,
or R¹ is —Z—X—Y, wherein X is a linker or a drug delivery system, Y is absent or is a ligand selected from the group consisting of a small molecule, an amino acid sequence, a nucleic acid sequence, an antibody, an oligomer, a polymer, genetically derived material, a liposome, a nanoparticle, dye, and a fluorescent probe, or a combination thereof, and Z is absent or is —C≡C—, —CH═CH—, —CH₂—, —CH₂—O—, —C(O)—N(R⁴)—, —CH₂—S—, —CH₂—S(O)—, —CH₂—S(O)₂—, —CH₂—S(O)₂—N(R⁴)—, —C(O)—O—, —CH₂—N(R⁴)—, —CH₂—N(R⁴)—C(O)—, —CH₂—N(R⁴)—S(O)₂—, —CH₂—N(R⁴)—C(O)—O—, —CH₂—N(R⁴)—C(O)—N(R⁴)—, —CH₂—O—C(O)—, —CH₂—O—C(O)—N(R⁴)—, —CH₂—O—C(O)—O—, or aryl or heteroaryl, wherein the aryl or heteroaryl is optionally substituted with R⁵;
R² is —OH, —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³; and wherein when R¹ is —CH₂—OH, R² is —N₃, —N(R³)₂, —N(R³)—C(O)—R³, —N(R³)—C(O)—N(R³)₂, —N(R³)—C(O)—OR³, tetrazole, or triazole, wherein the tetrazole and triazole are optionally substituted with R³;
each R³ is independently —H, (C₁-C₅)alkyl, halo-substituted (C₁-C₅)alkyl, or (C₃-C₆)cycloalkyl, wherein a —CH₂— group of the alkyl or cycloalkyl may be replaced with a heteroatom group selected from —O—, —S—, and —N(R⁴)— and —CH₃ of the alkyl may be replaced with a heteroatom group selected from —N(R⁴)₂, —OR⁴, and —S(R⁴) wherein the heteroatom groups are separated by at least 2 carbon atoms;
each R⁴ is independently —H, —(C₁-C₂₀)alkyl, or (C₃-C₆)cycloalkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴), wherein the heteroatom groups are separated by at least 2 carbon atoms; and wherein the alkyl and cycloalkyl may be substituted with one to six halo atoms; and
each R⁵ is independently —H, (C₃-C₂₀)cycloalkyl or (C₁-C₂₀)alkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴), wherein the heteroatom groups are separated by at least 2 carbon atoms; and wherein the alkyl and cycloalkyl may be substituted with one to six halo atoms.

52. The bifunctional compound of claim 1, wherein R_{G} is selected from: 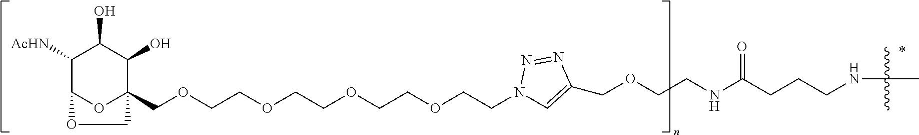
wherein n is 1, 2, or 3; 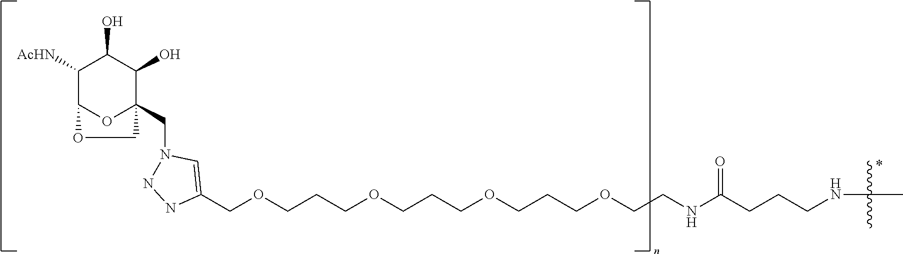
wherein n is 2 or 3; and
wherein R_{G} is attached to L at the terminal amine.

53. The bifunctional compound of claim 1, wherein R_{G} is selected from: 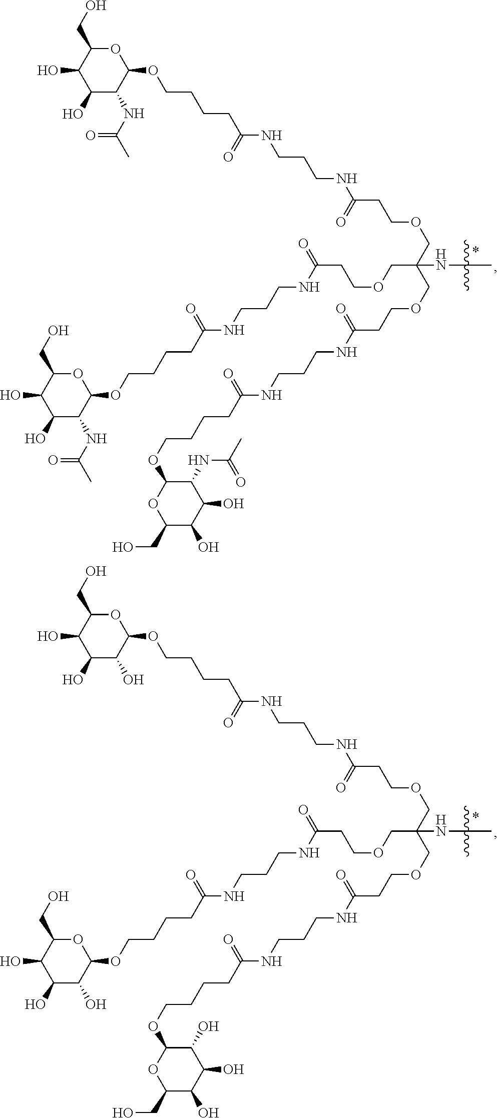 [SMILES: *NC(COCCC(=O)NCCCNC(=O)CCCCO[C@@H]1O[C@H](CO)[C@H](O)[C@H](O)[C@H]1NC(C)=O)(COCCC(=O)NCCCNC(=O)CCCCO[C@@H]1O[C@H](CO)[C@H](O)[C@H](O)[C@H]1NC(C)=O)COCCC(=O)NCCCNC(=O)CCCCO[C@@H]1O[C@H](CO)[C@H](O)[C@H](O)[C@H]1NC(C)=O ; *NC(COCCC(=O)NCCCNC(=O)CCCCO[C@@H]1O[C@H](CO)[C@H](O)[C@H](O)[C@H]1O)(COCCC(=O)NCCCNC(=O)CCCCO[C@@H]1O[C@H](CO)[C@H](O)[C@H](O)[C@H]1O)COCCC(=O)NCCCNC(=O)CCCCO[C@@H]1O[C@H](CO)[C@H](O)[C@H](O)[C@H]1O] 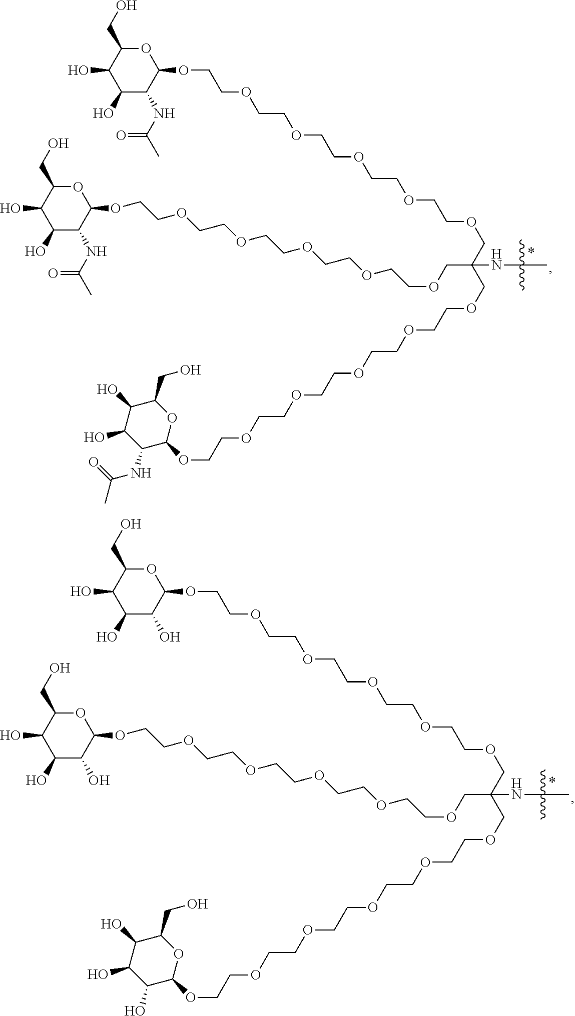 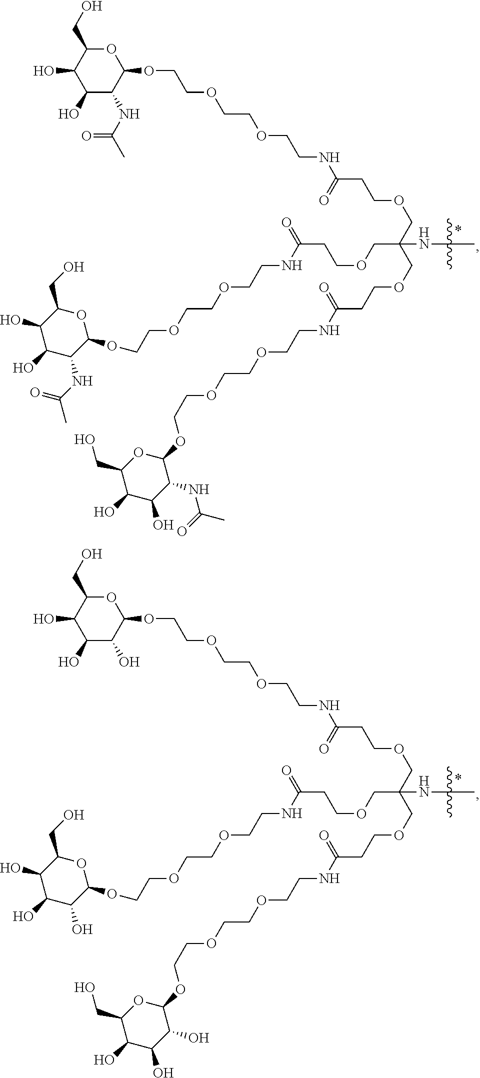 [SMILES: *NC(COCCC(=O)NCCOCCOCCO[C@@H]1O[C@H](CO)[C@H](O)[C@H](O)[C@H]1NC(C)=O)(COCCC(=O)NCCOCCOCCO[C@@H]1O[C@H](CO)[C@H](O)[C@H](O)[C@H]1NC(C)=O)COCCC(=O)NCCOCCOCCO[C@@H]1O[C@H](CO)[C@H](O)[C@H](O)[C@H]1NC(C)=O ; *NC(COCCC(=O)NCCOCCOCCO[C@@H]1O[C@H](CO)[C@H](O)[C@H](O)[C@H]1O)(COCCC(=O)NCCOCCOCCO[C@@H]1O[C@H](CO)[C@H](O)[C@H](O)[C@H]1O)COCCC(=O)NCCOCCOCCO[C@@H]1O[C@H](CO)[C@H](O)[C@H](O)[C@H]1O] 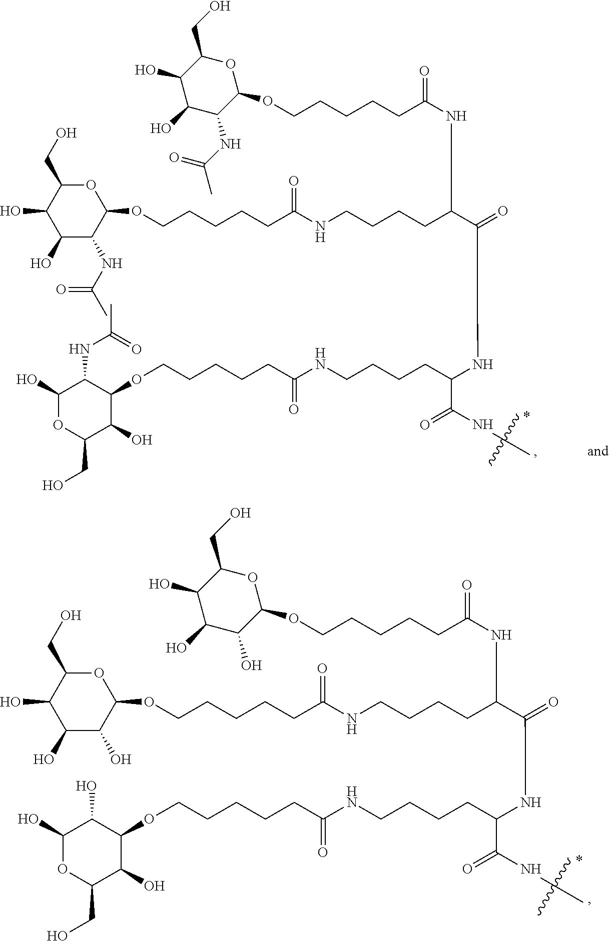 [SMILES: *NC(=O)C(CCCCNC(=O)CCCCCO[C@H]1[C@@H](O)[C@@H](CO)O[C@@H](O)[C@@H]1NC(C)=O)NC(=O)C(CCCCNC(=O)CCCCCO[C@@H]1O[C@H](CO)[C@H](O)[C@H](O)[C@H]1NC(C)=O)NC(=O)CCCCCO[C@@H]1O[C@H](CO)[C@H](O)[C@H](O)[C@H]1NC(C)=O ; *NC(=O)C(CCCCNC(=O)CCCCCO[C@@H]1[C@@H](O)[C@H](O)O[C@H](CO)[C@@H]1O)NC(=O)C(CCCCNC(=O)CCCCCO[C@@H]1O[C@H](CO)[C@H](O)[C@H](O)[C@H]1O)NC(=O)CCCCCO[C@@H]1O[C@H](CO)[C@H](O)[C@H](O)[C@H]1O] 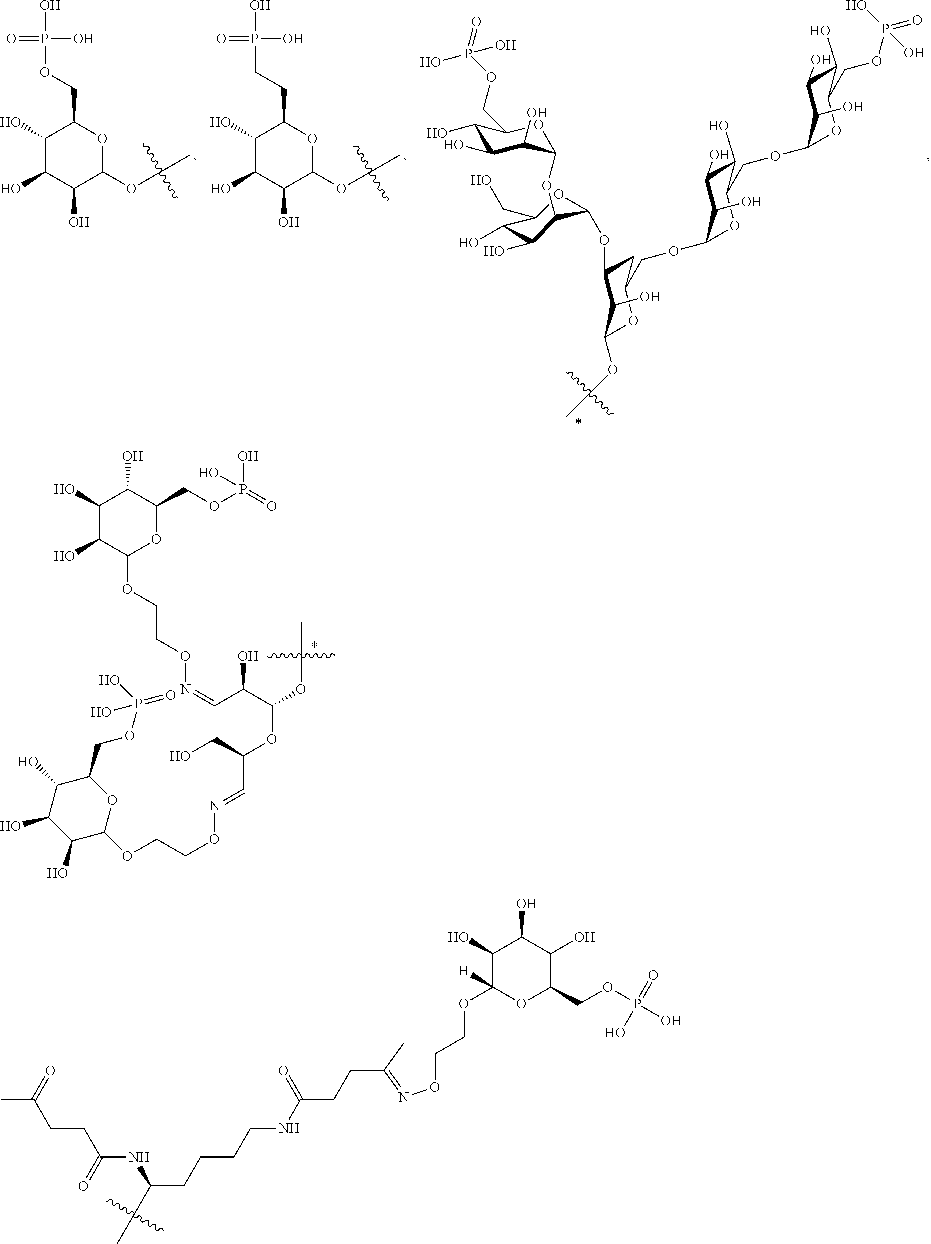 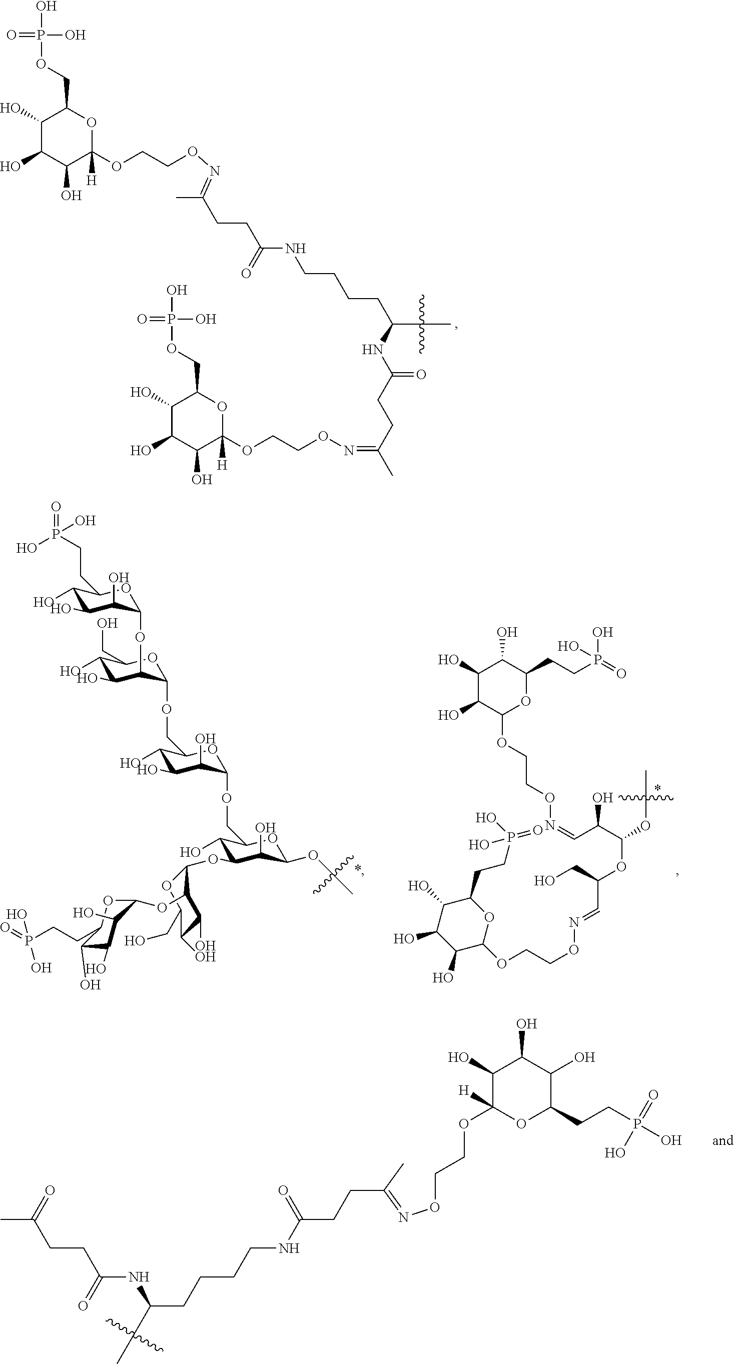 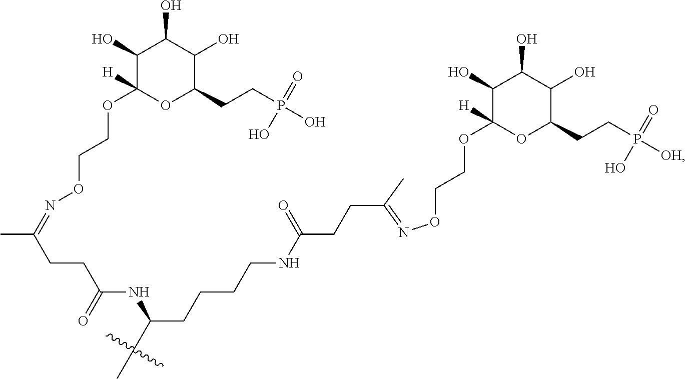 [SMILES: *[C@H](CCCCNC(=O)CC/C(C)=N/OCCO[C@H]1O[C@H](CCP(=O)(O)O)C(O)[C@H](O)[C@@H]1O)NC(=O)CC/C(C)=N\OCCO[C@H]1O[C@H](CCP(=O)(O)O)C(O)[C@H](O)[C@@H]1O]
wherein the * of R_{G} indicates the point of attachment to L.

54. The bifunctional compound of claim 1, wherein R_{G} comprises an M6PR or IGF2R binding moiety and the extracellular target is TNFR1, IL1R, progranulin, Tau, MUC5B, TREM2, or EGFR.

55. The bifunctional compound of claim 1, wherein L comprises a heteroalkylene moiety.

56.-69. (canceled)

70. A pharmaceutical composition comprising the bifunctional compound of claim 1 and a pharmaceutically acceptable excipient.

71.-75. (canceled)

76. A method of targeting an extracellular target for degradation or decreasing the level and/or activity of an extracellular target in a cell, comprising contacting the cell with the bifunctional compound of claim 1.

77.-81. (canceled)

82. A method of targeting an extracellular target for degradation or decreasing the level and/or activity of the extracellular target in a subject, comprising administering the bifunctional compound of claim 1 to the subject.

83.-89. (canceled)

90. A method of treating a disease, disorder, condition, or clinical situation in a subject, comprising administering an effective amount of the bifunctional compound of claim 1 to said subject.

91. The method of claim 90, wherein the disease, disorder, condition, or clinical situation is a cancer, a neurological disease or disorder, cardiovascular disease, atherosclerosis, arteriosclerosis, hyperlipidemia, familial hypercholesterolemia (heterozygous or homozygous), familial chylomicronemia syndrome, inflammation, autoimmunity, an infectious disease, a respiratory condition, urticaria, nephropathy, or a renal disease or disorder.

92. The method of claim 91, wherein the neurological disease or disorder is Alzheimer's disease or neurodegeneration, the respiratory condition is asthma, or the nephropathy is IgA nephropathy or membranous nephropathy.

93.-95. (canceled)

96. The method of claim 90, wherein said disease, disorder, condition, or clinical situation comprises acute inflammatory demyelinating polyradiculoneuropathy (Guillain-Barre syndrome), acute liver failure, anti-glomerular basement membrane disease (Goodpasture syndrome), chronic inflammatory demyelinating polyradiculoneuropathy (CIDP), cutaneous T cell lymphoma (CTCL); mycosis fungoides; Sezary syndrome, familial hypercholesterolemia, focal segmental glomerulosclerosis (FSGS), hereditary hemochromatosis, hyperviscosity in hypergammaglobulinemia, hyperviscosity in hypergammaglobulinemia, myasthenia gravis, N-methyl-D-aspartate receptor antibody encephalitis, paraproteinemic demyelinating neuropathies; chronic acquired demyelinating polyneuropathies, polycythemia vera; erythrocytosis; thrombotic microangiopathy, thrombotic thrombocytopenic purpura (TTP); vasculitis; Wilson disease, fulminant; dilated cardiomyopathy; Graft-versus-host disease (GVHD); Lipoprotein(a) hyperlipoproteinemia; multiple sclerosis; neuromyelitis optica spectrum disorders (NMOSD); pediatric autoimmune neuropsychiatric disorders associated with streptococcal infections (PANDAS); Sydenham's chorea; peripheral vascular diseases; sickle cell disease; voltage-gated potassium channel (VGKC) antibody related diseases.

97. The method of claim 90, wherein the bifunctional compound is administered via oral, parenteral, topical, mucosal, nasal, buccal, or ophthalmological administration.

98. (canceled)

99. The method of claim 97, wherein the parenteral administration is via intravenous, subcutaneous, intracutaneous, intramuscular, intraarticular, intraarterial, intraperitoneal, intrasynovial, intrasternal, intrathecal, intralesional or intracranial injection.

100.-115. (canceled)

116. A bispecific antibody or fragment thereof that binds to asialoglycoprotein receptor (ASGPR) and proprotein convertase subtilisin/kexin type 9 (PCSK9).

117. The bispecific antibody or fragment thereof of claim 116, wherein the bispecific antibody is a full-length antibody.

118. The bispecific antibody or fragment thereof of claim 116, wherein the bispecific antibody fragment thereof comprises a Fab, a Fab′, a F(ab′)₂, a F(ab)₂, variable fragment (Fv), a domain antibody (dAb), a single domain antibody, or a single chain variable fragment (scFv).

119. The bispecific antibody or fragment thereof of claim 116, wherein the bispecific antibody or fragment thereof comprises a linker.

120. The bispecific antibody or fragment thereof of claim 119, wherein the linker is a (G4S)ₙ linker, wherein n is an integer from 1 to 20.

121. The bispecific antibody or fragment thereof of claim 116, wherein the bispecific antibody or fragment thereof comprises one or more of the following:
(i) a heavy chain complementarity determining region 1 (HCDR1) amino acid sequence as set forth in SEQ ID NO: 9, a heavy chain complementarity determining region 2 (HCDR2) amino acid sequence as set forth in SEQ ID NO: 10, and a heavy chain complementarity determining region 3 (HCDR3) amino acid sequence as set forth in SEQ ID NO: 11;
(ii) a HCDR1 amino acid sequence as set forth in SEQ ID NO: 21, a HCDR2 amino acid sequence as set forth in SEQ ID NO: 22, and a HCDR3 amino acid sequence as set forth in SEQ ID NO: 11;
(iii) a HCDR1 amino acid sequence as set forth in SEQ ID NO: 31, a HCDR2 amino acid sequence as set forth in SEQ ID NO: 32, and a HCDR3 amino acid sequence as set forth in SEQ ID NO: 33;
(iv) a HCDR1 amino acid sequence as set forth in SEQ ID NO: 15, a HCDR2 amino acid sequence as set forth in SEQ ID NO: 16, and a HCDR3 amino acid sequence as set forth in SEQ ID NO: 17;
(v) a HCDR1 amino acid sequence as set forth in SEQ ID NO: 26, a HCDR2 amino acid sequence as set forth in SEQ ID NO: 27, and a HCDR3 amino acid sequence as set forth in SEQ ID NO: 17
(vi) a HCDR1 amino acid sequence as set forth in SEQ ID NO: 36, a HCDR2 amino acid sequence as set forth in SEQ ID NO: 37, and a HCDR3 amino acid sequence as set forth in SEQ ID NO: 38;
(vii) a light chain complementarity determining region 1 (LCDR1) amino acid sequence as set forth in SEQ ID NO: 12, a light chain complementarity determining region 2 (LCDR2) amino acid sequence as set forth in SEQ ID NO: 13, and a light chain complementarity determining region 3 (LCDR3) amino acid sequence as set forth in SEQ ID NO: 14;
(viii) a LCDR1 amino acid sequence as set forth in SEQ ID NO: 23, a LCDR2 amino acid sequence as set forth in SEQ ID NO: 24, and a LCDR3 amino acid sequence as set forth in SEQ ID NO: 25;
(ix) a LCDR1 amino acid sequence as set forth in SEQ ID NO: 34, a LCDR2 amino acid sequence as set forth in SEQ ID NO: 35, and a LCDR3 amino acid sequence as set forth in SEQ ID NO: 14;
(x) a LCDR1 amino acid sequence as set forth in SEQ ID NO: 18, a LCDR2 amino acid sequence as set forth in SEQ ID NO: 19, and a LCDR3 amino acid sequence as set forth in SEQ ID NO: 20;
(xi) a LCDR1 amino acid sequence as set forth in SEQ ID NO: 28, a LCDR2 amino acid sequence as set forth in SEQ ID NO: 29, and a LCDR3 amino acid sequence as set forth in SEQ ID NO: 30; and/or
(xii) a LCDR1 amino acid sequence as set forth in SEQ ID NO: 39, a LCDR2 amino acid sequence as set forth in SEQ ID NO: 29, and a LCDR3 amino acid sequence as set forth in SEQ ID NO: 20.

122. The bispecific antibody or fragment thereof of claim 116, wherein the bispecific antibody or fragment thereof comprises one or more of the following:
(i) a heavy chain variable region (VH) amino acid sequence having at least about 85%, at least about 90%, at least about 95%, at least about 96%, at least about 97%, at least about 98%, or at least about 99% sequence identity to SEQ ID NO: 1;
(ii) a VH amino acid sequence having at least about 85%, at least about 90%, at least about 95%, at least about 96%, at least about 97%, at least about 98%, or at least about 99% sequence identity to SEQ ID NO: 3;
(iii) a light chain variable region (VL) amino acid sequence having at least about 85%, at least about 90%, at least about 95%, at least about 96%, at least about 97%, at least about 98%, or at least about 99% sequence identity to SEQ ID NO: 2; and/or
(iv) a VL amino acid sequence having at least about 85%, at least about 90%, at least about 95%, at least about 96%, at least about 97%, at least about 98%, or at least about 99% sequence identity to SEQ ID NO: 4.

123. The bispecific antibody or fragment thereof of claim 116, wherein the bispecific antibody or fragment thereof comprises one or more of the following:
(i) a VH amino acid sequence as set forth in SEQ ID NO: 1;
(ii) a VH amino acid sequence as set forth in SEQ ID NO: 3;
(iii) a VL amino acid sequence as set forth in SEQ ID NO: 2; and/or
(iv) a VL amino acid sequence as set forth in SEQ ID NO: 4.

124. The bispecific antibody or fragment thereof of claim 116, comprising an amino acid sequence having at least about 85%, at least about 90%, at least about 95%, at least about 96%, at least about 97%, at least about 98%, or at least about 99% sequence identity to SEQ ID NO: 5, 6, 7, and/or 8, or comprising an amino acid sequence as set forth in SEQ ID NO: 5, 6, 7, and/or 8.

125.-126. (canceled)

127. A pharmaceutical composition comprising a bispecific antibody or fragment thereof of claim 116 and a pharmaceutically acceptable excipient.

128. A nucleic acid molecule encoding a bispecific antibody or fragment thereof of claim 116.

129. An expression vector comprising the nucleic acid of claim 128.

130. A host cell comprising the nucleic acid of claim 128.

131. A method of producing a bispecific antibody or fragment thereof, the method comprising culturing the host cell of claim 130 under conditions suitable for gene expression and thereafter purifying and collecting the bispecific antibody or fragment thereof from the cell culture.

132.-136. (canceled)

137. A method of targeting PCSK9 for degradation or for decreasing the level and/or activity of PCSK9 in a subject or a sample, comprising administering to the subject or contacting the sample with the bispecific antibody or fragment thereof of claim 116.

138.-148. (canceled)

149. A method of treating a disease, disorder, condition, or clinical situation in a subject, comprising administering an effective amount of the bispecific antibody or fragment thereof of claim 116 to the subject.

150. The method of claim 149, wherein the bispecific antibody or fragment thereof is administered via oral, parenteral, topical, mucosal, nasal, buccal, or ophthalmological administration.

151. (canceled)

152. The method of claim 150, wherein the parenteral administration is via intravenous, subcutaneous, intracutaneous, intramuscular, intraarticular, intraarterial, intraperitoneal, intrasynovial, intrasternal, intrathecal, intralesional or intracranial injection.

153-155. (canceled)

156. The method of claim 149, wherein the disease, disorder, condition, or clinical situation is a PCSK9-mediated disease or disorder.

157. The method of any one of claim 156, wherein the PCSK9-mediated disease or disorder is selected from hypercholesterolemia, hyperlipidemia, hypertriglyceridemia, sitosterolemia, atherosclerosis, arteriosclerosis, coronary heart disease, peripheral vascular disease (including aortic diseases and cerebrovascular disease), peripheral arterial disease, vascular inflammation, elevated Lp(a), elevated LDL, elevated TRL, elevated triglycerides, sepsis, and xanthoma.

158.-168. (canceled)


SEQUENCE LISTING (47 sequences)

SEQ ID NO: 1 - protein, 118 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 2 - protein, 107 aa, Artificial Sequence
  source: /note=“Description of Artificial Sequence Synthetic polypeptide”
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 3 - protein, 107 aa, Artificial Sequence; identical to SEQ ID NO: 2
  source: /note=“Description of Artificial Sequence Synthetic polypeptide”

SEQ ID NO: 4 - protein, 109 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSLERIGYLSYVFGGGTKLTVL

SEQ ID NO: 5 - protein, 249 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDFSSRRWYLEYWGQGTLVTVSSGGGGSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSLERIGYLSYVFGGGTKLTVL

SEQ ID NO: 6 - protein, 214 aa, Artificial Sequence
  source: /note=“Description of Artificial Sequence Synthetic polypeptide”
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 481 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDFSSRRWYLEYWGQGTLVTVSSGGGGSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSLERIGYLSYVFGGGTKLTVLGGGGSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 8 - protein, 448 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 5 aa, Artificial Sequence
  source: /note=“Description of Artificial Sequence Synthetic peptide”
SYAMS

SEQ ID NO: 10 - protein, 17 aa, Artificial Sequence
  source: /note=“Description of Artificial Sequence Synthetic peptide”
AISGSGGSTYYADSVKG

SEQ ID NO: 11 - protein, 11 aa, Artificial Sequence
  source: /note=“Description of Artificial Sequence Synthetic peptide”
DFSSRRWYLEY

SEQ ID NO: 12 - protein, 11 aa, Artificial Sequence
  source: /note=“Description of Artificial Sequence Synthetic peptide”
QGDSLRSYYAS

SEQ ID NO: 13 - protein, 7 aa, Artificial Sequence
  source: /note=“Description of Artificial Sequence Synthetic peptide”
GKNNRPS

SEQ ID NO: 14 - protein, 12 aa, Artificial Sequence
  source: /note=“Description of Artificial Sequence Synthetic peptide”
NSLERIGYLSYV

SEQ ID NO: 15 - protein, 5 aa, Artificial Sequence
  source: /note=“Description of Artificial Sequence Synthetic peptide”
SYYMH

SEQ ID NO: 16 - protein, 17 aa, Artificial Sequence
  source: /note=“Description of Artificial Sequence Synthetic peptide”
EISPFGGRTNYNEKFKS

SEQ ID NO: 17 - protein, 9 aa, Artificial Sequence
  source: /note=“Description of Artificial Sequence Synthetic peptide”
ERPLYASDL

SEQ ID NO: 18 - protein, 11 aa, Artificial Sequence
  source: /note=“Description of Artificial Sequence Synthetic peptide”
RASQGISSALA

SEQ ID NO: 19 - protein, 7 aa, Artificial Sequence
  source: /note=“Description of Artificial Sequence Synthetic peptide”
SASYRYT

SEQ ID NO: 20 - protein, 9 aa, Artificial Sequence
  source: /note=“Description of Artificial Sequence Synthetic peptide”
QQRYSLWRT

SEQ ID NO: 21 - protein, 7 aa, Artificial Sequence
  source: /note=“Description of Artificial Sequence Synthetic peptide”
GFTFSSY

SEQ ID NO: 22 - protein, 6 aa, Artificial Sequence
  source: /note=“Description of Artificial Sequence Synthetic peptide”
SGSGGS

SEQ ID NO: 23 - protein, 7 aa, Artificial Sequence
  source: /note=“Description of Artificial Sequence Synthetic peptide”
DSLRSYY

SEQ ID NO: 24 - protein, 3 aa, Artificial Sequence
  source: /note=“Description of Artificial Sequence Synthetic peptide”
GKN

SEQ ID NO: 25 - protein, 9 aa, Artificial Sequence
  source: /note=“Description of Artificial Sequence Synthetic peptide”
LERIGYLSY

SEQ ID NO: 26 - protein, 7 aa, Artificial Sequence
  source: /note=“Description of Artificial Sequence Synthetic peptide”
GYTFTSY

SEQ ID NO: 27 - protein, 6 aa, Artificial Sequence
  source: /note=“Description of Artificial Sequence Synthetic peptide”
SPFGGR

SEQ ID NO: 28 - protein, 7 aa, Artificial Sequence
  source: /note=“Description of Artificial Sequence Synthetic peptide”
SQGISSA

SEQ ID NO: 29 - protein, 3 aa, Artificial Sequence
  source: /note=“Description of Artificial Sequence Synthetic peptide”
SAS

SEQ ID NO: 30 - protein, 6 aa, Artificial Sequence
  source: /note=“Description of Artificial Sequence Synthetic peptide”
RYSLWR

SEQ ID NO: 31 - protein, 8 aa, Artificial Sequence
  source: /note=“Description of Artificial Sequence Synthetic peptide”
GFTFSSYA

SEQ ID NO: 32 - protein, 8 aa, Artificial Sequence
  source: /note=“Description of Artificial Sequence Synthetic peptide”
ISGSGGST

SEQ ID NO: 33 - protein, 13 aa, Artificial Sequence
  source: /note=“Description of Artificial Sequence Synthetic peptide”
AKDFSSRRWYLEY

SEQ ID NO: 34 - protein, 6 aa, Artificial Sequence
  source: /note=“Description of Artificial Sequence Synthetic peptide”
SLRSYY

SEQ ID NO: 35 - protein, 5 aa, Artificial Sequence
  source: /note=“Description of Artificial Sequence Synthetic peptide”
IYGKN

SEQ ID NO: 36 - protein, 8 aa, Artificial Sequence
  source: /note=“Description of Artificial Sequence Synthetic peptide”
GYTFTSYY

SEQ ID NO: 37 - protein, 8 aa, Artificial Sequence
  source: /note=“Description of Artificial Sequence Synthetic peptide”
ISPFGGRT

SEQ ID NO: 38 - protein, 11 aa, Artificial Sequence
  source: /note=“Description of Artificial Sequence Synthetic peptide”
ARERPLYASDL

SEQ ID NO: 39 - protein, 6 aa, Artificial Sequence
  source: /note=“Description of Artificial Sequence Synthetic peptide”
QGISSA

SEQ ID NO: 40 - protein, 540 aa, Artificial Sequence
  source: /note=“Description of Artificial Sequence Synthetic polypeptide”
DSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFREQGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHQLTQPRGVNWCERTTLSNGGCQYLCLPAPQINPHSPKFTCACPDGMLLARDMRSCLTEAEAAVALPETGGHHHHHH

SEQ ID NO: 41 - protein, 534 aa, Artificial Sequence
  source: /note=“Description of Artificial Sequence Synthetic polypeptide”
DSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFREQGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHQLTQPRGVNWCERTTLSNGGCQYLCLPAPQINPHSPKFTCACPDGMLLARDMRSCLTEAEAAVAHHHHHH

SEQ ID NO: 42 - protein, 20 aa, Artificial Sequence
  source: /note=“Description of Artificial Sequence Synthetic peptide”
  SITE (1)..(20): /note=“This sequence may encompass 1-4 ′Gly Gly Gly Gly Ser′ repeating units”
GGGGSGGGGSGGGGSGGGGS

SEQ ID NO: 43 - protein, 5 aa, Artificial Sequence
  source: /note=“Description of Artificial Sequence Synthetic peptide”
GGGGS

SEQ ID NO: 44 - protein, 10 aa, Artificial Sequence
  source: /note=“Description of Artificial Sequence Synthetic peptide”
GGGGSGGGGS

SEQ ID NO: 45 - protein, 15 aa, Artificial Sequence
  source: /note=“Description of Artificial Sequence Synthetic peptide”
GGGGSGGGGSGGGGS

SEQ ID NO: 46 - protein, 20 aa, Artificial Sequence
  source: /note=“Description of Artificial Sequence Synthetic peptide”
GGGGSGGGGSGGGGSGGGGS

SEQ ID NO: 47 - protein, 6 aa, Artificial Sequence
  source: /note=“Description of Artificial Sequence Synthetic 6xHis tag”
HHHHHH